(19) United States
(12) Patent Application Publication
(10) Pub. No.: US 2025/0163482 A1
(43) Pub. Date: May 22, 2025

(54) NOVEL SYNTHETIC PROMOTER, AND METHOD FOR PRODUCING DAPTOMYCIN USING SAME

(71) Applicant: KONKUK UNIVERSITY INDUSTRIAL COOPERATION CORP, Seoul (KR)

(72) Inventors: Hahk Soo KANG, Seoul (KR); Chang Hun JI, Busan (KR)

(73) Assignee: KONKUK UNIVERSITY INDUSTRIAL COOPERATION CORP, Seoul (KR)

(21) Appl. No.: 18/839,223

(22) PCT Filed: Nov. 1, 2022
(86) PCT No.: PCT/KR2022/016938

§ 371 Date: Aug. 16, 2024

(30) Foreign Application Priority Data
Feb. 16, 2022 (KR) 10-2022-0020435

(51) Int. Cl. C12P 17/10; C12N 1/20; C12N 9/00; C12N 15/113; C12N 15/76; C12R 1/465

(52) U.S. Cl. CPC C12P 17/10; C12N 1/205; C12N 9/93; C12N 15/113; C12N 15/76; C12Y 602/01003; C12N 2510/02; C12R 2001/465

(57) ABSTRACT

The present invention relates to a synthetic promoter operably linked to a daptomycin biosynthetic gene cluster; a gene construct for producing daptomycin in which the synthetic promoter and a daptomycin biosynthetic gene cluster are operably linked; a recombinant expression vector for producing daptomycin containing the genetic construct; and a transformant for producing daptomycin that has been transformed with the recombinant expression vector, wherein the synthetic promoter can increase the transcriptional activity of the daptomycin biosynthetic gene cluster, and the transformant that has been transformed with the recombinant expression vector containing the synthetic promoter and the daptomycin biosynthetic gene cluster can be effectively used in the field of medicine and pharmacy by enabling mass production of daptomycin.

### CROSS-REFERENCE TO RELATED APPLICATIONS

This application is a 35 U.S.C. § 371 National Stage of International Patent Application No. PCT/KR2022/016938, filed Nov. 1, 2022, claiming benefit from Korean Patent Application No. 10-2022-0020435, filed Feb. 16, 2022, the disclosures of which are incorporated herein in their entirety by reference, and priority is claimed to each of the foregoing.

### STATEMENT REGARDING PRIOR DISCLOSURES BY THE INVENTOR OR A JOINT INVENTOR

Applicant designates the following article as a grace period publication in order to expedite examination of the application in accordance with 37 CFR 1.77(b)(6) and MPEP 608.01(a): Ji et al., “Top-down Synthetic Biology Approach for Titer Improvement of Clinically Important Antibiotic Daptomycin in Streptomyces roseosporus” published in Metabolic Engineering, Volume 69, pages 40-49, on Nov. 2, 2021. The disclosures of the article are incorporated herein by reference in their entirety for all purposes.

### INCORPORATION OF SEQUENCE LISTING

The instant application contains a Sequence Listing which has been submitted electronically in XML format and is hereby incorporated by reference in its entirety. Said XML copy, created on Oct. 31, 2022, is named “PCT5041255.xml” and is 167,378 bytes in size.

### TECHNICAL FIELD

The present invention relates to a novel synthetic promoter for producing daptomycin; an artificial biosynthetic gene cluster construct for producing daptomycin in which the synthetic promoter and a daptomycin biosynthetic gene cluster are operably linked; a recombinant expression vector containing the artificial biosynthetic gene cluster construct; a transformant transformed with the recombinant expression vector; and a method for producing daptomycin.
[National Research and Development Project that supported this invention]
[Assignment Unique Number] 1711126964
[Assignment Number] 2018R1C1B3001028
[Implementing Ministry] Korea Ministry of Science and ICT
[Research Assignment Specialized Organization] National Research Foundation of Korea
[Project Name] Individual Basic Research (Korea Ministry of Science and ICT) (R&D)
[Assignment Name] Construction of genome mining platform for the discovery of new biologically active secondary metabolites from Actinomycetes
[Name of Project Carrying Out Organization] Konkuk University
[Research Period] 2018.03.01-2022.02.28

### BACKGROUND

Daptomycin is a secondary metabolite produced from the fermentation culture of Streptomyces roseosporus and is a lipopeptide antibiotic approved by the U.S. Food and Drug Administration (FDA). Daptomycin is evaluated as a high value-added drug because it exhibits excellent antibacterial activity against super bacteria and has a low resistance rate, making it used as a therapeutic agent for multidrug-resistant bacterial infections.

Daptomycin production is accomplished by the expression of a number of biosynthetic genes (dptE/F/G/H/I/J), including non-ribosomal peptide synthetase (dptA/BC/D), which consists of approximately 70 kb nucleotide sequence in the genome of the parent strain (wild-type or native strain); transporter genes (dptM/N/P); and transcriptional regulatory genes (dptR1/R2). However, the production yield of daptomycin from the parent strain is approximately 20 to 30 mg/L in flask culture, and the low production yield poses a challenge for mass production of daptomycin.

To improve the low daptomycin production yield of this parent strain, the development of highly efficient daptomycin-producing mutant strains using UV irradiation, NTG mutagenesis, and genome shuffling has been reported, and a mutant strain with an improved production yield of up to 380% of the crude drug has been developed.

However, the complexity of the mutant strain selection process and the difficulty in understanding the principles of increasing production yield limit the development of industrial strains with excellent production capacity. In addition, genetic manipulation methods such as promoter engineering for overexpression of biosynthetic genes have been developed, but the daptomycin biosynthetic gene cluster is composed of (1) multiple repeat nucleotide sequences within the non-ribosomal peptide synthetase (dptBC) and (2) multiple biosynthetic operons, making it difficult to manipulate the DNA nucleotide sequence, and thus could not be applied to studies on increasing production yield through optimization of transcriptional activity.

Accordingly, the inventors of the present invention completed the present invention by producing a novel daptomycin biosynthetic gene cluster from which repeat sequences have been removed, and then producing an artificial biosynthetic gene cluster construct in which the existing promoter in the biosynthetic gene cluster is replaced with a synthetic promoter, and thereby confirming that the daptomycin production yield can be significantly increased through transcription optimization of the genes involved in daptomycin biosynthesis.

### SUMMARY OF THE INVENTION

The present invention is directed to providing a synthetic promoter containing a nucleotide sequence selected from the group consisting of SEQ ID NO: 1 to SEQ ID NO: 25.

The present invention is also directed to providing a gene construct for producing daptomycin, in which the synthetic promoter and the daptomycin biosynthetic gene cluster are operably linked.

The present invention is also directed to providing a recombinant expression vector for producing daptomycin containing the gene construct.

The present invention is also directed to providing a transformant for producing daptomycin, transformed with the recombinant expression vector.

The present invention is also directed to providing a method for producing daptomycin, including culturing the transformant for producing daptomycin.

To achieve the above objects, the present invention provides a synthetic promoter containing a nucleotide sequence selected from the group consisting of SEQ ID NO: 1 to SEQ ID NO: 25.

In addition, the present invention provides a gene construct for producing daptomycin, in which the synthetic promoter and the daptomycin biosynthetic gene cluster are operably linked.

In addition, the present invention provides a recombinant expression vector for producing daptomycin containing the gene construct.

In addition, the present invention provides a transformant for producing daptomycin transformed with the recombinant expression vector.

In addition, the present invention provides a method for producing daptomycin, including culturing the transformant for producing daptomycin.

The novel synthetic promoter according to the present invention is operably linked to the daptomycin biosynthetic gene cluster and can increase the transcriptional activity of the biosynthetic gene cluster, and a transformant transformed with a recombinant expression vector containing an artificial biosynthetic gene cluster construct for producing daptomycin, in which the synthetic promoter and the daptomycin biosynthetic gene cluster are operably linked, can produce daptomycin in large quantities, and therefore, the novel synthetic promoter according to the present invention can be usefully used in the pharmaceutical field.

### BRIEF DESCRIPTION OF THE DRAWINGS

FIG. 1 is a schematic diagram showing a process of promoter engineering of the daptomycin biosynthetic gene cluster and development of a strain for mass production of daptomycin using the same.

FIG. 2 is a schematic diagram showing a strategy for optimizing transcriptional activity by refactoring the daptomycin biosynthetic gene cluster using DBTL (Design-Build-Test-Learn), a synthetic biology technique.

FIGS. 3A-3E show the results of quantitative analysis of the transcriptional activity of daptomycin biosynthetic gene cluster through transcriptome analysis of Streptomyces roseosporus strains, compared to other biosynthetic gene clusters or reporter genes.

FIGS. 4A-4B show shows the results of qualitative analysis of the operon structure and promoter location of the daptomycin biosynthetic gene cluster using dRNA-seq.

FIGS. 5A-5D show a schematic diagram showing the cloning and heterologous expression of a novel daptomycin biosynthetic gene cluster. This shows the location of the repeat nucleotide sequence within the daptomycin biosynthetic gene cluster and the codon-reprogramming process to remove the repeat nucleotide sequence.

FIG. 6A shows the structure of the CRISPR/Cas9 vector used for multiplex promoter engineering of the daptomycin biosynthetic gene cluster and FIG. 6B shows the level of transcriptional activity of the synthetic promoter.

FIGS. 7A-7C show the results of analyzing the production yield of daptomycin after producing a biosynthetic gene cluster refactoring construct (RD00) in which two transcriptional regulatory factors, dptR1 and dptR2 genes not involved in daptomycin biosynthesis, were deleted from the BAC vector (BAC-dpt*) in which the daptomycin biosynthetic gene cluster was cloned.

FIGS. 8A-8D show the analysis results of transcriptome and metabolome of a Streptomyces roseosporus strain transformed with an artificial biosynthetic gene cluster construct created by refactoring and the transcription optimization process of an operon within the biosynthetic gene cluster according to promoter engineering of the daptomycin biosynthetic gene cluster.

FIGS. 9A-9D show the results of comparative analysis of daptomycin production yield according to the supply of decanoic acid precursor.

FIGS. 10A-10C show a comparative analysis of the transcriptional activity, cell growth rate, and daptomycin production yield of the biosynthesis operon of a mass-producing strain transformed with an artificial daptomycin biosynthetic gene cluster refactored using a synthetic promoter, and a wild-type strain.

### DETAILED DESCRIPTION

Unless otherwise defined, all technical and scientific terms used in this specification have the same meaning as commonly understood by a person skilled in the art to which the present invention pertains. In general, the nomenclature used in this specification is well known and commonly used in the art.

The present invention provides a synthetic promoter containing a nucleotide sequence selected from the group consisting of SEQ ID NO: 1 to SEQ ID NO: 25.

In one embodiment of the present invention, the synthetic promoter may be operably linked to a daptomycin biosynthetic gene cluster.

The term “daptomycin” of the present invention is a lipopeptide antibiotic, which exhibits excellent antibacterial activity against super bacteria and has a low resistance rate, and is a drug used as a therapeutic agent for multidrug-resistant bacterial infections.

In addition, the present invention provides a gene construct for producing daptomycin, in which the synthetic promoter and the daptomycin biosynthetic gene cluster are operably linked.

The term “daptomycin biosynthetic gene cluster” of the present invention refers to a set of genes essential for daptomycin biosynthesis including a number of biosynthetic genes (dptE, dptF, dptG, dptH, dptI and dptJ), including non-ribosomal peptide synthetase (dptA, dptBC, dptD), which consists of approximately 70 kb nucleotide sequence in the genome; transporter protein genes (dptN, dptM and dptP), and transcriptional regulatory factor genes (dptRI and dptR2).

In one embodiment of the present invention, the daptomycin biosynthetic gene cluster may include a nucleotide sequence of SEQ ID NO: 40. Preferably, the daptomycin biosynthetic gene cluster may include a dptR2 (regulatory gene 2) gene encoding a polypeptide containing the amino acid sequence of SEQ ID NO: 26; a dptRI (transcriptional regulator 1) gene encoding a polypeptide containing the amino acid sequence of SEQ ID NO: 27; a dptJ (Tryptophan 1,3-dioxygenase) gene encoding a polypeptide containing the amino acid sequence of SEQ ID NO: 28; a dptI (Glutamine 3-methyl transferase) gene encoding a polypeptide containing the amino acid sequence of SEQ ID NO: 29; a dptH (alpha/beta Hydrolase) gene encoding a polypeptide containing the amino acid sequence of SEQ ID NO: 30; a dptG (mbtH family protein) gene encoding a polypeptide containing the amino acid sequence of SEQ ID NO: 31; a dptD (daptomycin non-ribosomal peptide synthetase D) gene encoding a polypeptide containing the amino acid sequence of SEQ ID NO: 32; a dptBC (daptomycin non-ribosomal peptide synthetase BC) gene encoding a polypeptide containing the amino acid sequence of SEQ ID NO: 33; a dptA (daptomycin non-ribosomal peptide synthetase A) gene encoding a polypeptide containing the amino acid sequence of SEQ ID NO: 34; a dptF (Acyl carrier protein) gene encoding a polypeptide containing the amino acid sequence of SEQ ID NO: 35; a dptE (Fatty acid ligase) gene encoding a polypeptide containing the amino acid sequence of SEQ ID NO: 36; a dptN (ABC-type transporter gene) gene encoding a polypeptide containing the amino acid sequence of SEQ ID NO: 37; a dptM (ABC-type transporter gene) gene encoding a polypeptide containing the amino acid sequence of SEQ ID NO: 38; and a dptP (Transporter gene) gene encoding a polypeptide containing the amino acid sequence of SEQ ID NO: 39.

In one embodiment of the present invention, the synthetic promoter may replace one or more promoters selected from the group consisting of a first promoter located at nucleotide sequence positions 75,318 to 75,707^{th} of the daptomycin biosynthetic gene cluster; a second promoter located at nucleotide sequence positions 73,171 to 73,185^{th}; a third promoter located at nucleotide sequence positions 26,456 to 26,528^{th}; a fourth promoter located at nucleotide sequence positions 25,292 to 25,528^{th}; and a fifth promoter and a sixth promoter located at nucleotide sequence positions 77,531 to 78,288^{th}.

In one embodiment of the present invention, the synthetic promoter replacing the first promoter of the daptomycin biosynthetic gene cluster may be a synthetic promoter including any one nucleotide sequence selected from the group consisting of B25 (SEQ ID NO: 2), A48 (SEQ ID NO: 3), A01 (SEQ ID NO: 4), A46 (SEQ ID NO: 5), A15 (SEQ ID NO: 8), A09 (SEQ ID NO: 9), B40 (SEQ ID NO: 11), A49 (SEQ ID NO: 12), B31 (SEQ ID NO: 13), B28 (SEQ ID NO: 14), A47 (SEQ ID NO: 15), A20 (SEQ ID NO: 16), B52 (SEQ ID NO: 19), R06 (SEQ ID NO: 18), A32 (SEQ ID NO: 20), R05 (SEQ ID NO: 22), A07 (SEQ ID NO: 23), A29 (SEQ ID NO: 24), and B45 (SEQ ID NO: 25).

In one embodiment of the present invention, the synthetic promoter replacing the second promoter of the daptomycin biosynthetic gene cluster may be a synthetic promoter including any one nucleotide sequence selected from the group consisting of A12 (SEQ ID NO: 1), B25 (SEQ ID NO: 2), A48 (SEQ ID NO: 3), A01 (SEQ ID NO: 4), A46 (SEQ ID NO: 5), A31 (SEQ ID NO: 6), A26 (SEQ ID NO: 7), A15 (SEQ ID NO: 8), A10 (SEQ ID NO: 10), A49 (SEQ ID NO: 12), B31 (SEQ ID NO: 13), B28 (SEQ ID NO: 14), A47 (SEQ ID NO: 15), B24 (SEQ ID NO: 17), R06 (SEQ ID NO: 18), B52 (SEQ ID NO: 19), B42 (SEQ ID NO: 21), R05 (SEQ ID NO: 22), A07 (SEQ ID NO: 23), and B45 (SEQ ID NO: 25).

In one embodiment of the present invention, the synthetic promoter replacing the third promoter of the daptomycin biosynthetic gene cluster may be a synthetic promoter including any one nucleotide sequence selected from the group consisting of A12 (SEQ ID NO: 1), B25 (SEQ ID NO: 2), A48 (SEQ ID NO: 3), A01 (SEQ ID NO: 4), A46 (SEQ ID NO: 5), A31 (SEQ ID NO: 6), A26 (SEQ ID NO: 7), A15 (SEQ ID NO: 8), A09 (SEQ ID NO: 9), A10 (SEQ ID NO: 10), B40 (SEQ ID NO: 11), A49 (SEQ ID NO: 12), B31 (SEQ ID NO: 13), B28 (SEQ ID NO: 14), A47 (SEQ ID NO: 15), A20 (SEQ ID NO: 16), R06 (SEQ ID NO: 18), B52 (SEQ ID NO: 19), A32 (SEQ ID NO: 20), B42 (SEQ ID NO: 21), R05 (SEQ ID NO: 22), A07 (SEQ ID NO: 23), A29 (SEQ ID NO: 24), and B45 (SEQ ID NO: 25).

In one embodiment of the present invention, the synthetic promoter replacing the fourth promoter of the daptomycin biosynthetic gene cluster may be a synthetic promoter including any one nucleotide sequence selected from the group consisting of A12 (SEQ ID NO: 1), A48 (SEQ ID NO: 3), A46 (SEQ ID NO: 5), A31 (SEQ ID NO: 6), A26 (SEQ ID NO: 7), A15 (SEQ ID NO: 8), A09 (SEQ ID NO: 9), A10 (SEQ ID NO: 10), B40 (SEQ ID NO: 11), A49 (SEQ ID NO: 12), B31 (SEQ ID NO: 13), B28 (SEQ ID NO: 14), A47 (SEQ ID NO: 15), A20 (SEQ ID NO: 16), R06 (SEQ ID NO: 18), B52 (SEQ ID NO: 19), A32 (SEQ ID NO: 20), R05 (SEQ ID NO: 22), A07 (SEQ ID NO: 23), A29 (SEQ ID NO: 24), and B45 (SEQ ID NO: 25).

In one embodiment of the present invention, the synthetic promoter replacing the fifth promoter and the sixth promoter of the daptomycin biosynthetic gene cluster may be a synthetic promoter including any one nucleotide sequence selected from the group consisting of A12 (SEQ ID NO: 1), B25 (SEQ ID NO: 2), A01 (SEQ ID NO: 4), A31 (SEQ ID NO: 6), A26 (SEQ ID NO: 7), A15 (SEQ ID NO: 8), A09 (SEQ ID NO: 9), A10 (SEQ ID NO: 10), B40 (SEQ ID NO: 11), A49 (SEQ ID NO: 12), A47 (SEQ ID NO: 15), A20 (SEQ ID NO: 16), B24 (SEQ ID NO: 17), R06 (SEQ ID NO: 18), A32 (SEQ ID NO: 20), B42 (SEQ ID NO: 21), R05 (SEQ ID NO: 22), and B45 (SEQ ID NO: 25).

In addition, the present invention provides a recombinant expression vector for producing daptomycin containing the gene construct.

The term “recombinant expression vector” of the present invention is a vector capable of expressing a target protein or target RNA in a suitable host cell, and refers to a gene construct including essential regulatory elements operably linked to enable expression of a genetic insert.

The term “operably linked” of the present invention means that a transcriptional regulatory sequence of a gene and a nucleic acid sequence encoding a target protein or RNA are functionally linked to perform a general function. For example, a promoter and a nucleotide sequence encoding a protein or RNA can be operably linked to affect transcription and expression of the nucleotide sequence that is encoded. The operational linkage with the recombinant vector can be produced using synthetic biology and genetic recombination techniques well known in the art, and the site-specific DNA cleavage and linkage uses RNA and enzymes, and the like generally known in the art.

In one embodiment of the present invention, the recombinant expression vector may be selected from the group consisting of a plasmid vector, a cosmid vector, a bacterial artificial chromosome (BAC) vector, and a yeast artificial chromosome (YAC) vector, but is not limited thereto.

In addition, the present invention provides a transformant for producing daptomycin transformed with the recombinant expression vector.

In one embodiment of the present invention, the transformant may be a strain of the genus Streptomyces. In addition, the strain of the genus Streptomyces may be selected from the group consisting of Streptomyces lividans, Streptomyces coelicolor, Streptomyces albidoflavus, and Streptomyces roseosporus, but is not limited thereto.

In one embodiment of the present invention, the transformant may be further transformed with a recombinant expression vector containing a gene encoding fatty acid ligase and acyl carrier protein.

In addition, the present invention provides a method for producing daptomycin, including culturing the transformant.

In one embodiment of the present invention, the method may be culturing a transformant in a culture medium containing decanoic acid.

### MODE FOR CARRYING OUT THE INVENTION

The present invention will be described in more detail through examples. These examples are intended to explain the present invention specifically and the scope of the present invention is not limited to these examples.

### Example 1. Strategy for Development of a Daptomycin Mass-Producing Strain Through Refactoring of the Daptomycin Biosynthetic Gene Cluster

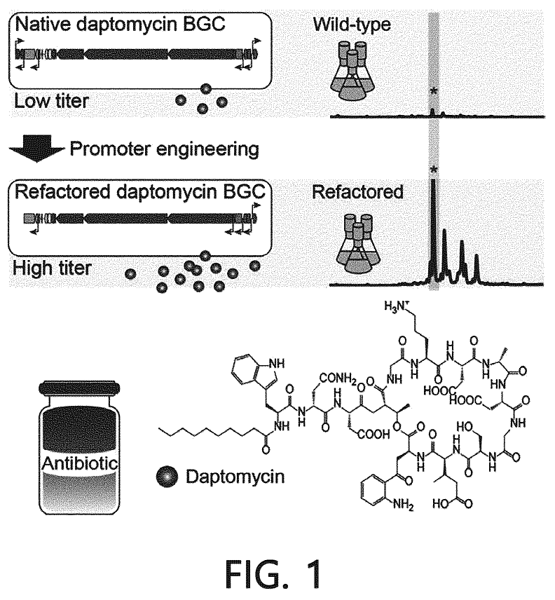

The present inventors produced a gene construct by replacing the existing promoter in the wild-type daptomycin biosynthetic gene cluster with a synthetic promoter through promoter engineering for the daptomycin biosynthetic gene cluster, and developed a daptomycin mass-producing strain transformed with an artificial daptomycin biosynthetic gene cluster containing the synthetic promoter (FIG. 1).

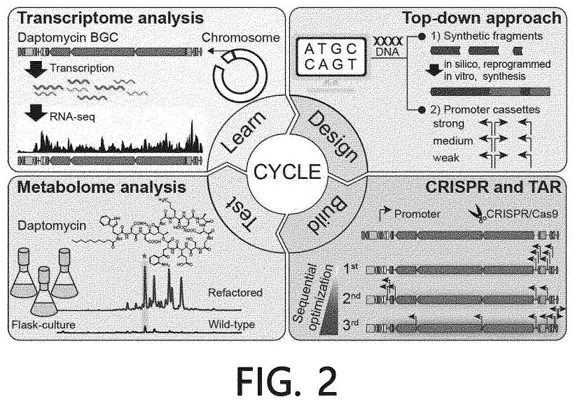

To produce a strain for mass production of daptomycin by refactoring the daptomycin biosynthetic gene cluster, the DBTL (Design-Build-Test-Learn) cycle, a synthetic biology technique, was utilized (FIG. 2). Specifically, in the design step, codon-reprogramming, operon re-structuring, and promoter sequence design were performed. In the Build step, the genome was replaced with the newly designed sequence using the CRISTAR (CRISPR/Cas9-mediated TAR) DNA refactoring tool. In the Test step, the production yield of daptomycin and associated lipopeptides produced from strains transformed with each of the prepared artificial biosynthetic gene cluster constructs was analyzed. In the Learn step, transcriptome analysis using RNA-seq was performed to design the refactoring locations, sequences, and methods to be performed in the next engineering step. This DBTL (Design-Build-Test-Learn) cycle was repeated until the optimal strain for mass production of daptomycin was obtained.

### Example 2. Transcriptome Analysis of Wild Strains of Streptomyces roseosporus

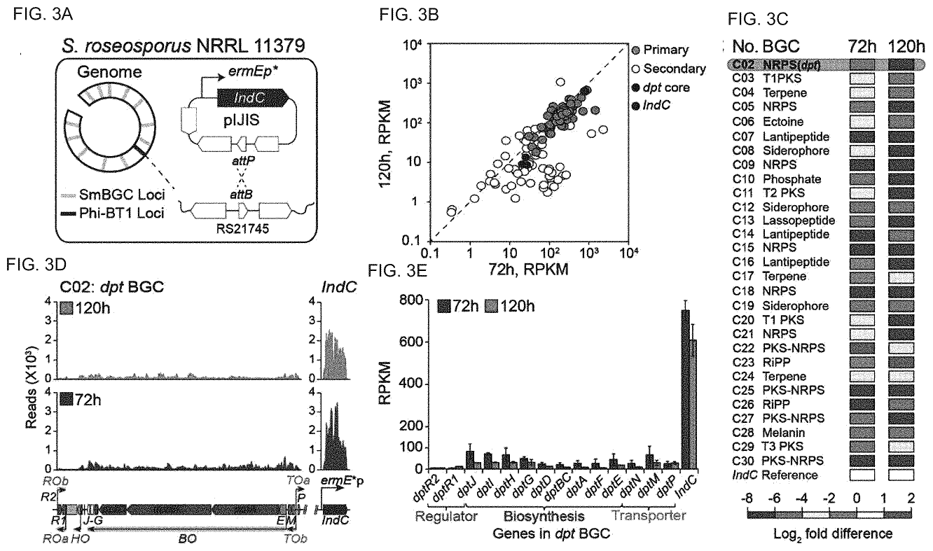

To perform transcriptome analysis of the daptomycin-producing strain, RNA-seq technology, a next-generation nucleotide sequence analysis method, was utilized. RNA-seq technology enables quantitative/qualitative analysis of the transcriptional activity of all genes in the genome. First, a strain in which an indigoidine reporter gene (IndC) transcribed by a reference promoter (ermE*p) was inserted into the genome of the wild parent strain Streptomyces roseosporus (NRRL 11379) was prepared (FIG. 3A). The strains were cultured in R5A liquid medium for 7 days, among which RNA was extracted at day 3 (72 h) and day 5 (120 h) after the start of culture and RNA-seq analysis was performed. The above RNA was subjected to RNA-seq analysis using the Illumina next-generation sequencing (NGS) analysis method through the cDNA library production process, and transcriptional activity was qualitatively and quantitatively analyzed using bioinformatics tools.

As a result, the transcriptional activity of genes related to the biosynthesis of secondary metabolites was generally lower than the transcriptional activity of genes related to the biosynthesis of primary metabolites (FIG. 3B). In particular, genes related to the biosynthesis of primary metabolites were confirmed to have transcriptional activity of approximately 10-100% compared to the IndC reporter gene (reference), and it was confirmed that most genes involved in secondary metabolite biosynthesis showed a low transcriptional activity of about 30% or less compared to the IndC reporter gene (FIGS. 3B and 3C). In addition, the non-ribosomal peptide synthetase (dptA/BC/D) genes, which are key biosynthetic genes within the daptomycin biosynthetic genes, were confirmed to have very low transcriptional activity, approximately 1-4% compared to the transcriptional activity of the IndC reporter gene (FIGS. 3D and 3E).

Accordingly, the present inventors confirmed that the low transcriptional activity of genes in the biosynthetic gene cluster of daptomycin, including non-ribosomal peptide synthetases (dptA/BC/D), is the main cause of the low production yield of daptomycin.

### Example 3. Analysis of Operon Structure and Promoter Using dRNA-Seq (Differential RNA-Seq)

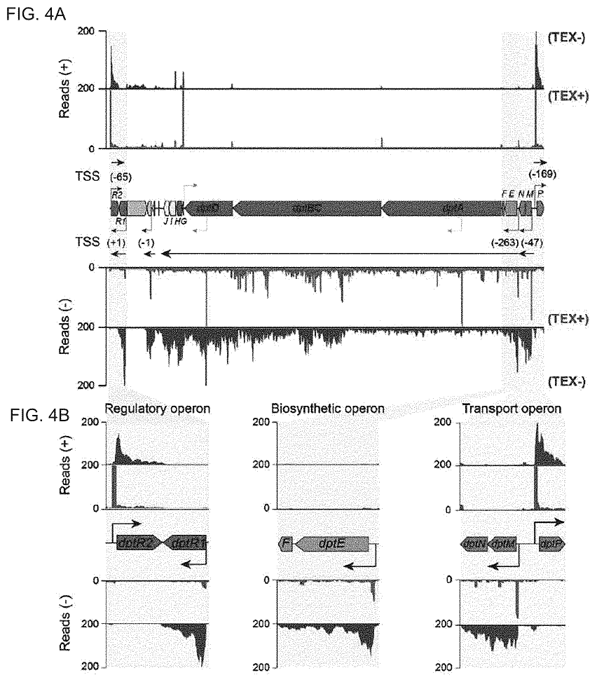

The daptomycin biosynthetic gene cluster consists of multiple operons containing transcriptional regulatory factors, transporter proteins, and biosynthetic enzyme genes. To achieve transcriptional activity optimization by promoter engineering, first, the precise operon structure and promoter location of the daptomycin biosynthetic gene cluster were identified using dRNA-seq (differential RNA-sequencing) technology. As a result, it was confirmed that the daptomycin biosynthetic gene cluster was composed of a total of six operons, including a biosynthesis operon (BO), a transporter operon (TOa, TOb), a regulatory operon (ROa, ROb), and a hypothetical operon (HO), and there was a promoter in the upstream nucleotide sequence of the genes in each operon (FIG. 4A).

### Example 4. BAC Vector Cloning of Daptomycin Biosynthetic Gene Cluster

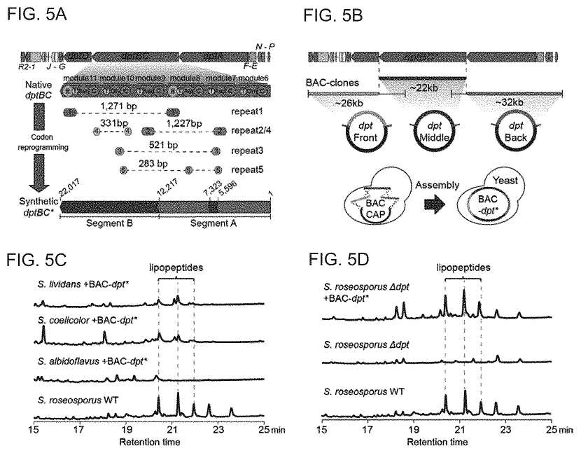

The present inventors confirmed that among the non-ribosomal peptide synthetase (NRPS) genes in the daptomycin biosynthetic gene cluster, the dptBC gene contains 28 repeat sequences consisting of DNA nucleotide sequences larger than 100 bp (FIG. 5A). The largest repeat sequence was identified as 1,271 bp (repeat 1) between module 8 and module 11, and most repeat sequences were identified in the first half (Segment A) of dptBC between module 6 and module 8 and the second half (Segment B) between module 9 and module 11 (FIG. 5A). These repeat sequences were removed by codon-reprogramming of the second half (Segment B) region, resulting in a novel dptBC* (˜22 kb) gene sequence that includes the codon-reprogrammed Segment B (˜10 kb) and part of Segment A (˜2 kb).

This newly codon-reprogrammed artificial dptBC* gene replaced the existing dptBC gene in the daptomycin biosynthetic gene cluster using the CRISTAR (CRISPR/Cas9-mediated TAR) refactoring tool (FIG. 5B). Specifically, the biosynthetic gene dptBC* (22 kb) with the repeat sequences removed was subcloned into a BAC vector to form BAC-dpt/Middle. The lower (26 kb) and upper (32 kb) portions of dptBC were also cloned into the BAC vector to form BAC-dpt/Front and BAC-dpt/Back, respectively. Three separate daptomycin biosynthetic gene cluster DNA fragments: dpt/Front, dpt/Middle, and dpt/Back, each cloned into a BAC vector, were assembled into a complete biosynthetic gene cluster DNA, dpt*, using homologous recombination in yeast, and finally cloned into a BAC vector to form an artificial daptomycin biosynthetic gene cluster construct, BAC-dpt* (FIG. 5B). BAC-dpt*, in which the artificial daptomycin biosynthetic gene cluster was cloned, was confirmed to be capable of stable replication and plasmid amplification in yeast and E. coli for a long period of time.

### Example 5. Daptomycin Production Strain Transformed with a BAC Vector Containing a Refactored Daptomycin Biosynthetic Gene Cluster

To determine a production strain suitable for lipopeptide production, various genus Streptomyces strains were transformed with a BAC vector (BAC-dpt*) in which the dpt* daptomycin biosynthetic gene cluster with the repeat sequences removed was cloned, and then, experiments were performed to compare and analyze the production yield of lipopeptide in each strain.

As a result, it was confirmed that lipopeptides were produced at a low production yield (10-12 mg/L) in Streptomyces lividans and Streptomyces coelicolor strains transformed with a BAC vector (BAC-dpt*) in which the dpt* daptomycin biosynthetic gene cluster with the repeat sequences removed was cloned (FIG. 5C). Therefore, it was confirmed that daptomycin biosynthetic gene clusters were expressed and daptomycin could be produced in heterologous strains.

In addition, it was confirmed that no lipopeptide was produced in the Streptomyces roseosporus mutant, where the existing native daptomycin biosynthetic gene cluster (dpt) present in the genome was removed. On the other hand, it was confirmed that the production of lipopeptides was restored at a production yield (20 to 30 mg/L) similar to that of the wild-type strain (WT) in the Streptomyces roseosporus transformant transformed with the BAC vector (BAC-dpt*) cloned with the daptomycin biosynthetic gene cluster (dpt*) from which the repeat sequences were removed (FIG. 5D).

### Example 6. Promoter Engineering of the Daptomycin Biosynthetic Gene Cluster

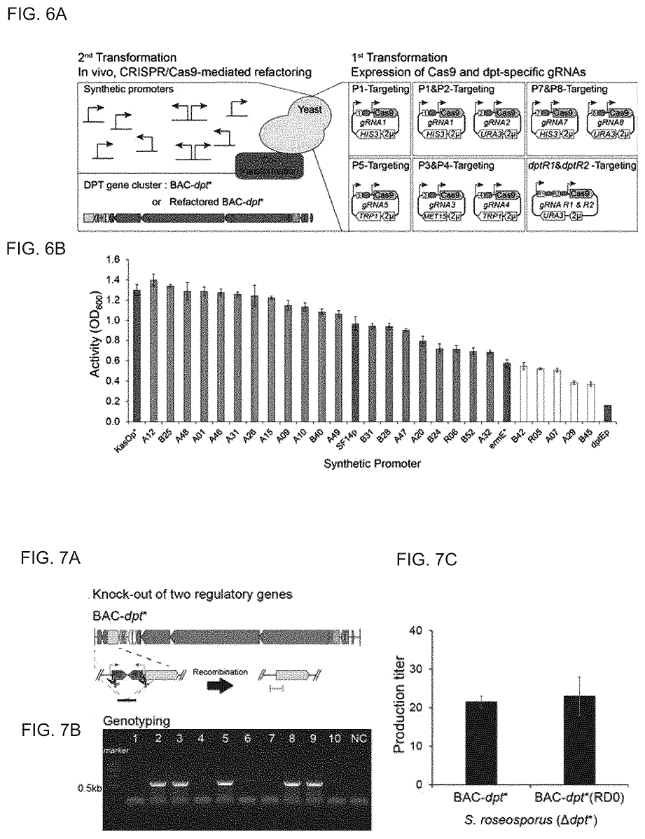

To optimize the transcriptional activity of the operon in the daptomycin biosynthetic gene cluster, promoter engineering was performed to replace the existing native promoter in the daptomycin biosynthetic gene cluster with a synthetic promoter nucleotide sequence. For high-efficiency multiplexing refactoring, Cas9 protein and gRNA expression vectors acting on yeast were prepared, and the structure of the CRISPR/Cas9 vector used in promoter engineering is shown in FIG. 6A.

In addition, the synthetic promoter was prepared by DNA synthesis and PCR amplification, and the transcriptional activity of the synthetic promoter was quantitatively analyzed by measuring absorbance at 600 nm to the degree of indigoidine biosynthesis according to the expression of the reporter gene (IndC). The synthetic promoters of A12 (SEQ ID NO: 1), B25 (SEQ ID NO: 2), A48 (SEQ ID NO: 3), A01 (SEQ ID NO: 4), A46 (SEQ ID NO: 5), A31 (SEQ ID NO: 6), A26 (SEQ ID NO: 7), A15 (SEQ ID NO: 8), A09 (SEQ ID NO: 9), A10 (SEQ ID NO: 10), B40 (SEQ ID NO: 11), and A49 (SEQ ID NO: 12) were found to be strong promoters (blue) with strong transcriptional activity; B31 (SEQ ID NO: 13), B28 (SEQ ID NO: 14), A47 (SEQ ID NO: 15), A20 (SEQ ID NO: 16), B24 (SEQ ID NO: 17), R06 (SEQ ID NO: 18), B52 (SEQ ID NO: 19), and A32 (SEQ ID NO: 20) were found to be middle promoters (green) with intermediate transcriptional activity; and B42 (SEQ ID NO: 21), R05 (SEQ ID NO: 22), A07 (SEQ ID NO: 23), A29 (SEQ ID NO: 24), and B45 (SEQ ID NO: 25) were identified as weak promoters (yellow) with week transcriptional activity (FIG. 6B). The sequence information of the above synthetic promoters are as shown in Table 1 below.

TABLE 1

| | Nucleotide sequence | |
|---|---|---|
| | (5′->3′) | SEQ ID NO |
| A12 | ATCGCTTGCAGGCGAGGTCCAGCGA | SEQ ID NO: 1 |
| promoter | CGAGCGTCGCACGCTGCCACAGCAG | |
| (269 | GACAGGTCGCAGGTGAGCGTGCACT | |
| bp | GACGGCACCTCGTCGGGACTGCTGA | |
| | GCACGGTCACGGCAGCCGAGTGGAG | |
| | CACGCGTGAGGGAGCGACGCAGGTG | |
| | ACGGTCTCAGCGCCATCCTGTGCGA | |
| | GTGACCAGCACACTACGGAGCGGAC | |
| | GAGTACGGCTTGTCAGTACTGACCC | |
| | TCTAGTGCTACGGTCAATAAGGCGA | |
| | AACACTCAGGAGGAAGACT | |
| B25 | CGCTATGCCACGCAGGTTAGCAGCC | SEQ ID NO: 2 |
| promoter | TGGCACGTGAGCTGCCGATGAGTCT | |
| (269 | AGGCAGCAGAAGAGCCGGATAGGCA | |
| bp) | CGAGCTCAGTGACGAGGCCGGTGAC | |
| | CGTCACGGACGGTACTAGCCGAGGA | |
| | GGGCTGAGCGAGGGTGCGTGAGACC | |
| | GCGAGCGGACGGTCCACGAGTGCGG | |
| | ACGCAGTGCAGCTGCGTGGAGCGAC | |
| | CCCCAATGCTTGACAAGAGCGTTCA | |
| | AACGCATCTAGCATTCGCTACAAAG | |
| | CCCCGCCAGGAGGTACAGC | |
| A48 | TCAGCAAGGCTCACCTACGGCCAGC | SEQ ID NO: 3 |
| promoter | TCGACACCTGGCTCACCCAGACCTG | |
| (269 | CCAACGCCTCCACGCCTGCTACCT | |
| bp) | GTCCCCATCCGACGAGGCCACCTCG | |
| | CCACCGCCACGGTCCCACCTACTCA | |
| | TCACCGTCTCCGTCACTCAGGGACT | |
| | GAAGCACCTCACGCGCACTCACCGC | |
| | ACCTTGCACCACAGGCACGCAGTCT | |
| | TCGAGACCCCTTGACGCCCCACTCG | |
| | CCTTACACGTACACTCTAAAGCATC | |
| | CCGTATCGAGGAGGTCAAGC | |
| A01 | CGCTATGCCACGCAGGTTCGACGTG | |
| promoter | AGCGTCACAGCGGTCGCGGTGGATG | |
| (269 | GTGAGGCGGATACTGAAGGGTGGTG | |
| bp) | CGCGGTCCATGGTGGATGGCAGGGC | |
| | GAACGGACACGCAACGGAGGCAGGG | |
| | TGGTAGTGCGGAGAGGCTCCAGGTG | |
| | ATGGTCCGTGTGTAGAGGGCCAGAG | |
| | GAGGTCGGCGGTAGTGTCCCGTAGG | |
| | TAACTTCATTTGGCGTGCATGAACT | |
| | CAACGTGCTATCCTTCCTAAAAAGG | EQ ID NO: 4 |
| | CCCCGCCAGGAGGTACAGC | |
| A46 | GTAGGTGAGCCTTGCTGACGGTCGA | EQ ID NO: 5 |
| promoter | AGAGGCGTGCGGGTCGTACGCAAGT | |
| (269 | CGGACACGGACGTAGTCGCGAGGTG | |
| bp) | AGTGGTGATGCAAGTGGTACCAGCC | |
| | GACAGAGTCGTCCGCAGTAGCAGAC | |
| | GGCTGTGGATCGATAGTGCGGGTAG | |
| | AGCGTGTGGAGAGCGTGACCCGTGG | |
| | ATCATGGCATGCGTCGAGGTACCGT | |
| | CACATTGGGTTGCCAGCCCGTTGCT | |
| | GGACGTACTACGGTTGAAGCCTCCC | |
| | CGGAATCAGGAGGACTTCC | |
| A31 | AAGTTCTCCTCGACCGTCGAGACGG | EQ ID NO: 6 |
| promoter | CTCGGCCAACGTCCGCCGTCTCCTC | |
| (270 | CGTCACGTCGCTGCTCACGCGGTAC | |
| bp) | TGGCTCAGCTGCGCCAGCTGGACTC | |
| | GGCTCAGTCGGTCCCACCCTTCGTG | |
| | CATCAGGGTCCAGCGACCGGTGACG | |
| | CTGCACGCCGTGGCTGCTAGTCGGC | |
| | CGACTCCCCGTTGCCACCTGGACCG | |
| | ACGTACTGAGCTTGCCTGAAGAACC | |
| | CACATCCAGCTAAAGTAAGCCAACC | |
| | GGACCTTTGAGGAGGGCTCA | |
| A26 | AAGTTCTCCTCGACCGTCGAGTGCG | EQ ID NO: 7 |
| promoter | GAGGAACAGAGTGGCCAGCCGTTGC | |
| (270 | CACGACGTCCGGACGGCACGACACC | |
| bp) | CCCGCAGGTACGATCAGTGGCACGC | |
| | GAGGCCTGCGACAGCGCCAAGACCG | |
| | GATACGACAGGAACCGTGGCAACAG | |
| | TCCGTAGACACCGCAGCCAGCGCAG | |
| | ATCGCGGGAGGAGATCCAGATCGAC | |
| | GCCCTGATGACTTGACGCTCGAGAA | |
| | GGTTCACGATTAGCATGCCGGTTAC | |
| | GGACCTTTGAGGAGGGCTCA | |
| A15 | ACCTCGCCTGCAAGCGATCCCCATA | EQ ID NO: 8 |
| promoter | TCTCCCGCTGGTCACGTCTGCCGAC | |
| (269 | TAGACGGTGGCGCACACCCTGGTTG | |
| bp) | CTCAAGCGTCTGCGGCCCATCCTCT | |
| | CAAGGTGTGCTCCGGACTGTGCCAG | |
| | GTCTGGCGCCTTCCACTCCGCGCCG | |
| | TGATACCGTTGGGTAGTGGTCGCTC | |
| | TGCGTACTGGCCTCGTCCTACAGCC | |
| | GCGAAGTACTTGACACATACCTGCT | |
| | AAGCCTTCTAATCTGCTTGCGTACT | |
| | CTACCACAGGAGGTAACTA | |
| A09 | ACTGGTGAATGATGGGCACTGTCGC | EQ ID NO: 9 |
| promoter | ACGAGAGCGTCAGACGGACCTAGAG | |
| (269 | CCTAGACCCTCGCTCACCTCGCACA | |
| bp) | ATCAGACGGCTCTGGGACGCTGGCT | |
| | GCCACGAGCGAGACGTCACGTCTGG | |
| | AGCAGCACTGGCGCAGCCTACCGCC | |
| | ACGCATCCATCCCGATCCGACGAGC | |
| | GAGCGGGTGACCGCTCTGGCTCAGA | |
| | CGCAGCCGCTTGACCACATGGTCCC | |
| | GGGCAGTATACGGTGGCTGCGACCC | |
| | CAACCAAAGGAGGCAGTGT | |
| A10 | ATCGCTTGCAGGCGAGGTGCGATCC | EQ ID NO: 10 |
| promoter | ACTCAGAGCTGCTGACAGTCCACCG | |
| (269 | TCGTCTGACCGTTCGCGACCACTCC | |
| bp) | GGACTGGCCAGCGTGTCCGAGGCCT | |
| | CAGTCGCTGTCGTGAGATAGGCTTG | |
| | CAGGGACGCCACTAGCGACGGTCGT | |
| | GGACAGGTGACACGCGCATCAAGGA | |
| | CGGCGATGTCTCCGTAGCTAGCGGA | |
| | GAACACCTCTTGACTCGCCTACCCG | |
| | CCGCTCGCTAAATTTGCTGTAGCCA | |
| | AACACTCAGGAGGAAGACT | |
| B40 | ACCTCGCCTGCAAGCGATAGCTCAC | EQ ID NO: 11 |
| promoter | ACGGCCATCCGCATCCACACAACGG | |
| (269 | CGCTGACCACGCTCTGGCACGACGC | |
| bp) | CTACGACGGTCCAGTTGGCAGCAGC | |
| | GACTGCGACCTCCCAGACCCCGCTT | |
| | GCGAGGCTCACACCCAGACCGCAGT | |
| | CACGACTTGCGACCGCAGCCTGCGG | |
| | ACCTAGGACCTGACTGCACGCTCGT | |
| | CACCACCATTTGTCCACCCTTCTCC | |
| | TTAGCTGGTACCATCCAGGTTCGCT | |
| | CTACCACAGGAGGTAACTA | |
| A49 | TGCCCATCATTCACCAGTAACCGAG | EQ ID NO: 12 |
| promoter | ACGGCATGTGGGACCCTGGCCGAGA | |
| (269 | GGAGCCACCGTAGGGAGAAGTAAGG | |
| bp) | CGGAGCGAGCAGGGATGGCACGGAG | |
| | AGGAGGTGAGCGGTGCGAGCAGTCG | |
| | TACGGACACGGAGGACACGCGGTGG | |
| | TGACGCCGGGCATCGCCGCACCGAA | |
| | GGAGCCGTGACAGGCTCTCGGCACT | |
| | ATAGTCCCCTTGACGAGCCCGAGAG | |
| | AGTGCAGCTAAGGTCCCACGTTCTT | |
| | ACGAACGAGGAGGCCCCAT | |
| B31 | TCAGCAAGGCTCACCTACTAGACGC | EQ ID NO: 13 |
| promoter | GTGCGGCGGCCTTGCCGTGGTGGTA | |
| (269 | CGTCGGAGTGCGTGAGGGTGTGGTT | |
| bp) | TGCGATAGGGCGAACTGGCGACAGT | |
| | GCTCGGCGACGGCTGGGATGTTGGG | |
| | TTGTGGTTCAGGGCTGCCAGTGCGT | |
| | TGGTCCTACGGGCTTAGGTGGTTGG | |
| | CGCGTGTGACTGGACGACTCGGACA | |
| | TTAACCACATTGACAGCCTGATGCC | |
| | GCGGCGATTACGATCGCTAATCACC | |
| | CGTATCGAGGAGGTCAAGC | |
| 28 | GTAGGTGAGCCTTGCTGATGACCCA | SEQ ID NO: 14 |
| promoter | CCAGCCACACCTCGCCTACGCCCGT | |
| 269 bp) | GCCACACCGACGCGCTCAACACCCA | |
| | CCAGTCATCACAACGCCACGCCGTA | |
| | CACCACAGCCACCACTTCACGAGCC | |
| | GAGTGCACGGCACACACGTCGCCAC | |
| | GAACGCACGACAACGACCACGTCCA | |
| | CACGCACCGACGGCAGTGCCGAGAC | |
| | ACTGATGCATTGACAAGAATCACGG | |
| | AAACACCTTACACTCGATGAGTGGC | |
| | CGGAATCAGGAGGACTTCC | |
| 47 | ACTGGTGAATGATGGGCAGTCCGTC | SEQ ID NO: 15 |
| promoter | CTGCTAGCGAGCCGTCTCGTCCCCA | |
| 269 bp) | CCGCACGTGAGGCACACCTACCTCC | |
| | TCCGTACGCCGAACTCTCCGCTCGC | |
| | GCGAGTCTGCTACGTCGGCTAGTCG | |
| | CTGGCTCATCGCAGCTGTATCTTGC | |
| | TTCGTTCGTACCTCGGACGCCTGTC | |
| | GCTGGCGCTGACCTCGGCACTGCTG | |
| | CGAATGCATTTGACTTCGATCTAGG | |
| | GAACCAGGTACGATAGAAAATAATC | |
| | CAACCAAAGGAGGCAGTGT | |
| 20 | ACCTCGCCTGCAAGCGATGGAGCTG | SEQ ID NO: 16 |
| promoter | GGTGCGACGGGACATTGCATGCCGG | |
| 269 bp) | TACGCTGGATCGCTGGGATCGCGAC | |
| | GCAGTGCTGCCGTGGAGCGGTGCCG | |
| | TGCAGTGGTCGCACCTCTCCAGCGT | |
| | CGCGAGCAGAGCGTGCCGCAGCCTT | |
| | GCCCAGTACTCTACGCGCCCACTGG | |
| | GCTGTCGCCACCTGACGGCACTTGA | |
| | CGGGATGTGTTGCCTCCGACTGTTC | |
| | ACCTCACCTATTATGCCTCCACACT | |
| | CTACCACAGGAGGTAACTA | |
| 24 | ATCGCTTGCAGGCGAGGTCTGGTCG | SEQ ID NO: 17 |
| promoter | GGTTCGTGTCGCGGAGAGCCGTGGT | |
| 269 bp) | GGTAAGGGTCGGGGTGCCGGCTGTC | |
| | TGGTGTGGGGGTGGCGCGTTCGCAC | |
| | CGTAGTCGTTGCGACCGTCGAAGCG | |
| | TGCCTGTGCCTCCCTGTTCTCGTGC | |
| | ACAGCCCGGTGTCGGGGTCGCAGTC | |
| | GGACTCGGTCCGAGCTCCATCCACA | |
| | AGCGAATACTTGCCGCCCCTTCCGT | |
| | CTGGATGTTAAGTTAATCTCAGACA | |
| | AACACTCAGGAGGAAGACT | |
| 06 | CGACGCTTGTCGCTACCAGAATCAT | SEQ ID NO: 18 |
| promoter | TCCCGAACGGTGCTGCCATAAGGCT | |
| 200 bp) | CCGCAGAGCAGCTAAGCGATACCAC | |
| | ACCAACGAGTGTAACGTGCCGAATC | |
| | GTTGTCGACATCACCGACACCTGCT | |
| | TGACTCCGTCCTTGGTTGGCCCCGT | |
| | GCAAAGCGTCATGCTAGACTACATG | |
| | ACTGGCCTATCATAGGAGGCAGTTT | |
| 52 | TGCCCATCATTCACCAGTGGACCAC | SEQ ID NO: 19 |
| promoter | AGCGGCATCGGACAGCGTGCAACCC | |
| 269 bp) | TCGCAACCAGGTCCCCCGACGA\|CA | |
| | CCTACGTGCAGCCACAACACAGCCA | |
| | CAGCCGCCAGCACGGTGACCCCACC | |
| | TCTGCCGACCCACCCAGATCCGAGC | |
| | TTGGACACCCACTGCGAGCCACCTG | |
| | ACCGACAACGGTCGCCAACGCTACA | |
| | GACTTCCTGTTTGACAAGGCCAATG | |
| | CAGCCACACTACCCTTAACGCGACT | |
| | TACGAACGAGGAGGCCCCAT | |
| 32 | GTAGCTGTACGCGTGGTACCAGGAG | SEQ ID NO: 20 |
| promoter | GCGCTGCACGCAAGAGCCGACCTCG | |
| 269 bp) | GACGGACCCACTCAGAGCATCGAGG | |
| | TTCGTAGGAGGCAAGGCGGTCGAGA | |
| | CACGACAGACCGCTAGAGGCAGCAG | |
| | GGTGGCCAACAGGCCGCATAGCAGC | |
| | GTCCACAGGGAACACGGTTGGACCA | |
| | GCGCAGGCACTCCAACGACACGCGG | |
| | TCCCACGCCTTGGCCCGAGGGCCGA | |
| | CCGGGTGCTATTTTCCTCAGAACAA | |
| | GAATACGAGGAGGCCCCAT | |
| 42 | CGCTATGCCACGCAGGTTGTCAGGC | SEQ ID NO: 21 |
| promoter | ACGTAGGTGCCAGACCGATCGCCCA | |
| 269 bp) | ACCCGTCACACTCCACTCGACTCTA | |
| | CTATGACTCATCCCTACACTCGCTA | |
| | CCTAGCTCACGAGGCCTAGCGCTCA | |
| | AGTGGCTAGCACCTGCGCCACTCGG | |
| | CTCTCACGTGCAAGCCCGTGCACCT | |
| | TCACGTGCGACTCACGCAGTGGTCA | |
| | CCCCAGGACTTGTCCGGAGCGGCCG | |
| | AAGGGTACTATGATAGAGGCCAGTG | |
| | CCCCGCCAGGAGGTACAGC | |
| 05 | TTCACTCGCGTCCACAGACTGGAGC | SEQ ID NO: 22 |
| promoter | GTGCACCGACTCGCACAGCGTCGGT | |
| 200 bp) | ACGGTGCGTGTGGAACGCATTAGTG | |
| | AACTGGTCCGACAGACTCTCGGTCA | |
| | CACGACTGATCAGCAGGTACCGTCT | |
| | TGCAACACAGTACCCTTGACGCCTC | |
| | ACTTGTACCAGCCCTAACATGCCTC | |
| | CTCCGTTGGCTTAAGGAGGCCCCAT | |
| 07 | GTAGGTGAGCCTTGCTGAGCGAGTG | SEQ ID NO: 23 |
| promoter | CCATCGGACGGACTAGCCAGAGGTA | |
| 269 bp) | GAGCGACGATTGCGACCGTGGTCGA | |
| | TGAGCTGGTTCGGAGCCGACGAGCC | |
| | GTCGCGTGACTACGCTGACGGCGTG | |
| | ACCACGAGCGAGCGTGATGGCGACT | |
| | GTGCACCGCAGAGTGCCGCACCGGG | |
| | CTACATACGACGACACGCGTCTGGA | |
| | GAATCTAAGTTGACGGGACGGTACG | |
| | GTGTGCGGTAACGTCTCCCCGGTGC | |
| | CGGAATCAGGAGGACTTCC | |
| 29 | TCAGCAAGGCTCACCTACCTCCTGC | SEQ ID NO: 24 |
| promoter | CTACGCACGGCCACCTTCGAGCGCC | |
| 269 bp) | TTGCACTACGCCACTGCGCATCCAC | |
| | TATCCCTGCCTGCACGGACACTCTG | |
| | ACTGGCACGGTCGAGCTCTGGTCGC | |
| | ACATCCCGCTCTCGTCCTCCACGCC | |
| | CCCTTCGAGATCCTAGCCCTGCACC | |
| | TGTACCGCGCAGCTCCGTCGTTGCA | |
| | CCCTAACACTTGACGCAACGCCTTC | |
| | CGTCCGCATAATCTGTTCGGCTGTC | |
| | CGTATCGAGGAGGTCAAGC | |
| 45 | ACTGGTGAATGATGGGCACTGGTCC | SEQ ID NO: 25 |
| promoter | GGATCGGTGCAGGCGGTGCACTCGT | |
| 269 bp) | GGAGTGGGTGGGGGTGACGGCTGAC | |
| | TGGTCTGCGGGTGGTGAGCTCGCCA | |
| | CGTAGTCGGTGTCGAGTCTGCCCAG | |
| | ACGCTGTGCCTCACTGATCCCGTGC | |
| | GACGCACGGTGTCGCGGTCGGACGC | |
| | TCCGGACGTAGCTCCGTCTCGTTCA | |
| | AACAGCCCATTGACACGGTTCACTG | |
| | CGCGCTTTTATGATGTACAGCCCTC | |
| | CAACCAAAGGAGGCAGTGT | |

First, before performing promoter engineering studies, a biosynthetic gene cluster refactoring construct (RD00) was prepared by deleting two transcriptional regulatory factors: dptR1 and dptR2 genes, which are not involved in daptomycin biosynthesis, from the BAC vector (BAC-dpt*: 91,447 bp (5′->3′), SEQ ID NO: 40) cloned with the daptomycin biosynthetic gene cluster (FIGS. 7A and 7B). It was confirmed that the Streptomyces roseosporus transformant transformed with a biosynthetic gene cluster in which the transcriptional regulatory factor gene was deleted produced lipopeptides in the same manner as the wild-type strain, and that there was no significant difference in production yield (FIG. 7C).

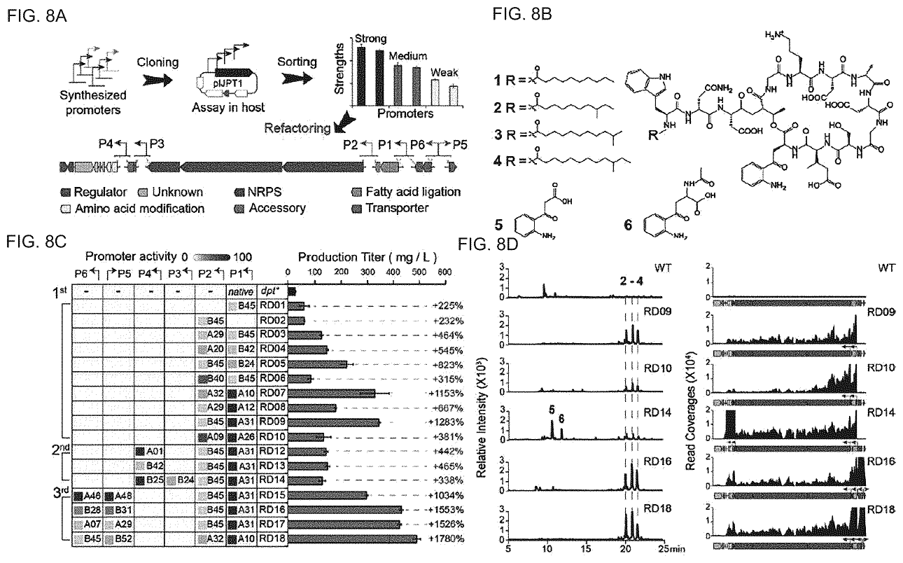

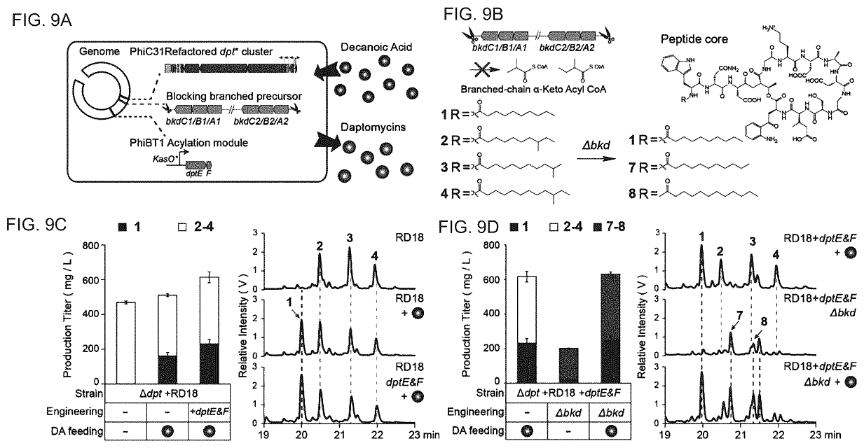

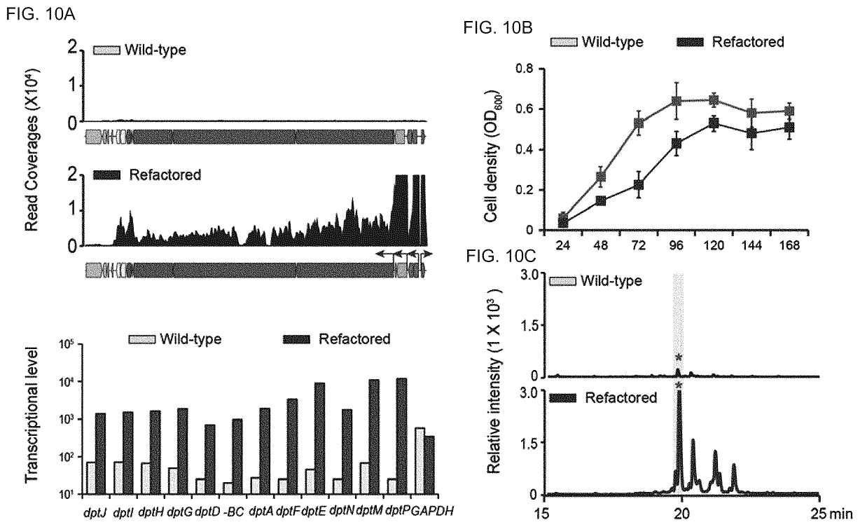

Subsequently, the goal of the first stage promoter engineering was to optimize the transcription efficiency of the biosynthesis operon by replacing the first promoter P1 or the second promoter P2 of the biosynthesis operon BO with a synthetic promoter (FIG. 8A), and to this end, biosynthetic gene cluster refactoring constructs RDO1 to RD10 were prepared by replacing with one or more synthetic promoters. Specifically, RD01 was obtained by replacing the first promoter (between the upper end of the dptE gene and the lower end of the dptN gene) located in the 75,318 to 75,707^{th} nucleotide sequence of the daptomycin biosynthetic gene cluster (BAC-dpt*: SEQ ID NO: 40) with the B45 synthetic promoter; RD02 was obtained by replacing the second promoter (between the upper end of the dptA gene and the lower end of the dptF gene) located in the 73,171 to 73,185th nucleotide sequence with the B45 synthetic promoter; RD03 was obtained by replacing the first promoter with the B45 synthetic promoter and the second promoter with the A29 synthetic promoter; RD04 was obtained by replacing the first promoter with the B42 synthetic promoter, and the second promoter with the A20 synthetic promoter; RD05 was obtained by replacing the first promoter with the B24 synthetic promoter, and the second promoter with the B45 synthetic promoter; RD06 was obtained by replacing the first promoter with the B45 synthetic promoter, and the second promoter with the B40 synthetic promoter; RD07 was obtained by replacing the first promoter with the A10 synthetic promoter, and the second promoter with the A32 synthetic promoter; RD08 was obtained by replacing the first promoter with the A12 synthetic promoter, and the second promoter with the A29 synthetic promoter; RD09 was obtained by replacing the first promoter with the A31 synthetic promoter, and the second promoter with the B45 synthetic promoter; and RD10 was obtained by replacing the first promoter with the A26 synthetic promoter, and the second promoter with the A09 synthetic promoter.

As a result, the daptomycin production yield increased significantly in all RDO1 to RD10 refactoring constructs, and in particular, the RD07 and RD09 refactoring constructs showed a production yield increase of 1,000% or more (FIG. 8C).

The goal of the second stage promoter engineering was to optimize the transcriptional efficiency of an operon containing a precursor synthesis gene required for daptomycin biosynthesis, and biosynthetic gene cluster refactoring constructs RD12 to RD14 were prepared by performing promoter engineering by targeting the third promoter P3 or fourth promoter P4 position of the biosynthesis operon BO. Specifically, RD12 was obtained by replacing the first promoter with the A31 synthetic promoter, replacing the second promoter with the B45 synthetic promoter, and replacing the fourth promoter (between the upper end of the dptI gene and the lower end of the dptH gene) located in the 25,292 to 25,374^{th} nucleotide sequence with the A01 synthetic promoter; RD13 was obtained by replacing the first promoter with the A31 synthetic promoter, replacing the second promoter with the B45 synthetic promoter, and replacing the fourth promoter with the B42 synthetic promoter; and RD14 was obtained by replacing the first promoter with the A31 synthetic promoter, replacing the second promoter with the B45 synthetic promoter, replacing the third promoter (between the upper end of the dptG gene and the lower end of the dptD gene) located in the 26,456 to 26,528^{th} nucleotide sequence with the B24 synthetic promoter, and replacing the fourth promoter with the B25 synthetic promoter.

As a result, the prepared RD12 to RD14 refactoring constructs showed an increase in production yield of about 400% compared to the wild-type strain (FIG. 8C).

The goal of second stage promoter engineering was to optimize the transcriptional efficiency of an operon (TOa/TOb) containing a transporter protein gene, and biosynthetic gene cluster refactoring constructs RD15 to RD18 were prepared by performing promoter engineering by targeting the fifth promoter P5 or sixth promoter P6 position. Specifically, RD15 was obtained by replacing the first promoter with the A31 synthetic promoter, replacing the second promoter with the B45 synthetic promoter, and replacing the fifth promoter and the sixth promoter (between the upper end of the dptM gene and the lower end of the dptP gene) located in the 77,531 to 78,288^{th} nucleotide sequence with the A48 synthetic promoter and the A46 synthetic promoter; RD16 was obtained by replacing the first promoter with the A31 synthetic promoter, replacing the second promoter with the B45 synthetic promoter, and replacing the fifth promoter and the sixth promoter with the B31 synthetic promoter and the B28 synthetic promoter; RD17 was obtained by replacing the first promoter with the A31 synthetic promoter, replacing the second promoter with the B45 synthetic promoter, and replacing the fifth promoter and the sixth promoter with the A29 synthetic promoter and the A07 synthetic promoter; and RD18 was obtained by replacing the first promoter with the A10 synthetic promoter, replacing the second promoter with the A32 synthetic promoter, and replacing the fifth promoter and the sixth promoter with the B52 synthetic promoter and the B45 synthetic promoter.

As a result, the prepared RD15 to RD18 refactoring constructs all showed significantly increased production yields of 1,000% or more, and a maximum production yield increase of 1,780% (489.1 mg/L) was confirmed (FIG. 8C). In particular, HPLC and RNA-seq analyses confirmed that the production yields of lipopeptides (2-4) in the RD09, RD10, RD14, RD16, and RD18 constructs were significantly increased compared to the wild-type strain (FIGS. 8B and 8D).

Accordingly, the present inventors have confirmed that by producing a construct in which the existing promoter for the daptomycin biosynthetic gene cluster is replaced with a novel synthetic promoter, a strain transformed with a vector including the construct can significantly increase the production of daptomycin.

### Example 7. Method for Producing Daptomycin by Supplying Decanoic Acid Precursor

Decanoic acid is an essential precursor for daptomycin production, and Streptomyces roseosporus strains cannot biosynthesize decanoic acid, so the only way to produce daptomycin is to supply decanoic acid to the medium. However, high concentrations of decanoic acid in the medium have cytotoxic effects that inhibit the growth of strains, which limits the mass production of daptomycin. Accordingly, in order to reduce the toxic effects of decanoic acid, the inventors of the present invention cloned fatty acid ligase (dptE) and acyl carrier protein (dptF), which activate decanoic acid and link it to a fatty acid carrier protein, into an overexpression plasmid.

As a result, the Streptomyces roseosporus strain transformed with the refactored novel daptomycin biosynthetic gene cluster and the fatty acid activation and transport protein overexpression vector showed an additional increase in production yield of about 26% (ultimately about ˜2,300%) in the production yield of the promoter engineering step.

Accordingly, in the present invention, it was confirmed that the production yield of daptomycin can be significantly increased through the step of codon-reprogramming of a non-ribosomal peptide synthesis gene in the daptomycin biosynthetic gene cluster derived from a wild-type Streptomyces roseosporus strain, cloning of the daptomycin biosynthetic gene cluster including the same, and the step of refactoring by promoter engineering. In addition, it was confirmed that the production yield of daptomycin was further significantly increased by transforming with an overexpression vector of fatty acid ligase and acyl carrier protein.

```
[Sequence Listing Free Text]
A12 promoter
SEQ ID NO: 1
atcgcttgca ggcgaggtcc agcgacgagc gtcgcacgct gccacagcag gacaggtcgc 60
aggtgagcgt gcactgacgg cacctcgtcg ggactgctga gcacggtcac ggcagccgag 120
tggagcacgc gtgagggagc gacgcaggtg acggtctcag cgccatcctg tgcgagtgac 180
cagcacacta cggagcggac gagtacggct tgtcagtact gaccctctag tgctacggtc 240
aataaggcga aacactcagg aggaagact 269
B25 promoter
SEQ ID NO: 2
cgctatgcca cgcaggttag cagcctggca cgtgagctgc cgatgagtct aggcagcaga 60
agagccggat aggcacgagc tcagtgacga ggccggtgac cgtcacggac ggtactagcc 120
gaggagggct gagcgagggt gcgtgagacc gcgagcggac ggtccacgag tgcggacgca 180
gtgcagctgc gtggagcgac ccccaatgct tgacaagagc gttcaaacgc atctagcatt 240
cgctacaaag ccccgccagg aggtacagc 269
A48 promoter
SEQ ID NO: 3
tcagcaaggc tcacctacgg ccagctcgac acctggctca cccagacctg ccaacgcctc 60
cacgcctgct acctgtcccc atccgacgag gccacctcgc caccgccacg gtcccaccta 120
ctcatcaccg tctccgtcac tcagggactg aagcacctca cgcgcactca ccgcaccttg 180
caccacaggc acgcagtctt cgagacccct tgacgcccca ctcgccttac acgtacactc 240
taaagcatcc cgtatcgagg aggtcaagc 269
A01 promoter
SEQ ID NO: 4
cgctatgcca cgcaggttcg acgtgagcgt cacagcggtc gcggtggatg gtgaggcgga 60
tactgaaggg tggtgcgcgg tccatggtgg atggcagggc gaacggacac gcaacggagg 120
cagggtggta gtgcggagag gctccaggtg atggtccgtg tgtagagggc cagaggaggt 180
cggcggtagt gtcccgtagg taacttcatt tggcgtgcat gaactcaacg tgctatcctt 240
cctaaaaagg ccccgccagg aggtacagc 269
A46 promoter
SEQ ID NO: 5
gtaggtgagc cttgctgacg gtcgaagagg cgtgcgggtc gtacgcaagt cggacacgga 60
cgtagtcgcg aggtgagtgg tgatgcaagt ggtaccagcc gacagagtcg tccgcagtag 120
cagacggctg tggatcgata gtgcgggtag agcgtgtgga gagcgtgacc cgtggatcat 180
ggcatgcgtc gaggtaccgt cacattgggt tgccagcccg ttgctggacg tactacggtt 240
gaagcctccc cggaatcagg aggacttcc 269
A31 promoter
SEQ ID NO: 6
taccacgcgt acagctacga cggctcggcc aacgtccgcc gtctcctccg tcacgtcgct 60
gctcacgcgg tactggctca gctgcgccag ctggactcgg ctcagtcggt cccacccttc 120
gtgcatcagg gtccagcgac cggtgacgct gcacgccgtg gctgctagtc ggccgactcc 180
ccgttgccac ctggaccgac gtactgagct tgcctgaaga acccacatcc agctaaagta 240
agccaacctg acctttgagg agggctca 268
A26 promoter
SEQ ID NO: 7
taccacgcgt acagctacat gcggaggaac agagtggcca gccgttgcca cgacgtccgg 60
acggcacgac acccccgcag gtacgatcag tggcacgcga ggcctgcgac agcgccaaga 120
ccggatacga caggaaccgt ggcaacagtc cgtagacacc gcagccagcg cagatcgcgg 180
gaggagatcc agatcgacgc cctgatgact tgacgctcga gaaggttcac gattagcatg 240
ccggttacgg acctttgagg agggctca 268
A15 promoter
SEQ ID NO: 8
acctcgcctg caagcgatcc ccatatctcc cgctggtcac gtctgccgac tagacggtgg 60
cgcacaccct ggttgctcaa gcgtctgcgg cccatcctct caaggtgtgc tccggactgt 120
gccaggtctg gcgccttcca ctccgcgccg tgataccgtt gggtagtggt cgctctgcgt 180
actggcctcg tcctacagcc gcgaagtact tgacacatac ctgctaagcc ttctaatctg 240
cttgcgtact ctaccacagg aggtaacta 269
A09 promoter
SEQ ID NO: 9
actggtgaat gatgggcact gtcgcacgag agcgtcagac ggacctagag cctagaccct 60
cgctcacctc gcacaatcag acggctctgg gacgctggct gccacgagcg agacgtcacg 120
tctggagcag cactggcgca gcctaccgcc acgcatccat cccgatccga cgagcgagcg 180
ggtgaccgct ctggctcaga cgcagccgct tgaccacatg gtcccgggca gtatacggtg 240
gctgcgaccc caaccaaagg aggcagtgt 269
A10 promoter
SEQ ID NO: 10
atcgcttgca ggcgaggtgc gatccactca gagctgctga cagtccaccg tcgtctgacc 60
gttcgcgacc actccggact ggccagcgtg tccgaggcct cagtcgctgt cgtgagatag 120
gcttgcaggg acgccactag cgacggtcgt ggacaggtga cacgcgcatc aaggacggcg 180
atgtctccgt agctagcgga gaacacctct tgactcgcct acccgccgct cgctaaattt 240
gctgtagcca aacactcagg aggaagact 269
B40 promoter
SEQ ID NO: 11
acctcgcctg caagcgatag ctcacacggc catccgcatc cacacaacgg cgctgaccac 60
gctctggcac gacgcctacg acggtccagt tggcagcagc gactgcgacc tcccagaccc 120
cgcttgcgag gctcacaccc agaccgcagt cacgacttgc gaccgcagcc tgcggaccta 180
ggacctgact gcacgctcgt caccaccatt tgtccaccct tctccttagc tggtaccatc 240
caggttcgct ctaccacagg aggtaacta 269
A49 promoter
SEQ ID NO: 12
tgcccatcat tcaccagtaa ccgagacggc atgtgggacc ctggccgaga ggagccaccg 60
tagggagaag taaggcggag cgagcaggga tggcacggag aggaggtgag cggtgcgagc 120
agtcgtacgg acacggagga cacgcggtgg tgacgccggg catcgccgca ccgaaggagc 180
cgtgacaggc tctcggcact atagtcccct tgacgagccc gagagagtgc agctaaggtc 240
ccacgttctt acgaacgagg aggccccat 269
B31 promoter
SEQ ID NO: 13
tcagcaaggc tcacctacta gacgcgtgcg gcggccttgc cgtggtggta cgtcggagtg 60
cgtgagggtg tggtttgcga tagggcgaac tggcgacagt gctcggcgac ggctgggatg 120
ttgggttgtg gttcagggct gccagtgcgt tggtcctacg ggcttaggtg gttggcgcgt 180
gtgactggac gactcggaca ttaaccacat tgacagcctg atgccgcggc gattacgatc 240
gctaatcacc cgtatcgagg aggtcaagc 269
B28 promoter
SEQ ID NO: 14
gtaggtgagc cttgctgatg acccaccagc cacacctcgc ctacgcccgt gccacaccga 60
cgcgctcaac acccaccagt catcacaacg ccacgccgta caccacagcc accacttcac 120
gagccgagtg cacggcacac acgtcgccac gaacgcacga caacgaccac gtccacacgc 180
accgacggca gtgccgagac actgatgcat tgacaagaat cacggaaaca ccttacactc 240
gatgagtggc cggaatcagg aggacttcc 269
A47 promoter
SEQ ID NO: 15
actggtgaat gatgggcagt ccgtcctgct agcgagccgt ctcgtcccca ccgcacgtga 60
ggcacaccta cctcctccgt acgccgaact ctccgctcgc gcgagtctgc tacgtcggct 120
agtcgctggc tcatcgcagc tgtatcttgc ttcgttcgta cctcggacgc ctgtcgctgg 180
cgctgacctc ggcactgctg cgaatgcatt tgacttcgat ctagggaacc aggtacgata 240
gaaaataatc caaccaaagg aggcagtgt 269
A20 promoter
SEQ ID NO: 16
acctcgcctg caagcgatgg agctgggtgc gacgggacat tgcatgccgg tacgctggat 60
cgctgggatc gcgacgcagt gctgccgtgg agcggtgccg tgcagtggtc gcacctctcc 120
agcgtcgcga gcagagcgtg ccgcagcctt gcccagtact ctacgcgccc actgggctgt 180
cgccacctga cggcacttga cgggatgtgt tgcctccgac tgttcacctc acctattatg 240
cctccacact ctaccacagg aggtaacta 269
B24 promoter
SEQ ID NO: 17
atcgcttgca ggcgaggtct ggtcgggttc gtgtcgcgga gagccgtggt ggtaagggtc 60
ggggtgccgg ctgtctggtg tgggggtggc gcgttcgcac cgtagtcgtt gcgaccgtcg 120
aagcgtgcct gtgcctccct gttctcgtgc acagcccggt gtcggggtcg cagtcggact 180
cggtccgagc tccatccaca agcgaatact tgccgcccct tccgtctgga tgttaagtta 240
atctcagaca aacactcagg aggaagact 269
R06 promoter
SEQ ID NO: 18
cgacgcttgt cgctaccaga atcattcccg aacggtgctg ccataaggct ccgcagagca 60
gctaagcgat accacaccaa cgagtgtaac gtgccgaatc gttgtcgaca tcaccgacac 120
ctgcttgact ccgtccttgg ttggccccgt gcaaagcgtc atgctagact acatgactgg 180
cctatcatag gaggcagttt 200
B52 promoter
SEQ ID NO: 19
tgcccatcat tcaccagtgg accacagcgg catcggacag cgtgcaaccc tcgcaaccag 60
gtcccccgac gacacctacg tgcagccaca acacagccac agccgccagc acggtgaccc 120
cacctctgcc gacccaccca gatccgagct tggacaccca ctgcgagcca cctgaccgac 180
aacggtcgcc aacgctacag acttcctgtt tgacaaggcc aatgcagcca cactaccctt 240
aacgcgactt acgaacgagg aggccccat 269
A32 promoter
SEQ ID NO: 20
gtagctgtac gcgtggtacc aggaggcgct gcacgcaaga gccgacctcg gacggaccca 60
ctcagagcat cgaggttcgt aggaggcaag gcggtcgaga cacgacagac cgctagaggc 120
agcagggtgg ccaacaggcc gcatagcagc gtccacaggg aacacggttg gaccagcgca 180
ggcactccaa cgacacgcgg tcccacgcct tggcccgagg gccgaccggg tgctattttc 240
ctcagaacaa gaatacgagg aggccccat 269
B42 promoter
SEQ ID NO: 21
cgctatgcca cgcaggttgt caggcacgta ggtgccagac cgatcgccca acccgtcaca 60
ctccactcga ctctactatg actcatccct acactcgcta cctagctcac gaggcctagc 120
gctcaagtgg ctagcacctg cgccactcgg ctctcacgtg caagcccgtg caccttcacg 180
tgcgactcac gcagtggtca ccccaggact tgtccggagc ggccgaaggg tactatgata 240
gaggccagtg ccccgccagg aggtacagc 269
R05 promoter
SEQ ID NO: 22
ttcactcgcg tccacagact ggagcgtgca ccgactcgca cagcgtcggt acggtgcgtg 60
tggaacgcat tagtgaactg gtccgacaga ctctcggtca cacgactgat cagcaggtac 120
cgtcttgcaa cacagtaccc ttgacgcctc acttgtacca gccctaacat gcctcctccg 180
ttggcttaag gaggccccat 200
A07 promoter
SEQ ID NO: 23
gtaggtgagc cttgctgagc gagtgccatc ggacggacta gccagaggta gagcgacgat 60
tgcgaccgtg gtcgatgagc tggttcggag ccgacgagcc gtcgcgtgac tacgctgacg 120
gcgtgaccac gagcgagcgt gatggcgact gtgcaccgca gagtgccgca ccgggctaca 180
tacgacgaca cgcgtctgga gaatctaagt tgacgggacg gtacggtgtg cggtaacgtc 240
tccccggtgc cggaatcagg aggacttcc 269
A29 promoter
SEQ ID NO: 24
tcagcaaggc tcacctacct cctgcctag cacggccacc ttcgagcgcc ttgcactacg 60
ccactgcgca tccactatcc ctgcctgcac ggacactctg actggcacgg tcgagctctg 120
gtcgcacatc ccgctctcgt cctccacgcc cccttcgaga tcctagccct gcacctgtac 180
cgcgcagctc cgtcgttgca ccctaacact tgacgcaacg ccttccgtcc gcataatctg 240
ttcggctgtc cgtatcgagg aggtcaagc 269
B45 promoter
SEQ ID NO: 25
actggtgaat gatgggcact ggtccggatc ggtgcaggcg gtgcactcgt ggagtgggtg 60
ggggtgacgg ctgactggtc tgcgggtggt gagctcgcca cgtagtcggt gtcgagtctg 120
cccagacgct gtgcctcact gatcccgtgc gacgcacggt gtcgcggtcg gacgctccgg 180
acgtagctcc gtctcgttca aacagcccat tgacacggtt cactgcgcgc ttttatgatg 240
tacagccctc caaccaaagg aggcagtgt 269
dptR2 regulatory gene 2
SEQ ID NO: 26
Met Ala Leu Ala Ser Glu Arg Gln Glu Phe Ile Leu Ala Thr Val Arg
    1 5 10 15
Glu Arg Gly Thr Val Arg Leu Ala Asp Leu Val Glu Arg Leu Gly Val
                              20 25 30
Thr Ala Val Thr Val Arg Arg Asp Val Thr Ile Leu Ala Asp Arg Gly
                      35 40 45
Leu Val Ser Arg Val His Gly Gly Val Thr Leu Pro Tyr Arg Gly Pro
          50 55 60
Thr Ala Asp Glu Arg Ala Thr Arg Gly Val Phe Thr His Gly Arg Leu
  65 70 75 80
Pro Asp Gln Ala Phe Val Gly Met Val Val Pro Thr Val Glu Tyr Tyr
                                      85 90 95
Trp Pro Ala Val Ile Gln Gly Ala Gln Ser Ala Val Ala Ala Ala Gly
                            100 105 110
Gly Arg Leu Val Leu Arg Ala Ser Ala Tyr Asp Ala Ala Glu Asp Arg
                  115 120 125
Arg Gln Val Thr Gly Leu Leu Asp Arg Gly Val Arg Thr Leu Leu Val
          130 135 140
Ala Pro Thr Asn Thr Gly Glu Ala Gly Gln Asp Leu Leu Arg Trp Leu
145 150 155 160
Gly Ser Leu Asn Val Pro Val Ile Leu Val Glu Arg Leu Pro Pro Pro
                                    165 170 175
Ala Leu Pro Thr Leu Pro Leu Asp Ala Val Thr Thr Ala His Ser Leu
                            180 185 190
Gly Ala Gly Leu Ala Val Arg His Leu Val Thr Leu Gly His His Arg
                  195 200 205
Ile Ala Phe Val Thr Ala Arg Phe Ser Pro Thr Thr Lys Ala Leu Arg
          210 215 220
Glu Gly Trp Gln Glu Thr Thr Ala Ser Leu Gly Leu Pro Ser Gly Asp
225 230 235 240
Gly Leu Ala His Asp Val Pro Ser Tyr Gly Ser Pro Gly Trp Ala Asp
                                    245 250 255
Ala Tyr Asp Ala Ala Leu Arg Ser Cys Arg Glu Ala Gly Ala Thr Ala
                            260 265 270
Leu Phe Val His Ser Asp Gly Glu Ala Val Gly Leu Val Glu Arg Ala
                    275 280 285
His Glu His Gly Leu Ala Val Pro Glu Asp Leu Ala Val Ile Thr Tyr
          290 295 300
Asp Asp Glu Val Ala Ala Ala Ala Asp Pro Pro Leu Thr Ala Val Arg
305 310 315 320
Pro Gln Lys His Arg Leu Gly Val Leu Ala Ala Glu Met Ala Leu Ala
                                  325 330 335
Arg Ala Val Asp Pro Val Glu Arg Pro Val His Arg Val Glu Leu Trp
                            340 345 350
Pro Asn Leu Ile Ile Arg Ala Ser Cys Gly Gly Thr Ala Pro Val Ser
                    355 360 365
Ala Gly Arg
370
dptR1_transcriptional regulator 1
SEQ ID NO: 27
Met Thr Ile Pro Pro Val Gly Arg Thr Gly Trp Asp Thr Gln Trp Gln
    1 5 10 15
Ala Leu Gly Leu Gly Ala Asp Glu Leu Arg Val Tyr Glu Ala Leu Leu
                              20 25 30
Asp Ala Pro Val Pro Ala Ser Arg Thr Ala Leu Ala Gln His Leu Gly
                      35 40 45
Leu Thr Val Arg Arg Val Thr Asn Ala Leu Asp His Leu Ala Glu Arg
          50 55 60
Arg Phe Thr His Pro Ala Arg Gly Ala Gly Leu Pro Val Ala Met Ala
  65 70 75 80
Pro Ala Thr Ala Leu Arg Asn Leu Ile His Leu His Gln Ala Glu Leu
                                      85 90 95
Leu His Arg Ser Ala Glu Leu Glu Glu Leu Ser Gly Ser Val Asp Arg
                            100 105 110
Ile Ala Ala Gln Leu Leu Ser Ser Val His Thr Pro Arg Ala Thr Gly
                  115 120 125
Ile Glu Thr Val Arg Gly Gly Ala Ala Ile Ala Ala Arg Val Ala Ser
          130 135 140
Leu Leu Val Ser Ala Ser Glu Glu Ile Ala Leu Leu Asp Arg Pro Pro
145 150 155 160
Tyr Ala Ser Ser Glu Pro Asp Gly Met Pro Val Pro Leu Asp Met Ala
                                    165 170 175
Glu Pro Val Arg Arg Gly Val Arg Val Arg Val Val Val Asp Arg Glu
                            180 185 190
Gly Leu Ser Phe His Gly Arg Ala Arg Gly Leu Gly Asp Leu Ala Val
                  195 200 205
Gln Gly Val Gln Ile Arg Val Gly Thr Asp Leu Pro Thr Lys Leu Ile
          210 215 220
Thr Val Asp Arg Arg Val Thr Leu Leu Pro Pro Thr Asp Ala Ala Asp
225 230 235 240
Pro Thr Ala Ser Ala Leu Val Val Ser Asp Ala Leu Leu Ser Asn Ala
                                    245 250 255
Leu Val Pro Leu Phe Glu Ser Val Trp Glu Arg Ala Leu Pro Ile Gly
                            260 265 270
Ser Val Thr His Asp Gln Ile Thr Asp Gly Asp Arg Glu Leu Leu Thr
                    275 280 285
Met Leu Ala Ser Gly Leu Lys Asp Glu Ala Met Ala Arg Arg Leu Asp
          290 295 300
Ile His Val His Thr Val Arg Arg Arg Ile Thr Arg Leu Met Gln Val
305 310 315 320
Leu Asn Ala Glu Thr Arg Phe Gln Ala Gly Val Gln Ala Ala Leu Arg
                                  325 330 335
Gly Trp Leu Thr Gly
                            340
dptJ Tryptophan 1,3-dioxygenase
SEQ ID NO: 28
Met Thr Ala Gln Asp Thr Arg Thr Thr Gly Ser Asp Gly Gly Gly Arg
    1 5 10 15
Gly Ala Thr Tyr His Glu Ser Pro Thr Tyr Gly Glu Leu Leu Arg Leu
                              20 25 30
Glu Asp Leu Leu Asn Val Ala His Leu Arg Asp Ala Ala Ala Pro Val
                      35 40 45
Leu Phe Leu Ala Thr His Gln Ser Ala Glu Ile Trp Phe Gly Ile Val
          50 55 60
Leu Arg His Leu Glu Glu Ile Arg Ala Ala Leu Thr Asp Asp Asp Pro
  65 70 75 80
Asp Thr Ala Leu His Leu Leu Pro Arg Leu Pro Glu Ile Phe Glu Leu
                                      85 90 95
Leu Val Arg His Phe Asp Met Leu Ala Thr Leu Ser Thr Glu Glu Phe
                            100 105 110
Gly Lys Ile Arg Ala Gly Leu Gly Thr Ala Ser Gly Phe Gln Ser Ala
                  115 120 125
Gln Tyr Arg Glu Ile Glu Phe Leu Cys Gly Leu Arg Asp His Arg His
          130 135 140
Ile Ser Thr Pro Gly Phe Thr Glu Thr Glu Arg Arg Arg Leu Arg Glu
145 150 155 160
Arg Ala Arg Gln Pro Ser Val Ala Glu Ala Tyr Asp Ala Phe Arg Thr
                                    165 170 175
Arg Cys Ala Asn Gly Lys Asp Ala Glu Arg Ile Gly Glu Ala Leu Leu
                            180 185 190
Arg Phe Asp Glu Arg Val Thr Val Trp Arg Ala Arg His Ala Ala Leu
                  195 200 205
Ala Glu Arg Phe Leu Gly Pro Leu Glu Gly Thr Ala Gly Thr Ala Gly
          210 215 220
Ala Asp Tyr Leu Trp Arg Val Thr Arg His Arg Leu Phe Pro Pro Glu
225 230 235 240
Ala Trp Gly Ala Gly
                                    245
dptI_Glutamine 3-methyl transferase
SEQ ID NO: 29
Met Thr Val His Asp Tyr His Val Thr Val Lys Glu Gln His Pro Ala
    1 5 10 15
Leu Phe Glu Leu Leu Asp Pro Ala Arg Leu Val Ala Val Thr Asp Glu
                              20 25 30
Pro Trp Val Thr Glu Gly Asn Glu Phe Asp Asp Asp His Thr Gly Arg
                      35 40 45
Gly Val Ser Tyr Arg Cys Ala Gln Gln His Gly Glu Ala Arg Arg Thr
          50 55 60
Gly Ile Glu Thr Ile Leu Gly Met Phe Ala Gly Pro Gly Gly Leu Arg
  65 70 75 80
Asp Met Gly Arg Val Leu Asp Val Leu Gly Gly Glu Gly Leu Leu Ser
                                      85 90 95
Arg Val Trp Arg Gln Leu Ala Gly Ala Gly Asp Gly Asp Ser Val Pro
                            100 105 110
Leu Val Thr Gly Asp Leu Ser Gly His Met Val Ala Ala Ala Leu Arg
                  115 120 125
Ser Gly Leu Pro Ala Val Arg Gln Pro Ala Asp Arg Met Leu Gln Arg
          130 135 140
Asp His Cys Leu Asp Gly Val Leu Phe Ala Tyr Gly Thr His His Val
145 150 155 160
Asp Arg Ser Val Arg Pro Arg Met Leu Thr Glu Ala Ser Arg Val Leu
                                    165 170 175
Ala Pro Gly Gly Arg Val Val Leu His Asp Phe Ala Glu Gly Ser Pro
                            180 185 190
Glu Glu Arg Trp Phe Arg Glu Val Val His Pro Arg Ser Leu Ala Gly
                  195 200 205
His Ala Tyr Asp His Phe Thr Ala His Glu Met Thr Gly Tyr Leu Ala
          210 215 220
Asp Ala Gly Phe Thr Asp Ile Thr Val Gly Pro Val Tyr Asp Pro Met
225 230 235 240
Thr Leu Thr Gly Glu Thr Glu Glu Ser Ala Leu Ala Arg Leu Val Ser
                                    245 250 255
Tyr Met Thr Ser Met Tyr Gly Ile Leu Pro Asp Gly Asp Arg Ser Asn
                            260 265 270
Glu Arg Thr Glu Ala Ala Leu Arg Asp Ile Phe Arg Phe Ser Ala Gly
                    275 280 285
Asp Leu Pro Glu Asp Val Pro Arg Asp Glu Ala Val Leu Glu Leu Thr
          290 295 300
Val Arg Pro His Gly Asn Ala Phe Arg Ala Glu Leu Pro Arg Ile Ala
305 310 315 320
Leu Val Ala His Gly Arg Lys Pro
                                  325
dptH alpha/beta Hydrolase
SEQ ID NO: 30
Met Arg Ala Thr Ser Arg Met Ile Gln Val Asn Gly Ala Arg Ile Ala
    1 5 10 15
Cys Ser Asp Ser Gly Cys Gly Asp Pro Val Leu Met Ile Ala Gly Thr
                              20 25 30
Gly Ser Thr Gly Arg Val Trp Asp Ala Tyr Gln Val Pro Asp Leu His
                      35 40 45
Ala Ala Gly Phe Arg Thr Ile Thr Phe Thr Asn Arg Gly Val Pro Pro
          50 55 60
Ser Asp Glu Cys Glu Arg Gly Phe Thr Leu Ala Asp Leu Ala Ala Asp
  65 70 75 80
Thr Ala Ala Leu Ile Glu Gln Val Ala Gly Gly Pro Cys Arg Val Val
                                      85 90 95
Gly Thr Ser Leu Gly Ala Gln Val Ala Gln Glu Val Ala Leu Ala Arg
                            100 105 110
Pro Asp Leu Val Thr Gln Ala Val Phe Met Ala Thr Arg Gly Arg Thr
                  115 120 125
Asp Ala Met Arg Ala Ala Ala Thr Arg Ala Ala Ala Ala Leu Tyr Asp
          130 135 140
Ser Gly Val Glu Leu Pro Pro Ala Tyr Ala Ala Ala Val Arg Ala Leu
145 150 155 160
Gln Asn Leu Ser Pro His Thr Leu Arg Asp Arg His Gln Val Glu Asp
                                    165 170 175
Trp Leu Pro Leu Phe Glu Tyr Ala Glu Arg Asp Gly Pro Gly Val Arg
                            180 185 190
Ala Gln Leu Glu Leu Gly Leu Leu Pro Asp Arg Leu Ala Asp Tyr Arg
                  195 200 205
Asp Ile Thr Val Pro Cys Leu Val Ile Ala Phe Glu Asp Asp Val Val
          210 215 220
Thr Pro Pro Tyr Leu Gly Arg Glu Val Ala Asp Ala Ile Pro Gly Ala
225 230 235 240
Arg Phe Glu Thr Val Pro Arg Cys Gly His Tyr Gly Tyr Leu Glu Asp
                                    245 250 255
Ala Ser Ala Val Asn Lys Ile Leu Arg Asp Phe Phe Arg Thr Ser
                            260 265 270
dptG_mbtH family protein
SEQ ID NO: 31
Met Ala Asn Pro Phe Glu Asn Asn Asp Gly Ser Tyr Leu Val Leu Val
    1 5 10 15
Asn Asp Glu Gly Gln Tyr Ser Leu Trp Pro Ala Phe Ala Asp Val Pro
                              20 25 30
Ala Gly Trp Thr Val Thr Phe Gly Glu Ser Ser Arg Gln Glu Cys Leu
                      35 40 45
Asp His Ile Asn Glu Asn Trp Thr Asp Met Arg Pro Lys Ser Leu Ile
          50 55 60
Arg Gln Met Glu Asn Asp Arg Thr Thr Ala Ala
  65 70 75
dptD daptomycin non-ribosomal peptide synthetase D
SEQ ID NO: 32
Met Thr Gln Arg Ala Met Glu Asp Ile Leu Pro Leu Thr Pro Leu Gln
    1 5 10 15
Glu Gly Leu Leu Phe His Ser Val Tyr Asp Glu Gln Ser Val Asp Val
                              20 25 30
Tyr Thr Val Gln Val Val Val Asp Leu Glu Gly Pro Val Asp Pro Glu
                      35 40 45
Ala Leu Arg Ala Ala Ala Ala Ala Leu Leu Arg Arg His Ala Asn Leu
          50 55 60
Arg Ala Ala Phe Arg Tyr Glu Arg Leu Gln Arg Pro Val Gln Ile Ile
  65 70 75 80
Pro Arg Glu Val Ala Val Pro Trp Glu His Thr Asp Val Ala Lys Leu
                                      85 90 95
Glu Gly Ala Glu Gln Lys Ala Glu Ile Glu Arg Leu Leu His Asp Gln
                            100 105 110
Arg Trp Arg Arg Phe Asp Leu Thr Ala Pro Pro Leu Leu Arg Phe Leu
                  115 120 125
Leu Val Arg Thr Gly His Asp Arg His Arg Phe Ala Leu Thr Phe His
          130 135 140
His Ile Leu Met Asp Gly Trp Ser Met Pro Val Leu Leu Arg Glu Leu
145 150 155 160
Ile Thr Leu Tyr Arg Thr Gly Asp Glu Thr Ala Leu Pro Trp Val Arg
                                    165 170 175
Pro Tyr Arg Asp Tyr Leu Ala Trp Ile Ser Arg Arg Asp Arg Asp Glu
                            180 185 190
Ala Gly Arg Ala Trp Ser Lys Ala Leu Ala Gly Val Asp Glu Ala Thr
                  195 200 205
Leu Val Ala Pro Gly Ala Asp Arg Ala Ala Glu Pro Pro Leu Trp Thr
          210 215 220
Glu Ser Arg Leu Glu Pro Asp Leu Ala Ala Thr Leu Ala Ala Arg Ala
225 230 235 240
Arg Glu Phe Gly Val Thr Leu Asn Thr Leu Val Gln Ala Ala Trp Ala
                                    245 250 255
Leu Val Leu Gly Arg Leu Thr Gly Arg Asp Asp Val Val Phe Gly Val
                            260 265 270
Thr Val Ser Gly Arg Pro Pro Glu Leu Ala Gly Val Glu Asp Met Val
                    275 280 285
Gly Leu Phe Ile Asn Thr Val Pro Leu Arg Ala Glu Leu Leu Pro His
          290 295 300
Glu Ser Leu Arg Asp Phe Thr Val Arg Leu Gln Arg Glu Gln Ile Gln
305 310 315 320
Leu Leu Asp His Gln Tyr Glu Arg Leu Ala Val Ile Gln Arg Leu Ala
                                  325 330 335
Gly Arg Thr Glu Leu Phe Asp Thr Val Met Val Phe Glu Asn Tyr Pro
                            340 345 350
Val Ala Ala Ala Ser Ser Ala Gly Ala Asp Gly Pro Ala Ala Glu Pro
                    355 360 365
Arg Val Ala Asp Val His Val Arg Asp Ala Met His Tyr Pro Leu Gly
            370 375 380
Leu Leu Val Leu Pro Gly Pro Pro Leu Arg Leu Arg Phe Gly His Arg
385 390 395 400
Pro Ser Ala Leu Pro Ala Glu Arg Val Thr Thr Ile Arg Asp Ser Leu
                                      405 410 415
Val Arg Ala Leu Glu Leu Met Ala Asp Gln Pro Asp Leu Ala Val Gly
                              420 425 430
Arg Ala Asp Ile Leu Gly Glu Glu Glu Lys Gln His Leu Leu Thr Gly
                    435 440 445
Leu Asn Asp Thr His Arg Asp Val Pro Pro Leu Thr Val Pro Gly Met
          450 455 460
Ile Glu Ala Gln Ala Ala Arg Thr Pro Gly Arg Pro Ala Val His Ala
465 470 475 480
Arg Asp Gly Glu Leu Ser Tyr Ala Glu Leu Asn Ala Arg Ala Asn Arg
                                    485 490 495
Leu Ala Arg His Leu Ala Ala Ala Gly Val Gly Pro Glu Gln Tyr Val
                          500 505 510
Thr Leu Leu Leu Pro Leu Ser Ala Arg Met Val Val Ala Ala Leu Ala
                  515 520 525
Val Met Lys Thr Gly Ala Ala Tyr Val Pro Val Asp Pro Glu Tyr Pro
        530 535 540
Ala Asp Arg Ile Ala Tyr Met Leu Gly Asp Ile Gly Pro Ala Leu Val
545 550 555 560
Leu Thr Asp Ser Arg Ser Ala Ala Ala Met Pro Ala Gly Pro Ala Arg
                                    565 570 575
Val Leu Thr Leu Asp Asp Asp Ala Leu Asp Thr Gly Val Arg Ala Leu
                          580 585 590
Pro Glu His Asp Leu Gly Thr Asp Gly Ile Ala Pro Leu Pro Asp Gln
                    595 600 605
Pro Ala Tyr Val Ile Tyr Thr Ser Gly Ser Thr Gly Arg Pro Lys Gly
          610 615 620
Val Val Ile Leu His Arg Ser Val Thr Gly Tyr Leu Leu Arg Thr Ile
625 630 635 640
Glu Glu Tyr Pro Glu Ala Ala Gly Lys Ala Phe Val His Ser Pro Val
                                    645 650 655
Ser Phe Asp Leu Thr Val Gly Ala Leu Tyr Ala Pro Leu Val Ser Gly
                            660 665 670
Gly Cys Leu Arg Leu Gly Ser Phe Thr Asp Asp Lys Ile Leu Asp Leu
                    675 680 685
Gly Glu Asp Ser Pro Thr Phe Met Lys Ala Thr Pro Ser His Leu Ala
          690 695 700
Val Leu Asp Ser Leu Pro Asp Glu Ile Ser Pro Thr Gly Ala Ile Thr
705 710 715 720
Leu Gly Gly Glu Gln Leu Leu Ser Glu Thr Leu Asp Pro Trp Arg Ala
                                    725 730 735
Arg His Pro Gly Val Thr Val Phe Asn Val Tyr Gly Pro Thr Glu Thr
                          740 745 750
Thr Ile Asn Cys Ala Glu His Arg Ile Ala Pro Gly Thr Thr Leu Pro
                  755 760 765
Pro Gly Pro Val Pro Ile Gly Arg Pro Leu Trp Asn Thr Arg Leu Tyr
          770 775 780
Val Leu Asp Gly Gly Leu Arg Val Val Pro Thr Gly Val Ala Gly Glu
785 790 795 800
Leu Tyr Val Ala Gly Ala Gly Leu Ala Arg Gly Tyr Leu Gly Arg Pro
                                        805 810 815
Gly Leu Thr Ala Glu Arg Phe Val Ala Cys Pro Phe Gly Ala Pro Gly
                              820 825 830
Glu Arg Met Tyr Arg Thr Gly Asp Leu Val Arg Trp Arg Thr Asp Gly
                    835 840 845
Thr Leu Glu Phe Val Gly Arg Val Asp Asp Gln Val Lys Val Arg Gly
            850 855 860
Phe Arg Ile Glu Leu Gly Glu Val Glu Ala Thr Val Ala Ala Thr Pro
865 870 875 880
Gly Val Ala Arg Ala Ile Val Ala Val Arg Glu Asp Arg Pro Gly Asp
                                    885 890 895
Gln Arg Leu Val Ala Tyr Val Thr Pro Ala Asp Val Asp Pro Thr Gly
                          900 905 910
Gly Leu Pro Ser Ala Val Thr Ala His Ala Ala Ala Arg Leu Pro Ala
                  915 920 925
Tyr Met Val Pro Ser Ala Val Val Val Leu His Glu Val Pro Leu Thr
        930 935 940
Pro Asn Gly Lys Ile Asn Arg Ala Ala Leu Pro Ala Pro Glu Ala Val
945 950 955 960
Ser Gly Ala Gly Phe Arg Ala Pro Gly Thr Ala Arg Glu Glu Val Leu
                                    965 970 975
Cys Gly Leu Phe Ala Glu Val Leu Gly Leu Glu Arg Val Gly Thr Ala
                            980 985 990
Asp Asp Phe Phe Glu Leu Gly Gly His Ser Leu Leu Ala Thr Arg Leu
                  995 1000 1005
Val Ser Arg Val Arg Ser Val Leu Gly Val Glu Leu Gly Val Arg Ala
      1010 1015 1020
Leu Phe Asp Ala Pro Thr Pro Gly Arg Leu Asp Arg Leu Leu Gly Glu
1025 1030 1035 1040
Arg Ser Gly Ala Pro Val Arg Ala Pro Leu Thr Ala Arg Glu Arg Thr
                                  1045 1050 1055
Gly Arg Asp Pro Leu Ser Tyr Ala Gln Gln Arg Leu Trp Phe Leu His
                          1060 1065 1070
Glu Leu Glu Gly His Gly Ala Thr Tyr Asn Ile Pro Leu Ala Leu Arg
                  1075 1080 1085
Leu Thr Gly Pro Leu Asp Val Thr Ala Leu Glu Ala Ala Leu Thr Asp
            1090 1095 1100
Val Val Ala Arg His Glu Ser Leu Arg Thr Leu Ile Ala Arg Asp Gly
1105 1110 1115 1120
Thr Gly Thr Ala Trp Gln His Ile Leu Pro Thr Gly Asp Pro Arg Ala
                                  1125 1130 1135
Arg Ile Thr Leu Glu Ala Val Pro Leu His Arg Asp Glu Leu Ala Gly
                          1140 1145 1150
Arg Leu Ala Glu Ala Ala Arg His Pro Phe Asp Leu Thr Ala Glu Ile
                1155 1160 1165
Pro Val Arg Ala Thr Val Phe Arg Thr Glu Arg Asp Asp His Thr Leu
        1170 1175 1180
Leu Val Val Thr His His Ile Ala Ser Asp Arg Trp Ser Arg Glu Pro
1185 1190 1195 1200
Phe Leu Arg Asp Leu Ser Ala Ala Tyr Ala Ala Arg Arg Ala His Ser
                                      1205 1210 1215
Ala Pro Glu Leu Pro Pro Leu Ser Val Gln Tyr Ala Asp Tyr Ala Ala
                            1220 1225 1230
Trp Gln Arg Asp Val Leu Gly Thr Glu Asp Asp Gly Thr Ser Glu Met
                  1235 1240 1245
Ala Gly Gln Leu Ala His Trp Arg Gly Arg Leu Ala Gly Leu Pro Gln
            1250 1255 1260
Gly Leu Asp Leu Pro Thr Asp Arg Pro Arg Arg Pro Asp Val Gly Arg
1265 1270 1275 1280
Arg Gly Gly Arg Cys Arg Leu Glu Ile Pro Ala Ala Leu His Arg Asp
                                1285 1290 1295
Ile Val Thr Leu Ala Arg Val Thr Ser Thr Thr Val Phe Met Val Val
                        1300 1305 1310
Gln Ala Ala Leu Ala Gly Leu Leu Ser Arg Leu Gly Ala Gly Thr Asp
                  1315 1320 1325
Ile Pro Ile Gly Thr Pro Ile Ala Gly Arg Thr Asp Glu Ala Thr Glu
              1330 1335 1340
His Leu Ile Gly Phe Phe Val Asn Thr Leu Val Leu Arg Thr Asp Val
1345 1350 1355 1360
Ser Gly Asp Pro Thr Phe Ala Glu Leu Leu Ala Arg Val Arg Ala Thr
                                    1365 1370 1375
Asp Leu Asp Ala Tyr Ala His Gln Asp Val Pro Phe Glu Arg Leu Val
                          1380 1385 1390
Glu Val Leu Asn Pro Glu Arg Ser Leu Leu Arg His Pro Leu Phe Gln
                1395 1400 1405
Ile Leu Leu Ala Phe Gln Asn Thr Glu Asp Arg Ser Ile Ser Asp Arg
          1410 1415 1420
Pro Gly Thr Leu Leu Pro Asp Leu Gln Val Thr Glu Gln Pro Leu Asp
1425 1430 1435 1440
Ala Gly Thr Ala Lys Phe Asp Leu Ala Phe Ala Phe Thr Glu Arg Pro
                                1445 1450 1455
Pro Glu Lys Gly Glu Pro Ser Gly Ile Thr Gly Ile Val Glu Tyr His
                    1460 1465 1470
Ala Asp Leu Tyr Asp Glu Gly Thr Val Arg Gln Ile Ala Asp Cys Phe
            1475 1480 1485
Val Gln Phe Leu Asp Ala Ala Val His Ala Pro Gly Thr Arg Val Asp
      1490 1495 1500
Ala Val Gly Leu Leu Pro Glu His Thr Leu His Lys Leu Leu Thr Arg
1505 1510 1515 1520
Ser Arg Gly Thr Val Thr Gly Leu Pro Pro Ala Thr Leu Pro Glu Leu
                                  1525 1530 1535
Phe Glu Ala Arg Val Ala Ala His Pro Gly His Ile Ala Val Glu Val
                          1540 1545 1550
Ala Gly Arg Arg Pro Ala Thr Thr Thr Tyr Asp Ala Leu Asn Arg Arg
                  1555 1560 1565
Ala Asn Arg Leu Ala Arg Leu Leu Thr Asp Arg Gly Val Arg Pro Glu
          1570 1575 1580
Gln Arg Val Ala Ile Ala Leu Pro Arg Ser Ala Asp Leu Val Thr Ala
1585 1590 1595 1600
Trp Leu Gly Ile Leu Lys Ala Gly Ala Val Cys Val Pro Val Asp Pro
                                  1605 1610 1615
Ala Tyr Pro Asp Asp Arg Ile Ala His Met Ala Ala Asp Ala Ala Pro
                        1620 1625 1630
Ala Leu Leu Ile Ala Ser Ala Ala Thr Arg Asp Arg Met Leu Pro Thr
                  1635 1640 1645
Gly Ile Pro Val Leu Asp Leu Asp Asp Pro Ala Val Thr Ala Ala Leu
          1650 1655 1660
Ala Ala Ala Pro Asp Gly Asn Pro Arg Gly Thr Gly Leu Leu Pro Ala
1665 1670 1675 1680
His Pro Ala Tyr Val Ile Tyr Thr Ser Gly Ser Thr Gly Thr Pro Lys
                                  1685 1690 1695
Gly Val Val Val Thr His Glu Gly Ile Pro Ala Leu Ala Ala Thr Gln
                          1700 1705 1710
Gln Glu Ala Leu Arg Ala Gly Pro Gly Asp Arg Val Leu Gln Leu Val
                1715 1720 1725
Ser Thr Ser Phe Asp Ala Ser Val Trp Asp Leu Cys Ser Ala Leu Leu
          1730 1735 1740
Ser Gly Ala Thr Leu Val Leu Ala Pro Asp Ala Asp Leu Phe Gly Asp
1745 1750 1755 1760
Glu Leu Ala Ala Ala Leu Thr Ala His Arg Ile Thr His Val Thr Leu
                                            1765 1770 1775
Pro Pro Ala Ala Leu Ala Ala Val Pro Ala Gly Ala Ala Pro Pro Arg
                                  1780 1785 1790
Leu Thr Val Thr Val Thr Gly Asp Val Cys Gly Pro Gln Leu Val Asp
                          1795 1800 1805
Arg Trp Ala Gly Gly Glu Arg Arg Ile Leu Asn Gly Tyr Gly Pro Thr
                  1810 1815 1820
Glu Val Thr Val Gly Ala Thr Tyr Ala Val Cys Glu Arg Thr Gly Asp
1825 1830 1835 1840
Gly Ala Pro Val Pro Ile Gly Ala Pro Trp Pro Asp Gln Arg Val Tyr
                                    1845 1850 1855
Val Leu Asp His Arg Leu Arg Pro Val Pro Ala Gly Cys Val Gly Glu
                          1860 1865 1870
Ile Tyr Val Ala Gly Ala Gly Leu Ala Arg Gly Tyr Leu Gly Arg Pro
                  1875 1880 1885
Gly Gln Thr Ala Glu Arg Phe Val Ala Asp Pro Phe Gly Ala Pro Gly
            1890 1895 1900
Glu Arg Met Tyr Arg Thr Gly Asp Leu Ala Arg Arg Arg Ser Asp Gly
1905 1910 1915 1920
His Leu Leu Phe Glu Gly Arg Ala Asp Thr Gln Val Lys Ile Arg Gly
                                        1925 1930 1935
Phe Arg Val Glu Leu Ala Glu Ile Glu Ala Ala Leu Ala Ser His Pro
                              1940 1945 1950
Gly Val Glu Asp Ala Val Val Thr Val Tyr Asp Asp Gly Leu Gly Asp
                    1955 1960 1965
Gln Arg Leu Val Ala Tyr Val Thr Gly Gly Pro Gly Thr Pro Ser Ala
          1970 1975 1980
Ala Ala Leu Arg Ala His Leu Ala Ser Arg Leu Pro Arg His Met Val
1985 1990 1995 2000
Pro Gly Asp Val Leu Thr Leu Asp Ala Leu Pro Leu Thr Ala Asn Gly
                                          2005 2010 2015
Lys Val Asp Arg Thr Ala Leu Pro Gly Pro Gly Thr Gln Thr Ala Ala
                                  2020 2025 2030
Pro Gly Arg Ala Pro Gln Ser Pro Gln Glu Arg Val Leu Cys Ala Leu
                        2035 2040 2045
Phe Ala Asp Val Leu Gly Arg Glu Thr Val Gly Val Asp Glu Gly Phe
            2050 2055 2060
Phe Asp Leu Gly Gly His Ser Leu Leu Ala Thr Arg Leu Ala Ala Arg
2065 2070 2075 2080
Val Arg Ala Ala Leu Gly Val Glu Ile Ser Val Arg Thr Leu Phe Glu
                                          2085 2090 2095
Ala Pro Thr Pro Ala Leu Leu Ala Ser Ala Cys Thr Ala Asp Ala Ala
                                  2100 2105 2110
Ala Tyr Asp Pro Phe Glu Thr Val Leu Pro Leu Arg Arg Thr Gly Ser
                        2115 2120 2125
Arg Pro Pro Leu Phe Cys Val His Ala Gly Met Gly Leu Ser Trp Ala
            2130 2135 2140
Tyr Ala Gly Leu Leu Ser His Leu Asp Ala Asp Val Pro Val Tyr Gly
2145 2150 2155 2160
Leu Gln Ala Arg Arg Leu Thr Ala Pro Gly Gly Leu Pro Gly Ser Val
                                          2165 2170 2175
Glu Glu Met Ala Glu Asp Tyr Ala Gly Glu Ile Arg Arg Leu Cys Pro
                                  2180 2185 2190
Asp Gly Pro Tyr Arg Leu Leu Gly Trp Ser Phe Gly Gly Thr Val Ala
                        2195 2200 2205
His Ala Val Ala Thr Arg Leu Gln Gln Gln Gly His Thr Val Glu Leu
                  2210 2215 2220
Leu Ala Val Leu Asp Ala Tyr Pro Val Thr Gly Ala Arg Pro Asp Ala
2225 2230 2235 2240
Glu Val Asp Glu Gln Arg Ile Val Ala Asp Tyr Leu Ala Gln Leu Gly
                                    2245 2250 2255
Ser Pro Val Ala Pro Glu Arg Leu Glu Gly Asp Ala Trp Leu Pro Glu
                          2260 2265 2270
Phe Leu Glu Phe Val Arg Arg Thr Asp Gly Pro Ala Arg Asp Phe Asp
                    2275 2280 2285
Ala Gly Arg Ile Leu Ala Met Lys Asp Val Phe Leu Asn Asn Ala Arg
            2290 2295 2300
Leu Thr Arg Arg Phe Thr Pro Gly Val Phe Thr Gly Asp Met Val Phe
2305 2310 2315 2320
Phe Ala Ser Ala Arg Pro Gly Ser Glu Gln Ala Ala Glu Arg Val Gly
                                    2325 2330 2335
Leu Trp His Pro His Val Thr Gly Asp Leu Asp Leu His Leu Ile Asp
                          2340 2345 2350
Cys Ala His Glu Glu Met Thr Asp Pro Ala Ala Leu Thr Arg Ile Gly
                  2355 2360 2365
Pro Val Leu Ala Ala Arg Leu Gly Ala Gly Thr
        2370 2375
dptBC daptomycin non-ribosomal peptide synthetase BC
SEQ ID NO: 33
Met Asn Arg Arg Ser Lys Val Val Glu Glu Ile Leu Pro Val Ser Ala
    1 5 10 15
Leu Gln Glu Gly Leu Leu Phe His Ser Ser Phe Ala Ala Ala Asp Gly
                              20 25 30
Val Asp Val Tyr Ala Gly Gln Leu Ala Phe Asp Leu Val Gly Ala Val
                      35 40 45
Asp Thr Gly Arg Leu Arg Ala Ala Val Glu Ser Leu Val Ala Arg His
          50 55 60
Gly Val Leu Arg Ser Ser Tyr Arg Gln Ala Arg Ser Gly Glu Trp Val
  65 70 75 80
Ala Val Val Ala Arg Arg Val Ala Thr Pro Trp Arg Ala Val Asp Ala
                                      85 90 95
Arg Asp Gly Ala Thr Asp Ala Ala Ala Val Ala Arg Glu Glu Arg Trp
                            100 105 110
Arg Pro Phe Asp Leu Gly Arg Ala Pro Leu Ala Arg Phe Val Leu Val
                  115 120 125
Arg Thr Asp Asp Asp Arg Phe Arg Phe Val Ile Thr Tyr His His Val
          130 135 140
Ile Leu Asp Gly Trp Ser Leu Pro Val Leu Leu Arg Glu Leu Leu Ala
145 150 155 160
Leu Tyr Gly Ser Gly Ala Asp Pro Ser Val Leu Pro Pro Val Arg Pro
                                    165 170 175
Tyr Gly Asp Phe Leu Arg Trp Ala Ala Ala Arg Asp Asp Ala Ala Ala
                            180 185 190
Glu Thr Ala Trp Arg Asp Ala Leu Thr Gly Leu Asp Glu Pro Ser Leu
                  195 200 205
Val Ala Pro Gly Ala Ser Pro Asp Gly Val Val Pro Ala Ser Val His
          210 215 220
Ala Glu Leu Asp Lys Ala Gly Thr Glu Asn Leu Ala Ala Trp Ala Arg
225 230 235 240
His Arg Gly Ile Thr Gln Ala Thr Ala Val Arg Ala Ala Trp Ala Leu
                                    245 250 255
Val Leu Gly Gln His Thr Gly Arg Asp Asp Val Val Phe Gly Val Thr
                            260 265 270
Val Ser Gly Arg Pro Ala Glu Leu Ala Gly Ala Glu His Met Val Gly
                    275 280 285
Leu Phe Ile Asn Thr Val Pro Leu Arg Thr Val Leu Asp Pro Ala Asp
          290 295 300
Thr Leu Gly Thr Phe Ala Ala Arg Leu Gln Ala Glu Gln Thr Thr Leu
305 310 315 320
Leu Glu His Gln His Val Arg Leu Ser Asp Ile Gln Arg Trp Ala Gly
                                  325 330 335
His Lys Glu Leu Phe Asp Thr Ile Val Val Phe Glu Asn Tyr Pro Ile
                            340 345 350
Gly His Ser Gly Pro Gly Ser Ile Arg Thr Asp Asp Phe Thr Val Thr
                    355 360 365
Ala Thr Glu Gly Ser Asp Ala Thr His Tyr Pro Leu Thr Leu Thr Ala
            370 375 380
Val Pro Gly Glu Thr Leu Arg Leu Lys Leu Asp His Arg Pro Asp Leu
385 390 395 400
Val Asp Thr Thr Thr Ala Thr Ala Leu Leu Arg Arg Val Thr Arg Val
                                      405 410 415
Leu Glu Thr Ala Thr Asp Asp Thr Gly His Thr Leu Ala Arg Leu Asp
                              420 425 430
Leu Leu Asp Asp Asp Glu Arg His Arg Leu Leu Arg Gly Trp Asn Asp
                    435 440 445
Thr Thr Arg Glu Gln Pro Pro Thr Tyr Tyr His Gln Glu Phe Glu Glu
          450 455 460
Gln Ala Arg Arg Arg Pro His Asp Thr Ala Leu Val Phe Thr Ser Thr
465 470 475 480
Ser Trp Thr Tyr Glu Glu Leu Asn Asp Arg Ala Asn Arg Leu Ala Arg
                                    485 490 495
Leu Leu Val Ala Ala Gly Ala Gly Ser Asp Asp Phe Val Ala Leu Ala
                          500 505 510
Phe Pro Arg Ser Ala Glu Ser Val Val Ala Ile Leu Ala Val Leu Lys
                  515 520 525
Ala Gly Ala Ala Tyr Leu Pro Leu Asp Met Asp Gln Pro Ala Glu Arg
        530 535 540
Leu Thr Gly Ile Leu Ala Asp Ala His Pro Thr Val Val Leu Thr Thr
545 550 555 560
Thr Thr Ala Thr Pro Leu Pro His Pro Gly Arg Thr Leu Val Leu Asp
                                    565 570 575
Ser Pro Thr Thr Ala Arg Ala Leu Ala Ala Ala Pro Ala His Asn Leu
                          580 585 590
Thr Asp Ala Asp Arg Arg Thr Pro Leu Asn Ala Arg Asn Ala Ala Tyr
                    595 600 605
Ile Ile His Thr Ser Gly Ser Thr Gly Arg Pro Lys Gly Val Val Ile
          610 615 620
Glu His Arg Ser Leu Ala Asn Leu Phe His Asp His Arg Arg Ala Leu
625 630 635 640
Ile Glu Pro His Ala Ala Gly Gly Ser Arg Leu Lys Ala Gly Leu Thr
                                    645 650 655
Ala Ser Leu Ser Phe Asp Thr Ser Trp Glu Gly Leu Ile Cys Leu Ala
                            660 665 670
Ala Gly His Glu Leu His Leu Ile Asp Asp Asp Thr Arg Arg Asp Ala
                    675 680 685
Glu Arg Val Ala Glu Leu Ile Asp Arg Gln Arg Ile Asp Val Ile Asp
          690 695 700
Val Thr Pro Ser Phe Ala Gln Gln Leu Val Glu Thr Gly Ile Leu Asp
705 710 715 720
Glu Gly Arg His His Pro Ala Ala Phe Met Leu Gly Gly Glu Gly Val
                                    725 730 735
Asp Ala Lys Leu Trp Thr Arg Leu Ser Asp Val Pro Gly Val Thr Ser
                          740 745 750
Tyr Asn Tyr Tyr Gly Pro Thr Glu Phe Thr Val Asp Ala Leu Ala Cys
                  755 760 765
Thr Val Gly Ile Ala Pro Arg Pro Val Ile Gly His Pro Leu Asp Asn
          770 775 780
Thr Ala Ala Tyr Ile Leu Asp Gly Phe Leu Arg Pro Val Pro Glu Gly
785 790 795 800
Val Ala Gly Glu Leu Tyr Leu Ala Gly Thr Gln Leu Ala Arg Gly Tyr
                                        805 810 815
Ala Gly Arg Pro Gly Leu Thr Ala Glu Arg Phe Val Ala Cys Pro Phe
                              820 825 830
Gly Ala Pro Gly Glu Arg Met Tyr Arg Thr Gly Asp Leu Val Arg Arg
                    835 840 845
Ser Pro Gly Gly Val Val Glu Tyr Leu Gly Arg Val Asp Asp Gln Ile
            850 855 860
Lys Leu Arg Gly Phe Arg Ile Glu Pro Ala Glu Ile Glu Leu Ala Leu
865 870 875 880
Ala Gly His Pro Ala Val Ala Gln Asn Val Val Leu Leu His Arg Ser
                                    885 890 895
Ala Thr Gly Glu Ala Arg Leu Val Ala Tyr Val Val Pro Gly Thr Pro
                          900 905 910
Val Asp Pro Arg Glu Leu Thr Gly His Leu Ala Ala Arg Leu Pro Ala
                  915 920 925
Tyr Met Val Pro Ser Ala Phe Val Leu Leu Asp Thr Leu Pro Leu Thr
        930 935 940
Pro Asn Gly Lys Leu Asp Arg Gly Ala Leu Pro Glu Pro Ala Phe Gly
945 950 955 960
Thr Ala Pro Arg Pro Glu Arg Pro Arg Thr Pro Val Glu Glu Ile Leu
                                    965 970 975
Cys Gly Leu Tyr Ala Asp Val Leu Gly Leu Pro Ser Phe Gly Ala Asp
                            980 985 990
Asp Asp Phe Phe Asp Ala Gly Gly His Ser Leu Leu Ala Ser Lys Leu
                  995 1000 1005
Val Ser Arg Ile Arg Thr Asn Leu Lys Thr Glu Leu Asn Val Arg Ala
      1010 1015 1020
Leu Phe Glu His Arg Thr Val Ser Ser Leu Ala Thr Ala Leu His Arg
1025 1030 1035 1040
Ala Ala Gln Ala Gly Pro Ala Leu Thr Ala Gly Pro Arg Pro Ala Arg
                                  1045 1050 1055
Ile Pro Leu Ser Tyr Ala Gln Arg Arg Leu Trp Phe Leu Asn Arg Leu
                          1060 1065 1070
Asp Arg Asp Ser Ala Ala Tyr Asn Met Pro Val Ala Leu Arg Leu Arg
                  1075 1080 1085
Gly Pro Leu Asp Ser Thr Ala Met Cys Ala Ala Leu Thr Asp Val Ala
            1090 1095 1100
Glu Arg His Glu Ala Leu Arg Thr Val Phe Glu Glu Asp Arg Asp Gly
1105 1110 1115 1120
Ala His Gln Ile Val Leu Pro Ala Thr Gly Leu Gly Pro Leu Leu Thr
                                  1125 1130 1135
Val Thr Gly Ala Asp Gly Thr Thr Leu Arg Ala Leu Ile Thr Glu Phe
                          1140 1145 1150
Val Arg Arg Pro Phe Asp Leu Ala Ala Glu Ile Pro Phe Arg Ala Ala
                1155 1160 1165
Leu Phe Arg Val Gly Asp Glu Glu His Val Leu Val Val Val Leu His
        1170 1175 1180
His Ile Ala Gly Asp Gly Trp Ser Met Gly Pro Leu Ala Arg Asp Val
1185 1190 1195 1200
Ala Glu Ala Tyr Arg Ala Arg Ala Ala Gly Arg Ala Pro Asp Trp Glu
                                      1205 1210 1215
Pro Leu Pro Val Gln Tyr Ala Asp Tyr Ala Leu Trp Gln Arg Glu Val
                            1220 1225 1230
Leu Gly Ala Glu Asp Asp Glu Thr Gly Glu Leu Ser Ala Gln Leu Ala
                  1235 1240 1245
His Trp Arg Thr Arg Leu Ala Gly Ala Pro Ala Glu Leu Thr Leu Pro
            1250 1255 1260
Thr Asp Arg Pro Arg Pro Ala Val Ala Ser Thr Ala Gly Asp Arg Val
1265 1270 1275 1280
Glu Phe Thr Val Pro Ala Gly Leu His Gln Ala Leu Ala Asp Leu Ala
                                1285 1290 1295
Arg Ala His Gly Ala Thr Val Phe Met Val Val Gln Ala Ala Leu Ala
                        1300 1305 1310
Val Leu Leu Ser Arg Leu Gly Ala Gly Asp Asp Ile Pro Ile Gly Thr
                  1315 1320 1325
Pro Val Ala Gly Arg Thr Asp Glu Ala Thr Glu Glu Leu Ile Gly Phe
              1330 1335 1340
Phe Val Asn Thr Leu Val Leu Arg Thr Asp Val Ser Gly Asp Pro Thr
1345 1350 1355 1360
Phe Ala Glu Leu Leu Ala Arg Val Arg Ala Thr Asp Leu Asp Ala Tyr
                                    1365 1370 1375
Ala His Gln Asp Val Pro Phe Glu Arg Leu Val Glu Val Leu Asn Pro
                          1380 1385 1390
Glu Arg Ser Leu Ala Arg His Pro Leu Phe Gln Val Met Leu Thr Phe
                1395 1400 1405
Asn Val Pro Asp Met Asp Gly Val Gly Ser Ala Leu Gly Asn Leu Gly
          1410 1415 1420
Glu Leu Glu Val Ser Gly Glu Ala Ile Arg Thr Asp Gln Thr Lys Val
1425 1430 1435 1440
Asp Leu Ala Phe Thr Cys Thr Glu Met Tyr Ala Ala Asp Gly Ala Ala
                                1445 1450 1455
Ser Gly Met Arg Gly Val Leu Glu Tyr Arg Leu Asp Val Phe Gly Ala
                    1460 1465 1470
Val Gln Ala Arg Glu Thr Thr Glu Arg Leu Val Arg Val Leu Glu Gly
            1475 1480 1485
Val Val Ser Gly Gly Gly Gly Val Ser Val Ser Gly Val Asp Val Leu
      1490 1495 1500
Gly Val Gly Glu Arg Glu Arg Leu Leu Gly Trp Gly Val Gly Gly Pro
1505 1510 1515 1520
Val Pro Val Val Pro Gly Gly Gly Leu Val Gly Leu Phe Glu Glu Arg
                                  1525 1530 1535
Val Arg Ala Asp Ala Asp Ala Val Ala Val Arg Gly Ala Gly Val Val
                          1540 1545 1550
Trp Ser Tyr Gly Glu Leu Asn Ala Arg Val Asn Val Val Ala Arg Trp
                  1555 1560 1565
Leu Val Gly Arg Gly Val Gly Ala Glu Cys Gly Val Gly Val Val Met
          1570 1575 1580
Gly Arg Gly Val Asp Val Val Val Met Leu Leu Ala Val Ala Lys Ala
1585 1590 1595 1600
Gly Gly Phe Tyr Val Pro Val Asp Pro Glu Trp Pro Val Glu Arg Val
                                  1605 1610 1615
Gly Trp Val Leu Ala Asp Ala Gly Val Gly Leu Val Val Val Gly Glu
                        1620 1625 1630
Gly Leu Ser His Val Val Gly Asp Phe Pro Gly Gly Glu Val Phe Glu
                  1635 1640 1645
Phe Ser Arg Val Val Arg Glu Ser Cys Leu Val Glu Leu Val Ala Ala
          1650 1655 1660
Asp Gly Val Glu Val Arg Asn Val Thr Asp Gly Glu Arg Ala Ser Arg
1665 1670 1675 1680
Leu Leu Pro Gly His Pro Leu Tyr Val Val Tyr Thr Ser Gly Ser Thr
                                  1685 1690 1695
Gly Arg Pro Lys Gly Val Val Val Thr His Ala Ser Val Gly Gly Tyr
                          1700 1705 1710
Leu Ala Arg Gly Arg Asp Val Tyr Ala Gly Ala Val Gly Gly Val Gly
                1715 1720 1725
Phe Val His Ser Ser Leu Ala Phe Asp Leu Thr Val Thr Val Leu Phe
          1730 1735 1740
Thr Pro Leu Val Ser Gly Gly Cys Val Val Leu Gly Glu Leu Asp Glu
1745 1750 1755 1760
Ser Ala Gln Gly Val Gly Ala Ser Phe Val Lys Val Thr Pro Ser His
                                            1765 1770 1775
Leu Gly Leu Leu Gly Glu Leu Glu Gly Val Val Ala Gly Asn Gly Met
                                  1780 1785 1790
Leu Leu Val Gly Gly Glu Ala Leu Ser Gly Gly Ala Leu Arg Glu Trp
                          1795 1800 1805
Arg Glu Arg Asn Pro Gly Val Val Val Val Asn Ala Tyr Gly Pro Thr
                  1810 1815 1820
Glu Leu Thr Val Asn Cys Ala Glu Phe Leu Ile Ala Pro Gly Glu Glu
1825 1830 1835 1840
Val Pro Asp Gly Pro Val Pro Ile Gly Arg Pro Phe Ala Gly Gln Arg
                                    1845 1850 1855
Met Phe Val Leu Asp Ala Ala Leu Arg Val Val Pro Val Gly Val Val
                          1860 1865 1870
Gly Glu Leu Tyr Val Ala Gly Val Gly Leu Ala Arg Gly Tyr Leu Gly
                  1875 1880 1885
Arg Ala Gly Leu Thr Ala Glu Arg Phe Val Ala Cys Pro Phe Gly Ala
            1890 1895 1900
Pro Gly Glu Arg Met Tyr Arg Thr Gly Asp Leu Val Arg Trp Arg Val
1905 1910 1915 1920
Asp Gly Ala Leu Glu Phe Val Gly Arg Ala Asp Asp Gln Val Lys Val
                                        1925 1930 1935
Arg Gly Phe Arg Val Glu Leu Gly Glu Val Glu Gly Ala Val Ala Ala
                              1940 1945 1950
His Pro Asp Val Val Arg Ala Val Val Val Val Arg Glu Asp Arg Pro
                    1955 1960 1965
Gly Asp His Arg Leu Val Ala Tyr Val Thr Gly Val Asp Thr Gly Gly
          1970 1975 1980
Leu Ser Ser Ala Val Met Arg Ala Val Ala Glu Arg Leu Pro Ala Tyr
1985 1990 1995 2000
Met Val Pro Ser Ala Val Val Val Leu Asp Glu Ile Pro Leu Thr Pro
                                          2005 2010 2015
Asn Gly Lys Val Asp Arg Ala Ala Leu Pro Val Pro Gly Val Glu Ala
                                  2020 2025 2030
Gly Ala Gly Tyr Arg Ala Pro Val Ser Pro Arg Glu Glu Val Leu Cys
                        2035 2040 2045
Gly Leu Phe Ala Glu Val Leu Gly Leu Glu Arg Val Gly Val Asp Asp
            2050 2055 2060
Asp Phe Phe Gly Leu Gly Gly His Ser Leu Leu Ala Thr Arg Leu Ile
2065 2070 2075 2080
Ser Arg Val Arg Ala Val Leu Gly Val Glu Ala Gly Val Arg Ala Leu
                                          2085 2090 2095
Phe Glu Ala Pro Thr Val Ser Arg Leu Glu Arg Leu Leu Arg Glu Arg
                                  2100 2105 2110
Ser Ala Leu Gly Val Arg Val Pro Leu Val Ala Arg Glu Arg Thr Gly
                        2115 2120 2125
Arg Glu Pro Leu Ser Phe Ala Gln Gln Arg Leu Trp Phe Leu Glu Glu
            2130 2135 2140
Leu Glu Gly Pro Gly Ala Ala Tyr Asn Ile Pro Met Ala Leu Arg Leu
2145 2150 2155 2160
Ala Gly Val Leu Asp Val Glu Ala Leu His Gln Ala Leu Ile Asp Val
                                          2165 2170 2175
Ile Ala Arg His Glu Ser Leu Arg Thr Leu Ile Ala Gln Asp Ala Gly
                                  2180 2185 2190
Thr Ala Trp Gln His Ile Leu Pro Val Asp Asp Pro Arg Thr Arg Pro
                        2195 2200 2205
Gly Leu Pro Leu Val Asp Ile Gly Ala Asp Ala Leu Gln Glu Arg Leu
                  2210 2215 2220
Asp Glu Ala Ala Gly Arg Pro Phe Asp Leu Ala Ala Asp Leu Pro Val
2225 2230 2235 2240
Arg Ala Thr Val Phe Arg Leu Thr Asp Asn Asp His Ile Leu Leu Val
                                    2245 2250 2255
Val Ala His His Val Ala Phe Asp Ala Met Ser Arg Val Pro Phe Ile
                          2260 2265 2270
Arg Asn Val Lys Arg Ala Phe Glu Ala Arg Thr Asn Gly Ala Ala Pro
                    2275 2280 2285
Asp Trp Arg Pro Leu Pro Val Gln Tyr Ala Asp Tyr Ala Ala Trp Gln
            2290 2295 2300
Arg Asp Val Leu Gly Thr Glu Asp Asp Glu Ser Ser Glu Leu Ser Ala
2305 2310 2315 2320
Gln Leu Ala Tyr Trp Arg Thr Gln Leu Ala Ser Leu Pro Ala Glu Leu
                                    2325 2330 2335
Ala Leu Pro Thr Asp Arg Ala Arg Pro Ala Val Ala Ser Tyr Glu Gly
                          2340 2345 2350
Gly Lys Val Glu Phe Thr Val Pro Ala Gly Val Tyr Asp Gly Leu Val
                  2355 2360 2365
Ala Leu Ala Arg Ala Glu Gly Val Thr Val Phe Met Val Val Gln Ala
        2370 2375 2380
Ala Leu Ala Ala Leu Leu Ser Arg Leu Gly Ala Gly Asp Asp Ile Pro
2385 2390 2395 2400
Ile Gly Thr Pro Ile Ala Gly Arg Thr Asp Gln Ala Thr Glu Asp Leu
                                    2405 2410 2415
Ile Gly Phe Phe Val Asn Thr Leu Val Leu Arg Thr Asp Val Ser Gly
                          2420 2425 2430
Asp Pro Thr Phe Ala Glu Leu Leu Ala Arg Val Arg Ala Thr Asp Leu
                  2435 2440 2445
Asp Ala Tyr Ala His Gln Asp Ile Pro Phe Glu Arg Leu Val Glu Ala
            2450 2455 2460
Val Asn Pro Glu Arg Ser Leu Ala Arg His Pro Leu Phe Gln Val Met
2465 2470 2475 2480
Leu Thr Phe Asp Asn Thr Ile Asp Arg Glu Val Thr Glu Gly Phe Ala
                                    2485 2490 2495
Gly Leu Gly Val Glu Gly Leu Pro Leu Gly Ala Gly Ala Val Lys Phe
                          2500 2505 2510
Asp Leu Leu Phe Gly Leu Ser Glu Val Gly Gly Glu Leu Arg Gly Ala
                  2515 2520 2525
Val Glu Tyr Arg Cys Asp Leu Phe Asp His Pro Thr Val Ala Gln Leu
        2530 2535 2540
Ala Glu Arg Leu Val Arg Val Leu Glu Arg Val Ala Ser Asp Ala Ser
2545 2550 2555 2560
Val Arg Thr Gly Glu Leu Pro Val Val Gly Glu Ala Glu Arg Ala Arg
                                  2565 2570 2575
Val Leu Thr Glu Trp Asn Asp Thr Gly Val Pro Gly Val Pro Glu Thr
                          2580 2585 2590
Phe Leu Glu Leu Phe Glu Ala Gln Val Ala Ala Arg Gly Asp Ala Pro
                    2595 2600 2605
Ala Val Val Tyr Glu Gly Glu Val Leu Ser Tyr Arg Glu Leu Asp Ala
          2610 2615 2620
Arg Ala Asn Arg Leu Ala Gly Leu Leu Val Gly Arg Gly Ala Gly Pro
2625 2630 2635 2640
Glu His Phe Val Gly Val Ala Leu Pro Arg Gly Leu Asp Leu Ile Val
                                      2645 2650 2655
Ala Leu Leu Ala Val Leu Lys Ser Gly Ala Ala Tyr Val Pro Leu Asp
                          2660 2665 2670
Pro Glu Tyr Pro Ala Glu Arg Leu Val His Met Val Thr Asp Ala Ala
                  2675 2680 2685
Pro Val Val Val Val Thr Ser Thr Asp Val Arg Thr Leu Arg Thr Val
          2690 2695 2700
Pro Arg Val Glu Leu Asp Asp Glu Ala Thr Arg Ala Thr Leu Val Ala
2705 2710 2715 2720
Ala Pro Ala Thr Gly Pro Asp Val Lys Met Ser Ala Ser His Pro Ala
                                        2725 2730 2735
Tyr Val Ile Tyr Thr Ser Gly Ser Thr Gly Arg Pro Lys Gly Val Val
                          2740 2745 2750
Ile Ser His Gly Ser Leu Ala Asn Phe Leu Ala Trp Ala Arg Glu Asp
                  2755 2760 2765
Leu Gly Ala Glu Arg Leu Arg His Val Val Leu Ser Thr Ser Leu Ser
          2770 2775 2780
Phe Asp Val Ser Val Val Glu Leu Phe Ala Pro Leu Ser Cys Gly Gly
2785 2790 2795 2800
Thr Val Glu Ile Val Arg Asn Leu Leu Ala Leu Val Asp Arg Pro Gly
                                  2805 2810 2815
Arg Trp Ser Ala Ser Leu Val Ser Gly Val Pro Ser Ala Phe Ala Gln
                  2820 2825 2830
Leu Leu Glu Ala Gly Leu Asp Arg Ala Asp Val Gly Met Ile Ala Leu
          2835 2840 2845
Ala Gly Glu Ala Leu Ser Ala Arg Asp Val Arg Arg Val Arg Ala Val
    2850 2855 2860
Leu Pro Gly Ala Arg Val Ala Asn Phe Tyr Gly Pro Thr Glu Ala Thr
2865 2870 2875 2880
Val Tyr Ala Thr Ala Trp Tyr Gly Asp Thr Pro Met Asp Ala Ala Ala
                                    2885 2890 2895
Pro Met Gly Arg Pro Leu Arg Asn Thr Cys Val Tyr Val Leu Asp Asp
                          2900 2905 2910
Gly Leu Arg Val Val Pro Val Gly Val Val Gly Glu Leu Tyr Val Ala
                  2915 2920 2925
Gly Val Gly Leu Ala Arg Gly Tyr Leu Gly Arg Val Gly Leu Thr Ala
          2930 2935 2940
Glu Arg Phe Val Ala Cys Pro Phe Gly Ala Arg Gly Glu Arg Met Tyr
2945 2950 2955 2960
Arg Thr Gly Asp Leu Val Arg Trp Arg Val Asp Gly Thr Leu Glu Phe
                                2965 2970 2975
Val Gly Arg Ala Asp Asp Gln Val Lys Val Arg Gly Phe Arg Val Glu
                        2980 2985 2990
Leu Gly Glu Val Glu Gly Ala Val Ala Ala His Pro Asp Val Val Arg
                    2995 3000 3005
Ala Val Val Val Val Arg Glu Asp Arg Pro Gly Asp His Arg Leu Val
            3010 3015 3020
Ala Tyr Val Thr Gly Val Asp Thr Gly Gly Leu Ser Ser Ala Val Met
3025 3030 3035 3040
Arg Ala Val Ala Glu Arg Leu Pro Ala Tyr Met Val Pro Ser Ala Val
                                    3045 3050 3055
Val Val Leu Asp Glu Ile Pro Leu Thr Pro Asn Gly Lys Val Asp Arg
                          3060 3065 3070
Ala Gly Leu Pro Val Pro Val Val Ser Val Ala Gly Phe Cys Ala Pro
            3075 3080 3085
Ser Ser Pro Arg Glu Glu Val Leu Cys Gly Leu Phe Ala Glu Val Leu
      3090 3095 3100
Gly Val Glu Arg Val Gly Val Asp Asp Gly Phe Phe Asp Leu Gly Gly
3105 3110 3115 3120
Asp Ser Ile Leu Ser Ile Gln Leu Val Ala Arg Ala Arg Arg Ala Gly
                                        3125 3130 3135
Leu Glu Leu Ser Val Arg Asp Val Phe Glu Gly Arg Thr Val Arg Ala
                            3140 3145 3150
Leu Ala Ala Val Val Arg Gly Ser Asp Ala Gly Ala Val Gly Val Val
                  3155 3160 3165
Gly Gly Ala Glu Ile Val Leu Pro Gly Val Gly Glu Val Glu Arg Trp
      3170 3175 3180
Pro Val Val Glu Trp Leu Ala Glu Arg Gly Gly Gly Ser Leu Gly Gly
3185 3190 3195 3200
Val Val Arg Gly Phe Asn Gln Ser Val Val Leu Ala Val Pro Ala Gly
                                        3205 3210 3215
Leu Val Trp Glu Glu Leu Arg Val Leu Leu Gly Ala Val Arg Asp Arg
                          3220 3225 3230
His Glu Ala Trp Arg Leu Arg Val Leu Asp Ser Gly Ala Leu Cys Val
                  3235 3240 3245
Asp Gly Val Val Pro Asp Asp Gly Ser Trp Ile Val Arg Cys Asp Leu
      3250 3255 3260
Ser Gly Met Gly Val Asp Gly Gln Val Asp Ala Val Arg Ala Ala Ala
3265 3270 3275 3280
Val Glu Ala Arg Ala Trp Leu Asp Pro Ser Val Gly Arg Val Val Arg
                                    3285 3290 3295
Ala Val Trp Leu Glu Arg Gly Gly Asp Arg Ser Gly Val Leu Val Leu
                        3300 3305 3310
Val Ala His His Leu Val Val Asp Gly Val Ser Trp Arg Val Val Leu
                3315 3320 3325
Gly Asp Leu Ala Glu Gly Trp Ala Gln Val Arg Ser Gly Gly Arg Val
      3330 3335 3340
Glu Leu Gly Val Val Gly Thr Ser Leu Arg Gly Trp Ala Ala Ala Leu
3345 3350 3355 3360
Ala Glu Gln Gly Arg Arg Gly Glu Arg Ala Gly Glu Val Glu Leu Trp
                                3365 3370 3375
Ser Arg Met Val Arg Gly Ala Asp Val Leu Val Gly Ser Arg Ala Val
                    3380 3385 3390
Asp Gly Ala Val Asp Val Phe Gly Gly Val Val Ser Val Asp Ser Arg
            3395 3400 3405
Ala Ser Val Ser Val Ser Arg Ala Leu Leu Thr Glu Val Pro Ser Val
    3410 3415 3420
Leu Gly Val Gly Val Gln Glu Val Leu Leu Ala Ala Phe Gly Leu Ala
3425 3430 3435 3440
Val Ala Arg Trp Arg Gly Arg Gly Gly Pro Val Val Val Asp Val Glu
                        3445 3450 3455
Gly His Gly Arg Asn Glu Asp Ala Val Arg Gly Ala Asp Leu Ser Arg
                  3460 3465 3470
Thr Val Gly Trp Phe Thr Ser Val Tyr Pro Val Arg Val Pro Val Glu
      3475 3480 3485
Ser Ala Ser Trp Asp Glu Val Arg Ala Gly Gly Pro Val Val Gly Arg
    3490 3495 3500
Val Val Arg Glu Val Lys Glu Thr Leu Arg Ser Leu Pro Asp Gln Gly
3505 3510 3515 3520
Leu Gly Tyr Gly Ile Leu Arg Tyr Leu Asp Pro Glu His Gly Pro Ala
                          3525 3530 3535
Leu Ala Arg His Ala Thr Pro Gln Phe Gly Phe Asn Tyr Leu Gly Arg
                  3540 3545 3550
Phe Thr Thr Gly Thr Asp Asp Thr Gly Asp Glu Gly Met Thr Asp Trp
          3555 3560 3565
Val Pro Val Ser Gly Pro Phe Ala Val Gly Ala Gly Gln Asp Pro Glu
      3570 3575 3580
Leu Pro Val Ala His Ala Val Glu Phe Asn Ala Ile Thr Leu Asp Thr
3585 3590 3595 3600
Pro Glu Gly Pro Arg Leu Gly Val Thr Trp Ser Trp Pro Thr Thr Leu
                                          3605 3610 3615
Leu Pro Glu Ser Arg Ile Arg Glu Leu Ala Arg Tyr Trp Asp Glu Ala
                                  3620 3625 3630
Leu Glu Gly Leu Val Glu His Ala Arg His Pro Glu Ala Gly Gly Leu
                          3635 3640 3645
Thr Pro Ser Asp Val Thr Leu Val Glu Val Asn Gln Val Glu Leu Asp
                    3650 3655 3660
Arg Leu Gln Ala Gly Val Ala Gly Gly Ala Glu Glu Ile Leu Pro Val
3665 3670 3675 3680
Ser Ala Leu Gln Glu Gly Leu Leu Phe His Ser Ala Leu Ala Ser Gly
                                          3685 3690 3695
Gly Val Asp Val Tyr Val Gly Gln Leu Val Phe Asp Leu Val Gly Pro
                                  3700 3705 3710
Val Asp Val Asp Arg Leu Arg Ala Ala Val Glu Gly Leu Val Ala Arg
                          3715 3720 3725
His Gly Val Leu Arg Ser Gly Tyr Arg Gln Leu Arg Ser Gly Glu Trp
                  3730 3735 3740
Val Ala Val Val Ala Arg Gln Val Asp Leu Pro Trp Gln Ser Ile Asp
3745 3750 3755 3760
Val Arg Asp Gly Gly Ile Asp Gly Leu Val Glu Glu Glu Arg Trp Arg
                                          3765 3770 3775
Arg Phe Asp Met Gly Arg Gly Pro Leu Ala Arg Phe Val Leu Ile Arg
                                  3780 3785 3790
Thr His Asp Asp Arg Phe Arg Phe Val Ile Thr Tyr His His Val Val
                          3795 3800 3805
Leu Asp Gly Trp Ser Val Pro Val Leu Leu Arg Glu Leu Leu Ala Leu
                  3810 3815 3820
Tyr Gly Ser Ser Gly Asp Val Ser Val Leu Pro Gly Val Arg Ser Tyr
3825 3830 3835 3840
Gly Asp Phe Leu Arg Trp Val Ala Ala Arg Asp Ala Ala Ala Ala Glu
                                  3845 3850 3855
Gly Ala Trp Arg Arg Ala Leu Thr Gly Leu Glu Glu Pro Ser Leu Val
                          3860 3865 3870
Ala Pro Gly Val Ser Arg Asp Gly Val Val Pro Ala Ala Phe His Gly
                    3875 3880 3885
Ala Val Asp Gly Asp Leu Ser Gln Lys Ile Val Ala Trp Ala Arg Gly
3890 3895 3900
Arg Gly Val Thr Val Ala Ser Val Val Gln Ala Ala Trp Ala Leu Val
3905 3910 3915 3920
Leu Gly Arg Leu Met Gly Arg Asp Asp Val Val Phe Gly Val Thr Val
                                  3925 3930 3935
Ser Gly Arg Pro Ala Glu Val Val Gly Val Glu Asp Met Val Gly Leu
                          3940 3945 3950
Phe Val Asn Thr Ile Pro Leu Arg Ala Arg Leu Asp Pro Ala Glu Ser
                    3955 3960 3965
Leu Gly Gly Phe Val Glu Arg Leu Gln Arg Glu Gln Thr Glu Leu Leu
      3970 3975 3980
Glu His Gln His Val Arg Leu Ala Glu Val Gln Arg Trp Ala Gly His
3985 3990 3995 4000
Lys Glu Leu Phe Asp Val Gly Met Val Phe Asp Asn Tyr Pro Val Ser
                                    4005 4010 4015
Ser Glu Ser Pro Glu Ala Glu Phe Gln Ile Ser Arg Thr Gly Gly Tyr
                            4020 4025 4030
Asn Gly Thr His Tyr Ala Leu Asn Leu Val Ala Ser Met His Gly Leu
                  4035 4040 4045
Glu Leu Glu Leu Glu Ile Gly Tyr Arg Pro Asp Val Phe Asp Ala Gly
            4050 4055 4060
Arg Val Arg Glu Val Trp Gly Trp Leu Val Arg Val Leu Glu Gly Val
4065 4070 4075 4080
Val Ser Gly Gly Gly Gly Val Ser Val Ser Gly Val Asp Val Leu Gly
                                  4085 4090 4095
Val Gly Glu Arg Glu Arg Leu Leu Gly Trp Gly Val Gly Gly Pro Val
                          4100 4105 4110
Pro Val Val Pro Gly Gly Gly Leu Val Gly Leu Phe Glu Glu Arg Val
                4115 4120 4125
Arg Ala Asp Ala Asp Ala Val Ala Val Arg Gly Ala Gly Val Val Trp
    4130 4135 4140
Ser Tyr Gly Glu Leu Asn Ala Arg Val Asn Val Val Ala Arg Trp Leu
4145 4150 4155 4160
Val Gly Arg Gly Val Gly Ala Glu Cys Gly Val Gly Val Val Met Gly
                                  4165 4170 4175
Arg Gly Val Asp Val Val Val Met Leu Leu Ala Val Ala Lys Ala Gly
                            4180 4185 4190
Gly Phe Tyr Val Pro Val Asp Pro Glu Trp Pro Val Glu Arg Val Gly
                  4195 4200 4205
Trp Val Leu Ala Asp Ala Gly Val Gly Leu Val Val Val Gly Glu Gly
          4210 4215 4220
Leu Ser His Val Val Gly Asp Phe Pro Gly Gly Glu Val Phe Glu Phe
4225 4230 4235 4240
Ser Arg Val Val Arg Glu Ser Cys Leu Val Glu Leu Val Ala Ala Asp
                                  4245 4250 4255
Gly Val Glu Val Arg Asn Val Thr Asp Gly Glu Arg Ala Ser Arg Leu
                            4260 4265 4270
Leu Pro Gly His Pro Leu Tyr Val Val Tyr Thr Ser Gly Ser Thr Gly
                  4275 4280 4285
Arg Pro Lys Gly Val Val Val Thr His Ala Ser Val Gly Gly Tyr Leu
          4290 4295 4300
Ala Arg Gly Arg Asp Val Tyr Ala Gly Ala Val Gly Gly Val Gly Phe
4305 4310 4315 4320
Val His Ser Ser Leu Ala Phe Asp Leu Thr Val Thr Val Leu Phe Thr
                                    4325 4330 4335
Pro Leu Val Ser Gly Gly Cys Val Val Leu Gly Glu Leu Asp Glu Ser
                          4340 4345 4350
Ala Gln Gly Val Gly Ala Ser Phe Val Lys Val Thr Pro Ser His Leu
                    4355 4360 4365
Gly Leu Leu Gly Glu Leu Glu Gly Val Val Ala Gly Asn Gly Met Leu
          4370 4375 4380
Leu Val Gly Gly Glu Ala Leu Ser Gly Gly Ala Leu Arg Glu Trp Arg
4385 4390 4395 4400
Glu Arg Asn Pro Gly Val Val Val Val Asn Ala Tyr Gly Pro Thr Glu
                                  4405 4410 4415
Leu Thr Val Asn Cys Ala Glu Phe Leu Ile Ala Pro Gly Glu Glu Val
                          4420 4425 4430
Pro Asp Gly Pro Val Pro Ile Gly Arg Pro Phe Ala Gly Gln Arg Met
                  4435 4440 4445
Phe Val Leu Asp Ala Ala Leu Arg Val Val Pro Val Gly Val Val Gly
            4450 4455 4460
Glu Leu Tyr Val Ala Gly Val Gly Leu Ala Arg Gly Tyr Leu Gly Arg
4465 4470 4475 4480
Val Gly Leu Thr Ala Glu Arg Phe Val Ala Cys Pro Phe Gly Val Pro
                                    4485 4490 4495
Gly Glu Arg Met Tyr Arg Thr Gly Asp Leu Val Arg Trp Arg Val Asp
                        4500 4505 4510
Gly Ala Leu Glu Phe Val Gly Arg Ala Asp Asp Gln Val Lys Val Arg
                  4515 4520 4525
Gly Phe Arg Val Glu Leu Gly Glu Val Glu Gly Ala Val Ala Ala His
      4530 4535 4540
Pro Asp Val Val Arg Ala Val Val Val Val Arg Glu Asp Arg Pro Gly
4545 4550 4555 4560
Asp His Arg Leu Val Ala Tyr Val Thr Ala Gly Gly Val Gly Gly Asp
                                  4565 4570 4575
Gly Leu Arg Ser Ala Ile Ser Gly Leu Val Ala Glu Arg Leu Pro Ala
                        4580 4585 4590
Tyr Met Val Pro Ser Ala Val Val Val Leu Asp Glu Ile Pro Leu Thr
                  4595 4600 4605
Pro Asn Gly Lys Val Asp Arg Ala Ala Leu Pro Val Pro Glu Val Glu
          4610 4615 4620
Ala Gly Thr Gly Tyr Arg Ala Pro Val Ser Pro Arg Glu Glu Val Leu
4625 4630 4635 4640
Cys Gly Leu Phe Ala Glu Val Leu Gly Val Glu Arg Val Gly Val Asp
                              4645 4650 4655
Asp Asp Phe Phe Glu Leu Gly Gly His Ser Leu Leu Ala Thr Arg Leu
                    4660 4665 4670
Ile Ser Arg Val Arg Ala Val Leu Gly Val Glu Ala Gly Val Arg Ala
            4675 4680 4685
Leu Phe Glu Ala Pro Thr Val Ser Arg Leu Glu Arg Leu Leu Arg Glu
      4690 4695 4700
Arg Ser Gly Leu Gly Val Arg Val Pro Leu Val Ala Arg Glu Arg Thr
4705 4710 4715 4720
Gly Arg Glu Pro Leu Ser Phe Ala Gln Gln Arg Leu Trp Phe Leu Glu
                                4725 4730 4735
Glu Leu Glu Gly Pro Gly Ala Ala Tyr Asn Ile Pro Met Ala Leu Arg
                    4740 4745 4750
Leu Ala Gly Val Leu Asp Val Glu Ala Leu His Gln Ala Leu Ile Asp
          4755 4760 4765
Val Ile Ala Arg His Glu Ser Leu Arg Thr Leu Ile Ala Gln Asp Ala
    4770 4775 4780
Gly Thr Ala Trp Gln His Ile Leu Pro Val Asp Asp Pro Arg Thr Arg
4785 4790 4795 4800
Pro Gly Leu Pro Leu Val Asp Ile Gly Ala Asp Ala Leu Gln Glu Arg
                                          4805 4810 4815
Leu Asp Glu Ala Ala Gly Arg Pro Phe Asp Leu Ala Ala Asp Leu Pro
                                  4820 4825 4830
Val Arg Ala Thr Val Phe Arg Leu Thr Asp Asn Asp His Ile Leu Leu
                          4835 4840 4845
Leu Val Leu His His Ile Ala Gly Asp Gly Trp Ser Met Gly Pro Leu
                  4850 4855 4860
Ala Arg Asp Leu Ser Thr Ala Tyr Ser Ala Arg Ala Ala Gly Ala Ala
4865 4870 4875 4880
Ser Ala Trp Arg Pro Leu Ser Val Gln Tyr Ala Asp Tyr Ala Ala Trp
                                    4885 4890 4895
Gln Arg Asp Val Leu Gly Thr Glu Asp Asp Glu Ser Ser Glu Leu Ser
                          4900 4905 4910
Ala Gln Leu Ala Tyr Trp Arg Thr Gln Leu Ala Ser Leu Pro Ala Glu
            4915 4920 4925
Leu Ala Leu Pro Thr Asp Arg Ala Arg Pro Ala Val Ala Thr Tyr Arg
    4930 4935 4940
Gly Gly Arg Ile Glu Phe Thr Ile Pro Ala Asp Val His Arg Ser Leu
4945 4950 4955 4960
Ala Asp Leu Ala Arg Ala Glu Gly Val Thr Val Phe Met Val Val Gln
                            4965 4970 4975
Ala Ala Leu Ala Ala Leu Leu Ser Arg Leu Gly Ala Gly Asp Asp Ile
                    4980 4985 4990
Pro Ile Gly Thr Pro Ile Ala Gly Arg Thr Asp Gln Ala Thr Glu Asp
            4995 5000 5005
Leu Ile Gly Phe Phe Val Asn Thr Leu Val Leu Arg Thr Asp Val Ser
    5010 5015 5020
Gly Asp Pro Thr Phe Ala Glu Leu Leu Ala Arg Val Arg Ala Thr Asp
5025 5030 5035 5040
Leu Asp Ala Tyr Ala His Gln Asp Ile Pro Phe Glu Arg Leu Val Glu
                                  5045 5050 5055
Ala Val Asn Pro Glu Arg Ser Leu Ala Arg His Pro Leu Phe Gln Val
                        5060 5065 5070
Met Leu Ala Phe Asn Asn Ala Glu Thr Ser Thr Pro Leu Pro Met Ala
              5075 5080 5085
Glu Gly Leu Ala Ala Ser Arg Gln Asp Ile Glu Pro Gly Val Ala Lys
    5090 5095 5100
Phe Asp Leu Ala Leu Tyr Cys Asn Glu Ser Arg Gly Glu Thr Gly Asp
5105 5110 5115 5120
His Gln Gly Ile Arg Ser Val Phe Glu Tyr Arg Arg Asp Leu Trp Asp
                                5125 5130 5135
Glu Asp Thr Val Arg Gln Leu Ala Asp Arg Phe Leu His Val Leu Ala
                    5140 5145 5150
Ala Phe Ala Ala Ala Pro Glu Gln Arg Ala Ser Ser Val Asp Val Leu
            5155 5160 5165
Arg Ala Gly Glu Arg Asp Gln Leu Leu His Glu Trp Asn Asp Thr Ala
    5170 5175 5180
Ala Ala Leu Pro Pro Ala Leu Leu Pro Gln Leu Phe Glu Glu Gln Val
5185 5190 5195 5200
Arg Arg Thr Pro His Asp Val Ala Leu Val Ser Gly Asn Ile Arg Leu
                                        5205 5210 5215
Thr Tyr Ala Glu Leu Asp Ala Arg Ala Asn Arg Leu Ala His Leu Leu
                            5220 5225 5230
Leu Ala Arg Gly Ala Ala Pro Glu Thr Phe Val Ala Val Ala Leu Pro
                5235 5240 5245
Arg Thr Glu Glu Leu Leu Val Ala Leu Leu Ala Val Gln Lys Thr Gly
    5250 5255 5260
Ala Gly His Leu Pro Leu Asp Pro Gly Phe Pro Ala Glu Arg Leu Ser
5265 5270 5275 5280
Tyr Met Leu Asp Asp Ala Arg Pro Ala Val Val Leu Thr Thr Glu Asp
                                  5285 5290 5295
Ile Ser Ala Arg Ile Pro Gly Gly Ser His Val Val Leu Asp Ser Glu
                          5300 5305 5310
Gln Val Thr Gly Glu Leu His Asp His Pro Ala Thr Ser Pro Ala Gly
                  5315 5320 5325
Arg Gly Asn Pro Ala Gly Pro Ala Tyr Val Ile Tyr Thr Ser Gly Ser
          5330 5335 5340
Thr Gly Gln Pro Lys Gly Val Val Val Pro Ser Ala Ala Leu Val Asn
5345 5350 5355 5360
Phe Leu Ala Asp Met Val Pro Arg Leu Gly Leu Arg Gly Gly Asp Arg
                                          5365 5370 5375
Leu Leu Ser Val Thr Thr Val Gly Phe Asp Ile Ala Ala Leu Glu Leu
                          5380 5385 5390
Phe Val Pro Leu Leu Ser Gly Ala Thr Val Val Leu Ala Asp Gly Glu
                  5395 5400 5405
Thr Val Arg Asp Pro Ala Leu Ala Arg Gln Thr Cys Glu Asp His Gly
      5410 5415 5420
Val Thr Met Val Gln Ala Thr Pro Ser Trp Trp His Gly Met Leu Ala
5425 5430 5435 5440
Asp Ala Gly Asp Ser Leu Arg Gly Val His Ala Val Val Gly Gly Glu
                                5445 5450 5455
Ala Leu Ser Pro Gly Leu Arg Asp Ala Leu Thr Arg Gly Ala Arg Ser
                        5460 5465 5470
Val Thr Asn Met Tyr Gly Pro Thr Glu Thr Thr Ile Trp Ser Thr Ser
          5475 5480 5485
Ala Gly Gln Ala Ala Gly Asp Ser Ala Pro Pro Ser Ile Gly Thr Pro
    5490 5495 5500
Ile Leu Asn Thr Arg Val Tyr Val Leu Asp Ala Ala Leu Cys Val Val
5505 5510 5515 5520
Pro Pro Gly Val Ala Gly Glu Leu Tyr Ile Ala Gly Asp Gly Leu Ala
                        5525 5530 5535
Arg Gly Tyr Leu Gly Arg Ala Gly Leu Thr Ala Glu Arg Phe Val Ala
                  5540 5545 5550
Cys Pro Phe Gly Ala Pro Gly Glu Arg Met Tyr Arg Thr Gly Asp Leu
            5555 5560 5565
Val Arg Trp Arg Val Asp Gly Ala Leu Glu Phe Val Gly Arg Ala Asp
  5570 5575 5580
Asp Gln Val Lys Val Arg Gly Phe Arg Val Glu Leu Gly Glu Val Glu
5585 5590 5595 5600
Gly Ala Val Ala Ala His Pro Asp Val Val Arg Ala Val Val Val Val
                                5605 5610 5615
Arg Glu Asp Arg Pro Gly Asp His Arg Leu Val Ala Tyr Val Thr Gly
                    5620 5625 5630
Val Asp Thr Gly Gly Leu Ser Ser Ala Val Met Arg Ala Val Ala Glu
            5635 5640 5645
Arg Leu Pro Ala Tyr Met Val Pro Ser Ala Val Val Val Leu Asp Glu
    5650 5655 5660
Ile Pro Leu Thr Pro Asn Gly Lys Val Asp Arg Ala Ala Leu Pro Val
5665 5670 5675 5680
Pro Gly Val Glu Ala Gly Ala Gly Tyr Arg Ala Pro Val Ser Pro Arg
                            5685 5690 5695
Glu Glu Val Leu Cys Gly Leu Phe Ala Glu Val Leu Gly Val Glu Arg
                    5700 5705 5710
Val Gly Val Asp Asp Asp Phe Phe Gly Leu Gly Gly His Ser Leu Leu
          5715 5720 5725
Ala Thr Arg Leu Ile Ser Arg Val Arg Ala Val Leu Gly Val Glu Ala
    5730 5735 5740
Gly Val Arg Ala Leu Phe Glu Ala Pro Thr Val Ser Arg Leu Glu Arg
5745 5750 5755 5760
Leu Leu Arg Glu Arg Ser Gly Leu Gly Val Arg Val Pro Leu Val Ala
                                  5765 5770 5775
Arg Glu Arg Thr Gly Arg Glu Pro Leu Ser Phe Ala Gln Gln Arg Leu
                      5780 5785 5790
Trp Phe Leu Glu Glu Leu Glu Gly Pro Gly Ala Ala Tyr Asn Ile Pro
            5795 5800 5805
Met Ala Leu Arg Leu Ala Gly Val Leu Asp Val Glu Ala Leu His Gln
    5810 5815 5820
Ala Leu Ile Asp Val Ile Ala Arg His Glu Ser Leu Arg Thr Leu Ile
5825 5830 5835 5840
Ala Arg Asp Ser Asp Gly Thr Ala Arg Gln Gln Val Leu Pro Val Gly
                                  5845 5850 5855
Asp Pro Ala Ala Arg Pro Ala Leu Pro Val Val Gln Thr Asp Ala Asp
                      5860 5865 5870
Thr Leu Val Ala Lys Leu Asn Glu Ala Val Gly Arg Pro Phe Asp Leu
            5875 5880 5885
Thr Ala Glu Met Pro Leu Arg Ala Thr Val Phe Arg Val Ala Asp Glu
    5890 5895 5900
Asp His Ala Leu Leu Leu Val Phe His His Ile Ala Gly Asp Gly Trp
5905 5910 5915 5920
Ser Thr Gly Leu Leu Ala Arg Asp Leu Ser Thr Ala Tyr Ala Ala Arg
                                  5925 5930 5935
Leu Glu Gly Arg Asp Pro Gln Leu Pro Pro Leu Pro Val Gln Tyr Ala
                      5940 5945 5950
Asp Tyr Ala Ala Trp Gln Arg Asp Val Leu Gly Thr Glu Asp Asp Glu
            5955 5960 5965
Ser Ser Glu Leu Ser Ala Gln Leu Ala Tyr Trp Arg Thr Gln Leu Ala
    5970 5975 5980
Asp Leu Pro Ala Glu Leu Ala Leu Pro Ala Asp Arg Val Arg Pro Ala
5985 5990 5995 6000
Arg Ala Ser Tyr Glu Gly Gly Arg Val Gly Phe Thr Val Pro Ala Gly
                                  6005 6010 6015
Val Leu Arg Asp Leu Thr Arg Leu Ala Arg Val Glu Gly Val Thr Val
                      6020 6025 6030
Phe Met Val Val Gln Ala Ala Leu Ala Ala Leu Leu Ser Arg Leu Gly
            6035 6040 6045
Ala Gly Asp Asp Ile Pro Ile Gly Thr Pro Ile Ala Gly Arg Thr Asp
    6050 6055 6060
Gln Ala Thr Glu Asp Leu Ile Gly Phe Phe Val Asn Thr Leu Val Leu
6065 6070 6075 6080
Arg Thr Asp Val Ser Gly Asp Pro Thr Phe Ala Glu Leu Leu Ala Arg
                                  6085 6090 6095
Val Arg Ala Thr Asp Leu Asp Ala Tyr Ala His Gln Asp Ile Pro Phe
                      6100 6105 6110
Glu Arg Leu Val Glu Ala Val Asn Pro Glu Arg Ser Leu Ala Arg His
            6115 6120 6125
Pro Leu Phe Gln Val Met Leu Ala Phe Asp Asn Thr Ala Asp Gly Gly
    6130 6135 6140
Pro Val Glu Asp Phe Pro Gly Leu Ser Ala Ala Gly Leu Pro Leu Gly
6145 6150 6155 6160
Ala Gly Ala Ala Lys Phe Asp Leu Leu Phe Gly Leu Ser Glu Val Gly
                                  6165 6170 6175
Gly Glu Leu Arg Gly Ala Val Glu Tyr Arg Cys Asp Leu Phe Asp His
                      6180 6185 6190
Pro Thr Ala Ala Arg Ile Ala Glu Arg Leu Val Arg Val Leu Glu Arg
            6195 6200 6205
Val Ala Ala Asp Ala Ser Val Arg Leu Gly Glu Leu Pro Val Val Ser
    6210 6215 6220
Asp Ala Glu Arg Ala Cys Val Leu Thr Glu Trp Asn Asp Thr Ala Val
6225 6230 6235 6240
Pro Gly Val Thr Gly Thr Leu Ser Ala Leu Phe Glu Ala Arg Ala Ala
                                  6245 6250 6255
Ala Arg Gly Asp Ala Pro Ala Val Val Tyr Glu Gly Glu Glu Leu Ser
                      6260 6265 6270
Tyr Arg Glu Leu Asn Thr Arg Ala Asn Arg Leu Ala His Val Leu Ala
            6275 6280 6285
Glu His Gly Ala Gly Pro Glu Arg Phe Val Gly Val Ala Leu Pro Arg
    6290 6295 6300
Ser Pro Asp Leu Val Val Ala Leu Leu Ala Val Val Lys Ser Gly Ala
6305 6310 6315 6320
Ala Tyr Val Pro Leu Asp Pro Glu Tyr Pro Ala Asp Arg Leu Ala Tyr
                                  6325 6330 6335
Met Ala Gly Asp Ala Ala Pro Val Ala Val Leu Thr Arg Gly Asp Val
                          6340 6345 6350
Glu Leu Pro Gly Ser Val Pro Arg Ile Gly Leu Asp Asp Thr Glu Ile
            6355 6360 6365
Arg Ala Thr Leu Ala Thr Ala Pro Gly Thr Asn Pro Gly Thr Pro Val
      6370 6375 6380
Thr Glu Ala His Pro Ala Tyr Met Ile Tyr Thr Ser Gly Ser Thr Gly
6385 6390 6395 6400
Arg Pro Lys Gly Val Val Val Ser His Gly Ala Ile Val Asn Arg Leu
                                  6405 6410 6415
Ala Trp Met Gln Ala Glu Tyr Arg Leu Asp Ala Thr Asp Arg Val Leu
                      6420 6425 6430
Gln Lys Thr Pro Ala Gly Phe Asp Val Ser Val Trp Glu Phe Phe Trp
          6435 6440 6445
Pro Leu Leu Glu Gly Ala Val Leu Val Phe Ala Arg Pro Gly Gly His
    6450 6455 6460
Arg Asp Ala Ala Tyr Leu Ala Gly Leu Ile Glu Arg Glu Arg Ile Thr
6465 6470 6475 6480
Thr Ala His Phe Val Pro Ser Met Leu Arg Val Phe Leu Glu Glu Pro
                                  6485 6490 6495
Gly Ala Ala Leu Cys Thr Gly Leu Arg Arg Val Ile Cys Ser Gly Glu
                      6500 6505 6510
Ala Leu Gly Thr Asp Leu Ala Val Asp Phe Arg Ala Lys Leu Pro Val
            6515 6520 6525
Pro Leu His Asn Leu Tyr Gly Pro Thr Glu Ala Ala Val Asp Val Thr
    6530 6535 6540
His His Ala Tyr Glu Pro Ala Thr Gly Thr Ala Thr Val Pro Ile Gly
6545 6550 6555 6560
Arg Pro Ile Trp Asn Ile Arg Thr Tyr Val Leu Asp Ala Ala Leu Arg
                                  6565 6570 6575
Pro Val Pro Pro Gly Val Pro Gly Glu Leu Tyr Leu Ala Gly Ala Gly
                      6580 6585 6590
Leu Ala Arg Gly Tyr His Gly Arg Pro Ala Leu Thr Ala Glu Arg Phe
          6595 6600 6605
Val Ala Cys Pro Phe Gly Val Pro Gly Glu Arg Met Tyr Arg Thr Gly
    6610 6615 6620
Asp Leu Val Arg Trp Arg Val Asp Gly Thr Leu Glu Phe Val Gly Arg
6625 6630 6635 6640
Ala Asp Asp Gln Val Lys Val Arg Gly Phe Arg Val Glu Leu Gly Glu
                                  6645 6650 6655
Val Glu Gly Ala Val Ala Ala His Pro Asp Val Val Arg Ala Val Val
                        6660 6665 6670
Val Val Arg Glu Asp Arg Pro Gly Asp His Arg Leu Val Ala Tyr Val
          6675 6680 6685
Thr Val Gly Gly Val Gly Gly Asp Gly Leu Arg Ser Ala Ile Ser Gly
    6690 6695 6700
Leu Val Ala Glu Arg Leu Pro Ala Tyr Met Val Pro Ser Ala Val Val
6705 6710 6715 6720
Val Leu Asp Glu Ile Pro Leu Thr Pro Asn Gly Lys Val Asp Arg Ala
                                  6725 6730 6735
Gly Leu Pro Val Pro Val Val Ser Val Ala Gly Phe Cys Ala Pro Ser
                      6740 6745 6750
Ser Pro Arg Glu Glu Val Leu Cys Gly Leu Phe Ala Glu Val Leu Gly
            6755 6760 6765
Val Glu Arg Val Gly Val Asp Asp Gly Phe Phe Asp Leu Gly Gly Asp
    6770 6775 6780
Ser Ile Leu Ser Ile Gln Leu Val Ala Arg Ala Arg Arg Ala Gly Leu
6785 6790 6795 6800
Glu Leu Ser Val Arg Asp Val Phe Glu Gly Arg Thr Val Arg Ala Leu
                                  6805 6810 6815
Ala Ala Val Val Arg Gly Ser Asp Ala Gly Ala Val Gly Val Val Gly
                      6820 6825 6830
Gly Ala Glu Ile Val Leu Pro Gly Val Gly Glu Val Glu Arg Trp Pro
          6835 6840 6845
Val Val Glu Trp Leu Ala Glu Arg Gly Gly Gly Ser Leu Gly Gly Val
    6850 6855 6860
Val Arg Gly Phe Asn Gln Ser Val Val Leu Ala Val Pro Ala Gly Leu
6865 6870 6875 6880
Val Trp Glu Glu Leu Arg Val Leu Leu Gly Ala Val Arg Asp Arg His
                                  6885 6890 6895
Glu Ala Trp Arg Leu Arg Val Leu Asp Ser Gly Ala Leu Cys Val Asp
                      6900 6905 6910
Gly Val Val Pro Asp Asp Gly Ser Trp Ile Val Arg Cys Asp Leu Ser
          6915 6920 6925
Gly Met Gly Val Asp Gly Gln Val Asp Ala Val Arg Ala Ala Ala Val
    6930 6935 6940
Glu Ala Arg Ala Trp Leu Asp Pro Ser Val Gly Arg Val Val Arg Ala
6945 6950 6955 6960
Val Trp Leu Glu Arg Gly Gly Asp Arg Ser Gly Val Leu Val Leu Val
                                  6965 6970 6975
Ala His His Leu Val Val Asp Gly Val Ser Trp Arg Val Val Leu Gly
                      6980 6985 6990
Asp Leu Ala Glu Gly Trp Ala Gln Val Arg Ser Gly Gly Arg Val Glu
          6995 7000 7005
Leu Gly Val Val Gly Thr Ser Leu Arg Gly Trp Ala Ala Ala Leu Ala
    7010 7015 7020
Glu Gln Gly Arg Arg Gly Glu Arg Ala Gly Glu Val Glu Leu Trp Ser
7025 7030 7035 7040
Arg Met Val Arg Gly Ala Asp Val Leu Val Gly Ser Arg Ala Val Asp
                                  7045 7050 7055
Gly Ala Val Asp Val Phe Gly Gly Val Val Ser Val Asp Ser Arg Ala
                        7060 7065 7070
Ser Val Ser Val Ser Arg Ala Leu Leu Thr Glu Val Pro Ser Val Leu
          7075 7080 7085
Gly Val Gly Val Gln Glu Val Leu Leu Ala Ala Phe Gly Leu Ala Val
    7090 7095 7100
Ala Arg Trp Arg Gly Arg Gly Gly Pro Val Val Val Asp Val Glu Gly
7105 7110 7115 7120
His Gly Arg Asn Glu Asp Ala Val Arg Gly Ala Asp Leu Ser Arg Thr
                                  7125 7130 7135
Val Gly Trp Phe Thr Ser Val Tyr Pro Val Arg Val Pro Val Glu Ser
                      7140 7145 7150
Ala Ser Trp Asp Glu Val Arg Ala Gly Gly Pro Val Val Gly Arg Val
          7155 7160 7165
Val Arg Glu Val Lys Glu Thr Leu Arg Ser Leu Pro Asp Gln Gly Leu
    7170 7175 7180
Gly Tyr Gly Ile Leu Arg Tyr Leu Asp Pro Glu His Gly Pro Ala Leu
7185 7190 7195 7200
Ala Arg His Ala Thr Pro Gln Phe Gly Phe Asn Tyr Leu Gly Arg Phe
                                  7205 7210 7215
Thr Thr Gly Thr Asp Glu Thr Thr Thr Ala Asp Ala Leu Asp Arg Ala
                      7220 7225 7230
Pro Ala Trp Ser Leu Leu Ala Arg Ser Ala Ala Gly Gln Asp Pro Glu
          7235 7240 7245
Leu Pro Val Ala His Ala Val Glu Phe Asn Ala Ile Thr Leu Asp Thr
    7250 7255 7260
Pro Glu Gly Pro Arg Leu Gly Val Thr Trp Ser Trp Pro Thr Thr Leu
7265 7270 7275 7280
Leu Pro Glu Ser Arg Ile Arg Glu Leu Ala Arg Tyr Trp Asp Glu Ala
                                  7285 7290 7295
Leu Glu Gly Leu Val Glu His Ala Arg His Pro Glu Ala Gly Gly Leu
                      7300 7305 7310
Thr Pro Ser Asp Val Gly Leu Ala Glu Leu Ser Phe Ala Glu Ile Glu
            7315 7320 7325
Leu Leu Glu Asp Asp Trp Arg Thr Gln Gly
    7330 7335
dptA daptomycin non-ribosomal peptide synthetase A
SEQ ID NO: 34
Met Asp Met Gln Ser Gln Arg Leu Gly Val Thr Ala Ala Gln Gln Ser
    1 5 10 15
Val Trp Leu Ala Gly Gln Leu Ala Asp Asp His Arg Leu Tyr His Cys
                              20 25 30
Ala Ala Tyr Leu Ser Leu Thr Gly Ser Ile Asp Pro Arg Thr Leu Gly
                      35 40 45
Thr Ala Val Arg Arg Thr Leu Asp Glu Thr Glu Ala Leu Arg Thr Arg
          50 55 60
Phe Val Pro Gln Asp Gly Glu Leu Leu Gln Ile Leu Glu Pro Gly Ala
  65 70 75 80
Gly Gln Leu Leu Leu Glu Ala Asp Phe Ser Gly Asp Pro Asp Pro Glu
                                      85 90 95
Arg Ala Ala His Asp Trp Met His Ala Ala Leu Ala Ala Pro Val Arg
                            100 105 110
Leu Asp Arg Ala Gly Thr Ala Thr His Ala Leu Leu Thr Leu Gly Pro
                  115 120 125
Ser Arg His Leu Leu Tyr Phe Gly Tyr His His Ile Ala Leu Asp Gly
          130 135 140
Tyr Gly Ala Leu Leu His Leu Arg Arg Leu Ala His Val Tyr Thr Ala
145 150 155 160
Leu Ser Asn Gly Asp Asp Pro Gly Pro Cys Pro Phe Gly Pro Leu Ala
                                    165 170 175
Gly Val Leu Thr Glu Glu Ala Ala Tyr Arg Asp Ser Asp Asn His Arg
                            180 185 190
Arg Asp Gly Glu Phe Trp Thr Arg Ser Leu Ala Gly Ala Asp Glu Ala
                  195 200 205
Pro Gly Leu Ser Glu Arg Glu Ala Gly Ala Leu Ala Val Pro Leu Arg
          210 215 220
Arg Thr Val Glu Leu Ser Gly Glu Arg Thr Glu Lys Leu Ala Ala Ser
225 230 235 240
Ala Ala Ala Thr Gly Ala Arg Trp Ser Ser Leu Leu Val Ala Ala Thr
                                    245 250 255
Ala Ala Phe Val Arg Arg His Ala Ala Ala Asp Asp Thr Val Ile Gly
                            260 265 270
Leu Pro Val Thr Ala Arg Leu Thr Gly Pro Ala Leu Arg Thr Pro Cys
                    275 280 285
Met Leu Ala Asn Asp Val Pro Leu Arg Leu Asp Ala Arg Leu Asp Ala
          290 295 300
Pro Phe Ala Ala Leu Leu Ala Asp Thr Thr Arg Ala Val Gly Thr Leu
305 310 315 320
Ala Arg His Gln Arg Phe Arg Gly Glu Glu Leu His Arg Asn Leu Gly
                                  325 330 335
Gly Val Gly Arg Thr Ala Gly Leu Ala Arg Val Thr Val Asn Val Leu
                            340 345 350
Ala Tyr Val Asp Asn Ile Arg Phe Gly Asp Cys Arg Ala Val Val His
                    355 360 365
Glu Leu Ser Ser Gly Pro Val Arg Asp Phe His Ile Asn Ser Tyr Gly
            370 375 380
Thr Pro Gly Thr Pro Asp Gly Val Gln Leu Val Phe Ser Gly Asn Pro
385 390 395 400
Ala Leu Tyr Thr Ala Thr Asp Leu Ala Asp His Gln Glu Arg Phe Leu
                                      405 410 415
Arg Phe Leu Asp Ala Val Thr Ala Asp Pro Asp Leu Pro Thr Gly Arg
                              420 425 430
His Arg Leu Leu Ser Pro Gly Thr Arg Ala Arg Leu Leu Asp Asp Ser
                    435 440 445
Arg Gly Thr Glu Arg Pro Val Pro Arg Ala Thr Leu Pro Glu Leu Phe
          450 455 460
Ala Glu Gln Ala Arg Arg Thr Pro Asp Ala Pro Ala Val Gln His Asp
465 470 475 480
Gly Thr Val Leu Thr Tyr Arg Asp Leu His Arg Ser Val Glu Arg Ala
                                    485 490 495
Ala Gly Arg Leu Ala Gly Leu Gly Leu Arg Thr Glu Asp Val Val Ala
                          500 505 510
Leu Ala Leu Pro Lys Ser Ala Glu Ser Val Ala Ile Leu Leu Gly Ile
                  515 520 525
Gln Arg Ala Gly Ala Ala Tyr Val Pro Leu Asp Pro Thr His Pro Ala
        530 535 540
Glu Arg Leu Ala Arg Val Leu Asp Asp Thr Arg Pro Arg Tyr Leu Val
545 550 555 560
Thr Thr Gly His Ile Asp Gly Leu Ser His Pro Thr Pro Gln Leu Ala
                                    565 570 575
Ala Ala Asp Leu Leu Arg Glu Gly Gly Pro Glu Pro Ala Pro Gly Arg
                          580 585 590
Pro Ala Pro Gly Asn Ala Ala Tyr Ile Ile Gln Thr Ser Gly Ser Thr
                    595 600 605
Gly Arg Pro Lys Gly Val Val Val Thr His Glu Gly Leu Ala Thr Leu
          610 615 620
Ala Ala Asp Gln Ile Arg Arg Tyr Arg Thr Gly Pro Asp Ala Arg Val
625 630 635 640
Leu Gln Phe Ile Ser Pro Gly Phe Asp Val Phe Val Ser Glu Leu Ser
                                    645 650 655
Met Thr Leu Leu Ser Gly Gly Cys Leu Val Ile Pro Pro Asp Gly Leu
                            660 665 670
Thr Gly Arg His Leu Ala Asp Phe Leu Ala Ala Glu Ala Val Thr Thr
                    675 680 685
Thr Ser Leu Thr Pro Gly Ala Leu Ala Thr Met Pro Ala Thr Asp Leu
          690 695 700
Pro His Leu Arg Thr Leu Ile Val Gly Gly Glu Val Cys Pro Pro Glu
705 710 715 720
Ile Phe Asp Gln Trp Gly Arg Gly Arg Asp Ile Val Asn Ala Tyr Gly
                                    725 730 735
Pro Thr Glu Thr Thr Val Glu Ala Thr Ala Trp His Arg Asp Gly Ala
                          740 745 750
Thr His Gly Pro Val Pro Leu Gly Arg Pro Thr Leu Asn Arg Arg Gly
                  755 760 765
Tyr Val Leu Asp Pro Ala Leu Glu Pro Val Pro Asp Gly Thr Thr Gly
          770 775 780
Glu Leu Tyr Leu Ala Gly Glu Gly Leu Ala Arg Gly Tyr Val Ala Ala
785 790 795 800
Pro Gly Pro Thr Ala Glu Arg Phe Val Ala Asp Pro Phe Gly Pro Pro
                                        805 810 815
Gly Ser Arg Met Tyr Arg Thr Gly Asp Leu Val Arg Arg Arg Ser Gly
                              820 825 830
Gly Met Leu Glu Phe Val Gly Arg Ala Asp Gly Gln Val Lys Leu Arg
                    835 840 845
Gly Phe Arg Ile Glu Leu Gly Glu Val Gln Ala Ala Leu Thr Ala Leu
            850 855 860
Pro Gly Val Arg Gln Ala Gly Val Leu Ile Arg Glu Asp Arg Pro Gly
865 870 875 880
Asp Pro Arg Leu Val Gly Tyr Ile Val Pro Ala Pro Gly Ala Glu Pro
                                    885 890 895
Asp Ala Gly Glu Leu Arg Ala Ala Leu Ala Arg Thr Leu Pro Pro His
                          900 905 910
Met Val Pro Trp Ala Leu Val Pro Leu Pro Ala Leu Pro Leu Thr Ser
                  915 920 925
Asn Gly Lys Leu Asp Arg Ala Ala Leu Pro Val Pro Ala Ala Arg Ala
        930 935 940
Gly Gly Ser Gly Gln Arg Pro Val Thr Pro Gln Glu Lys Thr Leu Cys
945 950 955 960
Ala Leu Phe Ala Asp Val Leu Gly Val Thr Glu Val Ala Thr Asp Asp
                                    965 970 975
Val Phe Phe Glu Leu Gly Gly His Ser Leu Asn Gly Thr Arg Leu Leu
                            980 985 990
Ala Arg Ile Arg Thr Glu Phe Gly Thr Asp Leu Thr Leu Arg Asp Leu
                  995 1000 1005
Phe Ala Phe Pro Thr Val Ala Gly Leu Leu Pro Leu Leu Asp Asp Asn
      1010 1015 1020
Gly Arg Gln His Thr Thr Pro Pro Leu Pro Pro Arg Pro Glu Arg Leu
1025 1030 1035 1040
Pro Leu Ser His Ala Gln Gln Arg Leu Trp Phe Leu Asp Gln Val Glu
                                  1045 1050 1055
Gly Pro Ser Pro Ala Tyr Asn Ile Pro Thr Ala Val Arg Leu Glu Gly
                          1060 1065 1070
Pro Leu Asp Ile Pro Ala Leu Ala Val Ala Leu Gln Asp Val Thr Asn
                  1075 1080 1085
Arg His Glu Pro Leu Arg Thr Leu Leu Ala Glu Asp Ser Glu Gly Pro
            1090 1095 1100
His Gln Val Ile Leu Pro Pro Glu Ala Ala Arg Pro Glu Leu Thr His
1105 1110 1115 1120
Ser Thr Val Ala Pro Gly Asp Leu Ala Ala Ala Leu Ala Glu Ala Ala
                                  1125 1130 1135
Arg Arg Pro Phe Asp Leu Ala Gly Glu Ile Pro Leu Lys Ala His Leu
                          1140 1145 1150
Phe Gly Cys Gly Pro Asp Asp His Thr Leu Leu Leu Leu Val His His
                1155 1160 1165
Thr Ala Gly Asp Gly Ala Ser Val Glu Val Leu Val Arg Asp Leu Ala
        1170 1175 1180
His Ala Tyr Gly Ala Arg Arg Ala Gly Asp Ala Pro His Phe Glu Pro
1185 1190 1195 1200
Leu Pro Leu Gln Tyr Ala Asp His Thr Leu Arg Arg Arg His Leu Leu
                                      1205 1210 1215
Asp Asp Pro Ser Asp Ser Thr Gln Leu Asp His Trp Arg Asp Ala Leu
                            1220 1225 1230
Ala Gly Leu Pro Glu Gln Leu Glu Leu Pro Thr Asp His Thr Arg Pro
                  1235 1240 1245
Ala Val Pro Thr Arg Arg Gly Glu Ala Ile Ala Phe Thr Val Pro Glu
            1250 1255 1260
His Thr His His Thr Leu Arg Ala Met Ala Gln Ala His Gly Val Thr
1265 1270 1275 1280
Val Phe Met Val Met Gln Ala Ala Leu Ala Ala Leu Leu Ser Arg His
                                1285 1290 1295
Gly Ala Gly His Asp Ile Pro Leu Gly Thr Pro Val Ala Gly Arg Ser
                        1300 1305 1310
Asp Asp Gly Thr Glu Asp Leu Val Gly Phe Phe Val Asn Thr Leu Val
                  1315 1320 1325
Leu Arg Asn Asp Val Ser Gly Asp Pro Thr Phe Ala Glu Leu Val Ser
              1330 1335 1340
Arg Val Arg Ala Ala Asn Leu Asp Ala Tyr Ala Tyr Gln Asp Val Pro
1345 1350 1355 1360
Phe Glu Arg Leu Val Asp Val Leu Lys Pro Glu Arg Ser Leu Ser Trp
                                    1365 1370 1375
His Pro Leu Phe Gln Ile Met Ile Ala Tyr Asn Gly Pro Ala Thr Asn
                          1380 1385 1390
Asp Thr Ala Asp Gly Ser Arg Phe Ala Gly Leu Thr Ser Arg Val His
                1395 1400 1405
Ala Val His Thr Gly Met Ser Lys Phe Asp Leu Ser Phe Phe Leu Thr
          1410 1415 1420
Glu His Ala Asp Gly Leu Gly Ile Asp Gly Ala Leu Glu Phe Ser Thr
1425 1430 1435 1440
Asp Leu Phe Thr Arg Ile Thr Ala Glu Arg Leu Val Gln Arg Tyr Leu
                                1445 1450 1455
Thr Val Leu Glu Gln Ala Ala Gly Ala Pro Asp Arg Pro Ile Ser Ser
                    1460 1465 1470
Tyr Glu Leu Leu Gly Asp Asp Glu Arg Ala Leu Leu Ala Gln Trp Asn
            1475 1480 1485
Asp Thr Ala His Pro Thr Pro Pro Gly Thr Val Leu Asp Leu Leu Glu
      1490 1495 1500
Ser Arg Ala Ala Arg Thr Pro Asp Arg Pro Ala Val Val Glu Asn Asp
1505 1510 1515 1520
His Val Leu Thr Tyr Ala Asp Leu His Thr Arg Ala Asn Arg Leu Ala
                                  1525 1530 1535
Arg His Leu Ile Thr Ala His Gly Val Gly Pro Glu Arg Leu Val Ala
                          1540 1545 1550
Val Ala Leu Pro Arg Ser Ala Glu Leu Leu Val Ala Leu Leu Ala Val
                  1555 1560 1565
Leu Lys Thr Gly Ala Ala Tyr Val Pro Leu Asp Leu Thr His Pro Ala
          1570 1575 1580
Glu Arg Thr Ala Val Val Leu Asp Asp Cys Arg Pro Ala Val Ile Leu
1585 1590 1595 1600
Thr Asp Ala Gly Ala Ala Arg Glu Leu Pro Arg Arg Asp Ile Pro Gln
                                  1605 1610 1615
Leu Arg Leu Asp Glu Pro Glu Val His Ala Ala Ile Ala Glu Gln Pro
                        1620 1625 1630
Gly Gly Pro Val Thr Asp Arg Asp Arg Thr Cys Val Thr Pro Val Ser
                  1635 1640 1645
Gly Glu His Val Ala Tyr Val Ile Tyr Thr Ser Gly Ser Thr Gly Arg
          1650 1655 1660
Pro Lys Gly Val Ala Val Glu His Arg Ser Leu Ala Asp Phe Val Arg
1665 1670 1675 1680
Tyr Ser Val Thr Ala Tyr Pro Gly Ala Phe Asp Val Thr Leu Leu His
                                  1685 1690 1695
Ser Pro Val Thr Phe Asp Leu Thr Val Thr Ser Leu Phe Pro Pro Leu
                          1700 1705 1710
Val Val Gly Gly Ala Ile His Val Ala Asp Leu Thr Glu Ala Cys Pro
                1715 1720 1725
Pro Ser Leu Ala Ala Ala Gly Gly Pro Thr Phe Val Lys Ala Thr Pro
          1730 1735 1740
Ser His Leu Pro Leu Leu Thr His Glu Ala Thr Trp Ala Ala Ser Ala
1745 1750 1755 1760
Lys Val Leu Leu Val Gly Gly Glu Gln Leu Leu Gly Arg Glu Leu Asp
                                            1765 1770 1775
Lys Trp Arg Ala Gly Ser Pro Glu Ala Val Val Phe Asn Asp Tyr Gly
                                  1780 1785 1790
Pro Thr Glu Ala Thr Val Asn Cys Val Asp Phe Arg Ile Asp Pro Gly
                          1795 1800 1805
Gln Pro Ile Gly Ala Gly Pro Val Ala Ile Gly Arg Pro Leu Arg Asn
                  1810 1815 1820
Thr Arg Val Phe Val Leu Asp Gly Gly Leu Arg Ala Val Pro Val Gly
1825 1830 1835 1840
Val Val Gly Glu Leu His Val Ala Gly Glu Gly Leu Ala Arg Gly Tyr
                                    1845 1850 1855
Leu Gly Gln Pro Gly Leu Thr Ala Glu Arg Phe Val Ala Cys Pro Phe
                          1860 1865 1870
Gly Asp Ala Gly Glu Arg Met Tyr Arg Thr Gly Asp Leu Val Arg Trp
                  1875 1880 1885
Arg Ala Asp Gly Met Leu Glu Phe Val Gly Arg Val Asp Asp Gln Val
            1890 1895 1900
Lys Val Arg Gly Phe Arg Ile Glu Leu Gly Glu Val Glu Ala Ala Val
1905 1910 1915 1920
Ala Ala Cys Pro Gly Val Asp Arg Ser Val Val Val Val Arg Glu Asp
                                        1925 1930 1935
Arg Pro Gly Asp Arg Arg Leu Val Ala Tyr Val Thr Ala Ala Gly Asp
                              1940 1945 1950
Glu Ala Glu Gly Leu Ala Pro Leu Ile Val Glu Thr Ala Ala Gly Arg
                    1955 1960 1965
Leu Pro Gly Tyr Met Val Pro Ser Ala Val Val Val Leu Asp Glu Ile
          1970 1975 1980
Pro Leu Thr Pro Asn Gly Lys Val Asp Arg Ala Ala Leu Pro Ala Pro
1985 1990 1995 2000
Arg Val Ala Pro Ala Ala Glu Phe Arg Val Thr Gly Ser Pro Arg Glu
                                          2005 2010 2015
Glu Ala Leu Cys Ala Leu Phe Ala Glu Val Leu Gly Val Glu Arg Val
                                  2020 2025 2030
Gly Val Asp Asp Gly Phe Phe Asp Leu Gly Gly Asp Ser Ile Leu Ser
                        2035 2040 2045
Ile Gln Leu Val Ala Arg Ala Arg Arg Ala Gly Leu Glu Val Ser Val
            2050 2055 2060
Arg Asp Val Phe Glu His Arg Thr Val Arg Ala Leu Ala Gly Val Val
2065 2070 2075 2080
Arg Glu Ser Gly Gly Val Ala Ala Ala Val Val Asp Ser Gly Val Gly
                                          2085 2090 2095
Ala Val Glu Arg Trp Pro Val Val Glu Trp Leu Ala Glu Arg Gly Gly
                                  2100 2105 2110
Gly Gly Leu Gly Gly Ala Val Arg Ala Phe Asn Gln Ser Val Val Val
                        2115 2120 2125
Ala Thr Pro Ala Gly Ile Thr Trp Asp Glu Leu Arg Thr Val Leu Asp
            2130 2135 2140
Ala Val Arg Glu Arg His Asp Ala Trp Arg Leu Arg Val Val Asp Ser
2145 2150 2155 2160
Gly Asp Gly Ala Trp Ser Leu Arg Val Asp Ala Pro Ala Pro Gly Gly
                                          2165 2170 2175
Glu Pro Asp Trp Ile Thr Arg His Gly Met Ala Ser Ala Asp Leu Glu
                                  2180 2185 2190
Glu Gln Val Asn Ala Val Arg Ala Ala Ala Val Glu Ala Arg Ser Arg
                        2195 2200 2205
Leu Asp Pro Leu Thr Gly Arg Met Val Arg Ala Val Trp Leu Asp Arg
                  2210 2215 2220
Gly Pro Asp Arg Arg Gly Val Leu Val Leu Val Ala His His Leu Val
2225 2230 2235 2240
Val Asp Gly Val Ser Trp Arg Ile Val Leu Gly Asp Leu Gly Glu Ala
                                    2245 2250 2255
Trp Thr Gln Ala Arg Ala Gly Gly His Val Arg Leu Asp Thr Val Gly
                          2260 2265 2270
Thr Ser Leu Arg Gly Trp Ala Ala Ala Leu Ala Glu Gln Gly Arg His
                    2275 2280 2285
Gly Ala Arg Ala Thr Glu Ala Asn Leu Trp Ala Gln Met Val His Gly
            2290 2295 2300
Ser Asp Pro Leu Val Gly Pro Arg Ala Val Asp Pro Ser Val Asp Val
2305 2310 2315 2320
Phe Gly Val Val Glu Ser Val Gly Ser Arg Ala Ser Val Gly Val Ser
                                    2325 2330 2335
Arg Ala Leu Leu Thr Glu Val Pro Ser Val Leu Gly Val Gly Val Gln
                          2340 2345 2350
Glu Val Leu Leu Ala Ala Phe Gly Leu Ala Val Thr Arg Trp Arg Gly
                  2355 2360 2365
Arg Gly Gly Ser Val Val Val Asp Val Glu Gly His Gly Arg Asn Glu
        2370 2375 2380
Asp Ala Val Pro Gly Ala Asp Leu Ser Arg Thr Val Gly Trp Phe Thr
2385 2390 2395 2400
Ser Ile Tyr Pro Val Arg Leu Pro Leu Glu Pro Ala Ala Trp Asp Glu
                                    2405 2410 2415
Ile Arg Ala Gly Gly Pro Ala Val Gly Arg Thr Val Arg Glu Ile Lys
                          2420 2425 2430
Glu Cys Leu Arg Thr Leu Pro Asp Gln Gly Leu Gly Tyr Gly Ile Leu
                  2435 2440 2445
Arg Tyr Leu Asp Pro Glu Asn Gly Pro Ala Leu Ala Gln His Pro Thr
            2450 2455 2460
Pro His Phe Gly Phe Asn Tyr Leu Gly Arg Val Ser Val Ser Ala Asp
2465 2470 2475 2480
Ala Ala Ser Leu Asp Glu Gly Asp Ala His Ala Asp Gly Leu Gly Gly
                                    2485 2490 2495
Leu Val Gly Gly Arg Ala Ala Ala Asp Ser Asp Glu Glu Gln Trp Ala
                          2500 2505 2510
Asp Trp Val Pro Val Ser Gly Pro Phe Ala Val Gly Ala Gly Gln Asp
                  2515 2520 2525
Pro Val Leu Pro Val Ala His Ala Val Glu Phe Asn Ala Ile Thr Leu
        2530 2535 2540
Asp Thr Pro Asp Gly Pro Arg Leu Ser Val Thr Trp Ser Trp Pro Thr
2545 2550 2555 2560
Thr Leu Leu Ser Glu Ser Arg Ile Arg Glu Leu Ala Arg Phe Trp Asp
                                  2565 2570 2575
Glu Ala Leu Glu Gly Leu Val Ala His Ala Arg Arg Pro Asp Ala Gly
                          2580 2585 2590
Gly Leu Thr Pro Ser Asp Leu Pro Leu Val Ala Leu Asp His Ala Glu
                    2595 2600 2605
Leu Glu Ala Leu Gln Ala Asp Val Thr Gly Gly Val His Asp Ile Leu
          2610 2615 2620
Pro Val Ser Pro Leu Gln Glu Gly Leu Leu Phe His Ser Ser Phe Ala
2625 2630 2635 2640
Ala Asp Gly Val Asp Val Tyr Val Gly Gln Leu Thr Phe Asp Leu Thr
                                      2645 2650 2655
Gly Pro Val Asp Ala Asp His Leu His Ala Val Val Glu Ser Leu Val
                          2660 2665 2670
Thr Arg His Asp Val Leu Arg Thr Gly Tyr Arg Gln Ala Gln Ser Gly
                  2675 2680 2685
Glu Trp Ile Ala Val Val Ala Arg Gln Val His Thr Pro Trp Gln Tyr
          2690 2695 2700
Ile His Thr Leu Asp Thr Asp Ala Asp Thr Leu Thr Asn Asp Glu Arg
2705 2710 2715 2720
Trp Arg Pro Phe Asp Met Thr Gln Gly Pro Leu Ala Arg Phe Thr Leu
                                        2725 2730 2735
Ala Arg Ile Asn Asp Thr His Phe Arg Phe Ile Val Thr Tyr His His
                          2740 2745 2750
Val Ile Leu Asp Gly Trp Ser Val Ala Val Leu Ile Arg Glu Leu Phe
                  2755 2760 2765
Thr Thr Tyr Arg Asp Thr Ala Leu Gly Arg Arg Pro Glu Val Pro Tyr
          2770 2775 2780
Ser Pro Pro Arg Arg Asp Phe Met Ala Trp Leu Ala Glu Arg Asp Gln
2785 2790 2795 2800
Thr Ala Ala Gly Gln Ala Trp Arg Ser Ala Leu Ala Gly Leu Ala Glu
                                  2805 2810 2815
Pro Thr Val Leu Ala Leu Gly Thr Glu Gly Ser Gly Val Ile Pro Glu
                  2820 2825 2830
Val Leu Glu Glu Glu Ile Ser Glu Glu Leu Thr Ser Glu Leu Val Ala
          2835 2840 2845
Trp Ala Arg Gly Arg Gly Val Thr Val Ala Ser Val Val Gln Ala Ala
    2850 2855 2860
Trp Ala Leu Val Leu Gly Arg Leu Val Gly Arg Asp Asp Val Val Phe
2865 2870 2875 2880
Gly Leu Thr Val Ser Gly Arg Pro Ala Glu Val Ala Gly Val Glu Asp
                                    2885 2890 2895
Met Val Gly Leu Phe Val Asn Thr Ile Pro Leu Arg Ala Arg Met Asp
                          2900 2905 2910
Pro Ala Glu Ser Leu Gly Ala Phe Val Glu Arg Leu Gln Arg Glu Gln
                  2915 2920 2925
Thr Glu Leu Leu Glu His Gln His Val Arg Leu Ala Glu Val Gln Arg
          2930 2935 2940
Trp Ala Gly His Lys Glu Leu Phe Asp Val Gly Met Val Phe Glu Asn
2945 2950 2955 2960
Tyr Pro Met Asp Ser Leu Leu Gln Asp Ser Leu Phe His Gly Ser Gly
                                2965 2970 2975
Leu Gln Ile Asp Gly Ile Gln Gly Ala Asp Ala Thr His Phe Ala Leu
                        2980 2985 2990
Asn Leu Ala Val Val Pro Leu Pro Ala Met Arg Phe Arg Leu Gly Tyr
                    2995 3000 3005
Arg Pro Asp Val Phe Asp Ala Gly Arg Val Arg Glu Leu Trp Gly Trp
            3010 3015 3020
Ile Val Arg Ala Leu Glu Cys Val Val Cys Glu Arg Asp Val Pro Val
3025 3030 3035 3040
Ser Gly Val Asp Val Leu Gly Ala Gly Glu Arg Glu Thr Leu Leu Gly
                                    3045 3050 3055
Trp Gly Ala Gly Ala Glu Pro Gly Val Arg Ala Leu Pro Gly Ala Gly
                          3060 3065 3070
Ala Gly Ala Gly Ala Gly Leu Val Gly Leu Phe Glu Glu Arg Val Arg
            3075 3080 3085
Thr Asp Pro Asp Ala Val Ala Val Arg Gly Ala Gly Val Glu Trp Ser
      3090 3095 3100
Tyr Ala Glu Leu Asn Ala Arg Ala Asn Ala Val Ala Arg Trp Leu Ile
3105 3110 3115 3120
Gly Arg Gly Val Gly Pro Glu Arg Gly Val Gly Val Val Met Asp Arg
                                        3125 3130 3135
Gly Pro Asp Val Val Ala Met Leu Leu Ala Val Ala Lys Ser Gly Gly
                            3140 3145 3150
Phe Tyr Leu Pro Val Asp Pro Gln Trp Pro Thr Glu Arg Ile Asp Trp
                  3155 3160 3165
Val Leu Ala Asp Ala Gly Ile Asp Leu Ala Val Val Gly Glu Asn Leu
      3170 3175 3180
Ala Ala Ala Val Glu Ala Val Arg Asp Cys Glu Val Val Asp Tyr Ala
3185 3190 3195 3200
Gln Ile Ala Arg Glu Thr Arg Leu Asn Glu Gln Ala Ala Thr Asp Ala
                                        3205 3210 3215
Gly Asp Val Thr Asp Gly Glu Arg Val Ser Ala Leu Leu Ser Gly His
                          3220 3225 3230
Pro Leu Tyr Val Ile Tyr Thr Ser Gly Ser Thr Gly Leu Pro Lys Gly
                  3235 3240 3245
Val Val Val Thr His Ala Ser Val Gly Ala Tyr Leu Arg Arg Gly Arg
      3250 3255 3260
Asn Ala Tyr Arg Gly Ala Ala Asp Gly Leu Gly His Val His Ser Ser
3265 3270 3275 3280
Leu Ala Phe Asp Leu Thr Val Thr Val Leu Phe Thr Pro Leu Val Ser
                                    3285 3290 3295
Gly Gly Cys Val Thr Leu Gly Asp Leu Asp Asp Thr Ala Asn Gly Leu
                        3300 3305 3310
Gly Ala Thr Phe Leu Lys Ala Thr Pro Ser His Leu Pro Leu Leu Gly
                3315 3320 3325
Gln Leu Asp Arg Val Leu Ala Pro Asp Ala Thr Leu Leu Leu Gly Gly
      3330 3335 3340
Glu Ala Leu Thr Ala Gly Ala Leu His His Trp Arg Thr His His Pro
3345 3350 3355 3360
His Thr Thr Val Ile Asn Ala Tyr Gly Pro Thr Glu Leu Thr Val Asn
                                3365 3370 3375
Cys Ala Glu Tyr Arg Ile Pro Pro Gly His Cys Leu Pro Asp Gly Pro
                    3380 3385 3390
Val Pro Ile Gly Arg Pro Phe Thr Gly His His Leu Phe Val Leu Asp
            3395 3400 3405
Pro Ala Leu Arg Leu Thr Pro Pro Asp Thr Ile Gly Glu Leu Tyr Val
    3410 3415 3420
Ala Gly Asp Gly Leu Ala Arg Gly Tyr Leu Gly Arg Pro Asp Leu Thr
3425 3430 3435 3440
Ala Glu Arg Phe Val Ala Cys Pro Phe Arg Ser Pro Gly Glu Arg Met
                        3445 3450 3455
Tyr Arg Thr Gly Asp Leu Ala Arg Trp Arg Ser Asp Gly Thr Leu Glu
                  3460 3465 3470
Phe Ile Gly Arg Ala Asp Asp Gln Val Lys Ile Arg Gly Phe Arg Ile
      3475 3480 3485
Glu Leu Gly Glu Val Glu Ala Ala Val Ala Ala His Pro His Val Ala
    3490 3495 3500
Arg Ala Ile Ala Val Val Arg Glu Asp Arg Pro Gly Asp Gln Arg Leu
3505 3510 3515 3520
Val Ala Tyr Val Thr Gly Ser Asp Pro Ser Gly Leu Ser Ser Ala Val
                          3525 3530 3535
Thr Asp Thr Val Ala Gly Arg Leu Pro Ala Tyr Met Val Pro Ser Ala
                  3540 3545 3550
Val Val Val Leu Asp Gln Ile Pro Leu Thr Pro Asn Gly Lys Val Asp
          3555 3560 3565
Arg Ala Ala Leu Pro Ala Pro Gly Thr Ala Ser Gly Thr Thr Ser Arg
      3570 3575 3580
Ala Pro Gly Thr Ala Arg Glu Glu Ile Leu Cys Thr Leu Phe Ala Asp
3585 3590 3595 3600
Val Leu Gly Leu Asp Gln Val Gly Val Asp Glu Asp Phe Phe Asp Leu
                                          3605 3610 3615
Gly Gly His Ser Leu Leu Ala Thr Arg Leu Thr Ser Arg Ile Arg Ser
                                  3620 3625 3630
Ala Leu Gly Ile Asp Leu Gly Val Arg Ala Leu Phe Lys Ala Pro Thr
                          3635 3640 3645
Val Gly Arg Leu Asp Gln Leu Leu Gln Gln Gln Thr Thr Ser Leu Arg
                    3650 3655 3660
Ala Pro Leu Val Ala Arg Glu Arg Thr Gly Cys Glu Pro Leu Ser Phe
3665 3670 3675 3680
Ala Gln Gln Arg Leu Trp Phe Leu His Gln Leu Glu Gly Pro Asn Ala
                                          3685 3690 3695
Ala Tyr Asn Ile Pro Met Ala Leu Arg Leu Thr Gly Arg Leu Asp Leu
                                  3700 3705 3710
Thr Ala Leu Glu Ala Ala Leu Thr Asp Val Ile Ala Arg His Glu Ser
                          3715 3720 3725
Leu Arg Thr Val Ile Ala Gln Asp Asp Ser Gly Gly Val Trp Gln Asn
                  3730 3735 3740
Ile Leu Pro Thr Asp Asp Thr Arg Thr His Leu Thr Leu Asp Thr Met
3745 3750 3755 3760
Pro Val Asp Ala His Thr Leu Gln Asn Arg Val Asp Glu Ala Ala Arg
                                          3765 3770 3775
His Pro Phe Asp Leu Thr Thr Glu Ile Pro Leu Arg Ala Thr Val Phe
                                  3780 3785 3790
Arg Val Thr Asp Asp Glu His Val Leu Leu Leu Val Leu His His Ile
                          3795 3800 3805
Ala Gly Asp Gly Trp Ser Met Ala Pro Leu Ala His Asp Leu Ser Ala
                  3810 3815 3820
Ala Tyr Thr Val Arg Leu Glu His His Ala Pro Gln Leu Pro Ala Leu
3825 3830 3835 3840
Ala Val Gln Tyr Ala Asp Tyr Ala Ala Trp Gln Arg Asp Val Leu Gly
                                  3845 3850 3855
Thr Glu Asn Asn Thr Ser Ser Gln Leu Ser Thr Gln Leu Asp Tyr Trp
                          3860 3865 3870
Tyr Ser Lys Leu Glu Gly Leu Pro Ala Glu Leu Thr Leu Pro Thr Ser
                    3875 3880 3885
Arg Val Arg Pro Ala Val Ala Ser His Ala Cys Asp Arg Val Glu Phe
3890 3895 3900
Thr Val Pro His Asp Val His Gln Gly Leu Thr Ala Leu Ala Arg Thr
3905 3910 3915 3920
Gln Gly Ala Thr Val Phe Met Val Val Gln Ala Ala Leu Ala Ala Leu
                                  3925 3930 3935
Leu Ser Arg Leu Gly Ala Gly Thr Asp Ile Pro Ile Gly Thr Pro Ile
                          3940 3945 3950
Ala Gly Arg Thr Asp Gln Ala Met Glu Asn Leu Ile Gly Leu Phe Val
                    3955 3960 3965
Asn Thr Leu Val Leu Arg Thr Asp Val Ser Gly Asp Pro Thr Phe Ala
      3970 3975 3980
Glu Leu Leu Ala Arg Val Arg Thr Thr Ala Leu Asp Ala Tyr Ala His
3985 3990 3995 4000
Gln Asp Ile Pro Phe Glu Arg Leu Val Glu Ala Ile Asn Pro Glu Arg
                                    4005 4010 4015
Ser Leu Thr Arg His Pro Leu Phe Gln Val Met Leu Ala Phe Asn Asn
                            4020 4025 4030
Thr Asp Arg Arg Ser Ala Leu Asp Ala Leu Asp Ala Met Pro Gly Leu
                  4035 4040 4045
His Ala Arg Pro Ala Asp Val Leu Ala Val Thr Ser Pro Tyr Asp Leu
            4050 4055 4060
Ala Phe Ser Phe Val Glu Thr Pro Gly Ser Thr Glu Met Pro Gly Ile
4065 4070 4075 4080
Leu Asp Tyr Ala Thr Asp Leu Phe Asp Arg Ser Thr Ala Glu Ala Met
                                  4085 4090 4095
Thr Glu Arg Leu Val Arg Leu Leu Ala Glu Ile Ala Arg Arg Pro Glu
                          4100 4105 4110
Leu Ser Val Gly Asp Ile Gly Ile Leu Ser Ala Asp Glu Val Lys Ala
                4115 4120 4125
Leu Ser Pro Glu Ala Pro Pro Ala Ala Glu Glu Leu His Thr Ser Thr
    4130 4135 4140
Leu Pro Glu Leu Phe Glu Glu Gln Val Ala Ala Arg Gly His Ala Val
4145 4150 4155 4160
Ala Val Val Cys Glu Gly Glu Glu Leu Ser Tyr Lys Glu Leu Asn Ala
                                  4165 4170 4175
Arg Ala Asn Arg Leu Ala Arg Val Leu Met Glu Arg Gly Ala Gly Pro
                            4180 4185 4190
Glu Arg Phe Val Gly Val Ala Leu Pro Arg Gly Leu Asp Leu Ile Val
                  4195 4200 4205
Ala Leu Leu Ala Val Thr Lys Thr Gly Ala Ala Tyr Val Pro Leu Asp
          4210 4215 4220
Pro Glu Tyr Pro Thr Asp Arg Leu Ala Tyr Met Val Thr Asp Ala Asn
4225 4230 4235 4240
Pro Thr Ala Val Val Thr Ser Thr Asp Val His Ile Pro Leu Ile Ala
                                  4245 4250 4255
Pro Arg Ile Glu Leu Asp Asp Glu Ala Ile Arg Thr Glu Leu Ala Ala
                            4260 4265 4270
Ala Pro Asp Thr Ala Pro Cys Val Gly Ser Gly Pro Ala His Pro Ala
                  4275 4280 4285
Tyr Val Ile Tyr Thr Ser Gly Ser Thr Gly Arg Pro Lys Gly Val Val
          4290 4295 4300
Ile Ser His Ala Asn Val Val Arg Leu Phe Thr Ala Cys Ser Asp Ser
4305 4310 4315 4320
Phe Asp Phe Gly Pro Asp His Val Trp Thr Leu Phe His Ser Tyr Ala
                                    4325 4330 4335
Phe Asp Phe Ser Val Trp Glu Ile Trp Gly Ala Leu Leu His Gly Gly
                          4340 4345 4350
Arg Leu Val Val Val Pro Phe Glu Val Thr Arg Ser Pro Ala Glu Phe
                    4355 4360 4365
Leu Ala Leu Leu Ala Glu Gln Gln Val Thr Leu Leu Ser Gln Thr Pro
          4370 4375 4380
Ser Ala Phe His Gln Leu Thr Glu Ala Ala Arg Gln Glu Pro Ala Arg
4385 4390 4395 4400
Cys Ala Gly Leu Ala Leu Arg His Val Val Phe Gly Gly Glu Ala Leu
                                  4405 4410 4415
Asp Pro Ser Arg Leu Arg Asp Trp Phe Asp Leu Pro Leu Gly Ser Arg
                          4420 4425 4430
Pro Thr Leu Val Asn Met Tyr Gly Ile Thr Glu Thr Thr Val His Val
                  4435 4440 4445
Thr Val Leu Pro Leu Glu Asp Arg Ala Thr Ser Leu Ser Gly Ser Pro
            4450 4455 4460
Ile Gly Arg Pro Leu Ala Asp Leu Gln Val Tyr Val Leu Asp Glu Arg
4465 4470 4475 4480
Leu Arg Pro Val Pro Pro Gly Thr Val Gly Glu Met Tyr Val Ala Gly
                                    4485 4490 4495
Ala Gly Leu Ala Arg Gly Tyr Leu Gly Arg Pro Ala Leu Thr Ala Glu
                        4500 4505 4510
Arg Phe Val Ala Asp Pro Asn Ser Arg Ser Gly Gly Arg Leu Tyr Arg
                  4515 4520 4525
Thr Gly Asp Leu Ala Lys Val Arg Pro Asp Gly Gly Leu Glu Tyr Val
      4530 4535 4540
Gly Arg Gly Asp Arg Gln Val Lys Ile Arg Gly Phe Arg Ile Glu Leu
4545 4550 4555 4560
Gly Glu Ile Glu Ala Ala Leu Val Thr His Ala Gly Val Val Gln Ala
                                  4565 4570 4575
Val Val Leu Val Arg Asp Glu Gln Thr Asp Asp Gln Arg Leu Val Ala
                        4580 4585 4590
His Val Val Pro Ala Leu Pro His Arg Ala Pro Thr Leu Ala Glu Leu
                  4595 4600 4605
His Glu His Leu Ala Ala Thr Leu Pro Ala Tyr Met Val Pro Ser Ala
          4610 4615 4620
Tyr Arg Thr Leu Asp Glu Leu Pro Leu Thr Ala Asn Gly Lys Leu Asp
4625 4630 4635 4640
Arg Ala Ala Leu Ala Gly Gln Trp Gln Gly Gly Thr Arg Thr Arg Arg
                              4645 4650 4655
Leu Pro Arg Thr Pro Gln Glu Glu Ile Leu Cys Glu Leu Phe Ala Asp
                    4660 4665 4670
Val Leu Arg Leu Pro Ala Ala Gly Ala Asp Asp Asp Phe Phe Ala Leu
            4675 4680 4685
Gly Gly His Ser Leu Leu Ala Thr Arg Leu Leu Ser Ala Val Arg Gly
      4690 4695 4700
Thr Leu Gly Val Glu Leu Gly Ile Arg Asp Leu Phe Ala Ala Pro Thr
4705 4710 4715 4720
Pro Ala Gly Leu Ala Thr Val Leu Ala Ala Ser Gly Thr Ala Leu Pro
                                4725 4730 4735
Pro Val Thr Arg Ile Asp Arg Arg Pro Glu Arg Leu Pro Leu Ser Phe
                    4740 4745 4750
Ala Gln Arg Arg Leu Trp Phe Leu Ser Lys Leu Glu Gly Pro Ser Ala
          4755 4760 4765
Thr Tyr Asn Ile Pro Val Ala Val Arg Leu Thr Gly Ala Leu Asp Val
    4770 4775 4780
Pro Ala Leu Arg Ala Ala Leu Gly Asp Val Thr Ala Arg His Glu Ser
4785 4790 4795 4800
Leu Arg Thr Val Phe Pro Asp Asp Gly Gly Glu Pro Arg Gln Leu Val
                                          4805 4810 4815
Leu Pro His Ala Glu Pro Pro Phe Leu Thr His Glu Val Thr Val Gly
                                  4820 4825 4830
Glu Val Ala Glu Gln Ala Ala Ser Ala Thr Gly Tyr Ala Phe Asp Ile
                          4835 4840 4845
Thr Ser Asp Thr Pro Leu Arg Ala Thr Leu Leu Arg Val Ser Pro Glu
                  4850 4855 4860
Glu His Val Leu Val Val Val Ile His His Ile Ala Gly Asp Gly Trp
4865 4870 4875 4880
Ser Met Gly Pro Leu Val Arg Asp Leu Val Thr Ala Tyr Arg Ala Arg
                                    4885 4890 4895
Thr Arg Gly Asp Ala Pro Glu Tyr Thr Pro Leu Pro Val Gln Tyr Ala
                          4900 4905 4910
Asp Tyr Ala Leu Trp Gln His Ala Val Ala Gly Asp Glu Asp Ala Pro
            4915 4920 4925
Asp Gly Arg Thr Ala Arg Arg Leu Gly Tyr Trp Arg Glu Met Leu Ala
    4930 4935 4940
Gly Leu Pro Glu Glu His Thr Leu Pro Ala Asp Arg Pro Arg Pro Val
4945 4950 4955 4960
Arg Ser Ser His Arg Gly Gly Arg Val Arg Phe Glu Leu Pro Ala Gly
                            4965 4970 4975
Val His Arg Ser Leu Leu Ala Val Ala Arg Asp Arg Arg Ala Thr Leu
                    4980 4985 4990
Phe Met Val Val Gln Ala Ala Leu Ala Gly Leu Leu Ser Arg Leu Gly
            4995 5000 5005
Ala Gly Asp Asp Ile Pro Ile Gly Thr Pro Val Ala Gly Arg Gly Asp
    5010 5015 5020
Glu Ala Leu Asp Asp Val Val Gly Phe Phe Val Asn Thr Leu Val Leu
5025 5030 5035 5040
Arg Thr Asn Leu Ala Gly Asp Pro Ser Phe Ala Asp Leu Val Asp Arg
                                  5045 5050 5055
Val Arg Thr Ala Asp Leu Asp Ala Phe Ala His Gln Asp Val Pro Phe
                        5060 5065 5070
Glu Arg Leu Val Glu Ala Leu Ala Pro Arg Arg Ser Leu Ala Arg His
              5075 5080 5085
Pro Leu Phe Gln Ile Trp Tyr Thr Leu Thr Asn Ala Asp Gln Asp Ile
    5090 5095 5100
Thr Gly Gln Ala Leu Asn Ala Leu Pro Gly Leu Thr Gly Asp Glu Tyr
5105 5110 5115 5120
Pro Leu Gly Ala Ser Ala Ala Lys Phe Asp Leu Ser Phe Thr Phe Thr
                                5125 5130 5135
Glu His Arg Thr Pro Asp Gly Asp Ala Ala Gly Leu Ser Val Leu Leu
                    5140 5145 5150
Asp Tyr Ser Ser Asp Leu Tyr Asp His Gly Thr Ala Ala Ala Leu Gly
            5155 5160 5165
His Arg Leu Thr Gly Phe Phe Ala Ala Leu Ala Ala Asp Pro Thr Ala
    5170 5175 5180
Pro Leu Gly Thr Val Pro Leu Leu Thr Asp Asp Glu Arg Asp Arg Ile
5185 5190 5195 5200
Leu Gly Asp Trp Gly Ser Gly Thr His Thr Pro Leu Pro Pro Arg Ser
                                        5205 5210 5215
Val Ala Glu Gln Ile Val Arg Arg Ala Ala Leu Asp Pro Asp Ala Val
                            5220 5225 5230
Ala Val Ile Thr Ala Glu Glu Glu Leu Ser Tyr Arg Glu Leu Glu Arg
                5235 5240 5245
Leu Ser Gly Glu Thr Ala Arg Leu Leu Ala Asp Arg Gly Ile Gly Arg
    5250 5255 5260
Glu Ser Leu Val Ala Val Ala Leu Pro Arg Thr Ala Gly Leu Val Thr
5265 5270 5275 5280
Thr Leu Leu Gly Val Leu Arg Thr Gly Ala Ala Tyr Leu Pro Leu Asp
                                  5285 5290 5295
Thr Gly Tyr Pro Ala Glu Arg Leu Ala His Val Leu Ser Asp Ala Arg
                          5300 5305 5310
Pro Asp Leu Val Leu Thr His Ala Gly Leu Ala Gly Arg Leu Pro Ala
                  5315 5320 5325
Gly Leu Ala Pro Thr Val Leu Val Asp Glu Pro Gln Pro Pro Ala Ala
          5330 5335 5340
Ala Ala Pro Ala Val Pro Thr Ser Pro Ser Gly Asp His Leu Ala Tyr
5345 5350 5355 5360
Val Ile His Thr Ser Gly Ser Thr Gly Arg Pro Lys Gly Val Ala Ile
                                          5365 5370 5375
Ala Glu Ser Ser Leu Arg Ala Phe Leu Ala Asp Ala Val Arg Arg His
                          5380 5385 5390
Asp Leu Thr Pro His Asp Arg Leu Leu Ala Val Thr Thr Val Gly Phe
                  5395 5400 5405
Asp Ile Ala Gly Leu Glu Leu Phe Ala Pro Leu Leu Ala Gly Ala Ala
      5410 5415 5420
Ile Val Leu Ala Asp Glu Asp Ala Val Arg Asp Pro Ala Ser Ile Thr
5425 5430 5435 5440
Ser Leu Cys Ala Arg His His Val Thr Val Val Gln Ala Thr Pro Ser
                                5445 5450 5455
Trp Trp Arg Ala Met Leu Asp Gly Ala Pro Ala Asp Ala Ala Ala Arg
                        5460 5465 5470
Leu Glu His Val Arg Ile Leu Val Gly Gly Glu Pro Leu Pro Ala Asp
          5475 5480 5485
Leu Ala Arg Val Leu Thr Ala Thr Gly Ala Ala Val Thr Asn Val Tyr
    5490 5495 5500
Gly Pro Thr Glu Ala Thr Ile Trp Ala Thr Ala Ala Pro Leu Thr Ala
5505 5510 5515 5520
Gly Asp Asp Arg Thr Pro Gly Ile Gly Thr Pro Leu Asp Asn Trp Arg
                        5525 5530 5535
Val His Ile Leu Asp Ala Ala Leu Gly Pro Val Pro Pro Gly Val Pro
                  5540 5545 5550
Gly Glu Ile His Ile Ala Gly Ser Gly Leu Ala Arg Gly Tyr Leu Arg
            5555 5560 5565
Arg Pro Asp Leu Thr Ala Glu Arg Phe Val Ala Asn Pro Phe Ala Pro
  5570 5575 5580
Gly Glu Arg Met Tyr Arg Thr Gly Asp Leu Gly Arg Phe Arg Pro Asp
5585 5590 5595 5600
Gly Thr Leu Glu His Leu Gly Arg Val Asp Asp Gln Val Lys Val Arg
                                5605 5610 5615
Gly Phe Arg Ile Glu Leu Gly Asp Val Glu Ala Ala Leu Ala Arg His
                    5620 5625 5630
Pro Asp Val Gly Arg Ala Ala Ala Ala Val Arg Pro Asp His Arg Gly
            5635 5640 5645
Gln Gly Arg Leu Val Ala Tyr Val Val Pro Arg Pro Gly Thr Arg Gly
    5650 5655 5660
Pro Asp Ala Gly Glu Leu Arg Glu Thr Val Arg Glu Leu Leu Pro Asp
5665 5670 5675 5680
Tyr Met Val Pro Ser Ala Gln Val Thr Leu Thr Thr Leu Pro His Thr
                            5685 5690 5695
Pro Asn Gly Lys Leu Asp Arg Ala Ala Leu Pro Ala Pro Val Phe Gly
                    5700 5705 5710
Thr Pro Ala Gly Arg Ala Pro Ala Thr Arg Glu Glu Lys Ile Leu Ala
          5715 5720 5725
Gly Leu Phe Ala Asp Ile Leu Gly Leu Pro Asp Val Gly Ala Asp Ser
    5730 5735 5740
Gly Phe Phe Asp Leu Gly Gly Asp Ser Val Leu Ser Ile Gln Leu Val
5745 5750 5755 5760
Ser Arg Ala Arg Arg Glu Gly Leu His Ile Thr Val Arg Asp Val Phe
                                  5765 5770 5775
Glu His Gly Thr Val Gly Ala Leu Ala Ala Ala Ala Leu Pro Ala Pro
                      5780 5785 5790
Ala Asp Asp Ala Asp Asp Thr Val Pro Gly Thr Asp Val Leu Pro Ser
            5795 5800 5805
Ile Ser Asp Asp Glu Phe Glu Glu Phe Glu Leu Glu Leu Gly Leu Glu
    5810 5815 5820
Gly Glu Glu Glu Gln Trp
5825 5830
dptF Acyl carrier protein
SEQ ID NO: 35
Met Asn Pro Pro Glu Ala Val Ser Thr Pro Ser Glu Val Thr Ala Trp
    1 5 10 15
Ile Thr Gly Gln Ile Ala Glu Phe Val Asn Glu Thr Pro Asp Arg Ile
                              20 25 30
Ala Gly Asp Ala Pro Leu Thr Asp His Gly Leu Asp Ser Val Ser Gly
                      35 40 45
Val Ala Leu Cys Ala Gln Val Glu Asp Arg Tyr Gly Ile Glu Val Asp
          50 55 60
Pro Glu Leu Leu Trp Ser Val Pro Thr Leu Asn Glu Phe Val Gln Ala
  65 70 75 80
Leu Met Pro Gln Leu Ala Asp Arg Thr
                                      85
ptE Fatty acid ligase
SEQ ID NO: 36
Met Ser Glu Ser Arg Cys Ala Gly Gln Gly Leu Val Gly Ala Leu Arg
    1 5 10 15
Thr Trp Ala Arg Thr Arg Ala Arg Glu Thr Ala Val Val Leu Val Arg
                              20 25 30
Asp Thr Gly Thr Thr Asp Asp Thr Ala Ser Val Asp Tyr Gly Gln Leu
                      35 40 45
Asp Glu Trp Ala Arg Ser Ile Ala Val Thr Leu Arg Gln Gln Leu Ala
          50 55 60
Pro Gly Gly Arg Ala Leu Leu Leu Leu Pro Ser Gly Pro Glu Phe Thr
  65 70 75 80
Ala Ala Tyr Leu Gly Cys Leu Tyr Ala Gly Leu Ala Ala Val Pro Ala
                                      85 90 95
Pro Leu Pro Gly Gly Arg His Phe Glu Arg Arg Arg Val Ala Ala Ile
                            100 105 110
Ala Ala Asp Ser Gly Ala Gly Val Val Leu Thr Val Ala Gly Glu Thr
                  115 120 125
Ala Ser Val His Asp Trp Leu Thr Glu Thr Thr Ala Pro Ala Thr Arg
          130 135 140
Val Val Ala Val Asp Asp Arg Ala Ala Leu Gly Asp Pro Ala Gln Trp
145 150 155 160
Asp Asp Pro Gly Val Ala Pro Asp Asp Val Ala Leu Ile Gln Tyr Thr
                                    165 170 175
Ser Gly Ser Thr Gly Asn Pro Lys Gly Val Val Val Thr His Ala Asn
                            180 185 190
Leu Leu Ala Asn Ala Arg Asn Leu Ala Glu Ala Cys Glu Leu Thr Ala
                  195 200 205
Ala Thr Pro Met Gly Gly Trp Leu Pro Met Tyr His Asp Met Gly Leu
          210 215 220
Leu Gly Thr Leu Thr Pro Ala Leu Tyr Leu Gly Thr Thr Cys Val Leu
225 230 235 240
Met Ser Ser Thr Ala Phe Ile Lys Arg Pro His Leu Trp Leu Arg Thr
                                    245 250 255
Ile Asp Arg Phe Gly Leu Val Trp Ser Ser Ala Pro Asp Phe Ala Tyr
                            260 265 270
sp Met Cys Leu Lys Arg Val Thr Asp Glu Gln Ile Ala Gly Leu Asp
                    275 280 285
Leu Ser Arg Trp Arg Trp Ala Gly Asn Gly Ala Glu Pro Ile Arg Ala
          290 295 300
Ala Thr Val Arg Ala Phe Gly Glu Arg Phe Ala Arg Tyr Gly Leu Arg
305 310 315 320
Pro Glu Ala Leu Thr Ala Gly Tyr Gly Leu Ala Glu Ala Thr Leu Phe
                                  325 330 335
Val Ser Arg Ser Gln Gly Leu His Thr Ala Arg Val Ala Thr Ala Ala
                            340 345 350
Leu Glu Arg His Glu Phe Arg Leu Ala Val Pro Gly Glu Ala Ala Arg
                    355 360 365
Glu Ile Val Ser Cys Gly Pro Val Gly His Phe Arg Ala Arg Ile Val
            370 375 380
Glu Pro Gly Gly His Arg Val Leu Pro Pro Gly Gln Val Gly Glu Leu
385 390 395 400
Val Leu Gln Gly Ala Ala Val Cys Ala Gly Tyr Trp Gln Ala Lys Glu
                                      405 410 415
Glu Thr Glu Gln Thr Phe Gly Leu Thr Leu Asp Gly Glu Asp Gly His
                              420 425 430
Trp Leu Arg Thr Gly Asp Leu Ala Ala Leu His Glu Gly Asn Leu His
                    435 440 445
Ile Thr Gly Arg Cys Lys Glu Ala Leu Val Ile Arg Gly Arg Asn Leu
          450 455 460
Tyr Pro Gln Asp Ile Glu His Glu Leu Arg Leu Gln His Pro Glu Leu
465 470 475 480
Glu Ser Val Gly Ala Ala Phe Thr Val Pro Ala Ala Pro Gly Thr Pro
                                    485 490 495
Hly Leu Met Val Val His Glu Val Arg Thr Pro Val Pro Ala Asp Asp
                          500 505 510
His Pro Ala Leu Val Ser Ala Leu Arg Gly Thr Ile Asn Arg Glu Phe
                  515 520 525
Gly Leu Asp Ala Gln Gly Ile Ala Leu Val Ser Arg Gly Thr Val Leu
        530 535 540
Arg Thr Thr Ser Gly Lys Val Arg Arg Gly Ala Met Arg Asp Leu Cys
545 550 555 560
Leu Arg Gly Glu Leu Asn Ile Val His Ala Asp Lys Gly Trp His Ala
                                    565 570 575
Ile Ala Gly Thr Ala Gly Glu Asp Ile Ala Pro Thr Asp His Ala Pro
                          580 585 590
His Pro His Pro Ala
                    595
ptN ABC-type transporter gene
SEQ ID NO: 37
Met Leu Thr Thr Arg Arg Thr Gly Pro Gly Thr Ser Pro Val Ala Asp
    1 5 10 15
Gly Pro Gly Trp Arg Gly Gly Gly Ala Gly Ile Gly Thr Gln Phe Arg
                              20 25 30
Val Leu Thr Gly Arg Gln Phe Arg Ile Ile Tyr Gly Asp Arg Arg Ile
                      35 40 45
Ala Leu Phe Ser Leu Leu Gln Pro Ile Ile Met Leu Met Leu Phe Ser
          50 55 60
Gln Val Leu Gly Arg Met Ala Asn Pro Glu Ile Phe Pro Pro Gly Val
  65 70 75 80
Arg Tyr Leu Asp Tyr Leu Val Pro Ala Leu Leu Leu Thr Thr Gly Ile
                                      85 90 95
Gly Ser Ala Gln Gly Gly Gly Leu Gly Leu Val Arg Asp Met Glu Ser
                            100 105 110
Gly Met Met Val Arg Leu Arg Val Met Pro Val Arg Leu Pro Leu Val
                  115 120 125
Leu Val Ala Arg Ser Leu Ala Asp Leu Ala Arg Val Ala Leu Gln Leu
          130 135 140
Val Ala Leu Leu Ala Cys Ala Met Gly Pro Leu Gly Tyr Arg Pro Ala
145 150 155 160
Gly Gly Val Ser Gly Ile Val Gly Ala Thr Leu Leu Ala Leu Leu Val
                                    165 170 175
Ala Trp Ser Leu Ile Trp Val Phe Leu Ala Leu Ala Ala Trp Leu Arg
                            180 185 190
Ser Ile Glu Val Leu Ser Ser Ile Gly Phe Leu Val Thr Phe Pro Leu
                  195 200 205
Met Phe Ala Ser Ser Ala Phe Val Pro Leu Asp Ile Leu Pro Gly Trp
          210 215 220
Leu Arg Val Ile Ala Thr Val Asn Pro Leu Thr Tyr Ala Val Glu Ala
225 230 235 240
Ser Arg Asp Leu Ala Leu Asp His Ser Ala Leu Gly Ala Ala Leu Ala
                                    245 250 255
Ala Val Gly Thr Ser Leu Ala Leu Leu Ala Val Thr Gly Leu Leu Ala
                            260 265 270
Val Arg Gly Leu Arg Arg Pro Pro Gly Ala Gly Gly Pro His Arg Thr
                    275 280 285
Pro
dptM_ABC-type transporter gene
SEQ ID NO: 38
Met Gly Gly Ser Ala Ile Arg Thr Arg Gln Leu Thr Lys His Phe Gly
    1 5 10 15
Ala Val Gln Ala Leu Val Gly Val Asp Leu Glu Val Pro Ala Gly Ser
                              20 25 30
Val Leu Gly Leu Leu Gly His Asn Gly Ala Gly Lys Thr Thr Leu Ile
                      35 40 45
Gln Ile Leu Ser Thr Val Leu Pro Pro Ser Gly Gly Ser Ala Glu Val
          50 55 60
Ala Gly Phe Asp Ile Val Arg Asp Ala Arg Arg Val Arg Ala Cys Ile
  65 70 75 80
Gly Val Thr Gly Gln Phe Ala Ala Leu Asp Glu His Leu Ser Gly Leu
                                      85 90 95
Hla Asn Leu Val Leu Ile Ser Arg Leu Leu Gly Ala Arg Pro Arg Glu
                            100 105 110
Gla Arg Arg Arg Ala Ala Glu Leu Val Glu Gln Phe Gly Leu Thr Glu
                  115 120 125
Gla Ala Asp Arg Pro Met Arg Thr Tyr Ser Gly Gly Met Arg Arg Arg
          130 135 140
Ile Asp Leu Ala Ala Ser Leu Val Ala Arg Pro Ser Val Leu Phe Leu
145 150 155 160
Asp Glu Pro Thr Thr Gly Leu Asp Pro Val Ser Arg Thr Ala Leu Trp
                                    165 170 175
Glu Thr Val Glu Gly Leu Val Ala Glu Gly Thr Thr Val Leu Leu Thr
                            180 185 190
Thr Gln Tyr Leu Asp Glu Ala Asp Arg Leu Ala Asp Arg Ile Ala Val
                  195 200 205
Leu Ser Ser Gly His Val Val Thr Val Gly Thr Ala Ala Glu Leu Lys
          210 215 220
Ala Ala Gly Thr Arg Ser Val Arg Leu Thr Phe Gly Ser Ala Ala Asp
225 230 235 240
Leu Glu Ser Ala Glu Gly Ala Leu Arg Leu Glu Gly Leu Gly Leu Thr
                                    245 250 255
Thr Asp Pro Val Ser Arg Thr Val Ser Leu Pro Leu Ala Ala Thr Ala
                            260 265 270
Glu Leu Ala Gly Ile Phe Arg Ile Leu Gly Ala Ala Gly Val Glu Leu
                    275 280 285
Ala Glu Leu Ala Leu Lys Glu Pro Thr Leu Asp Asp Val Tyr Leu Ser
          290 295 300
Leu Ala Glu Ser Trp Glu Thr Thr Ser Gly Gly Thr Val Arg Cys
305 310 315
ptP Transporter gene
SEQ ID NO: 39
Met Ser Trp Met Asn Asp Val Leu Thr Ala Val Ser Asp Met Asn Pro
    1 5 10 15
Val Thr Arg Phe Ala Leu Ala Ser Val Phe Ala Phe Ala Glu Ser Gly
                              20 25 30
Leu Gly Ala Gly Met Ala Val Pro Gly Glu Val Ala Val Leu Ala Leu
                      35 40 45
Ser Ala Gly Thr Glu Gly Thr Arg Pro Leu Leu Ala Leu Phe Leu Val
          50 55 60
Val Thr Leu Ser Ser Ser Ala Gly Asp His Ile Gly Tyr Phe Leu Gly
  65 70 75 80
Ale Arg Tyr Gly Gln Arg Met Arg Glu Thr Arg Leu Val Arg Arg Ile
                                      85 90 95
Gly Gln His His Trp Asp Arg Ala Gln Glu Leu Cys His Arg Tyr Gly
                            100 105 110
Ala Arg Ala Val Phe Leu Thr Arg Leu Leu Pro Val Val Arg Thr Leu
115 125
Thr Pro Ala Thr Ala Gly Val Gly Ser Val Arg Tyr Leu Arg Phe Leu
          130 135 140
Pro Ala Ser Leu Ala Gly Ala Ala Met Trp Ser Ala Leu Tyr Val Ser
145 150 155 160
Ala Gly Thr Leu Val Ser Thr Ser Leu Arg Glu Ala Glu Ser Val Leu
                                    165 170 175
Ser Thr Ile Leu Trp Ala Leu Leu Gly Val Ala Ala Ala Phe Thr Leu
                            180 185 190
Ala Ile Val Trp Trp Arg Arg Arg His Arg Arg Arg Ser Ser
                  195 200 205
BAC-dpt*
SEQ ID NO: 40
gtctgataat cagactggga ccacggtccc actcgtatcg tcggtctgat tattagtctg 60
ggaccacggt cccactcgta tcgtcggtct gattattagt ctgggaccac ggtcccactc 120
gtatcgtcgg tctgattatt agtctggaac cacggtccca ctcgtatcgt cggtctgatt 180
attagtctgg gaccacggtc ccactcgtat cgtcggtctg attattagtc tgggaccacg 240
atcccactcg tgttgtcggt ctgattatcg gtctgggacc acggtcccac ttgtattgtc 300
gatcagacta tcagcgtgag actacgattc catcaatgcc tgtcaagggc aagtattgac 360
atgtcgtcgt aacctgtaga acggagtaac ctcggtgtgc ggttgtatgc ctgctgtgga 420
ttgctgctgt gtcctgctta tccacaacat tttgcgcacg gttatgtgga caaaatacct 480
ggttacccag gccgtgccgg cacgttaaat cgccctatag tgagtcgtat tacaattcac 540
actagtggta ccaacccagg caggtagcac agtgcgcgcc cactgcctta cggatagcac 600
gacgcatctt cgccgcagcg ttgttcccac gggcgatagc agcacgccgc ttcgcatggc 660
tcttcacact gcgctgtgcc tgataggtgg cgtggtgctg agcacagcgc ccaccatgag 720
tagcccagtc cctacagtcg agacaccggg tacgcatagg gcaccccccc cccccttccg 780
gggacacccc cctatccgtc gacacccccc tacctactca cgtgagtagg ctgggcgcgg 840
cagcgctaga agcgaccgcc cgtcatgaag tgggcagtga gccaacccat gaaggccagt 900
agcgcaaagc gtcgagcacg cacccaaccc gaaggcttgc ctgtactgcc gctttgcgtg 960
gcgaaccatg accagacgtg ttcgctcagc gtgtcgcccg gcttcttatt gaagagcgct 1020
cgaccctcaa tgacgccgaa cgcggcaagc catgcgagcc aagcgattgt gtatccgctc 1080
attgagcccc ccgacggatt cgaaccgccg accccctgat tacgaaacag gcgctctaac 1140
cgaactgagc tagggaggca acgccggtcg ctcaaccctg ggagagatca ggggagtgac 1200
cggcctgagt gggtagcgct cgacgccgtt ccttagcggt gcttgatgcg tggcgcgtaa 1260
acccctctac ctatatgaag gttgtcggca acctgattcg gggtcacagc gaagggcgcc 1320
cgggtgacgc tgtgatgaag tggcgtgaag tctggattac ccaatgagaa gtcattagga 1380
atgtgtgttt cgagtcattc cgtcacagca tcacttcccc caggtcaacg gctcgccccg 1440
ctgtgctgtg gtgacggagt cactgttact taggctaact aactaaaccg agacaagaga 1500
aatgggccga ccgggggacc ggccgaccct tcccgctgcc ccaggaagcc cgctgtgcgc 1560
ccgtctcagc gcctaacagg cttcccgggt gtctcgctac gccgctacgt cttccgtgcc 1620
gtcctgggcg tcgtcttcgt cgtcgtcggt cggcggcttc gcccacgtga tcgaagcgcg 1680
cttctcgatg ggcgttccct gccccctgcc cgtagtcgac ttcgtgacaa cgatcttgtc 1740
tacgaagagc ccgacgaaca cgcgcttgtc gtctactgac gcgcgccccc accacgactt 1800
agggccggtc gggtcagcgt cggcgtcttc ggggaaccat tggtcaaggg gaagcttcgg 1860
ggcttcggcg gcttcaagtt cggcaagccg ctcttccgcc ccttgctgcc ggagcgtcag 1920
cgctgcctgt tgcttccgga agtgcttcct gccaacgggt ccgtcgtacg cgcctgccgc 1980
gcggtcttcg tacagctctt caagggcgtt cagggcgtcg gcgcgctccg caacaaggtt 2040
cgcccgttcg ccgctcttct caggcgcctc agtgagcttg ccgaagcgtc gggcggcttc 2100
ccacagaagc gccaacgtct cttcgtcgcc ttcggcgtgc ctgatcttgt tgaagatgcg 2160
ttccgcaacg aacttgtcga gtgccgccat gctgacgttg cacgtgcctt cgtgctgccc 2220
aggtgcggac gggtcgacca ccttccggcg acggcagcgg taagagtcct tgatcgattc 2280
ttccccgcgc ttcgaagtca tgacggcgcc acactcgcag tacagcttgt ccatggcgga 2340
cagaatggct tgcccccggg aaagcccctt gccgcgcccc ctgccgtcca accacgcctg 2400
aagctcatac cactcagcgg gctcgatgat cggtccgcaa tcaagctcga ccggccggag 2460
cgtgatcggg tcgcgctgaa tgcggtaacc ctcaatcttc gtggtcggcg tgccgtccgg 2520
cttcttcttg tagatcacct cagcggcgaa gcccgcaata cgcgggtccc gaaggattcg 2580
cataacggtt gccgggtccc aggcgcttga agcggtcttc ttcccaatcg tctcgccccg 2640
ggtcggcacg gcgtcagcgt ccatgcgctt acaaagcccc gtgatgctgc ccgggtgaat 2700
ggcggcttga ctgcccggct tgaagggaag gtgtttgtgc gtcttgatct cacgccacca 2760
ccaccggatt acgtcgggct cgaactcgaa gggtccggta aggggagtgg tcgagtgcgc 2820
aagcttgttg atgacgacat tgaccattcg gccgttgcgc gtgatctcct tcgtctccga 2880
aacaagctcg aagccgtaag gcgccttccc gccgacgtac ccgcccaatt cgcgctgaag 2940
gttcttcgtg tcgagaatct tcgccgactt cagcgaagat tctttgtgcg acgcgtcgag 3000
ccgcataatc aggtgaatca ggtccatgac gtttccctgc cggaagacgc cttcctgagt 3060
ggaaacaatc gtcacgccca gggcgagcaa ttccgagaca atcggaatcg cgtccatgac 3120
cttcaggcgc gagaagcgcg acacgtcata gacaatgatc atgttgagcc gcccggcgcg 3180
gcattcgttc aggatgcgtt cgaactccgg gcgctccgcc gtcccgaacg ccgacgtgcc 3240
cggcgcttcg ctgaaatgcc cgacgaacct gaaccggccc ccgtcgcgct cgacttcgcg 3300
ctgaaggtcg gccgccttgt cttcgttggc gctacgctgt gtcgctgggc ttgctgcgct 3360
cgaattctcg cgctcgcgcg actgacggtc gtaagcaccc gcgtacgtgt ccaccccggt 3420
cacaacccct tgtgtcatgt cggcgaccct acgcccccaa ctgagagaac tcaaaggtta 3480
ccccagttgg ggcactactc ccgaaaaccg cttctgacct gggaaaacgt gaagccccgg 3540
ggcatccgct gagggttgcc gccggggctt cggtgtgtcc gtcagtacgg gccatagagg 3600
ggcgtcgtgt aacccaccca gggcgattgc tccggcacgg ggaagccgcg ccacgccttc 3660
gggacgtcag cggctcgccc cgtgcgctcc cacatctgcc acgtctgaag gcagtgggcg 3720
agccagtcat acgccgccga ccgcgaacgc cattcgagcg tcaccggctc gccgtcgtcg 3780
tcctggggca gcgcgcagta atcgcccatg acttcatcga agatcacgaa cggcggattc 3840
ttgcgcacga cccaggttcc gtttttccgg cgctgtagcg cgcgttccat gaccctgtat 3900
ctcttcaccg gccgacccgc tcacgcgcga cgttgtacgg gacggcgccc ccgccgacaa 3960
ggggcacttc gccccacgtc cggtagcacc gcattagcca cgcctgggcg ccatgctggg 4020
tgacccattc gagcggcatg agggttgagg ggtcgccgtc gtcgggcagc gcgcaatagc 4080
ccttcatggc tccgtcgaag atcacccagg ggcgcacgac gggcgggttg ccgtcttcgg 4140
ttcgctgtag ttcctgccgc cactcaggcg gacgctgtgt gactcgatac cgacgcatag 4200
ggaacacgct aggggacttc cctcagttgg gggagagcgt gacgaaaatt ccaccttcgc 4260
gggcttccca ggtgcgcccc cgctcgacct caaaccggcc atcggtcaca agcttcatgc 4320
gcaacgggcc gaccacgttg aagagaatgt gcgcgagcgt cgtcgtgtct tcgtcggctt 4380
cggcttcctc gcgcgcccag gtgtcggcgg gtgagtggcc cgtgtagcac gtcggcgagt 4440
agggcacgcc cagggcttcc gctagttcct tgtcgcacac aacgcccggg tcggcggcag 4500
tctccggggt gtcgccccag gcttcttcat acgggtactc aggcgagccg ccttcagcga 4560
tcaagggcgt ctcagcgtcg gcttcgtggt tgtgccccgg gaagggcagc atgacgtctg 4620
gaatcgatga taagctgtca aacatgagaa ttccccaatg tcaagcactt ccggaatcgg 4680
gagcgcggcc gatgcaaagt gccgataaac ataacgatct ttgtagaaac catcggcgca 4740
gctatttacc cgcaggacat atccacgccc tcctacatcg aagctgaaag cacgagattc 4800
ttcgccctcc gagagctgca tcaggtcgga gacgctgtcg aacttttcga tcagaaactt 4860
ctcgacagac gtcgcggtga gttcaggctt tttcatatct cattgccccc ggacgagcgt 4920
ctgctccgcc attcgccgtc cgccgtgcca atcggatcag ccgtccaaat gcgggatttt 4980
cgttagtcgg aggccaaacg gcattgagcg tcagcatatc atcagcgagc tgaagaaaga 5040
caatccccga tccgctccac gtgttgcccc agcaatcagc gcgaccttgc ccctccaacg 5100
tcatctcgtt ctccgctcat gagctcagcc aatcgactgg cgagcggcat cgcattcttc 5160
gcatcccgcc tctggcggat gcaggaagat caacggatct cggcccagtt gacccagggc 5220
tgtcgccaca atgtcgcggg agcggatcaa ccgagcaaag gcatgaccga ctggaccttc 5280
cttctgaagg ctcttctcct tgagccacct gtccgccaag gcaaagcgct cacagcagtg 5340
gtcattctcg agataatcga cgcgtaccaa cttgccatcc tgaagaatgg tgcagtgtct 5400
cggcacccca tagggaacct ttgccatcaa ctcggcaaga tgcagcgtcg tgttggcatc 5460
gtgtcccacg ccgaggagaa gtacctgccc atcgagttca tggacacggg cgaccgggct 5520
tgcaggcgag tgaggtggca ggggcaatgg atcagagatg atctgctctg cctgtggccc 5580
cgctgccgca aaggcaaatg gatgggcgct gcgctttaca tttggcaggc gccagaatgt 5640
gtcagagaca actccaaggt ccggtgtaac gggcgacgtg gcaggatcga acggctcgtc 5700
gtccagacct gaccacgagg gcatgacgag cgtccctccc ggacccagcg cagcacgcag 5760
ggcctcgatc agtccaagtg gcccatcttc gaggggccgg acgctacgga aggagctgtg 5820
gaccagcagc acaccgccgg gggtaacccc aaggttgaga agctgaccga tgagctcggc 5880
ttttcgccat tcgtattgca cgacattgca ctccaccgct gatgacatca gtcgatcata 5940
gcacgatcaa cggcactgtt gcaaatagtc ggtggtgata aacttatcat ccccttttgc 6000
tgatggagct gcacatgaac ccattcaaag gccggcattt tcagcgtgac atcattctgt 6060
gggccgtacg ctggtactgc aaatacggca tcagttaccg tgagctgcag gtcgacggat 6120
cttttccgct gcataaccct gcttcggggt cattatagcg attttttcgg tatatccatc 6180
ctttttcgca cgatatacag gattttgcca aagggttcgt gtagactttc cttggtgtat 6240
ccaacggcgt cagccgggca ggataggtga agtaggccca cccgcgagcg ggtgttcctt 6300
cttcactgtc ccttattcgc acctggcggt gctcaacggg aatcctgctc tgcgaggctg 6360
gccggctacc gccggcgtaa cagatgaggg caagcggatg gctgatgaaa ccaagccaac 6420
caggaagggc agcccaccta tcaaggtgta ctgccttcca gacgaacgaa gagcgattga 6480
ggaaaaggcg gcggcggccg gcatgagcct gtcggcctac ctgctggccg tcggccaggg 6540
ctacaaaatc acgggcgtcg tggactatga gcacgtccgc gagctggccc gcatcaatgg 6600
cgacctgggc cgcctgggcg gcctgctgaa actctggctc accgacgacc cgcgcacggc 6660
gcggttcggt gatgccacga tcctcgccct gctggcgaag atcgaagaga agcaggacga 6720
gcttggcaag gtcatgatgg gcgtggtccg cccgagggca gagccatgac ttttttagcc 6780
gctaaaacgg ccggggggtg cgcgtgattg ccaagcacgt ccccatgcgc tccatcaaga 6840
agagcgactt cgcggagctg gtgaagtaca tcaccgacga gcaaggcaag accgatcccc 6900
ggggacctgc agcaatggca acaacgttgc gcaaactatt aactggcgaa ctacttactc 6960
tagcttcccg gcaacaatta atagactgga tggaggcgga taaagttgca ggaccacttc 7020
tgcgctcggc ccttccggct ggctggttta ttgctgataa atctggagcc ggtgagcgtg 7080
ggtctcgcgg tatcattgca gcactggggc cagatggtaa gccctcccgt atcgtagtta 7140
tctacacgac ggggagtcag gcaactatgg atgaacgaaa tagacagatc gctgagatag 7200
gtgcctcact gattaagcat tggtaactgt cagaccaagt ttactcatat atactttaga 7260
ttgatttacc gggctgcatc cgatgcaagt gtgtcgctgt cgacgagctc gcgagctcgg 7320
acatgaggtt gccccgtatt cagtgtcgct gatttgtatt gtctgaagtt gtttttacgt 7380
taagttgatg cagatcaatt aatacgatac ctgcgtcata attgattatt tgacgtggtt 7440
tgatggcctc cacgcacgtt gtgatatgta gatgataatc attatcactt tacgggtcct 7500
ttccggtgat ccgacaggtt acgggcgggc gacctcgcgg gttttcgcta tttatgaaaa 7560
ttttccggtt taaggcgttt ccgttcttct tcgtcataac ttaatgtttt tatttaaaat 7620
accctctgaa aagaaaggaa acgacaggtg ctgaaagcga gctttttggc ctctgtcgtt 7680
tcctttctct gtttttgtcc gtggaatgaa caatggaagt ccgagctcat cgctaataac 7740
ttcgtatagc atacattata cgaagttata ttcgatgcgg ccgcaagggg ttcgcgtcag 7800
cgggtgttgg cgggtgtcgg ggctggctta actatgcggc atcagagcag attgtactga 7860
gagtgcacca tatgcggtgt gaaataccac acagatgcgt aaggagaaaa taccgcatca 7920
ggcgccattc gccattcagc tgcgcaactg ttgggaaggg cgatcggtgc gggcctcttc 7980
gctattacgc cagctggcga aagggggatg tgctgcaagg cgattaagtt gggtaacgcc 8040
agggttttcc cagtcacgac gttgtaaaac gacggccagt gaattgtaat acgactcact 8100
atagggcgaa ttccatttcc ccgaaaagtg ccacctgggt ccttttcatc acgtgctata 8160
aaaataatta taatttaaat tttttaatat aaatatataa attaaaaata gaaagtaaaa 8220
aaagaaatta aagaaaaaat agtttttgtt ttccgaagat gtaaaagact ctagggggat 8280
cgccaacaaa tactaccttt tatcttgctc ttcctgctct caggtattaa tgccgaattg 8340
tttcatcttg tctgtgtaga agaccacaca cgaaaatcct gtgattttac attttactta 8400
tcgttaatcg aatgtatatc tatttaatct gcttttcttg tctaataaat atatatgtaa 8460
agtacgcttt ttgttgaaat tttttaaacc tttgtttatt ttttttctct tcattccgta 8520
actcttctac cttctttatt tactttctaa aatccaaata caaaacataa aaataaataa 8580
acacagagta aattcccaaa ttattccatc attaaaagat acgaggcgcg tgtaagttac 8640
aggcaagcga tccgtctaag aaaccattat tatcatgaca ttaacctata aaaataggcg 8700
tatcacgagg ccctttcgtc tcgcgcgttt cggtgatgac ggtgaaaacc tctgacacat 8760
gcagctcccg gagacggtca cagcttgtct gtaagcggat gccgggagca gacaagcccg 8820
tcagggaatt cccgttttaa gagcttggtg agcgctagga gtcactgcca ggtatcctgt 8880
gcggtatttc acaccgcata taattccact tgcaattaca taaaaaattc cggcggtttt 8940
tcgcgtgtga ctcaatgtcg aaatacctgc ctaatgaaca tgaacatcgc ccaaatgtat 9000
ttgaagaccc gctgggagaa gttcaagata tataagtaac aagcagccaa tagtataaaa 9060
aaaaatctga gtttattacc tttcctggaa tttcagtgaa aaactgctaa ttatagagag 9120
atatcacaga gttactcact aatgactaac gaaaaggtct ggatagagaa gttggataat 9180
ccaactcttt cagtgttacc acatgacttt ttacgcccac aacaagaacc ttatacgaaa 9240
caagctacat attcgttaca gctacctcag ctcgatgtgc ctcatgatag tttttctaac 9300
aaatacgctg tcgctttgag tgtatgggct gcattgatat atagagtaac cggtgacgat 9360
gatattgttc tttatattgc gaataacaaa atcttaagat tcaatattca accaacgtgg 9420
tcatttaatg agctgtattc tacaattaac aatgagttga acaagctcaa ttctattgag 9480
gccaattttt cctttgacga gctagctgaa aaaattcaaa gttgccaaga tctggaaagg 9540
acccctcagt tgttccgttt ggcctttttg gaaaaccaag atttcaaatt agacgagttc 9600
aagcatcatt tagtggactt tgctttgaat ttggatacca gtaataatgc gcatgttttg 9660
aacttaattt ataacagctt actgtattcg aatgaaagag taaccattgt tgcggaccaa 9720
tttactcaat atttgactgc tgcgctaagc gatccatcca attgcataac taaaatctct 9780
ctgatcaccg catcatccaa ggatagttta cctgatccaa ctaagaactt gggctggtgc 9840
gatttcgtgg ggtgtattca cgacattttc caggacaatg ctgaagcctt cccagagaga 9900
acctgtgttg tggagactcc aacactaaat tccgacaagt cccgttcttt cacttatcgc 9960
gacatcaacc gcacttctaa catagttgcc cattatttga ttaaaacagg tatcaaaaga 10020
ggtgatgtag tgatgatcta ttcttctagg ggtgtggatt tgatggtatg tgtgatgggt 10080
gtcttgaaag ccggcgcaac cttttcagtt atcgaccctg catatccccc agccagacaa 10140
accatttact taggtgttgc taaaccacgt gggttgattg ttattagagc tgctggacaa 10200
ttggatcaac tagtagaaga ttacatcaat gatgaattgg agattgtttc aagaatcaat 10260
tccatcgcta ttcaagaaaa tggtaccatt gaaggtggca aattggacaa tggcgaggat 10320
gttttggctc catatgatca ctacaaagac accagaacag gtgttgtagt tggaccagat 10380
tccaacccaa ccctatcttt cacatctggt tccgaaggta ttcctaaggg tgttcttggt 10440
agacattttt ccttggctta ttatttcaat tggatgtcca aaaggttcaa cttaacagaa 10500
aatgataaat tcacaatgct gagcggtatt gcacatgatc caattcaaag agatatgttt 10560
acaccattat ttttaggtgc ccaattgtat gtccctactc aagatgatat tggtacaccg 10620
ggccgtttag cggaatggat gagtaagtat ggttgcacag ttacccattt aacacctgcc 10680
atgggtcaat tacttactgc ccaagctact acaccattcc ctaagttaca tcatgcgttc 10740
tttgtgggtg acattttaac aaaacgtgat tgtctgaggt tacaaacctt ggcagaaaat 10800
tgccgtattg ttaatatgta cggtaccact gaaacacagc gtgcagtttc ttatttcgaa 10860
gttaaatcaa aaaatgacga tccaaacttt ttgaaaaaat tgaaagatgt catgcctgct 10920
ggtaaaggta tgttgaacgt tcagctacta gttgttaaca ggaacgatcg tactcaaata 10980
tgtggtattg gcgaaatagg tgagatttat gttcgtgcag gtggtttggc cgaaggttat 11040
agaggattac cagaattgaa taaagaaaaa tttgtgaaca actggtttgt tgaaaaagat 11100
cactggaatt atttggataa ggataatggt gaaccttgga gacaattctg gttaggtcca 11160
agagatagat tgtacagaac gggtgattta ggtcgttatc taccaaacgg tgactgtgaa 11220
tgttgcggta gggctgatga tcaagttaaa attcgtgggt tcagaatcga attaggagaa 11280
atagatacgc acatttccca acatccattg gtaagagaaa acattacttt agttcgcaaa 11340
aatgccgaca atgagccaac attgatcaca tttatggtcc caagatttga caagccagat 11400
gacttgtcta agttccaaag tgatgttcca aaggaggttg aaactgaccc tatagttaag 11460
ggcttaatcg gttaccatct tttatccaag gacatcagga ctttcttaaa gaaaagattg 11520
gctagctatg ctatgccttc cttgattgtg gttatggata aactaccatt gaatccaaat 11580
ggtaaagttg ataagcctaa acttcaattc ccaactccca agcaattaaa tttggtagct 11640
gaaaatacag tttctgaaac tgacgactct cagtttacca atgttgagcg cgaggttaga 11700
gacttatggt taagtatatt acctaccaag ccagcatctg tatcaccaga tgattcgttt 11760
ttcgatttag gtggtcattc tatcttggct accaaaatga tttttacctt aaagaaaaag 11820
ctgcaagttg atttaccatt gggcacaatt ttcaagtatc caacgataaa ggcctttgcc 11880
gcggaaattg acagaattaa atcatcgggt ggatcatctc aaggtgaggt cgtcgaaaat 11940
gtcactgcaa attatgcgga agacgccaag aaattggttg agacgctacc aagttcgtac 12000
ccctctcgag aatattttgt tgaacctaat agtgccgaag gaaaaacaac aattaatgtg 12060
tttgttaccg gtgtcacagg atttctgggc tcctacatcc ttgcagattt gttaggacgt 12120
tctccaaaga actacagttt caaagtgttt gcccacgtca gggccaagga tgaagaagct 12180
gcatttgcaa gattacaaaa ggcaggtatc acctatggta cttggaacga aaaatttgcc 12240
tcaaatatta aagttgtatt aggcgattta tctaaaagcc aatttggtct ttcagatgag 12300
aagtggatgg atttggcaaa cacagttgat ataattatcc ataatggtgc gttagttcac 12360
tgggtttatc catatgccaa attgagggat ccaaatgtta tttcaactat caatgttatg 12420
agcttagccg ccgtcggcaa gccaaagttc tttgactttg tttcctccac ttctactctt 12480
gacactgaat actactttaa tttgtcagat aaacttgtta gcgaagggaa gccaggcatt 12540
ttagaatcag acgatttaat gaactctgca agcgggctca ctggtggata tggtcagtcc 12600
aaatgggctg ctgagtacat cattagacgt gcaggtgaaa ggggcctacg tgggtgtatt 12660
gtcagaccag gttacgtaac aggtgcctct gccaatggtt cttcaaacac agatgatttc 12720
ttattgagat ttttgaaagg ttcagtccaa ttaggtaaga ttccagatat cgaaaattcc 12780
gtgaatatgg ttccagtaga tcatgttgct cgtgttgttg ttgctacgtc tttgaatcct 12840
cccaaagaaa atgaattggc cgttgctcaa gtaacgggtc acccaagaat attattcaaa 12900
gactacttgt atactttaca cgattatggt tacgatgtcg aaatcgaaag ctattctaaa 12960
tggaagaaat cattggaggc gtctgttatt gacaggaatg aagaaaatgc gttgtatcct 13020
ttgctacaca tggtcttaga caacttacct gaaagtacca aagctccgga actagacgat 13080
aggaacgccg tggcatcttt aaagaaagac accgcatgga caggtgttga ttggtctaat 13140
ggaataggtg ttactccaga agaggttggt atatatattg catttttaaa caaggttgga 13200
tttttacctc caccaactca taatgacaaa cttccactgc caagtataga actaactcaa 13260
gcgcaaataa gtctagttgc ttcaggtgct ggtgctcgtg gaagctccgc agcagcttaa 13320
ggttgagcat tacgtatgat atgtccatgt acaataatta aatatgaatt aggagaaaga 13380
cttagcttct tttcgggtga tgtcacttaa aaactccgag aataatatat aataagagaa 13440
taaaatatta gttattgaat aagaactgta aatcagctgg cgttagtctg ctaatggcag 13500
cttcatcttg gtttattgta gcatgaatca tatttgcctt tttttcctgt aattcaatga 13560
ttcttgcttc tatactatcc tcaatgcaaa accttgtgat cttcacaggt cgatactgac 13620
caattctatg aactctatca ccactttgcc attcaacact agggttccac catgggtcta 13680
aaatgaatac ttgcgaagct atatggtgca ctctcagtac aatctgctct gatgccgcat 13740
agttaagcca gccccgacac ccgccaacac ccgctgacgc gatgcgtaag gagaaaatat 13800
gggagggtaa tcagccgtgt ccaccaacac aacgctgtcg ggcggttcta taggattccg 13860
cattgcgcct acctacaggg tgcctcaacg gtatccgcga cttgcgatgt gcctgctatg 13920
aacatgacgg ggatgtggat cgcggcgtag cgggcgacgt tggccacgct gtcgcgggcg 13980
gccgggtcgg cgaggacgtc ggtgtagttg tcgaagccgg tgaggacctg gcggtacccg 14040
tcgaagccga gcccggagag gcggtcggcg aagaggctga ggtagaaggc gtacccgatc 14100
gggatcagca cggtacagag cagcagcagt gcgaacggtg ccgccagtgc ggccccggtg 14160
aagcggcgcc ggcgggctgc ccggcgtgac cgcgcggagg ccgggccagg gggcgtcgtc 14220
gccggtgtgg ggacggctgt catgtccggc tcccttcggt gacggccagg ccgcgggccc 14280
gcatcgcggc gacggtcgag gtgtgaaccc ggtgcagcgc ccgggggatg gtggtggctc 14340
cggtggagac cgggccgagc gcgtcggcga tcgtggcgaa ggtgctcagc gaggtcgggc 14400
cccacgtcca cccggggacc cgggtcgcgg actcgtccag cacgtcgaag acggggtcgc 14460
cggtgaagta cccgtcctcg taggcggtac gggccacctt ccggttggcg aggaaagcgg 14520
gcgaggggaa ggtgaacgcc gagccgatcc ggggaaccgc ggggtcggtg ctgagccagc 14580
gcaggaacgt cagggcggcc tcgggcacct cgctctcggc ggagaccccg aacgccgacc 14640
ctccccagac cccggtggac gggtcgtcgc cccaggtggg caggggtgcg accgcccagc 14700
ggccctggtg ggcgggcagg gacttcttca gcatgcccac gccccaggtg gccccgagca 14760
tcccccagag ccggccgtgg tgcatcgcgg agatccagct ctgggtgccg gtggcgtcgc 14820
gcgcggcaag accggaggac accatgtcct gccagtactc ggccacgcgc atcgtgcccg 14880
cgccgtcgat cccgacccgc cacgcgttcc cggcgatgtc ccaccagggg tcgccggcct 14940
ggcaggccat cccggcgaag aaggcgccgt cgttgagcgg gaaggtggtg atccgcgcat 15000
cccggtcggc cgtgcggacg gccgaggcgg cccggcggaa ctcctcccag gtacggggca 15060
ctccgatgcc ggcccggtcg aacaggtcct tgcggtagaa gaacgccatc gggcagaagt 15120
ccatgggcac cgcccaggtc ttcccgccgg ggcggacgct ctgccacgcg gcgggcagga 15180
agccgtcggc gagatcgtcc acgtccgcgc tcagatcgcg cagcccgccg gtggtcgcga 15240
tctgcggcag ggcctggtac tcgacgtgca ggatgtccgg ggcgttgtgg gcgcggatcg 15300
cgttggtctg ctgggcgtag cccccggacg taccggcggc gatcacgctc aggtcgaccc 15360
ggacgtcgcg ctgggaggag ttgaacgcgg cgacgtaccg gtccatgccg gtcagccacg 15420
accacatacg gacccggacg gttccggagg tccggcccga acagccggtg agtgtgccgc 15480
cggcgagaag cgctgccccc aggcccgcgg aggtccgcag cagggacctc cgcgatcggt 15540
gcgtccgagg ggcgtgtgcg ctgtcaccgg cgtcggagga gaagccgagc accaggggtt 15600
tcctttcgtg caggaaggtg ttcgcacact tcagcccctg gccgattttc ggtcaacggt 15660
agcgaaatga atcgatcagg atcgatagga aagcaaagct caacgagctc cggagaggcc 15720
ccgctcccga tccgagagcc ctcccgctag ctcgcattcc cggcaaagcg ggtagcgcct 15780
tcctggatcg ggggcgcctg cgatagaaca gacgacatcc acgagaagtc gaagagaagt 15840
cgaacgacac agttcgatca gaaggaaaag gagaggccgg gatggcactc gcatcggaac 15900
ggcaggagtt catcctcgcc accgtgcggg aacgcggcac cgtgcgcctg gccgatctgg 15960
tcgagcggct gggcgtcacc gccgtgacgg tccgccggga cgtcacgatc ctggccgacc 16020
gggggctggt gtcgcgcgtc cacggaggcg tcaccctccc ctatcgcggg ccgacggccg 16080
acgagcgggc cacccggggc gtgttcaccc acggccgcct gcccgaccag gcgttcgtcg 16140
gcatggtcgt ccccaccgtc gagtactact ggccggcggt catccagggc gcccagtcgg 16200
cggtggccgc cgccggggga cggctggtcc tgcgcgcttc cgcctacgac gcggcggagg 16260
accgccgcca ggtcaccggc ctcctcgacc gcggtgtgcg cacgctgctc gtcgccccca 16320
ccaacaccgg cgaggccggc caggacctgc tgcgctggct gggctcgctg aacgtccccg 16380
tcatcctcgt cgaacggctg ccgccgcccg ctctgcccac cctgccgctc gacgccgtca 16440
ccaccgccca cagcctcggc gcgggcctgg ccgtacggca tctggtcacc ctgggccatc 16500
accgtatcgc cttcgtcacc gcgcgcttca gccccaccac caaggccctg cgcgaggggt 16560
ggcaggagac caccgcctct ctcggcctgc cctccggcga cggcctcgca cacgacgtac 16620
cgtcgtacgg ctcccccggc tgggcggacg cctacgacgc ggccctccgc agctgccgcg 16680
aggccggcgc caccgccctg ttcgtccact ccgacggtga ggccgtcggc ctggtggagc 16740
gcgcccacga acacggactc gcggtccccg aggacctcgc cgtcatcacc tacgacgacg 16800
aggtggcagc cgccgccgac cctcccctga ccgccgtgcg cccgcagaag caccggctcg 16860
gcgtcctggc cgccgaaatg gccctggcac gcgccgtcga tcccgtcgaa cgcccggtgc 16920
accgagtcga gttgtggccc aacctgatca tccgtgcgtc ctgcggcggc accgcgcccg 16980
tctcggcggg acggtgaagg cgggctgagg gggccggggc tggctcaccc ggtgagccag 17040
ccccggagcg cggcctgtac cccggcctgg aaacgggtct ccgcgttgag gacctgcatc 17100
agacgggtga tgcggcgccg gaccgtgtgc acatggatgt cgaggcgccg ggccatcgcc 17160
tcgtccttca ggccggaggc gagcatcgtc agcaactccc ggtcgccgtc cgtgatctga 17220
tcatgcgtca cggagccgat ggggagcgcc cgttcccaga ccgactcgaa cagcggcacc 17280
agcgcgttgc tcagcagcgc gtcgctcacc accagtgcgg aggccgtcgg gtccgccgcg 17340
tccgtcggcg gcaggagggt cacccgccgg tcgaccgtga tcagcttcgt cggcagatcc 17400
gtgcccaccc ggatctgcac cccctgcacg gccagatcgc ccaggccgcg ggcgcgcccg 17460
tggaagctca gcccctcgcg gtccaccacg acccggacgc gtaccccccg acgtaccggt 17520
tcggccatgt cgagggggac gggcatcccg tccggctcgc tcgacgcgta cggcggccgg 17580
tccaggaggg cgatctcctc gctcgccgac accagcaggg aggccacccg ggcggcgatc 17640
gccgctccgc cgcggaccgt ctcgatgccg gtggcgcggg gggtgtgcac ggagctcagc 17700
agttgggccg cgatccggtc gacggatccg ctgagctcct ccaactccgc cgatcggtgc 17760
aggagttccg cctggtgcag atggatcaga ttgcgcaggg cggtggcggg ggccatggcc 17820
acgggcagcc cggcgccccg ggccgggtgg gtgaagcggc gctcggccag gtgatccagg 17880
gcgttcgtga cccggcgcac ggtcagcccc aggtgctggg cgagggcggt acgggaggcg 17940
ggtacgggtg cgtcgaggag cgcttcgtac acccggagtt cgtccgctcc gagcccgagc 18000
gcctgccatt gcgtgtccca cccagtccgc cccaccggcg gaattgtcac ctgtcaatgc 18060
aggccacaca agtgtgtacg tccacaagtg cacgcgcgga acgcattgtt cccacccccg 18120
ccccggtact tccatgtcgg catccgagat ccgttcatgc agcaatcgca ttcgggaggg 18180
gcaactgtgc ggagttcatc gcgcagcaga tcggccggac caggtcaggg gtccggcacc 18240
acgttcgtcg gccagggcgt ggtggcggta ctggccgccg cgggcctgtt gatgacggcc 18300
acgcccgccg ctcacgcgga caccgccacc acgtccgccg cgtccgaggt gacgccgggt 18360
gccgggaccg ggaccccctc gctcgtcacg ggactcaacg aggaggccga cgccaccggc 18420
accgcggcgg acgccgcccg tggccacctc aaggcgaaga agagccgcta ccgcatcgcg 18480
cacacctcgg cgaaggacct ggccgcggcc gggaccacga ccgggagggg cggcagggag 18540
acggtccgtc tccagcagaa gtaccgcggc gtcacggtcc tcggcggcca gtacctggtc 18600
cggatgacga agaaggacgg caagcgcacc gtcaccggca cgtccggcag ctacttcacc 18660
gagctgaagc tcgacacggt gacgccggag gtctccgaga agaccgcgat cgagcgcgcg 18720
gtcggcgccg tctccgcgca gctgggcggg ggcctgctca gggccccggc caagggcgag 18780
aagcccggcg gggttcagct gtcgggtacg gccggtgacg tgaccgtcct gccccaggga 18840
tccggtgtgc tgacccggca catcaccgtc accggcgtca gcccggccga cggcacgccg 18900
gtgaagcagg aggtgtacat cgacgcccac tccggcttcc ccgtgatgca gtacagcggc 18960
atcaagtcgt tcggcgcacc gggttcgggc gccggtacga gcagcggcgc caccggcgac 19020
gcgcccacgg ccgacgccac cgcggaggcc acgcagcagc tggtcgtcaa gggctcgggc 19080
acccgctaca acggcgagga ggccgagatc aacctgtacc agggcgccga cggcgcctac 19140
cagatgtacg actacagcaa gcggggcgcc gacagcccgt acgacggtcc gctgctgtcg 19200
acctgggacg cgcgtggccg ggaggccgcc acggcgtccg gcacgtggcc cagcggcatc 19260
cagacgttcc gctcggacac ccccgacctg ggcaaggagt tcaccgactc cggggcggtg 19320
gacgcccact gggcggccgg gaaggtctac gactactaca agaaccactt cggccgtaag 19380
agcctggacg gcaaggacgg gttcatgtac tcgctcgtgg gtgtggtgaa cggcgggaag 19440
ccgttcaaca acgccttctg ggacggcacc aagatggtgt acggccaggg cggcggcgac 19500
taccgcacct tctcggcgga caccgacgtg gtcggccacg agatgacgca cggtgtcatc 19560
gagcacaccg cgaacctggt gtacgcgggc cagtccggcg ccgtgaacga ggcgatagcc 19620
gactacttcg gcaacgccat cgacctggag gccgacggcc ggtcgatgga cgacccggac 19680
gccggtctgc tcggcgagga cctgtgcacc acgctcggcc cgcgcgactg cgcgctgcgg 19740
gacctcaacg acggtgccac cacgtcgaag gacttcatcg gcgtcagcca caacggcgac 19800
aacggtggcg tacacctcaa ctccacgatc ttctcgggcg ccctgtggga catgcgcgag 19860
gacctcggcg ccgagccggc cgaccagatc gtctaccgcg cgctgtccgc gtacatgacg 19920
ccgctcgacg gcttcaccga gggccgcgcg gcggtcatcg ccgcagcgca ggagctgggc 19980
gccaccaagg cgcagacgaa gatcgtgaag cgttcgttcg acgcgcacgg catcgtcccc 20040
ggctgggaga aggcgctcgg tgtcgacacg gacaccctcg tgtccaaggt caacgtggcg 20100
ggcaccggcg tcggcgccgg cggtggcaag tacgccgtct cccgctccga cgaggaaggc 20160
agcgaaccgt actcggtgtg gctcggcaac accgacggca agggcaagcc gcagctggtg 20220
agcggcaaca acggcaacta caacgtctac gcgacgacgg acggcaagaa cgtcgcctgg 20280
gccgagttct ccccggggtc ggtcctcttc catgtccgtc cggtgggcgg cggcccggcc 20340
agggtcgtcg gcggcgtcgg agccaatgtc tcgcagctcg tcctcgaagg cgacttcctc 20400
gcctacacgg tgacgaaccc gcagaccggc cggcagcaga tcggctacat caacatcagg 20460
acccgggagg cgggcatgat cctcgccaag ggcatcgagg tcacggccat gccctcgatc 20520
cgggacggga agatcgccta cgccacgatg tggatcgacg gcagcgacgt ccaactgggc 20580
atcggggtcc tggacaccgc caccggcacc acccggatga tgccggccgg agccgagggc 20640
gcgcagggca tcggccagac ggccttcacc gacgacggtg tcttctggat cgaggacggc 20700
gccggcaccg accccgccaa ggccgccgtc gtgcgcgccg acgccgacgg cacgaaccgc 20760
accacggtcg tccccgaaac cggccaaggc tccctgcacg cctaccagct gacggcctcc 20820
gacgacgcgg tcaacgtcac ctcgctgccg ccggccacct catgggccaa cgagaccctg 20880
ccgaagctgt tccaggtggc acccgacggc aagggcggcg cccagcgcgt gtcctgcaac 20940
cgcggcgacc aggtctacgc ctcggccgac gagggcaagc gcgtcgtctg gctggacggc 21000
acgaccggtt acacgaacgt cgtgaagcgg gaccgcccgg cgggcgcgtg ctgacctgac 21060
ctgagggaaa cgggagaggc ggtcgacccc gatgggccga ccgcctctcc tgcgtggctc 21120
tacggcgaca gtggccgatg gagtggagtg atccaggcgg cggtcagtga tccgcgagga 21180
ggccgggcag tctgcgcatg tcgtcgaaga cgacggtgcc ggggccttcg agacggtccg 21240
cgggagtcag tcccccggcg taggcgaagg ctcgcatgcc cgcggcccgg gctgcctgaa 21300
gaccgtactg actgtcctcg accacggcgc acgcctcggg cacgaccccc atcttccgcg 21360
cggcgtgtag gaacagatcc ggggccggct tgccgtgctc gacctcggtg gcactgaaaa 21420
tgcggccttc gaagcgcggg cggagaccgg tcatccccag cgtgaaacgc atcttgtcgt 21480
ggctcccgct ggatgccaca caggtgggga gatgcgtgag cgcgtcgagg gcgtcgacga 21540
tgccctcgac cggcgtgagt tcggcggcga gcgcctcgtc gtagaggggc ttgaactctt 21600
cctcccagtc ggccggcaga cggcgcccga ggtggtcctc gatctgccgc gtcatcgacc 21660
ggctcgacag gcccatgaac cggtcgacga tctcggcttc ggtgagattc caccccagct 21720
cggccaggac gagtgcgtcc acgcgcaccg cgatgcgttc gctgtcgacg agtacgccgt 21780
cgcagtcgaa tatgacgagt tcaatgggtt gggccatgcc cgcaggatag gcgggccgtc 21840
cccgtcacac gaggacggcg ttcggccacg cccggcagag agtcggacgg tcagctcccg 21900
tccacgtaga acccgaaacc gggttgctgc gggatcagcc aggcttccgc ggccggggcg 21960
agatcgacga tcagacgctc cccgacgccg gaggccgtgg tggcgtccgg agtgaggtag 22020
ccgaggccgc gcagctcacg gaccgccgtg cggaaggcct cgcggccctc tgcagccaga 22080
cgctcgtcgc ggttgagcgc gaccagctcg ctgatcgtgg ggtcctgccc ggggccgtag 22140
ccgacggaga gttcggccag gatcccgcgg gcacgccacg tcaggcgcat gtcccgcatc 22200
gcccgcgcga agaggacggg gtcatgggcg ggcggggcgg gaggtaaagg ccaccgcggg 22260
ggacgagtgt cgggggtgct catgtgcagg tggggtcctt cggtgaggct catggtccga 22320
acgggactga gcggcgggac ggtgtgtgga tcaactgtcc cggaaaactc aaccacatct 22380
ggcggtccgg gttgttccgg ttcgtgtcca ccaaactctg tcagggcaac cctgaggatg 22440
gctggccgca tcgatccagt cggcacttcg tacggcttca actcggcgga gcaagtcgcc 22500
cgccaacgtg acgctcgtga tcgcgcaagg cttctgacca aggtcagcgc gccccctctc 22560
aggcgcggga accgcgcgca gtcccagcag tggtcggcgg cctcggcgag ccggtcctga 22620
gcgtcccagg tgaagtgcca gaccgctgtg cggccacgga gcgttgagct ccgacggatc 22680
accggccgcc cttgcgcgtc gtgcgcggcc cggtgtcggg ttccggctct ccataggcac 22740
aggacgagga gaggatgccg tcggggcctt cggtccgcac cacccggccg gcctggtcgt 22800
agacgtacgg aaggtggcgt cgttgcagtc ggtccaggag gtgacgcgtc catcggcgtc 22860
gcaggtgaac cgcactggcg aacctgatga gttcacggcg gcgtcgaggt tgccgacggt 22920
cggcagccga gcacggatga gtccgcccca ggaccagctg caggacgccg gggagcccca 22980
ggacggtctg cgggagcagc atctcaccgg tggcgacgtc cacggggtcg ttcttgcacg 23040
tcttggcgcc cgatggtcgc caaagccctt ggtcgccttg aggtcgccca tcagccggcc 23100
cgtcgatccc ggcggcggac cgccgtccac cgaggcgagt cgcagtcctt ccttcgcacc 23160
cgcgactccg tcctggaacc cctccttgcc ggcgtggacg gcctgaccgg tgtcgatccc 23220
gttctgcagc ccgaccgcgt tggctgcggc ctgaaccgcc aggtcggcgg cgacgttctc 23280
gatcgcctgc actgcgggct cggtcagcgt ggacatgatg caaccggccg cttcctcgcc 23340
cgcctccttc accagcttcc tgaccagggt gccggtggcc gtgacggtgc ccgccggtat 23400
gagcgtcgac agaccgaacg tcagagtggc gacgcttgct cgacgaaggg ctcggaccct 23460
cgtgtgggac cggttgacgg ccttctgacg gcacggcaag gccgtttccc gcccccgggg 23520
ccggggtgcg ggaaccggcc tgtcccgtcc ccggggccgg ggcggtgccg tcagccggcg 23580
ccccacgcct ccggggggaa gagcctgtgc cgggtgaccc gccacaagta gtcggctccg 23640
gcggtgccgg ccgtcccttc aagggggccc aggaagcgtt ccgccagggc cgcgtggcgg 23700
gcgcgccaga cggtgacccg ttcgtcgaac ctcaggagcg cttccccgat ccgttccgcg 23760
tccttcccgt tggcgcatcg ggtccggaag gcgtcgtagg cctccgccac ggagggctgg 23820
cgggcccgtt cccgcagtcg ccgacgttcg gtttccgtga agcccggtgt ggagatgtgg 23880
cggtggtcgc gcagaccgca caggaactcg atctcccggt actgcgccga ctggaagccg 23940
ctcgccgtgc ccagccccgc gcggatcttg ccgaattcct ccgtactcag cgtggccagc 24000
atgtcgaagt ggcggacgag cagttcgaag atctccggca gtcgcggcag cagatgcagt 24060
gccgtgtccg ggtcgtcgtc cgtgagggcc gcgcggattt cctccaggtg gcgcagcacg 24120
atgccgaacc agatctccgc cgactggtgc gtggcaagga agaggaccgg ggcggccgcg 24180
tcgcgcaggt gcgcgacgtt cagcaggtcc tccaggcgca gcagctcccc gtaggtcggg 24240
ctctcgtggt acgtggcgcc ccggccgcca ccgtcactcc cggtcgtccg ggtgtcctgc 24300
gctgtcatgg tttgcgtccg tgggcgacga gggctatccg ggggagctcg gcccggaagg 24360
cattgccgtg cggacggacg gtaagttcca ggaccgcctc gtcgcggggg acgtcctcgg 24420
ggaggtcgcc ggccgagaaa cggaagatgt cgcggagggc ggcttccgtc cgctcgttgc 24480
tccggtcgcc gtcgggcagg atgccgtaca tcgaggtcat gtaggagacg agccgagcca 24540
gtgcgctctc ctcggtctcc ccggtcaggg tcatcgggtc gtacacgggg ccgacggtga 24600
tgtcggtgaa gcccgcgtcg gcgaggtagc cggtcatctc gtgggcggtg aagtggtcgt 24660
acgcgtggcc cgcgagggag cgggggtgga cgacttcgcg gaaccagcgt tcttcggggg 24720
atccctccgc gaagtcgtgg aggacgacgc ggcctccagg ggccaggacc cgggaggcct 24780
ctgtcagcat gcgggggcgt acagagcggt cgacgtggtg agtgccgtac gcgaagagca 24840
cgccgtccag gcagtggtct cgctgcagca tgcggtcggc cggctggcgt acggcgggca 24900
ggccggaccg gagggctgcg gccaccatgt ggccgctgag gtctccggtg accagtggca 24960
cggagtcccc gtcgccggcg ccggccagtt gccgccacac gcggctgagc aggccttcgc 25020
ctccgagtac atcgaggaca cggcccatgt cgcgcagccc gccggggccg gcgaacatgc 25080
cgagaatcgt ctcaatgccg gtgcggcggg cttcgccgtg ctgctgggca cagcggtagg 25140
agacgccgcg gccggtgtgg tcgtcgtcga actcgtttcc ctccgtgacc caaggctcgt 25200
ccgtgacggc gacgaggcgt gcggggtcca ggagctcgaa gagcgccggg tgctgttcct 25260
tgacggtcac gtggtagtcg tgcacggtca tgccggggag gtaggtggtg gtgcgggttt 25320
cgccggtcac gacgggcccc tctctgccgg tactggacaa ggtcgtcgtg cctttcagct 25380
cgttcggaag aaatcgcgaa gaatcttgtt gaccgcgctc gcatcctcga ggtagccgta 25440
gtggccgcag cggggaacgg tctcgaagcg ggcgccgggg atcgcgtcgg ccacttcgcg 25500
gcccaggtac ggcggggtga cgacgtcgtc ctcgaacgcg atgaccaggc aggggacggt 25560
gatgtcccgg tagtccgcga ggcggtcggg cagcaggccg agttccaact gcgcacggac 25620
ccccggcccg tcccgttcgg cgtactcgaa gagtgggagc cagtcctcga cctgatggcg 25680
gtcccggagg gtgtgggggg agaggttctg cagcgcgcgg acagccgccg cgtaggcggg 25740
gggcagttcg acgccgctgt cgtacagggc ggcggccgcc ctggtggcgg cggcccgcat 25800
cgcgtcggtg cgaccccggg tggccatgaa caccgcctgg gtcaccaggt ccgggcgggc 25860
cagggcgact tcctgggcca cctgggcgcc cagggacgtg cccacgacgc ggcagggtcc 25920
gcccgccacc tgttcgatca gcgcggcggt gtcggcggcg aggtcggcga gggtgaagcc 25980
ccgctcgcac tcgtcggacg gcggtacgcc gcgattggtg aacgtgatgg tgcggaatcc 26040
ggccgcgtgc aggtcaggca cctggtaggc gtcccacacc cggccggtac tgccggtgcc 26100
ggcgatcatc agcaccgggt caccgcagcc gctgtcggag caggcgatcc gggcgccgtt 26160
gacctggatc atcctggatg tcgccctcat gtcggccctc cttccgtctc cgctgtccgg 26220
ctgcgggtca ggccgcggtc gtccggtcgt tctccatctg ccggatgagg ctcttggggc 26280
gcatatcggt ccagttctcg ttgatgtggt cgaggcattc ctgccgactg ctctcgccga 26340
aggtgacggt ccagcccgcc gggacatcgg cgaacgcggg ccaaagggag tactggccct 26400
cgtcgttgac cagtacgagg tagctgccgt cgttgttctc gaaggggttg gccatggcgg 26460
tatgtttcct ttcgtgtacg gacagggggg cgcccccgtg tccggtgtcc cgtgtggggt 26520
cctgggggtc aggtgccggc gcccagccgt gcggcgagca cggggccgat ccgggtgagt 26580
gcggctggat cggtcatctc ctcgtgtgcg cagtcgatca ggtgcaggtc gaggtcgccg 26640
gtgacgtggg ggtgccacag gccgacgcgt tcggcggcct gctcggaacc gggccgtgcg 26700
gaggcgaaga acaccatgtc gccggtgaac acgccgggtg tgaaacggcg ggtgagccgg 26760
gcgttgttga ggaagacgtc cttcatcgcg aggatccgcc cggcgtcgaa gtccctcgcg 26820
ggcccgtcgg tgcgccgtac gaactcgagg aactccggga gccacgcgtc gccctcgagg 26880
cgctcggggg cgacggggga accgagctgg gcgaggtagt cggcgacgat gcgctgttcg 26940
tccacctcgg cgtcgggccg ggccccggtg acggggtagg cgtcgaggac ggcgaggagt 27000
tcgacggtgt ggccctgctg ttgcaggcgg gtcgcgacgg cgtgggcgac cgtgccgccg 27060
aaggaccagc cgagcagccg gtacggccca tccgggcaca ggcgccggat ctcaccggcg 27120
tagtcctcag ccatctcctc gacgctcccg ggcagcccgc cgggcgcggt gagcctccgg 27180
gcctgcagtc cgtaaaccgg cacgtccgcg tccagatggc tgagcaggcc ggcgtacgcc 27240
cagctcaggc ccattccggc gtggacgcag aacagcggtg gccggctgcc cgtgcgccgc 27300
agcggcagca ccgtctcgaa cgggtcgtac gccgcggcgt ccgccgtgca cgccgacgcg 27360
agcagggcag gggtcggcgc ctcgaacagg gtgcgcacgg agatctccac gcccagcgcc 27420
gcgcggaccc gggccgcgag gcgagtggcg agcagcgagt gaccgcccag gtcgaagaac 27480
ccctcgtcca cgccgacggt ctcccggccg agcacgtcgg cgaacaaggc gcacagcacc 27540
cgttcctgcg gcgactgggg tgcgcggccg ggggcggcgg tctgggtgcc ggggccgggc 27600
agcgccgtgc ggtccacctt gccgttggcg gtgagcggca gggcgtccag ggtgaggacg 27660
tcaccgggca ccatgtgccg gggcagccgg gacgccaggt gggcgcgcag cgcggcggcc 27720
gacggtgtgc cggggccgcc ggtgacgtac gcgacgagcc gctggtcgcc gagcccgtcg 27780
tcgtacacgg tgaccaccgc gtcctcgacg ccggggtgcg atgcgagggc cgcctcgatc 27840
tcggcgagtt cgacgcggaa gccgcggatt ttgacctgcg tgtcggcgcg tccctcgaac 27900
agcaggtggc cgtcgctgcg gcggcgggcc aggtcaccgg tgcggtacat gcgctcgccg 27960
ggggcgccga aggggtcggc gacgaagcgt tcggcggtct gtccggggcg gcccagatag 28020
ccgcgggcca gtccggcccc ggcgacgtag atctcgccga cgcagccggc gggtacgggc 28080
cggagccggt ggtcgaggac gtacacacgc tggtcgggcc agggtgcgcc gatcggcacg 28140
ggcgcgccgt caccggtccg ttcgcacacg gcgtaggtgg cgccgacggt gacctcggtg 28200
gggccgtagc cgttgaggat ccgccgttcg ccaccggccc agcggtcgac gagttggggt 28260
ccgcacacgt cgccggtgac ggtgaccgtc agccgggggg gtgccgcgcc tgccgggaca 28320
gcggccagcg cggccggggg cagggtgacg tgcgtgatgc ggtgtgcggt gagcgcggcg 28380
gcgagttcgt caccgaagag gtccgcgtcc ggggcgagga cgagggtcgc gcccgacagc 28440
agcgcggagc acaggtccca gacggaggcg tcgaagctgg tcgacaccag ttgcaggacc 28500
cggtctccgg ggcccgcgcg cagtgcctcc tgctgggtgg cggccagcgc cgggatgcct 28560
tcgtgggtga cgacgacgcc cttgggtgtg ccggtggagc cggaggtgta gatgacgtag 28620
gcgggatggg cgggcagcag tcccgtgccg cgcggattgc cgtcgggcgc ggcggcgagt 28680
gcggcggtga cggccgggtc gtcgaggtcc agtacgggga tgccggtggg gagcatgcgg 28740
tcgcgggtgg ctgcggaggc gatgaggagc gccggggccg cgtcggcggc catgtgggcg 28800
atgcggtcgt cggggtaggc ggggtcgacg ggcacgcaca cggcgccggc cttgaggatc 28860
ccgagccagg ccgtcaccag gtccgcggag cggggcaggg cgatcgccac gcgctgttcg 28920
ggccgtacgc cccggtcggt gagcagccgg gcgagccggt tggcccgccg gttcagtgcg 28980
tcgtacgtcg tagtggcggg ccggcggccg gcgacctcga ccgcgatgtg accggggtgc 29040
gccgccaccc gggcctcgaa cagctcgggc agggtggcgg gcggcaggcc ggtgacagtg 29100
ccgcggctgc gggtcagcag tttgtggagg gtgtgttccg ggagcagccc gaccgcgtcg 29160
acgcgggtgc ccggggcgtg gacggccgcg tcgaggaact gcacgaagca gtccgcgatc 29220
tgccggacgg tgccctcgtc gtacaggtcg gcgtggtatt cgacgattcc ggtgatgccg 29280
gagggttcgc ccttctccgg gggccgctcg gtgaacgcga acgcgaggtc gaacttggcc 29340
gtcccggcgt cgaggggctg ttcggtgacc tgcaggtcgg gcagcagggt cccggggcgg 29400
tcggagatgc tgcggtcctc ggtgttctgg aaggcgagca gtatctggaa gagggggtgg 29460
cgcagcagtg agcgttccgg gttgaggacc tccaccaggc gttcgaaggg cacgtcctgg 29520
tgtgcgtacg cgtcgaggtc ggtggcccgc acgcgcgcga ggagttcggc gaacgtcgga 29580
tcgccggaga cgtcggtgcg caggacgagg gtgttcacga agaacccgat gaggtgctcg 29640
gtggcctcgt cggtgcggcc cgcgatcggc gtgccgatgg ggatgtcggt gcccgcgccc 29700
agccgcgaca gcagaccggc gagggccgcc tggaccacca tgaacacggt ggtactggtg 29760
acgcgggcga gggtgacgat gtcgcggtgc agcgcggcgg ggatctccag ccggcaccgg 29820
ccgccgcggc ggccgacgtc ggggcgtcgg gggcggtcgg tgggcaggtc caggccctgc 29880
gggaggccgg cgagtctgcc ccgccagtgg gcgagctggc cggccatctc gctcgtcccg 29940
tcgtcctcgg tgccgagtac gtcgcgctgc caggcggcgt agtcagcgta ctgcacggac 30000
agcgggggca gttccggcgc ggagtgtgcg cgccgggctg cgtaggcggc ggacaggtca 30060
cggaggaacg gctcgcggga ccaacggtcg cttgcgatgt ggtgggtgac gacgagcagg 30120
gtgtggtcgt cgcgctcggt gcggaagacg gtggcgcgga cggggatctc ggcggtgagg 30180
tcgaaggggt ggcgggccgc ttcggcgagg cgcccggcca gttcgtccct gtgcaggggt 30240
acggcctcaa gggtgattcg ggcgcgaggg tcgccggtgg gcaggatgtg ctgccacgcg 30300
gtgccggtgc cgtcccgggc gatgagtgtg cgcaggctct cgtggcgggc gacgacatcc 30360
gtcagggcgg cttcgagggc ggtcacgtcg agaggaccgg tgaggcgcag cgcgagaggg 30420
atgttgtatg tggcgccgtg gccctcgagt tcgtggagga accacaggcg ctgctgggcg 30480
tacgacaggg ggtcccgccc ggtgcgttcc cgcgcggtca ggggggcgcg gacgggggcg 30540
ccggagcgtt cccccaggag ccggtcgagg cggccggggg taggggcgtc gaagagggcg 30600
cggacgccga gttcgacgcc gaggaccgaa cggacgcggg acaccaggcg ggtggcgagc 30660
agcgagtggc cgccgagttc gaagaagtcg tcggccgtgc cgacccgttc gaggccgagg 30720
acttcggcga acaggccgca cagaacttcc tcacgggccg tgccgggggc acggaagccg 30780
gcgccggaga cggcctcggg cgcgggcagg gccgccctgt tgatcttgcc gttgggggtg 30840
aggggtacct cgtgcagtac cacgacggcg gacggcacca tgtacgcggg caggcgggcg 30900
gcggcatggg cggtcaccgc cgacggcagg ccgccggtgg ggtcgacgtc ggcaggtgtc 30960
acgtacgcca cgagccgctg gtcgccgggg cggtcctcgc ggacagcgac gatcgcgcgc 31020
gccacaccgg gggtggcggc gacggtggcc tcgacctcac cgagctcgat gcggaagccg 31080
cgtaccttga cctggtcgtc gacgcggccg acgaactcca gcgtgccgtc ggttctccac 31140
cgcaccaggt caccggtgcg gtacatgcgt tcgcccggtg cgccgaaggg gcaggccacg 31200
aagcgttcgg ccgtcaggcc ggggcgtccg agatagccgc gggccaggcc cgcgccggcc 31260
acgtacagct cgccggcgac gcccgtgggc acgacgcgca ggccgccgtc gaggacgtac 31320
aggcgggtgt tccacagggg ccggccgatg gggacggggc cgggaggcag ggtggtgccg 31380
ggggcgatgc ggtgttcggc gcagttgatc gtggtctcgg tggggccgta cacgttgaag 31440
acggtcacgc cggggtggcg ggcgcgccac gggtcgaggg tctcgctcag gagttgctca 31500
ccgccgaggg tgatggcccc ggtgggggag atctcgtcgg ggagggagtc gaggacggcg 31560
agatggctgg gggtggcctt catgaaggtg gggctgtcct cgcccaggtc gaggatcttg 31620
tcgtcggtga acgatccgag gcgcaggcag ccaccgctca ccaggggtgc gtacagcgct 31680
ccgacggtga ggtcgaagga cacgggcgag tgcacgaatg ccttgccggc ggcttcgggg 31740
tattcctcga tcgtgcgcag gaggtagccg gtgacggaac ggtgcaggat cacgacgccc 31800
ttggggcggc cggtggagcc cgaggtgtag atgacgtacg cgggctggtc gggaagcggc 31860
gcgataccgt cggtgccgag gtcgtgttcg ggcagggcgc gaacgcccgt gtcgagggcg 31920
tcgtcgtcga gggtgaggac gcgggccggg ccggcgggca tggccgcggc cgagcgggag 31980
tcggtgagga cgagcgcggg gccgatgtcg ccaagcatgt acgcgatgcg gtcggccgga 32040
tactccgggt ccacgggaac gtacgcggcg ccggtcttca tcacggcgag agcggccacg 32100
accatgcggg cggagagcgg gagcagcagg gtgacgtact gctcggggcc cacgccggcc 32160
gcggcgaggt ggcgtgcgag ccggttggcg cgcgcgttga gttcggcgta ggagagttcg 32220
ccgtcgcggg catggaccgc cggcctgccg ggggtcgggg ccgcctgggc ctcgatcatt 32280
ccgggcacgg tgagcggggg cacgtcgcgg tgggtgtcgt tgaggccggt gaggagatgc 32340
tgtttctcct cctcgccgag gatgtcggcc ctgccgacgg cgaggtccgg ctggtcggcc 32400
atgagctcca gggctcgcac gaggctgtcg cggatcgtcg tgacgcgttc ggcgggcagg 32460
gcgctcggcc ggtggccaaa gcgcaggcgc agcggcgggc cgggcaggac cagcagaccg 32520
agggggtagt gcatggcgtc gcgtacgtgg acgtcggcga cccggggttc ggccgcgggg 32580
ccgtcggcgc cggcggagga tgcggcggcg acggggtagt tctcgaagac catcaccgtg 32640
tcgaagagtt ctgtccggcc ggcgagccgc tggatgaccg ccagtcgttc gtactggtgg 32700
tcgaggagct gtatctgttc gcgctggagg cggacggtga agtcccggag gctctcgtgc 32760
ggcagcagct cggcacgcag cggcacggtg ttgatgaaga ggcccaccat gtcctcgaca 32820
cctgcgagct ccggcggccg gccggacacg gtcacgccga acacgacgtc gtcgcggccg 32880
gtgaggcggc cgaggacgag cgcccaggcg gcctggacga gggtgttgag ggtgacgccg 32940
aactcgcggg cgcgggcggc gagcgtcgcc gccaggtccg gttcgagccg ggactcggtc 33000
cacagcggcg gctcggcggc ccggtcggca cccggggcga cgagggtggc ctcgtcaacc 33060
ccggccagtg ccttggacca ggcccgcccg gcctcgtccc ggtcgcggcg ggagatccag 33120
gccaggtagt cccggtacgg ccggacccag ggcagggcgg tctcgtcgcc ggtgcggtag 33180
agggtgatga gttcccgcag caggacgggc atcgaccagc cgtccatgag gatgtgatgg 33240
aaagtcagcg cgaaacggtg ccggtcgtgg cctgtgcgca cgagcaggaa ccgcagcagg 33300
ggcggagccg tcagatcgaa gcggcgccac cgctggtcgt gcagcaggcg ttcgatctcg 33360
gccttctgct cggcgccctc gagcttcgcg acgtcggtgt gctcccacgg caccgcaacc 33420
tcgcgcggga tgatctgcac ggggcgctgc agccgctcgt accggaaggc cgcccgcagg 33480
ttggcgtgcc gacgcagcag ggcggccgcg gcggcgcgca gtgcttcggg gtcgacgggc 33540
ccctcgaggt cgacgaccac ctgcacggtg tacacgtcga cggactgctc gtcgtaaaca 33600
ctgtggaaca gcagtccctc ctgcagcgga gtgagaggaa gtatgtcctc catcgcgcgc 33660
tgcgtcatcc ctgtgtcctc cagtcgtctt cgagcagttc gatctcagca aaggagagtt 33720
ccgcgaggcc gacgtccgac ggggtcagcc cacctgcctc cgggtggcga gcgtgctcca 33780
cgaggccctc gagcgcctca tcccagtacc gtgcgagctc gcggatgcgc gactccggga 33840
ggagggtggt gggccaggac cacgtgaccc cgagacgggg accttcgggc gtgtcgaggg 33900
tgatggcgtt gaactccacg gcgtggcgga ccgggagctc cgggtcctga cccgcagcgc 33960
tccgtgccag caggctccag gcaggcgcac ggtccagggc gtccgcggtc gtcgtctcgt 34020
ccgtgcccgt cgtgaaccga cccaggtagt tgaacccgaa ctggggcgtc gcgtggcgag 34080
cgagggcagg tccgtgttcc gggtccaggt accggaggat cccgtagccg agtccctggt 34140
ccgggaggct gcggagcgtc tccttgacct cccggacgac ccgaccgacg acgggaccac 34200
ctgcccggac ctcatcccag gaagccgact cgaccgggac acgcacgggg tagacgctcg 34260
tgaaccagcc caccgtccgc gagaggtcgg caccgcggac ggcgtcctcg ttccgaccgt 34320
ggccctccac gtcgacgacc acgggaccgc cacgaccccg ccagcgtgcc acggcgaggc 34380
cgaacgctgc gagcaggacc tcctggacac ccacaccgag cacgctgggg acctcggtca 34440
ggagggcacg ggagacggag acggaggcac ggctgtcgac ggagacgacg ccaccgaaga 34500
cgtcgacggc accgtcgacg gcacgggagc cgacgagcac gtccgcaccg cggaccatgc 34560
gggaccagag ctcgacctcg cctgcacgct ccccacggcg accctgctcg gcgagagcgg 34620
cagcccatcc gcggagggag gtgccgacga cgccgagctc gacacgccca ccggagcgga 34680
cctgggccca gccctccgcc aggtcgccga ggacgacgcg ccaggagacg ccgtcgacga 34740
cgaggtggtg ggcgacgagg acgaggacac cggaacggtc accaccgcgc tcgagccaga 34800
cggcacggac gacgcgtccg acggacgggt cgagccacgc acgggcctcg acggctgcgg 34860
cacggacggc gtcgacctgg ccgtcgacac ccatgcccga gaggtcgcac cgcacgatcc 34920
aggagccgtc gtcgggcacg acaccgtcca cgcagagggc tcccgagtcg aggacccgga 34980
gccgccaggc ctcgtgccgg tcccggacgg cacccaggag gacgcggagc tcctcccaga 35040
ccaggcctgc cgggacagcc aggaccaccg actggttgaa gccacggacg acgccaccga 35100
gggatccacc gccacgctcg gcgagccact cgacgaccgg ccagcgctcg acctcgccga 35160
cacccgggag gacgatctca gcgccaccga cgacacccac cgccccagcg tcggaaccac 35220
ggacgacggc agcgagagca cgcacggtac gcccttcgaa cacgtcccgc acggacagct 35280
cgagacctgc acggcgagcg cgagcgacga gctggatgga caggatcgag tcgccaccga 35340
ggtcgaagaa gccgtcgtcg acgccgacgc gttccacgcc gaggacttcg gcgaagagtc 35400
cgcagaggac ttcctcacgc gggctggagg gtgcgcagaa accggcgacg gagacgaccg 35460
ggaccgggag gcctgcgcga tcgaccttgc cgttgggggt gagcgggatc tcgtcgagca 35520
cgacgaccgc ggaggggacc atgtaggcag ggagacgctc ggcgacgaga cccgagatgg 35580
ccgaacgcag cccgtcgcca ccgacgccac cgaccgtgac gtaggcgacg aggcggtggt 35640
caccgggacg gtcctcccgc acgaccacca cggcacggac gacgtccggg tgggcagcca 35700
cggcaccctc gacctcgccg agctcgaccc ggaaaccccg gaccttgacc tgatcgtctg 35760
cccgacccac gaattccagc gtcccgtcga cgcgccagcg gacgaggtca ccggtccggt 35820
acatgcgctc accggggaca ccgaaggggc aggcgacgaa gcgctcggcg gtcagtgccg 35880
ggcggccgtg gtagccgcgg gccaggccgg cgccggccag atacagctcg ccgggcacgc 35940
ccggtggcac aggacgcagg gcggcgtcga ggacgtaggt gcggatgttc cagatggggc 36000
ggccaatggg gaccgtggcc gtgccggtgg cgggctcata cgcgtggtgg gtgacatcga 36060
cagccgcttc ggtcgggccg tacagattgt gcagggggac gggcagtttc gcgcggaagt 36120
ccacggccag gtccgtgccg agggcctcgc cgctgcatat cacccgcctc agtccggtgc 36180
agagtgccgc gccgggctct tcgaggaaga cgcgcagcat ggagggcacg aaatgtgccg 36240
tggtgatgcg ctcgcgctcg atgagtccgg ccagatacgc cgcgtcccgg tggccgccgg 36300
gccgggcgaa cacgaggacc gcgccctcga gcagcggcca gaagaactcc cagaccgaca 36360
cgtcgaaacc ggccggagtc ttctgcaaga cacgatcggt cgcgtcgaga cggtactccg 36420
cctgcatcca ggcgagccgg ttgacgatgg cgccgtggga gacgacgacg cccttggggc 36480
ggccggtgga tccggaggtg tagatcatgt acgcggggtg ggcctcggtc accggcgtgc 36540
cggggttcgt gccgggggcg gtggcgagtg tcgcgcggat ctctgtgtcg tccagcccga 36600
tccgcgggac ggacccgggg agttcgacgt ccccgcgggt caggaccgcc acgggggcag 36660
cgtcgccggc catgtacgcg agccggtcgg ccgggtactc ggggtcgagc ggtacgtagg 36720
ccgcgcccga tttcacgacc gccagcagtg ccactacgag gtccggactg cggggcaggg 36780
ccacaccgac gaaccgctcg gggcctgcgc cgtgctcggc caggacatgg gcgaggcggt 36840
tggcgcgtgt gttcagttca cggtacgaca gttcttcacc ctcgtacacg accgccggcg 36900
cgtcgccccg ggctgcggcc cgtgcctcga acagcgccga cagcgttccc gtcacgccgg 36960
ggacggcggt gtcgttccac tccgtcagga cgcaggcccg ctcggcgtcg ctcaccacgg 37020
gcagctcgcc caggcgtacc gacgcgtcgg cggcgacccg ctccagcacc cgcaccaggc 37080
gctccgcgat ccgtgcggcg gtggggtgat cgaacaggtc gcaccggtac tcgaccgctc 37140
cccggagctc accgccgacc tccgacaggc cgaacaggag gtcgaacttg gcggctccgg 37200
caccgagcgg gagacccgct gccgagagac ccgggaagtc ctccaccggt ccgccatccg 37260
cggtgttgtc gaacgccagc atcacctgga acagcgggtg ccgtgccagc gaccgctccg 37320
ggttcacggc ctcgacgagc cgttcgaacg ggatatcctg gtgcgcgtat gcgtccaggt 37380
cggtcgcacg cacacgggcg agcagctcgg cgaaggtcgg atcgcccgac acgtccgtgc 37440
gcaggaccag ggtgttgacg aagaatccga tcaggtcctc cgtcgcctga tcggtccggc 37500
ctgcgatggg cgtcccgatc gggatgtcat cgcccgcacc caggcggctc agcagcgctg 37560
cgagggcagc ctggaccacc atgaacacgg tcacgccctc cacgcgagcc agccgggtca 37620
ggtcgcgcag cacgcctgcc ggcaccgtga agcccacgcg accgccctcg taggaggcgc 37680
gtgcggggcg cacgcggtcg gcaggcagcg cgagctccgc aggcaggtcc gccagctgcg 37740
tccgccagta cgccagctgc gcggagagct cggagctctc atcgtcttcg gtgcccagca 37800
catcccgctg ccacgctgcg tagtcagcgt actgcaccgg caggggcggg agctgcgggt 37860
cgcgaccctc caggcgtgcg gcgtaggccg tcgagaggtc ccgagccagg aggccggtgc 37920
tccacccgtc ccctgcgatg tggtggaaga cgagcaggag ggcgtgatcc tcgtccgcga 37980
cccggaacac cgtcgcacgg agcggcatct ccgcggtcag gtcgaacggc cgacccaccg 38040
cctcgttgag cttggccacc agcgtgtccg cgtccgtctg caccaccggc agcgcagggc 38100
gagctgccgg gtcacccacc gggaggacct gctggcgagc ggtcccgtcg ctgtcccgag 38160
cgatgagcgt ccgcagcgac tcgtgccgtg cgatcacgtc gatcagggcc tggtggaggg 38220
cctccacgtc gagcacgcca gcgagccgga gcgccatggg gatgttgtag gccgcaccgg 38280
gtccctccag ctcctccagg aaccagaggc gctgctgcgc gaaggagagc ggctcgcgac 38340
cggtgcgctc gcgtgcgacg agcgggacac ggacaccgag cccgctgcgc tcgcggagga 38400
ggcgctcgag gcgcgacacg gtcggggcct cgaacagcgc acggacaccg gcctccacgc 38460
cgaggacggc acgcacacgg gagatgagcc gcgtcgcgag cagcgagtgg ccaccgaggc 38520
cgaagaagtc gtcgtcgaca ccgacgcgct cgacgccgag gacctcggcg aagaggccac 38580
agaggacctc ctcgcgaggg ctcacgggag cccggtagcc ggcaccagcc tcgacacccg 38640
ggaccggcag agcggcacgg tcgaccttgc cgttcggggt gagcgggatc tcgtcgagca 38700
cgaccaccgc gctcgggacc atgtacgccg ggagacgttc ggcgaccgca cgcatgacgg 38760
cggacgagag gccaccggtg tccacacccg tgacgtaggc cacgaggcgg tggtcacccg 38820
ggcggtcctc gcggacgacc accaccgcac ggacgacgtc agggtgggca gccacagcgc 38880
cctcgacctc gcccagctcg acgcggaacc cgcgcacctt gacctggtcg tcggcacgac 38940
ccacgaactc cagggcaccg tcgacgcgcc accgcacgag gtcgccggtc cggtacatgc 39000
gctcgccagg ggcaccgaac gggcacgcga cgaagcgctc ggcggtgaga ccggcacgac 39060
ccaggtaccc acgggcgagg ccgtcgcccg cgatgtacag ctcgcctgcg acgcccggtg 39120
gcacgacaca caaagcagcg tcgagcacat acacgcgagt gttgaggatg ggtgtgccga 39180
tcgaaggggg agcgctgtcc ccggcggcct gcccggcgct ggtggaccag atggtcgtct 39240
ccgtcgggcc gtacatgttc gtgacggacc gcgcgcctcg tgtcagcgcg tcgcgcaacc 39300
cggggctcag ggcctcaccg cccacgacgg cgtgcacgcc gcgcaggctg tcgcccgcgt 39360
cggcgagcat gccgtgccac cagctcggtg tcgcctggac catggtgacg ccgtggtcct 39420
cgcacgtctg gcgggccagc gccgggtcgc ggaccgtctc cccgtccgcg aggacgacgg 39480
tggcgccgct cagtagcggg acgaagagct cgagggccgc gatgtcgaag cccacggtgg 39540
tcacggacag caggcggtcg ccaccgcgga gcccgagcct gggcaccatg tcggccagga 39600
agttcaccag ggcggccgac ggtacgacga cgcccttggg ctggccggtg gatccggagg 39660
tgtagatcac gtacgccggg ccggcggggt tgccccggcc ggcgggggac gtggccgggt 39720
ggtcgtggag ctcgccggtc acctgctcgg agtcgagtac cacatggctt ccgccgggta 39780
tgcgggcgct gatgtcctcc gtggtgagga ccaccgcagg gcgggcgtca tccagcatgt 39840
agctgagccg ctcggccggg aagccgggat ccagcggcag atgtccggca cctgttttct 39900
gtacggccag cagggccacc aggagctctt cggtccgggg cagggccacc gcgacgaacg 39960
tctcgggggc cgcgccccgg gcgagcagca agtgggccag gcggttcgcg cgcgcgtcca 40020
gctccgcgta cgtgagccgg atgttccccg agacgagagc gacatcgtgc ggggtgcgcc 40080
gcacctgctc ctcgaacagc tggggcagca gtgccggggg gagagcggca gccgtgtcgt 40140
tccactcgtg cagcagttgg tcgcgctcgc ccgcccggag cacgtcgacg ctgctcgcac 40200
gttgctccgg ggctgccgca aaagcagcga gaacatgcag gaaccggtcg gcgagctgcc 40260
gcacggtgtc ctcgtcccac aggtcgcggc ggtactcgaa gacacttctg atgccctggt 40320
ggtcgcccgt ctcaccgcgg gattcgttgc aatacagggc cagatcgaat ttcgccacgc 40380
ccggttcgat gtcctgccgg gaggcagcca ggccttcggc catgggcagc ggggtgctcg 40440
tctcggcgtt gttgaacgcg agcatcacct ggaagagcgg gtgacgggcc aggctccgct 40500
ccgggttcac cgcctccacc agccgctcga acgggatatc ctggtgcgcg tacgcgtcca 40560
ggtccgtggc gcgcacacgc gccagcagct ccgcgaaggt ggggtccccg ctcacgtccg 40620
tccgcaggac gagcgtgttg acgaagaacc cgatcaggtc ctccgtcgcc tggtccgtcc 40680
gacccgcgat gggcgtcccg atcgggatat cgtccccggc acccagccgc gacagcagcg 40740
cggccagcgc ggcctggacc accatgaaca ccgtcacccc ttccgcacgc gccaggtccg 40800
cgagcgaccg gtgcacgtca gcggggatcg tgaactcgat ccggccaccc cggtacgtcg 40860
ccacggcagg gcgagcccgg tcggtgggca gcgcgagctc cgccgggagc gacgccagct 40920
gcgtccgcca gtacgcgagc tgcgcggaga gctccgagga ctcgtcatcc tccgtcccca 40980
gcacgtcccg ctgccacgcg gcgtagtcgg cgtactggac cgacagcgga cgccacgcgg 41040
aagccgctcc ggctgcgcga gccgagtagg ccgtcgacag gtcgcgagcc aggggaccca 41100
tgctccagcc gtcacccgcg atgtggtgga gcacgaggag caggatgtga tcgttgtccg 41160
tgagccggaa caccgtggca cgcaccggca ggtccgccgc caggtcgaac ggacgaccag 41220
ccgcctcgtc caggcgctcc tgcagggcat ccgcaccgat gtcgaccaga ggcagacccg 41280
gacgcgtccg aggatcgtcg accgggagga tgtgctgcca cgccgtaccg gcgtcctggg 41340
cgatcagcgt ccggagcgac tcgtggcgcg cgatcacgtc gatcagggcc tggtggagcg 41400
cctccacgtc gagcacaccc gcgagccgca gggccatggg gatgttgtag gccgcacccg 41460
gaccctccag ctcctccagg aaccagagcc gctgctgcgc gaaggacagc ggctcacgac 41520
cggtgcgctc gcgtgcgacg agcggcacgc ggaccccgag accggagcgc tcacgcagca 41580
ggcgctccag gcgcgacacg gtcggggcct cgaagagggc acggacaccg gcctcgacgc 41640
cgagcacggc acgcacacgg gagatgagac gggtcgcgag cagggagtga ccgccgagct 41700
cgaagaagtc gtcatccaca ccgacccgct ccacaccgag gacctcggcg aagagaccgc 41760
agaggacctc ctcgcgaggg ctcaccggag cgcggtaccc cgtcccggcc tcgacctcgg 41820
ggaccggcag cgcagcacgg tcgaccttac cgttcggggt gagggggatc tcgtccagga 41880
ccacgacggc ggacgggacc atgtaggcag ggaggcgctc ggccacgaga ccggagatgg 41940
ccgaccgcag gccgtcacca cccacgccac cagcggtgac gtaggcgacc aagcggtggt 42000
caccgggacg atcctcccgg acgaccacca cggcacggac cacgtccggg tgcgcagcga 42060
ccgcaccctc gacctcgccg agctcgacgc ggaaaccgcg caccttgacc tggtcgtcgg 42120
cgcgaccgac gaactccagc gcaccgtcga cgcgccaacg gaccaggtca cccgtccggt 42180
acatgcgttc gccgggcacg ccgaacgggc aggcgacgaa gcgctcggcg gtgagaccga 42240
cgcgacccag gtaaccccga gcgaggccga caccggcgac gtagagctcg cccacgacgc 42300
ccaccgggac gacgcgcagg gccgcgtcga gcacgaacat ccgctggcca gcgaacgggc 42360
gaccgatcgg gaccgggccg tcgggcacct cctcgccagg ggcgatcagg aactccgcgc 42420
agttgacggt gagctcggtg ggaccgtagg cgttgacgac cacgacaccg gggttgcgct 42480
cgcgccactc gcggagagcc ccaccggaga gggcctcgcc accgacgagg agcatcccgt 42540
taccggcgac gacgccctcg agctcgccga ggagaccgag gtgggacggg gtgaccttga 42600
cgaaggaggc cccgacaccc tgggcggact cgtcgagctc gcccagcacc acgcagccac 42660
ccgagaccaa cggggtgaag agcacggtga cggtcaggtc gaaggcgagg gacgagtgga 42720
cgaacccgac accgcccacg gcgcctgcgt agacgtcccg gccacgggcc aggtaaccgc 42780
cgaccgaggc gtgggtgacg acgacgccct tgggacgacc cgtggacccg gaggtgtaca 42840
cgacgtagag ggggtgaccg gggaggagac gggaggcgcg ctcgccgtcg gtgacgttgc 42900
gcacctcgac accgtcagcc gcgacgagct cgaccaggca ggactcgcgc accacgcggg 42960
agaattcgaa cacctcgcca ccggggaagt cacccacgac gtgggagagg ccctcaccga 43020
cgaccacgag gccgacaccg gcgtcggcga ggacccagcc gacgcgctcg acgggccact 43080
cggggtcgac gggcacgtag aagccaccgg ccttggcgac ggcgagcagc atgacgacca 43140
cgtcgacacc gcgacccatg acgacgccga caccgcactc ggcaccgacg ccgcgtccga 43200
cgagccagcg ggcgaccacg ttgacccggg cgttgagctc gccgtaggac cagacgacgc 43260
ctgcaccacg gacggcgacg gcatcggcgt ccgcacggac gcgctcttcg aacaggccga 43320
cgagaccgcc tccgggcacg accgggaccg gtccgccgac gccccaaccg agcaaccgtt 43380
cgcgctcgcc gacaccgagg acatccacac cgctcacgga gacaccgcca ccgccgctga 43440
cgacgccctc gaggacccgg accagccatc cccacacctc acgcacccga cccgcatcaa 43500
acacatccgg ccgataaccg atttccagtt ccagctccag gccgtgcatg gaagcaacga 43560
ggttcagtgc gtagtgggtt ccgttgtatc cgcctgttcg tgagatctgg aattccgctt 43620
ccggggattc agaagaaacc gggtagttgt cgaagaccat tccgacatcg aagagttcct 43680
tgtgcccggc ccaccgctgg acttccgcca gccggacatg ctgatgctcc agcagctccg 43740
tctgctcccg ctgcagccgc tccacgaaac cacccagcga ctccgccgga tccagccgcg 43800
cccgcaacgg aatggtgttc acgaacagac cgaccatgtc ctccacaccc accacctcgg 43860
caggccgacc cgacaccgtc accccgaaca ccacatcgtc ccgacccatc aaccgcccca 43920
gcaccaaggc ccacgccgcc tgtaccaccg acgcaaccgt cacaccacgc ccgcgcgccc 43980
acgccacgat cttctgcgag aggtcgccgt cgaccgcacc gtggaacgcc gccgggacga 44040
ccccgtctcg ggaaacgcct ggcgcgacga gcgacggctc ctccaggccc gtcagcgccc 44100
gccgccatgc gccttcggcg gctgcggcgt ctcgcgcggc gacccatcgc aggaaatcgc 44160
cgtacgagcg gacccccggc agaaccgata cgtcccccga gctgccgtac agggccagca 44220
gctcacgcag cagcaccggg acggaccagc cgtcgaggac gacgtggtgg tacgtgatga 44280
cgaaccggaa acgatcgtcg tgcgtccgga tgagcacgaa gcgcgccagt ggaccccggc 44340
ccatgtcgaa ccggcgccag cgctcctctt ccaccaaccc gtcgataccg ccgtcgcgca 44400
cgtcgatgga ctgccacggc agatccacct gtcgtgcgac gaccgcaacc cattcgcccg 44460
accgcagttg gcggtatccc gaccgcagca ccccgtgccg cgccaccaga ccttcgacag 44520
ccgcgcgcag ccggtcgacg tccaccggac cgaccagatc gaacaccagc tgccccacat 44580
acacgtccac cccaccagag gccaacgcgc tgtggaacag cagcccctct tgcagggccg 44640
acaccggcag aatctcctcc gcaccaccgg cgacccccgc ctgcagacgg tcgagctcca 44700
cctggttcac ttccaccagc gtcacgtcgg acggcgtgag gccgccggct tcggggtgcc 44760
gggcgtgttc gaccagccct tccagggcct cgtcccagta gcgggccagc tcccgtatcc 44820
gggactccgg cagcagcgtc gtcggccacg accatgtcac gcccaggcgc gggccctccg 44880
gggtgtccag cgtgatcgcg ttgaactcga ccgcgtgcgc cacgggcagt tcggggtcct 44940
ggccggctcc caccgcgaac ggccctgaca cggggaccca gtccgtcatc ccctcgtcac 45000
cggtgtcgtc ggttccggtg gtgaagcggc cgaggtagtt gaaaccgaac tgcggggtgg 45060
catgccgggc cagagcagga ccgtgctcgg gatcgagata gcgcaggatg ccataaccca 45120
gaccctggtc aggcagcgaa cgcagagtct ccttcacctc acgcaccaca cggcccacca 45180
ccggaccacc cgcacgcacc tcgtcccacg aagcggactc caccggcaca cggaccggat 45240
acacactggt gaaccaaccg acagtacgag acagatcagc accccgcaca gcgtcctcat 45300
tacgcccgtg cccctcaaca tccaccacaa ccggcccacc ccggccgcgc caccgcgcga 45360
ccgccagccc gaatgccgcc agcaacacct cctgcacacc aacacccaga accgacggca 45420
cctccgtcag caacgcacgc gacaccgaca ccgacgcccg cgaatcaacc gacaccaccc 45480
cgccgaaaac atccaccgca ccatccacag cacgcgaccc caccagaaca tccgcacccc 45540
gaaccatccg cgaccacaac tccacctccc ccgcacgctc gccccgccgg ccctgctccg 45600
ccaacgccgc cgcccaaccc cgcaacgacg tccccaccac acccaactcc acacggccac 45660
ccgaacgcac ctgcgcccac ccctccgcca gatcccccag caccacccgc cacgacacac 45720
cgtccaccac caggtgatgc gccaccagca ccaacacccc cgaacgatca ccaccacgct 45780
ccagccacac cgcccgcacc acccggccca ccgacggatc cagccacgca cgcgcctcca 45840
cagccgcagc ccgcacagca tccacctgac catccacacc cataccgctc aggtcacacc 45900
ggacaatcca cgacccgtca tccggaacaa caccatcaac acacaacgcc ccggaatcca 45960
gcacccgcaa ccgccacgcc tcatgccgat cccgcaccgc acccaacaac acccgcaact 46020
cctcccacac caacccagca ggcacagcaa gcacaacaga ctgattgaaa ccccgaacca 46080
caccacccag cgacccccca ccacgctccg ccagccactc caccaccggc caccgctcca 46140
cctcacccac acccggcagc acaatctcag caccccccac cacaccaacc gccccagcgt 46200
ccgaaccacg caccacagcc gccagagcac gtaccgtacg gccctcgaaa acatcccgaa 46260
ccgacaactc cagacccgcc cgacgagccc gcgccaccaa ctgaatcgac agaatgctgt 46320
ccccgcccag atcgaagaac ccatcgtcca cccccacccg ctcaacaccc agcacctccg 46380
cgaacagacc acacaacacc tcctcccgcg gcgacgacgg cgcacagaac cccgccaccg 46440
acaccaccgg caccggaaga cccgcccggt ccaccttccc gttcggcgtc aacgggatct 46500
catccagaac caccaccgcc gacggcacca tgtacgcagg cagacgctca gcaacggcac 46560
gcatcaccgc agaggacagt ccacccgtgt caacaccggt gacatacgca accaaccggt 46620
gatcacccgg ccggtcctca cgcaccacaa caaccgcacg caccacatca ggatgcgccg 46680
caacagcacc ctccacctca cccaactcca cacggaaacc acggaccttc acctgatcat 46740
ccgcacgacc aacaaactca agcgtgccgt ccacccgcca ccgcaccaaa tcccccgtgc 46800
gatacatacg ctcaccccgc gcaccgaacg gacacgccac aaaccgctcc gccgtcagac 46860
ccacacgccc gagatagccc cgcgccagac ccacacccgc cacatacagc tcacccacca 46920
caccgaccgg caccacgcgc agcccgtcgt ccagcacata cacacacgtg ttgcgcaggg 46980
gccggcccat gggggccgcg gcgtccatgg gggtgtcgcc gtaccaggcc gtggcgtaga 47040
cggtggcttc ggtcgggccg tagaagttgg ccacgcgggc cccgggcagc acagcgcgga 47100
cgcggcgcac gtcgcgagcg gacagcgcct cgccggccag ggcgatcatg cccacgtcgg 47160
cccggtcgag gccggcttcc agcagctgcg cgaaggccga cggcacgccg ctgaccaggc 47220
tcgcggacca tcggccgggg cggtcgacga gggccagcag attccggacg atctcgacgg 47280
tgccgccgca ggacagcggg gcgaagagtt cgaccacgga gacgtcgaag ctgagggacg 47340
tggacaacac gacgtgccgg agccgctcgg cacccaggtc ttcccgcgcc caggcgagga 47400
agttggccag gctgccgtgg ctgatgacga cgcccttggg gcggcccgtg gacccggagg 47460
tgtagatcac gtacgcgggg tgggaggcgg acatcttcac gtcgggccct gtggcggggg 47520
ctgcgaccag ggtggcgcgg gtcgcctcgt cgtccagctc gacccgggga acggtccgca 47580
gagtacgtac gtcggtggag gtcacgacca cgacgggggc ggcgtcggtg accatgtgga 47640
ccagccgctc ggccgggtac tccgggtcca ggggaacgta cgcggcaccg gacttgagca 47700
cggccagcag ggccacgatc agatccagcc cacgcggcag cgccaccccc acgaaatgct 47760
ccgggcccgc accgcgcccc accagcagcc cggccaggcg gttcgcccgc gcgtcgagtt 47820
cccggtacga cagaacctca ccctcgtaca cgaccgccgg cgcgtcaccc cgggccgcga 47880
cctgcgcctc gaacaactcc aggaatgttt ccggcacacc ggggacgccc gtgtcattcc 47940
actccgtcag gacacgggcg cgctccgcct cgccgacgac cggcagttca cccgtgcgta 48000
ccgaagcgtc ggaagccacg cgctccagta cccgcaccag gcgctccgcg agctgcgcca 48060
ccgtcgggtg gtcgaagaga tcgcagcggt actccacggc tccgcgcagc tcgccgccca 48120
cctcggagag accgaagagc agatcgaatt tgaccgctcc cgcacccagc ggcaggcctt 48180
ccaccccgag gcccgcgaag ccctccgtga cctcacggtc aatcgtgttg tcgaaggtca 48240
gcatgacctg gaagaggggg tggcgggcga gggagcgctc ggggttgacc gcttcgacca 48300
gtcgttcgaa ggggatgtcc tggtgggcgt aggcgtcgag gtcggtggcc cggacgcggg 48360
ccagcagctc cgcgaaggtc gggtcaccgc tgacgtccgt gcggaggacg agggtgttca 48420
cgaagaagcc gatgagatct tcggtggcct ggtcggtgcg gccggcgatc ggcgtcccga 48480
tcgggatgtc gtcgccggca cccagccggg agaggagcgc ggccagcgcc gcctgcacga 48540
ccatgaagac cgtgacaccc tcggcacggg cgagagccac caggccgtcg tacacaccag 48600
cggggacggt gaactcgacc ttgccgcctt cgtacgaggc gacggcggga cgggcacggt 48660
ccgtcgggag cgccagctcg gcaggcagcg aggctagctg ggtgcgccag taggcgagct 48720
gggccgacag ctcgctcgac tcgtcgtcct ccgtgccgag cacgtcgcgc tgccaggccg 48780
cgtagtccgc gtactgcacg ggcagcggac gccagtcggg ggctgcgccg ttcgtacggg 48840
cctcgaaggc gcgcttgacg ttccggatga acggcacacg ggacatcgcg tcgaaggcca 48900
cgtggtgggc cacgaccagg aggatgtggt cgttgtcggt gaggcggaag accgtggcac 48960
ggaccgggag gtcggcagcg aggtcgaagg gccgaccagc ggcctcgtcg agccgctcct 49020
gcagggcgtc ggcaccgatg tccaccagag ggagaccggg acgggtgcga gggtcgtcca 49080
cgggcaggat gtgctgccag gccgtacccg catcctgcgc gatgagggtg cggaggctct 49140
cgtggcgagc gatgacgtcg atgagcgcct ggtgcagcgc ctcgacgtcc agcacaccgg 49200
ccagacgcag cgccatcggg atgttgtacg cagcaccggg accctccagc tcctccagga 49260
accacagacg ctgctgagcg aagctcagcg gctcccgacc cgtccgctcc cgtgccacca 49320
gcggcacccg caccccgaga gcgctccgct cccgcagcag ccgctccaga cggctcaccg 49380
tcggcgcctc gaagagcgca cgcacacccg cctccacacc gagcaccgca cggacacgcg 49440
agatcagacg cgtcgccagc agcgagtgac cacccaaccc gaagaaatca tcgtccaccc 49500
ccacccgctc cagcccgagc acttcggcga agagaccgca caggacttcc tcacgcgggc 49560
taacaggcgc ccggtagccc gcgcccgcct ccacccccgg caccggaagc gccgcccggt 49620
ccaccttccc gttcggggtc agcgggatct cgtccagcac gaccacggcg ctcgggacca 49680
tgtaggcagg gagacgctcc gcgaccgcac gcatgaccgc gctgctgagg ccaccggtgt 49740
cgacacccgt cacgtacgcc accaggcggt ggtcaccggg acggtcttcc cggacgacca 49800
cgaccgcacg caccacgtcc gggtgggctg ccacggcacc ctcgacctca cccaactcga 49860
cccggaagcc acgcaccttg acctggtcgt ccgcacgacc cacgaactcc agggcgccgt 49920
ccacccgcca ccgcaccaga tcccccgtac ggtacatacg ctcacccggc gcaccgaacg 49980
ggcacgccac gaaccgctcg gccgtgagac cggcacgacc gaggtacccc cgtgcgagac 50040
cgacaccggc cacgtacagc tcaccgacga cacccaccgg gacgacccgc agcgccgcat 50100
ccagaacaaa catccgctga cccgcgaaag gacgcccgat cggcacaggc ccatccggaa 50160
cctcctcacc aggcgcgata aggaactcgg cacagttcac cgtcagctcc gtcggaccat 50220
aagcattcac caccaccaca cccggattac gctcacgcca ctcacgcagc gcaccacccg 50280
acaacgcctc accccccacc agcagcatgc cgttccccgc caccacaccc tccagctcac 50340
ccagcaaacc cagatgcgac ggagtcacct tcacgaacga ggcacccacc ccctgcgccg 50400
actcgtccaa ctcacccaac acaacacaac cgccagacac caaaggcgtg aacagaaccg 50460
tcaccgtcag atcgaacgca agcgacgaat gcacaaaccc cacaccacca acggcacccg 50520
catacacatc ccgaccacgc gccaaatacc cacccaccga agcatgcgtc accacaacac 50580
ccttcggccg ccccgtcgaa cccgacgtat aaaccacata caacggatgc cccggcaaca 50640
gacgcgacgc ccgctcacca tccgtcacat tccgaacctc aaccccatcc gcagccacca 50700
actccacaag acacgactca cgaacaaccc gcgaaaactc gaaaacctca cccccaggaa 50760
aatcccccac cacatgcgac aacccctccc ccaccacaac cagccccacc ccggcatccg 50820
ccagcaccca ccccacccgc tccaccggcc actccggatc caccggcaca taaaacccac 50880
ccgccttcgc caccgccagc aacatcacca ccacatccac cccgcggccc atcaccacac 50940
ccacaccaca ctccgccccc acaccccgac ccaccaacca ccgcgccacc acattcaccc 51000
gcgcattcaa ctccccataa ctccacacca cccccgcgcc acgcacggcc accgcgtccg 51060
cgtcggcccg cacccgctcc tcgaacaacc ccaccaaccc accacccggc accacaggca 51120
ccggcccacc cacaccccac cccaacaacc tctcccgctc acccacaccc aacacatcaa 51180
cccccgacac agacacccca cccccaccag aaaccacacc ctccaacacc cgcaccaacc 51240
gctccgtcgt ttcccgggcc tgtaccgcac cgaacacatc aagccggtat tccagcaccc 51300
cgcgcattcc cgaggccgca ccgtccgcgg cgtacatctc cgtgcacgtg aaagcgagat 51360
ccaccttggt ctgatccgtc cggatggcct caccggagac ctccagttcc cccagattcc 51420
ccagcgcgct tccgaccccg tccatgtccg ggacgttgaa cgtcagcatg acctggaaca 51480
gtggatgccg tgccagtgac cgctccgggt tcaacacctc gaccagacgt tcgaatggca 51540
cgtcctggtg tgcgtacgcg tcgaggtcgg tggcccgcac ccgcgcgagg agttcggcga 51600
acgtcgggtc gccggacacg tcggtgcgca gcaccagcgt gttgacgaag aacccgatca 51660
gttcctccgt ggcctcgtcg gtgcggccgg cgaccggggt gccgatgggg atgtcgtcgc 51720
cggcgccgag acgtgacagc aggacggcga gggcggcctg gacgaccatg aagaccgtcg 51780
cgccgtgggc ccgtgccagg tcggcgaggg cctggtggag tccggcgggc acggtgaatt 51840
cgacgcggtc tccggcggtg gaggcgacag cggggcgtgg gcggtcggtg ggcagcgtga 51900
gttctgcggg ggcccctgcg aggcgggtgc gccagtgggc gagttgggcg gagagttcgc 51960
cggtctcgtc gtcctccgcg cccagcacct cccgctgcca gagcgcgtag tcggcgtact 52020
gcacgggcag cggttcccag tcgggtgccc tcccggccgc ccgcgcccgg taggcctcgg 52080
ccacgtcgcg tgcgagcggt cccatggacc agccgtcccc ggcgatgtgg tgcaggacga 52140
cgaccagtac atgttcctcg tcgccgacgc ggaacagtgc ggcgcggaag gggatctccg 52200
ccgccaggtc gaagggcctg cgtacgaact cggtgatgag ggcacgcagg gtcgtcccgt 52260
cggccccggt cacggtgagc agagggccga ggccggtcgc gggcagcacg atctggtggg 52320
caccgtcccg gtcctcctcg aacacggtgc gcagcgcctc gtggcgttcg gcgacgtcgg 52380
tgagtgcggc gcacatggcg gtgctgtcca gggggccacg caggcggagt gcgacgggca 52440
tgttgtacgc ggcgctgtcg cggtcgagcc ggttgaggaa ccacaggcgg cgctgggcgt 52500
acgacagcgg gatccgtgcg gggcgcggtc cggcggtgag cgcggggccg gcctgcgcgg 52560
cccggtggag ggcggtggcc agggaggaga ccgtgcggtg ctcgaagagg gcgcggacgt 52620
tgagttcggt tttcaggttc gtacggatac ggctgacgag tttgctggcc agcagcgagt 52680
gcccgccggc gtcgaagaag tcgtcgtcgg cgccgaacga gggaagcccg agcacgtcgg 52740
cgtacaggcc gcagaggatc tcctcgacgg gtgtgcgggg gcgctcgggg cggggcgcgg 52800
taccgaaggc gggctccggc agggcgccgc ggtccagttt gccgttgggg gtgagcggga 52860
gggtgtcgag gagaacgaaa gccgagggca ccatgtacgc gggcagccgg gcggcgaggt 52920
gcccggtgag ttcccgcggg tcgacgggtg tgccggggac gacgtacgcc acgaggcgag 52980
cctctccggt ggcggagcgg tgcaggagga cgacgttctg ggcgacggcg gggtggccgg 53040
ccagggcgag ctcgatctcg gcgggttcga tgcggaagcc gcggagtttg atctgatcgt 53100
ccacgcgtcc gaggtattcg accacgccgc cgggactgcg ccggacgagg tcgccggtgc 53160
ggtacatgcg ttcgcccggc gcgccgaagg ggcaggccac gaagcgttcg gccgtcaggc 53220
cgggccggcc ggcgtagccg cgggcgagct gggtgccggc gaggtagagc tcgccggcga 53280
cgccttcggg tacgggacgc aggaagccgt cgaggatgta ggcggccgtg ttgtcgaggg 53340
ggtggccgat gacggggcgg ggtgcgatgc cgaccgtgca ggcgagggcg tcgacggtga 53400
attcggtggg gccgtagtag ttgtacgagg tgacgccggg gacgtcggag agcctggtcc 53460
agagtttcgc gtcgacgcct tcaccgccga gcatgaaggc ggcggggtgg tggcggccct 53520
cgtcgaggat tccggtctct acgagttgct gggcgaagga gggggtgacg tcgatgacgt 53580
cgatgcgctg ccggtcgatg agttcggcga cgcgttcggc gtctcggcgg gtgtcgtcgt 53640
caataaggtg cagttcgtgg ccggcggcca ggcagatcag accttcccag gaggtgtcga 53700
aggagaggga ggcggtgagg ccggccttga gccgtgatcc tccggcggca tggggttcta 53760
tgagggcgcg ccgatggtcg tggaagaggt tggcgagact gcggtgttcg atgacgacgc 53820
ccttggggcg tccggtggag ccggaggtgt ggatgatgta ggcggcgttg cgggcgttga 53880
gcggggtacg gcggtcggcg tcggtgaggt tgtgtgcggg tgccgcagcg agggcgcggg 53940
cggtggtggg gctgtcgagg acgagggtgc ggccggggtg cggcagcggg gtggcggtgg 54000
tggtcgtgag gacgacggtc gggtgtgcgt cggcgaggat gccggtgagc cgttcggcgg 54060
gctggtccat gtcgagcggc aggtaggcgg cgcccgcttt gagtacggcg aggatggcga 54120
cgacggattc cgcggaacgg gggaaggcga gcgcgacgaa gtcgtcggag ccggcgccgg 54180
cggcgacgag caggcgggcg agccggttgg cgcggtcgtt gagttcttcg tacgtccagg 54240
aggtgctggt gaagacaagg gccgtgtcgt ggggccgcct ccgcgcctgt tcctcgaatt 54300
cctggtggta gtaggtgggc ggctgctcgc gcgtggtgtc gttccagccg cgcagcaggc 54360
ggtggcgttc gtcgtcgtcg aggaggtcga ggcgggcgag ggtgtgcccg gtgtcgtcgg 54420
tggcggtttc caggacgcgg gtcacgcggc gcagcagggc ggtggcggtg gtggtgtcga 54480
cgaggtcggg gcggtggtcg agcttgaggc gcagggtttc gccgggtacg gcggtgaggg 54540
tgagggggta gtgggtggcg tcggagcctt cggtggcggt gacggtgaag tcgtcggtgc 54600
ggatggagcc ggggccgctg tggccgatgg ggtagttctc gaagacgaca atggtgtcga 54660
agagttcttt gtgtccggcc cagcgctgga tgtcggagag ccgcacgtgc tggtgttcga 54720
ggagggtggt ctgttcggcc tggaggcgag cggcgaacgt gccgagggtg tcggcggggt 54780
cgaggaccgt gcgcaggggg acggtgttga tgaagagtcc gaccatgtgc tcggcgccgg 54840
cgagttcggc gggccgtccg gagacggtga cgccgaacac gacgtcgtcg cggccggtgt 54900
gctggccgag aacgagggcc cacgcggcgc ggacggcggt ggcctgggtg atgccgcggt 54960
gcctggccca ggcggcgagg ttctcggtgc cggccttgtc gagttcggcg tggacggagg 55020
ccggcacgac gccgtcgggg gaagcgccgg gtgcgaccag ggagggctcg tccaggccgg 55080
tgagcgcgtc gcgccaggcg gtttcggcgg cggcgtcgtc gcgcgcggcg gcccaccgga 55140
gaaagtcgcc gtaggggcgg acgggcggca gcaccgacgg gtcggcgccg cttccgtaca 55200
gggcaaggag ttcgcgcagc agtaccggca gcgaccagcc gtcgaggatg acgtggtggt 55260
acgtgatcac gaaccggaaa cggtcgtcgt cggtccgtac gagcacgaac cgagccagcg 55320
gggcccggcc caggtcgaac gggcgccagc gttcctcccg ggccacggcg gcagcgtccg 55380
tggcaccgtc gcgggcgtcg acggcgcgcc atggcgtcgc gacgcgccgc gccacgaccg 55440
cgacccactc cccggagcgc gcctgacggt agcttgagcg caggacgccg tgccgcgcca 55500
cgaggctttc gacggcggcc cgcagccgac cggtgtccac cgcgccgacc aggtcgaacg 55560
cgagctgtcc cgcgtacacg tcgactccgt cggcggcggc gaaggagctg tggaacagca 55620
gtccttcctg gagcgccgag acaggcagga tctcctcgac taccttcgac cggcggttca 55680
ccactgctct tcctccccct cgagtccgag ctccagctcg aactcctcga attcgtcgtc 55740
gctgatcgaa ggcagtacgt ccgtgccggg gacggtgtcg tccgcgtcgt cggccggtgc 55800
cggaagggcc gcggcggcga gtgcgccgac cgtcccgtgc tcgaacacgt ctcgtacggt 55860
gatgtgcagt ccttccctcc gggcgcggct cacgagctgg atggacagca cgctgtcgcc 55920
gccgaggtcg aagaagccgc tgtcggctcc cacgtcgggc aggcccagga tgtccgcgaa 55980
gagcccggcg aggatctttt cctcgcgggt ggcgggggcg cgtccggcag gggtgccgaa 56040
cacgggggcg ggcagcgcgg cgcggtcgag tttgccgttc ggggtgtgag gcagggtggt 56100
gagagtcacc tgggcggagg ggaccatgta gtcaggcaga agttcgcgta ccgtctcgcg 56160
cagttcgccg gcgtccggtc cccgggtgcc gggacggggg acgacgtacg cgacaaggcg 56220
gccctggccg cggtggtcgg ggcggacggc ggcggcggcg cgccccacgt cgggatgccg 56280
ggcgagggcg gcctcgacgt cgccgagttc gatgcggaag ccccgtacct tgacctggtc 56340
gtccacgcgg ccgaggtgtt cgagcgtgcc gtccggggg aaccggccga ggtcgccggt 56400
gcggtacatc cgctcgccgg gggcgaacgg gttggcgacg aagcgttcgg cggtgaggtc 56460
cgggcggcgc agatagccgc gggcgagccc ggacccggcg atgtggatct cgcccggaac 56520
acccggggga acgggtccga gggccgcgtc gagtatgtgg acgcgccagt tgtccagggg 56580
ggtgccgatg ccgggtgtgc ggtcgtcgcc ggcggtgagt ggggcggcgg tggcccagat 56640
ggtggcttcg gtgggtccgt acacgttggt gacggcggcg ccggttgcgg tcaggacacg 56700
ggccaggtcg gcgggcagcg gttcgccgcc gaccaggatc cgtacgtgct cgagccgggc 56760
ggcggcgtcg gccggtgctc cgtcgagcat ggcccgccac caactgggcg tggcctggac 56820
gacggtgacg tggtggcgtg cgcacaggga ggtgatcgag gcggggtcgc gtacggcgtc 56880
ctcgtcggcc agcacgatcg cggcaccggc gaggagcggg gcgaacagtt cgaggccggc 56940
gatgtcgaag ccgacggtgg tcaccgcgag caaccggtcg tgcggggtca ggtcgtggcg 57000
ccggaccgcg tccgcgagga aggcgcgcag ggaggactcg gcgatcgcga cgcccttggg 57060
cctgccggtg gagccggagg tgtggatgac gtacgcgagg tggtcgcccg acggggacgt 57120
gggaaccgcg ggggcggctg cggcgggcgg ctgcggctcg tcgacgagga cggtcggcgc 57180
gaggccggcc ggcagccgtc cggcgaggcc ggcgtgggtg aggacgaggt cgggacgggc 57240
gtcggagagc acgtgcgcga gtcgctcggc ggggtacccg gtgtcgagcg ggaggtaggc 57300
ggcgccggtg cgcaggacgc cgagcagggt ggtgaccagg ccggccgtgc ggggcagggc 57360
gacggcgacg aggctctcgc ggccgatccc ccggtcggcc agcagccgcg ccgtctcacc 57420
gctgagcctt tccagttccc ggtacgagag ttcctcttcc gcggtgatga cggcgacggc 57480
gtccgggtcc agcgcggccc ggcggacgat ctgctcggcc acgctgcgcg ggggcagcgg 57540
ggtgtgcgta ccgctgcccc agtcaccgag gatgcggtcc cgctcgtcgt cggtgaggag 57600
cgggacggtg cccagggggg cggtggggtc ggcggccagt gctgcgaaga atccggtcag 57660
ccggtggccc agtgcggcgg cagtgccgtg gtcgtacagg tcgctgctgt agtcgagcag 57720
aacggacagg ccggcggcgt ctccgtcggg ggtgcggtgt tcagtgaagg tgaacgacag 57780
gtcgaacttg gcggcactgg cccccagcgg gtactcgtcc ccggtcaggc ccgggagggc 57840
gttgagtgcc tggccggtga tgtcctggtc ggcgttggtg agggtgtacc agatctggaa 57900
cagcgggtgg cgggcgaggg aacgccgtgg cgcaagcgcc tccacgagcc gttcgaaggg 57960
cacgtcctgg tgcgcgaacg cgtcgaggtc ggcggtcctg acccggtcca ccaggtcggc 58020
gaaggacgga tcccccgcga gattcgtccg aaggaccagg gtattgacga aaaacccgac 58080
gacgtcgtcc agcgcttcat cgccccgccc ggcgaccggg gtgccgatgg ggatgtcgtc 58140
gcccgcgccg agccgggaca acagaccggc gagcgcagcc tgcaccacca tgaacagcgt 58200
ggcccgacgg tcacgcgcca cggccagcag actccggtgc acgccggcgg gcagttcgaa 58260
ccgtacccgg ccgccccggt gcgaggaccg aacgggccgg ggccggtcgg cgggcagcgt 58320
gtgctcctcg ggcagcccgg ccagcatctc gcgccagtac ccgagccgac gcgccgtccg 58380
gccgtccggg gcgtcctcgt cgcccgcaac agcgtgttgc cacagggcgt agtcggcgta 58440
ctgcacggga agcggggtgt actccggggc gtcgccccgc gttcgggccc ggtaggcggt 58500
gaccaggtca cgcaccaacg gccccatgga ccagccgtcg ccggcgatgt ggtggatgac 58560
caccacgagg acgtgttcct ccggtgagac gcgcaacagg gtggcccgca gcggcgtatc 58620
gctggtgatg tcgaaggcgt acccggtggc ggacgccgcc tgttccgcca cctctccgac 58680
ggtcacctcg tgcgtgagga aggggggttc ggcgtgtggg agcaccagct ggcggggttc 58740
gcccccgtcg tcggggaaga ccgtacgcag tgattcgtgc cgtgcggtga cgtcccccag 58800
ggcggcccgg agagccggga cgtcgagggc gccggtgagc cggacggcga ccgggatgtt 58860
gtaggtggcg ctgggccctt ccagcttgct caggaaccac agtcgccgct gtgcgaagga 58920
cagcgggagc cgttcagggc gccggtcgat cctggtcaca ggtggcaggg cggtgccgga 58980
ggccgccagt acggtcgcga gcccggcagg cgtgggcgcg gcgaagaggt cgcggatgcc 59040
gagttccaca cccagggtgc ccctgacagc cgacaggagg cgcgtcgcca gcagggaatg 59100
gcctcccagg gcgaagaagt cgtcgtcggc cccggcggcg ggcaaccgga ggacgtcggc 59160
gaacaactcg cacaggatct cttcctgcgg cgtccgaggc agtctccggg tgcgggttcc 59220
gccctgccac tgcccggcca gcgccgcgcg gtcgagcttt ccgttggccg tcagcggcag 59280
ctcgtccagg gtccggtacg cggacggcac catgtacgcc ggcagggtcg ccgcgaggtg 59340
ctcgtggagt tcggccaggg tcggcgcccg gtgcggcagc gcgggcacca cgtgcgcgac 59400
aagccgttgg tcgtcggtct gctcgtcccg caccaggacc accgcctgga cgacacccgc 59460
gtgtgtgacc agcgcggcct cgatctcgcc gagttcgatc cggaagccgc ggatcttcac 59520
ctgccggtcc ccgcggccca catactccag tcccccgtcg ggccgcacct tggccaggtc 59580
gcctgtgcgg tacagacggc cgccggaacg ggaattcggg tcggccacga accgctcggc 59640
ggtcagagcg gggcgtccca gatagccgcg ggccagaccg gcgcctgcca cgtacatctc 59700
gccgacggtg cctgggggca ccgggcggag ccgttcgtcg aggacgtaca cctgcagatc 59760
ggccaagggc cgaccgatcg ggctgccgga aagactcgtc gcgcgatcct ccagcgggag 59820
caccgtgacg tggacggtgg tctcggtgat gccgtacatg ttcacgagcg tcggccgtga 59880
gccgagcggc aggtcgaacc agtcgcgcag tcgcgacggg tcgagcgcct cgccgccgaa 59940
gaccacatgt cgcagggcca gcccggcgca gcgcgccggc tcctggcggg cggcctccgt 60000
cagctgatgg aacgcggacg gtgtctggct cagcagcgtg acctgctgct cggcgagcag 60060
cgcgaggaat tcggcgggag aacgagtcac ctcgaacggc acgacgacga gccgcccgcc 60120
gtgaagcagc gcgccccaga tctcccagac cgagaagtcg aaggcgtacg agtggaagag 60180
cgtccagacg tggtccggtc cgaagtcgaa actgtcggag catgcggtga acaggcgtac 60240
gacattggcg tggctgatga cgacgccctt ggggcgaccg gtggagccgg aggtgtagat 60300
gacgtaggcg gggtgggcgg ggccgctccc gacacagggg gctgtgtcgg gagcggcggc 60360
gagttcggtg cggattgcct cgtcgtcgag ctcgatgcgg ggggcgatca gggggatgtg 60420
tacgtccgtt gaggtcacga ccgcggtggg gttggcgtcg gtgaccatgt acgcgaggcg 60480
gtcggtgggg tattcggggt cgagcggaac gtatgcggcg ccggttttgg tcacggccag 60540
gagtgccacg atgaggtcca ggccacgcgg tagtgccacg cccacgaacc gttcggggcc 60600
tgcgccgcgc tccatcagca ccctggccag gcgattcgcc cgcgcgttca actccttgta 60660
cgacagctcc tctccttcgc acaccaccgc gaccgcatgg ccccgagccg ccacctgctc 60720
ctcgaacagc tcaggcagtg tggaggtgtg aagttcctcg gctgccgggg gagcctcggg 60780
gctgagggcc ttcacctcgt cggccgacag gatgccgatg tcgcccacgg acagctcggg 60840
ccggcgggcg atctccgcga ggaggcgcac cagacgttcg gtcatggcct cggccgtgga 60900
gcggtcgaac aggtcggttg cgtagtccag gatgccgggc atctccgtgc tgccgggtgt 60960
ctccacgaac gagaacgcga gatcgtaggg gctggtcaca gccaggacgt cggccggtcg 61020
tgcgtgaagg ccgggcatgg cgtcgagcgc gtcgagcgcg gatcggcggt ccgtgttgtt 61080
gaaggcgagc atgacctgga agagggggtg ccgggtgagg gatcgttcgg ggttgatggc 61140
ttctaccagg cgttcgaagg ggatgtcctg gtgtgcgtat gcgtcgagag cagtggtgcg 61200
cacacgggcc aggagctcgg cgaaggtcgg gtccccggag acgtcggtgc gcagtacgag 61260
ggtgttgacg aagagtccga tcaggttctc catcgcctgg tcggtgcggc cggcgatggg 61320
ggtgccgatg gggatgtcgg tgccggcgcc gagtcgggac agcagggccg ccagggccgc 61380
ctgcaccacc atgaagacgg tggcgccctg ggtgcgggcg agtgcggtca ggccttggtg 61440
cacgtcgtgg ggcacggtga actcgacgcg gtcgcatgcg tgggaggcca cggcgggccg 61500
gacgcgactg gtggggaggg tcagttcggc ggggaggcct tcgagtttgc tgtaccagta 61560
gtcgagttgg gtggagagtt ggctcgatgt gttgttctcg gtgcccagga cgtcgcgttg 61620
ccaggcggcg tagtcggcgt attggacggc cagagcgggc agttgcggtg cgtggtgctc 61680
gagtctgacg gtgtaggcgg cggacaggtc gtgggccagg ggggccatgg accagccgtc 61740
gccggcgatg tggtggagca cgagcaggag gacgtgctcg tcgtcggtga cgcggaagac 61800
ggtggcgcgg agggggatct cggtggtgag gtcgaacgga tggcgggcgg cctcgtccac 61860
ccgattctgc agggtgtgcg cgtcgaccgg catggtgtcg agggtgaggt gggtgcgggt 61920
gtcgtcggtg ggcaggatgt tctgccacac gccgcccgaa tcgtcctggg cgatgaccgt 61980
tcgcaggctt tcgtggcggg cgatcacatc cgtcagggcc gcttccagcg cggtcaggtc 62040
caggcggccg gtgagtcgca gagccatggg gatgttgtac gcggcgttgg ggccttcgag 62100
ctggtggagg aaccacaggc gctgctgcgc gaacgacagc ggctcacaac cggtgcgctc 62160
ccgcgcgacc aggggtgccc ggaggctggt ggtctgttgc tggagcagct ggtccaggcg 62220
gccgacggtc ggggctttga agagggctcg gacaccgagg tcgatgccga gggccgaccg 62280
gatccgtgag gtgaggcggg tggcgagcag ggaatggccg ccgaggtcga agaagtcctc 62340
gtccacgccg acctgatcca gaccgagtac gtcggcgaac agggtgcaca ggatctcttc 62400
acgggctgtg ccgggtgctc gggaggtggt tccggaggcg gtcccgggcg cggggagggc 62460
ggcgcggtcg accttgccgt tgggggtgag ggggatctgg tccagtacga cgacggccga 62520
cggcaccatg tacgcgggca ggcggccggc gacggtgtcc gtcaccgccg aggacaggcc 62580
gctcgggtcg ctgcctgtca cgtacgcgac caggcgctgg tcgccgggcc ggtcctcgcg 62640
tacgacggcg atggcccgcg ccacgtgcgg atgcgccgcg acagccgcct cgacttcgcc 62700
gagttcgatg cggaagccgc ggatcttgac ctggtcgtcg gcacggccga tgaattcgag 62760
tgttccgtcg ctgcgccagc gtgcgaggtc gccggtgcgg tacatgcgtt cgccggggct 62820
gcggaagggg caggccacga agcgttcggc ggtcaggtcc gggcgcccga gatagccccg 62880
cgccaggccg tcaccggcca catacagttc gccgatggtg tcggggggtg tgaggcggag 62940
ggcggggtcg aggacgaaga ggtggtggcc ggtgaagggg cgtccgatgg ggacggggcc 63000
gtcggggagg cagtggccgg gggggatgcg gtattcggcg cagttgacgg tgagttcggt 63060
cgggccgtag gcgttgatga ccgtggtgtg ggggtggtgg gtgcgccagt ggtgcagggc 63120
gccggcggtg agggcttcgc cgccgaggag gagggtggcg tcgggggcga gtacccggtc 63180
gagttggccg agcaggggca ggtgggaagg agtggccttg aggaaggtgg cgcccaggcc 63240
gttggcggtg tcgtcgagat cgccgagggt gacgcagccg ccggagacca ggggggtgaa 63300
cagaacggtc acggtcaggt cgaacgcgag tgaggagtgc acgtggccca ggccgtcggc 63360
ggcgccgcgg taggcgttgc ggccgcgccg cagataggcg ccgaccgagg cgtgggtgac 63420
caccacgccc ttgggcaggc ccgtcgagcc ggaggtgtag atgacataca gcggatgccc 63480
ggacagcaga gccgacacgc gctccccgtc cgtcacatca ccggcgtcgg tggccgcctg 63540
ctcgttcagc cgtgtttcgc gggcgatctg cgcgtagtcg accacctcgc agtcgcggac 63600
ggcctcgacc gcagcggcca ggttctcgcc cacgacggcc aggtcgatgc cggcgtcggc 63660
gagtacccag tcgatgcgtt cggtgggcca ttgcgggtcg acgggcaggt agaagccgcc 63720
gcttttggcg accgcgagga gcatggccac cacgtccggg ccgcggtcca tcaccacccc 63780
gacaccgcgc tcgggtccca cgccccggcc gatcagccac cgggccaccg cattcgcccg 63840
cgcgttcagc tccgcgtaac tccattccac tcccgcgccg cgcacggcca ccgcgtccgg 63900
gtcggtccgc acccgctcct cgaacaaccc gaccagcccc gcacccgcac ccgcacccgc 63960
acccggcagc gcacgcacgc cgggttccgc gcccgcaccc cagcccagca gcgtctcccg 64020
ctcaccggca cccagcacat cgacaccgga caccggcaca tcacgctcgc agaccacgca 64080
ctccaaggcc cggacgatcc aaccccacag ctcacgcacc cgacccgcgt caaacacgtc 64140
cggccgatag ccgagccgga atcgcatggc gggaagggga accactgcca ggttcaaagc 64200
gaaatgcgtc gcatcggcac cctgtattcc gtcgatctgc aggccactgc cgtggaacag 64260
tgaatcctgc agcagggaat ccatcgggta gttctcgaag accattccga cgtcgaagag 64320
ttccttgtgt ccggcccagc gctggacctc ggccagccgg acgtgctggt gctcgagcag 64380
ttccgtctgt tcccgctgca gccgctccac gaaggcgccc agtgactccg ccgggtccat 64440
ccgggcccgc aacggaatgg tgttcacgaa cagaccgacc atgtcctcca cacccgccac 64500
ttcggcgggc cggcccgaca cggtcaggcc gaacaccacg tcgtcccggc ccaccagccg 64560
ccccagcacc aacgcccagg ccgcctgcac caccgacgcg accgtcacac cacgcccacg 64620
cgcccacgcc accagttccg aggtcagttc ctcgctgatc tcttcctcaa ggacttcggg 64680
aatcacccca ctgccctccg ttccgagggc gagcactgtg ggctccgcga gtccggccag 64740
cgcggaacgc catgcctgtc ccgcagcggt ctggtcgcgt tcggcgagcc acgccatgaa 64800
gtcacggcgc ggtggggagt acggaacctc cggccggcgg ccgagggcgg tgtcgcgata 64860
ggtggtgaag agttcgcgta tgagaaccgc cacggaccag ccgtcgagga tgacgtggtg 64920
gtacgtgacg atgaagcgga agtgggtgtc gttgatgcgt gcgagggtga atcgtgcgag 64980
tgggccctgc gtcatgtcga acggccgcca gcgctcgtcg tttgtgaggg tgtcggcgtc 65040
cgtgtcgagt gtgtggatgt actgccaggg ggtgtggact tgtcgtgcca cgacggcgat 65100
ccattcgccg gactgtgcct ggcggtagcc ggtgcgcagg acgtcgtggc gtgtcaccag 65160
gctttcgacc acggcgtgca ggtggtcggc gtcgactggt ccggtcaggt cgaacgtgag 65220
ttgtcccacg tagacgtcga ccccgtcggc ggcgaaggag ctgtggaaga gcagtccttc 65280
ctgaagcggt gatacgggca ggatgtcgtg cacgccaccg gtgacgtcgg cctgcagggc 65340
ctccagttcc gcgtggtcga gggcgaccag cggcaggtcc gagggggtca gtccgcccgc 65400
gtcgggacgg cgggcgtgtg cgaccagccc ttcgagggct tcgtcccaga agcgggcgag 65460
ttctcgtatc cgggattcgg acagcagtgt cgtcggccac gaccatgtca cgctgaggcg 65520
ggggccgtcg ggtgtgtcca gggtgatcgc gttgaactcc accgcgtggg ccaccggcag 65580
aacggggtcc tgacccgcgc ccaccgcgaa cggacccgac accggaaccc agtcggccca 65640
ctgttcctcg tcggagtccg ccgctgccct gccgccgacg aggccgccga gcccgtcggc 65700
atgggcgtcg ccttcgtcca gtgaggcagc gtccgccgag accgagaccc gtccgaggta 65760
gttgaagccg aagtgcgggg tggggtgctg ggcgagggcg ggtccgtttt cggggtcgag 65820
gtagcgcagg atgccgtagc ccaggccctg gtcgggcagg gtgcggaggc attccttgat 65880
ctcgcggacg gtgcgtccga cggcgggacc gccggcgcgt atctcgtccc aggccgccgg 65940
ctcgaggggg aggcggacgg ggtagatgct ggtgaaccac cccacggtgc gggagaggtc 66000
cgcgccgggt acggcgtctt cgttgcggcc gtgaccctcg acgtccacga cgacgcttcc 66060
gccgcggccg cgccagcgcg tcactgccag gccgaatgcc gccagcagca cttcctgcac 66120
gcccacaccc aggaccgacg ggacctccgt cagcagggca cgcgacaccc ccaccgacgc 66180
ccgtgaaccc accgactcca ccacgccgaa gacgtccacc gaagggtcca ccgcgcgtgg 66240
gccgaccaga gggtccgagc cgtggaccat ctgtgcccac aggtttgctt cggtggcgcg 66300
ggcgccgtgg cggccctgtt ccgccagcgc cgccgcccag ccgcgcagcg atgtgccgac 66360
cgtgtccaac cgcacatgcc cgccagcgcg tgcctgtgtc caggcttcgc cgaggtcgcc 66420
gaggacgatg cgccaggaga cgccgtcgac gaccaggtgg tgcgccacca ggaccaggac 66480
tccccggcgg tcgggtccac ggtccagcca taccgcgcgg accatccgtc cggtcagtgg 66540
atcgagccgg ctccgggcct ccacggcggc ggcccgcacg gcgttcacct gctcctccag 66600
gtcggcgctg gccatgccgt gccgggtgat ccagtcgggc tcaccgccgg gggcgggcgc 66660
gtcgacgcgc agggaccagg cgccgtcacc ggaatccact acccgtagcc gccaggcgtc 66720
gtggcgttcg cgtaccgcgt ccaggaccgt ccgcagttcg tcccaggtga taccggccgg 66780
tgtggcgacc acgacggact ggttgaaggc cctgaccgca ccgccgagcc caccgccacc 66840
acgctccgcc agccactcca ccaccggcca ccgctccacc gcacccacac cggagtccac 66900
gacggcggca gcgacgcctc cggactcccg caccacaccg gccagcgccc gtacggtgcg 66960
gtgctcgaaa acgtcccgca ccgacacctc cagacccgcc cggcgcgccc gcgccaccag 67020
ttgaatggac agaatgtcgt ctccgccgag gtcgaagaac ccgtcgtcca cgccgacccg 67080
ttccacgccc agcacttccg cgaacagggc gcacagagcc tcttcacggg gtgatccggt 67140
gacgcggaac tccgcggccg gggcgacgcg cggcgcgggc agcgcggcac ggtccacctt 67200
gccgttcggc gtcaggggaa tctcgtccag tacgaccacg gccgacggca ccatgtaccc 67260
gggcagacgg cccgcggccg tctccacgat cagcggtgcc agcccctccg cctcgtcacc 67320
ggcggccgtc acatacgcca ccagccggcg gtctcccggt cggtcctccc gtaccaccac 67380
cacggagcgg tccacacccg ggcaggccgc gacagcggcc tccacctcgc ccagctcgat 67440
ccggaaaccc cgcaccttga cctgatcgtc gacccggccg acgaactcca gcatcccatc 67500
cgcacgccac cgcaccaggt cacccgtgcg gtacatccgc tccccggcat caccgaacgg 67560
acacgccacg aaccgctccg ccgtcagacc cggctgcccg agataacccc gcgccagccc 67620
ctcgcccgcc acatggagct caccgaccac accgaccggc accgcccgca acccaccgtc 67680
gagcacgaac acccgcgtgt tccgcaacgg gcggccgatc gccaccggcc ccgcaccgat 67740
cggttgtccc ggatcgatac ggaagtccac gcagttgacc gtggcctcgg tggggccgta 67800
gtcgttgaag acgacggcct ccggcgaccc ggcccgccac ttgtccagct cccttcccag 67860
caactgctcg cccccgacga gcagcacctt cgcggacgcg gcccatgtcg cctcgtgcgt 67920
gagcagtggc agatggctcg gtgtggcctt gacgaacgtc ggcccgcccg ccgcggccag 67980
gctcggtggg cacgcctcgg tcaggtccgc gacatggatg gcgccaccga cgaccagtgg 68040
cgggaacagc gaggtcacgg tgaggtcgaa ggtcacgggg ctgtgcagca gggtgacgtc 68100
gaaggctccg gggtacgcgg tcacggagta ccgcacgaag tcggccagtg aacggtgttc 68160
caccgccaca cccttgggcc ggcccgtgga gccggatgtg tagatcacgt atgccacgtg 68220
ctcgccgctg accggagtga cgcacgtgcg gtcccggtcg gtgaccggac cccccggttg 68280
ttccgcgatc gccgcgtgga cctcgggttc gtcgaggcgg agctgtggga tgtcgcgccg 68340
cggcagttca cgggccgcac cggcgtcggt gaggatcacg gccggccggc agtcgtcgag 68400
gacgacggcg gtgcgctcgg cggggtgggt gaggtcgaga gggacgtagg cggctccggt 68460
cttgaggacc gcgagaagtg ccaccagcag ctcggcggac cggggcaggg cgacggcgac 68520
gagacgttcg ggaccgacgc cgtgggcggt gatcaggtgg cgggcgagcc ggttggcccg 68580
ggtgtgcagg tcggcgtagg tgaggacgtg gtcgttctcg acgacggccg gccggtcggg 68640
ggtccgcgcc gcacggcttt cgaggagatc gagcaccgtg cctggggggg tggggtgggc 68700
ggtgtcgttc cattgggcga ggagtgcgcg ttcgtcgtcg ccgaggagtt cgtacgaact 68760
gatggggcgg tccggtgctc cggcggcttg ctccaggacg gtgaggtagc gctggaccag 68820
gcgctccgcg gtgatccgcg tgaagagatc ggtgctgaac tcgagagcgc cgtcgatgcc 68880
gaggccgtcc gcgtgctcgg tgaggaagaa cgacaggtcg aacttggaca tgccggtgtg 68940
gacggcatgg acgcggctgg tgaggcccgc gaagcgggac ccgtcggcgg tgtcgttcgt 69000
cgccgggccg ttgtacgcga tcatgatctg gaagagcggg tgccaggaca gggaccgctc 69060
cggtttgagt acgtcgacga gacgctcgaa gggaacgtcc tggtaggcgt acgcgtccag 69120
gttggcggcc cgcacccgcg acacgagttc cgcgaacgtc gggtccccgg agacgtcgtt 69180
gcgcagtacg agcgtgttga cgaagaaccc gacgaggtct tccgtgccgt cgtcggagcg 69240
gcccgcgacg ggtgttccga gggggatgtc gtggcccgcg ccgtgccgcg acagcagggc 69300
ggcgagcgcg gcctgcatga ccatgaacac ggtgacgccg tgggcctggg ccatggcccg 69360
cagcgtgtgg tgcgtgtgct cgggcacggt gaaggcgatc gcctcgcccc ggcgggtggg 69420
aacggcgggc cgggtgtggt cggtgggcag ttcgagctgc tcgggcaggc cggccagggc 69480
gtcgcgccag tggtcgagct gtgtgctgtc cgacggatcg tccagcaggt gccgtcggcg 69540
cagggtgtgg tcggcgtact gcaggggcag cggctcgaag tgcggggcgt cgccggcgcg 69600
gcgggcgccg taggcgtggg cgagatcgcg tacgaggacc tcgacggagg ctccgtcgcc 69660
ggcggtgtgg tggacgagga gcagcagggt gtggtcgtcc gggccgcagc cgaacaggtg 69720
ggctttgagt gggatctcac cggcgaggtc gaaggggcgg cgtgcggctt cggcgagggc 69780
tgcggcgaga tcgccgggcg cgacggtgct gtgggtcagt tcggggcggg cggcctcggg 69840
gggcaggatg acctggtggg ggccttcgga gtcctcggcg aggagagtac gcaagggctc 69900
gtggcggttg gtgacgtcct gcagggcgac agcgagggcc gggatgtcga gcgggccttc 69960
gagccggacg gcggtgggga tgttgtacgc ggggctgggg ccttcgacct ggtcgaggaa 70020
ccacagtcgc tgctgcgcgt gcgacagggg gaggcgctcc gggcgcggag gcagcggcgg 70080
ggtggtgtgc tgccgtccgt tgtcgtccag gagcgggaga aggccggcga cggtggggaa 70140
ggcgaacagg tcgcggaggg tgaggtcggt gccgaactcg gtcctgatcc gggcgagcag 70200
ccgggtgccg ttgagggagt ggccgccgag ctcgaagaac acgtcgtccg tggcgacctc 70260
cgttacgccg aggacgtcgg cgaacagggc gcagagtgtc ttctcctgtg gggtgaccgg 70320
gcgttgcccg gatccgccgg cgcgggcggc ggggacggga agggccgccc tgtccagttt 70380
gccgttggac gtcagcggca gtgcggggag ggggacgagc gcccagggca ccatgtgggg 70440
cgggagggta cgggccaggg ccgcacggag ctcaccggcg tccggttcgg cgccgggcgc 70500
gggcacgatg tacccgacga gccgggggtc cccggggcgg tcctcgcgga tcaggacgcc 70560
ggcctgacgt accccgggga gagcggtgag cgcggcctgg acctcgccga gttcgatgcg 70620
gaagccgcgg agtttgacct gtccgtcggc tcgtccgacg aattccagca tgccgccgga 70680
gcgccgccgc accaggtcac cggtgcggta catgcggctg ccgggcgggc cgaacgggtc 70740
ggcgacgaaa cgctcggcgg tgggcccggg agcagcgacg tagccccggg cgaggccctc 70800
gccggccagg tacagttcgc cggtcgtccc gtcggggacg ggttcgagcg ccgggtcgag 70860
gacgtagccg cgccggttga gggtggggcg gccgagcggg acggggccgt gggtggcacc 70920
gtcacggtgc caggcggtcg cctcgacggt tgtctcggtg ggcccgtacg cgttgacgat 70980
gtcccggccc cggccccact ggtcgaagat ctccggcggg cagacctctc cgccgacgat 71040
cagagtccgc aggtgcggga gatctgtggc gggcatggtg gcgagtgcgc cgggggtgag 71100
ggatgtggtg gtgacggcct ccgcggcaag gaagtcggcg aggtgacggc cggtcaggcc 71160
gtccggcggt atcaccaggc agccgccgga caggagggtc atgctcagtt cggagacgaa 71220
gacgtcgaac cccggggaga tgaactgcag tacgcgggcg tccggtcccg tgcggtagcg 71280
ccggatctgg tcggcggcga gggtggccag cccttcgtga gtgacgacga cacccttcgg 71340
ccgtccggtg gagccggagg tctggatgat gtacgccgcg ttgccgggtg ccgggcggcc 71400
cggggcgggc tctgggccgc cctcacggag gaggtcggcg gcggccaact gcggcgtggg 71460
gtgggacagg ccgtcgatgt gtccggtggt gacgaggtac cggggtcggg tgtcgtcgag 71520
tacacgggcc agccgctcgg ccggatgggt ggggtccagc ggcacgtagg cggcgccggc 71580
ccgctggatg ccgagcagga tcgcgacgct ctcggcggac ttggggaggg cgagggcgac 71640
gacgtcctcg gtacgcaggc cgaggccggc cagccgtccg gccgcccgtt cgacactccg 71700
gtgcaggtcg cggtaggtga ggacggtgcc gtcgtgctgg acggcgggcg cgtcgggggt 71760
gcgccgggcc tgttcggcga agagttccgg caaggtggca cgcggtacgg ggcgttccgt 71820
gccgcgggag tcgtcgagca gccgggcgcg ggtgcccggc gacaggaggc ggtgtcttcc 71880
ggtcggcagg tccgggtcgg cggtcacagc gtcgaggaag cgcaggaacc gctcctggtg 71940
gtcggccaga tcggtggccg tgtacagggc ggggttaccg ctgaagacca gctggacgcc 72000
gtcgggggtg ccgggggtgc cgtaggagtt gatgtggaag tcgcggaccg gtcccgagga 72060
caactcgtgg accacggccc ggcagtcgcc gaaccggatg ttgtcgacat acgccaggac 72120
gttgacggtg acccgcgcca ggcccgcggt gcggccgacg ccccccaggt tccggtggag 72180
ttcttccccg cggaaccgct ggtggcgcgc cagcgtgccg acggcgcggg tggtgtcggc 72240
aaggagcgcg gcgaacgggg catcgagccg ggcgtcgagg cgcagcggca cgtcgttggc 72300
gagcatgcac ggggtacgca gcgccggccc ggtgagccgg gcggtgacgg gcaggccgat 72360
gacggtgtcg tcggcggcag cgtggcggcg tacgaacgcg gcggtggcgg cgacgagcag 72420
tgacgaccag cgagctccag tggccgcggc cgaggcggcc agcttctccg tccgttcgcc 72480
ggacagctcc acggtgcggc gcagcgggac ggcgagagcg ccggcctccc gctcgctcag 72540
cccgggggcc tcgtccgcac cggcgaggga ccgggtccag aattccccgt cgcgccgatg 72600
gttgtcggag tcacggtagg ccgcctcctc cgtgaggaca ccggccaggg ggccgaacgg 72660
gcaggggccg gggtcgtccc cgttgctgag ggcggtgtag acgtgggcga ggcggcgcag 72720
gtggagcagg gcaccgtagc cgtcgagcgc gatgtggtgg tagccgaagt acagcaggtg 72780
gcgggacggg ccgagggtga gcagggcgtg ggtggcggtc ccggcgcggt cgaggcggac 72840
cggtgcggcg agcgccgcgt gcatccagtc gtgtgccgcc cgctcggggt ccgggtcgcc 72900
ggagaagtcg gcttccagca ggagctgtcc ggcaccgggt tcgaggatct gcagcagttc 72960
cccgtcctgc ggtacgaacc gggtacgcag cgcctcggtc tcgtcgaggg tccgccggac 73020
cgccgtgccg agtgtccgcg ggtcgatgga cccggtgagt gacaggtacg ccgcacagtg 73080
gtacaggcgg tggtcgtccg ccagctggcc ggcgagccag acgctctgtt gggcggcggt 73140
gacgccgagg cgctgcgact gcatgtccat ctctcgcgga tcccctcagg tgcggtcggc 73200
caactggggc atcagtgcct ggacgaactc gttgagtgtg gggacgctcc acagcagctc 73260
cgggtcgacc tcgatcccgt agcggtcctc gacctgcgcg cagagggcaa ctccggagac 73320
ggagtcgagg ccatggtcgg tcaggggtgc gtcaccggcg atccggtcgg gtgtctcgtt 73380
cacgaactcg gcgatctgtc cggtgatcca cgcggtgacc tcgctgggcg tgctgaccgc 73440
ttcgggcggg ttcatgtgga gctactcctc gctgtcggcg ggcaccgcgg tgcccgttcc 73500
agggcagggc cgccctccgg cgattacgcg gggtgcggat gtggagcgtg gtcagtgggg 73560
gcgatgtcct ctccggccgt gccggcgatg gcgtgccagc ccttgtccgc gtggacgatg 73620
ttcagctccc cgcggaggca gaggtcacgc atggcgcccc ggcggacctt gccgctggtg 73680
gtacgcagta cggtgccgcg gctcaccagg gcgatgccct gggcgtcgag tccgaattcg 73740
cggttgatcg tcccccgcag ggcgctgacc agggccgggt ggtcgtcggc ggggaccggg 73800
gtgcggactt cgtggaccac catcaagccc ggcgtgccag gtgccgccgg gacggtgaac 73860
gcggcgccga cgctctcaag ttccgggtgt tgcaggcgga gttcgtgctc gatgtcctgc 73920
gggtacagat tgcgtcctcg tatcaccagg gcctctttgc agcggccggt gatgtggaga 73980
ttcccttcgt gcagggcggc gagatcgccg gtgcgcagcc agtgaccgtc ctcgccgtcg 74040
agggtgaggc cgaaggtctg ctcggtctcc tccttggcct gccagtagcc ggcgcagacg 74100
gcggctccct ggaggaccag ctcgccgacc tggccgggcg gcagaacacg gtgcccgccg 74160
ggttcgacga tgcgggcgcg gaagtggccg acgggaccgc agctgacgat ctcccgggct 74220
gcctcgccgg gtacggcgag gcggaattcg tggcgttcga gggcggcggt ggcgactcgt 74280
gccgtgtgca gcccctgcga cctcgacacg aacagggtgg cctcggccag cccgtagccg 74340
gcggtgagcg cctcggggcg caggccgtac cgggcgaacc gttcgccgaa ggcccgtacg 74400
gtggctgccc ggatgggctc cgcgccgttg ccggcccacc gccagcggga caggtccagc 74460
ccggcgatct gctcgtcggt gacgcgcttc agacacatgt cgtacgcgaa gtcgggagcc 74520
gacgaccaga ccaggccgaa ccggtcgatg gtccgtagcc acaggtgcgg ccgtttgatg 74580
aatgccgtgg agctcatcag cacgcacgtg gtgccgaggt acagggccgg tgtcagcgtg 74640
cccaggagcc ccatgtcgtg gtacatgggc agccagccgc ccatgggagt ggcggcggtc 74700
agctcgcagg cctcggcgag attccgcgcg ttcgccagca ggttggcgtg ggtcacgacc 74760
acgcccttgg ggttgccggt cgagcccgag gtgtactgga tgagagccac gtcgtcgggc 74820
gcgacgcccg ggtcgtccca ctgcgccggg tcgccgagcg ccgcccggtc gtccacggcc 74880
acgacgcgag tagccggggc cgtggtctcg gtcagccagt cgtggacgga ggcggtctca 74940
cccgcgacgg tcagcaccac gccggctccg ctgtcggcgg cgatggccgc gacacggcgg 75000
cgttcgaagt ggcgcccccc gggcagcggc gccggtacgg cggccagacc cgcgtacagg 75060
cagccgaggt acgcggccgt gaactccggg ccggacggca gcagcagaag tgcccgtccc 75120
cccggcgcga gttgctgtcg gagggtcacc gcgatgcttc tggcccactc gtccagctgt 75180
ccgtagtcca ccgacgccgt gtcgtcggtg gttccggtgt cccgtacgag aaccacggca 75240
gtctcccggg cacgtgtccg tgcccaggtc cgcagtgccc ccaccaggcc ctgcccggca 75300
cagcggctct cactcacggc aggcactccc cctcctgact ccggtcgaac gacgcatccg 75360
cgctcgcata cctgtaaggc agggagtcat tccgcgtccg gcttgcgacc ggtcgccgct 75420
gcgtgaggtg tgccgtcccg tcatcaggca tgtgcgaatg ccctgttgcc gtcatgtgag 75480
gtccaaccgc ttcggcggac caaaccactt gatatggacg aaacaccaac cccccctgta 75540
acgcctgtgg ttggtgtgcc tgttggttcc tgtgcgactt gatcgccagt atgttccacg 75600
cccctgcgaa cgtcaatggg tcccggcagg gacgatcttt gcgtatgcgg cgtttacgca 75660
cgttctcatc tgcgcaaacg tcacactggg caggtggtgg ggaggggtca gggcgtccgg 75720
tgcgggccgc ccgcacccgg cgggcgccgc agcccgcgta ccgccagcag accggtcacc 75780
gccaagagcg caagactggt gccgacggcc gcgagcgccg cgcccagcgc gctgtggtcc 75840
agcgccagat cgcgggacgc ctccaccgcg tacgtgaggg gattgaccgt cgcgatgacc 75900
ctgagccatc ccggcagaat gtcgagcggg acgaaggcac tcgacgcgaa catcaggggg 75960
aaggtgacga ggaacccgat gctggacagc acctcgatgc tccgcagcca cgcggcgagg 76020
gccaggaaca cccagatcag cgaccacgcg acgagcaacg cgagcagcgt cgcgccgacg 76080
atccccgaca cgcccccggc cggccggtag cccagcggcc ccatcgcaca ggcgagcaac 76140
gccacgagct gcagggcgac ccgcgccaga tcggccagcg accgggccac caggaccagc 76200
ggcagccgta ccggcatcac ccgcagccgg accatcatcc cggactccat gtccctgacg 76260
agacccagcc cgccgccctg cgcggaaccg atcccggtcg tcagcagcag agccggcacc 76320
aggtagtcga ggtagcgcac acccggcggg aagatctccg gattggccat gcggcccagc 76380
acctgactga acagcatgag catgatgatc ggctggagca ggctgaacag cgcgatccgc 76440
cggtccccgt agatgatccg gaactgccgg ccggtcagca cccggaactg ggtgccgatc 76500
cccgcacccc cgccgcgcca cccgggcccg tccgccaccg gcgaggtccc cggacccgta 76560
cgtcgtgtgg tcagcaccgg accgttcccc cgctcgtggt ctcccagctc tccgccaggc 76620
tcagatacac gtcgtccagc gtgggctcct tgagcgccag ttcggcgagc tccacgcccg 76680
ccgcgccgag aatccggaag atcccggcca gctcggccgt tgccgccagc ggcagtgaca 76740
ccgtccggga caccggatcc gttgtgaggc cgaggccctc caggcgcagc gctccttccg 76800
cgctctccag atccgccgcg gacccgaagg tcaggcggac ggaccgggtg cccgccgcct 76860
tgagctccgc cgccgtgccg accgtcacca cgtggccgga cgacagcacc gctatccggt 76920
ccgccagccg gtcggcctcg tcgaggtact gggtggtcag cagaaccgtc gtgccctcgg 76980
cgaccagccc ttccaccgtc tcccagagtg cggtgcggct caccgggtcc agcccggtgg 77040
tgggctcgtc gaggaacagc accgagggcc tggccaccag actcgccgcc aggtcgatgc 77100
gccgccgcat tccgccggag taggtccgca tcggtctgtc cgctgcctcg gtgagaccga 77160
attgttcgac cagttcggcc gcccggcgtc tggcctccct cggccgggca cccagcagcc 77220
gggagatcag caccagattg gcgagcccgg acagatgctc gtccagggca gcgaactgcc 77280
ccgtcacccc gatacaggcg cgtacccgtc gggcatcgcg cacgatgtcg aagccggcga 77340
cctcggcgga cccaccggac ggggggagca ccgtcgagag gatctggatc agcgtggtct 77400
tcccggcacc gttgtgtccc aggagcccca gcacgctccc cgcgggcacc tccagatcca 77460
cgccgaccag cgcctgcacc gcaccgaagt gcttggtcag ctgccgggtc ctgatcgcgc 77520
tcccgcccat gcttctccgc gtcgtcggtg ccggtgggcg tgtgcactgc ccgcagtcaa 77580
cgcactgtag cccacggggt agttgaccgc gatcgcctga gccgacctcg gacgcccgta 77640
cacggtcagg agcgggtccc aggaacaagg ccctccaccc cggactcggt cacccccgca 77700
ccgtgtgccc gcgggaggac atgatgtgcc tgcgggagga catgatctgc ccggcactcg 77760
accagtggat cgcccgtgcc ttcgcgccgg aacgtctgac cgccaccctc accgccctga 77820
cgcacgcggc tgccgcatcc gatggccgcg ccccggaggt cgagcaggcc cggcgggcgg 77880
tgagaaaggg cgagaagcgt ctgtctcact accgggccgc cctcgcggcc ggggccggcc 77940
ccgccgttct cacccagtgg atcaacgagg cccggatctg tacggcgagc ccccgtgctc 78000
cgtcggcgga cggggtgggt gaaggggaat ggttgccggg ggcggtctgc ggggtccgtc 78060
gcggggtggt cgtggccggg agggaggagt cgtccagcgg ttccctggta ggttggtgtc 78120
ccttcgttct ttttcaggcg ctcccaccgg ccgcccgggt atgcgtccgg tgacgccggg 78180
aagtcgttcc gctgttctct tcggtgtgtt ctcggaagaa gggcgcggga atccccgtgg 78240
cgccgtcggg agggctccac gaacgaggag agacagagcg gggctgccgt gtcgtggatg 78300
aacgatgtac tgacagcggt gtcggacatg aacccggtca ccagattcgc gctggcctcc 78360
gtattcgcgt tcgcggaatc cggccttggg gccggcatgg ccgtccccgg cgaggtcgcg 78420
gtactggccc tgagcgccgg aacggagggg acacgcccgc tgttggccct cttcctcgtc 78480
gtcacactca gcagctcggc cggcgaccac atcggctact tcctcggcat ccgctacggg 78540
cagcggatgc gggagacgcg cctggtacgg cggattgggc agcaccactg ggaccgcgcg 78600
caagagctct gccaccggta cggggcgcgc gccgtcttcc tgacccgcct tttgcccgtg 78660
gtacgcaccc tcacccccgc gaccgccggc gtcggctccg tgcgctacct ccgcttcctg 78720
cccgcctccc tcgccggagc cgcgatgtgg tccgcgctct acgtgtccgc gggaacactg 78780
gtctcgacct ccctgcgcga ggccgagagc gtgctctcca ccatcctgtg ggccctgctg 78840
ggcgttgcgg ccgccttcac gctcgcgatc gtgtggtggc ggcgcaggca ccgccgccgc 78900
agctcgtgac cgcggcgagc tcctcggcgg ctgtgggacg gcccaggaag ccgtacgacg 78960
gcttgcgttt cgggcggccg gcggcacagg tcttgacggt ggcgtcacgg tccctggagt 79020
ggagcgcgca cagttccatc tgcgcacccc aggcctccgt ggtgaaacgg ttctcctcgg 79080
cagggtcgtc aaggttgcag gctccgacga cagcaacgcc tgccggaaca cgaatccggc 79140
aggcgttgcg ctcctcgggg cccgaggaaa agagggaacg ggacaggtca gtgaccggcg 79200
accgtgccgg acagttcgtt ggtgcccttc ggcagggtga cggatgccag cttcttcccc 79260
gattccaggt cgatggagtg gagttggcgc ttgcccggtt cggagacgta cgccgtgtgg 79320
tcccggacga acagggtggg cctgggctgc tgccagtcca ggggctcggt ccagtcgccg 79380
accgcgtcga tcttcttctc gacctttccg gtctccgggt cgatgacgtg gagggtgccg 79440
ttggtgccga gcacgagggc ttcgccgtgc gggccgcggg cgagggagcg gaaggagtag 79500
ctggtgccga ggtcgaccag cttcatcttc gccgtacggg tgtcgatcag ggatatgcgg 79560
gtggggcgtt ccagttcggc gtcggggtcg gtcttgtagt cgccgaggag gatcggggag 79620
gcgtcgctgc cggcctggtt gccggtgcgg gcgtagtcgc cgggggcgtc gaccttggtg 79680
aacttgccgt ccttgtagag caggacgccg tcctcgcagc cgaagccggc cacctcgccc 79740
tgggcggcgg cctcgccgtg cactccgggg cagttctcgg cgcgtgcgat ctccttgttg 79800
tccttgtcca ggacgagggc tccggtgcgc ttctcctcgg tgccgagggt ggtgacgagt 79860
tctccgccgg ccagttcgat ggcgacaccg tggtggggct tcgcggacgt gtaggtgcgg 79920
ccgtccggct tcttcccgtc ggacaggtcg gcggggtcga agacgttcac ctctcccgtg 79980
ccgtcggtga acaggaccgt cttgccgccg tgccggacga cgtgccccgg cttggaaccc 80040
ttgaactcgg cgtcggtgaa ctcctgtcgg gtggcgtcga acacgcggaa gccggagtcc 80100
gtggagacga cgacgtgctc gttgtcgccc gccgggttga cccggttgaa gccgggcagt 80160
gcgatcgtct tcgcgagctt cagggtctcg ccgtcgagga cgtacagtcc cccgtcgtac 80220
gaggcgacga tcgggttccc gaccgccgcg gcctcgctgc tcttctgctc cttgggctct 80280
ttgccggaag gggcgtcgtc gttgccgttg ccgccgcagg cggtcagcgc catcgacgcc 80340
gccacgacca gcgccgtgcc cgtgaagact ctggcgcggg tgggcttgtt catgatcggt 80400
gtttcctctc cggccctcag gcgcgggttt gtggggtgga gcggccggcg ctcgaagtgc 80460
cgtgccgcgg ggaagctcgt tcagtcgccg gtgaggccct cggccatggc ggaggtgttg 80520
gcgcgcatca tctccaggta ggttccggcg cccttgccct tctcggtcag cgactcggag 80580
tagagcgaga cgacgtccac gtcgccgccc atctcctggc gcaggacctc ggcgagccgg 80640
gtgggctggg aggagtcggc gaagacggtg cgcaccttgg ccttctccat ggcctgggtg 80700
agagagcgca ggtcggagga gctgggcgag gcgagcgtgg ttccgctggg gatgaccgcg 80760
ccgatgacgc ggaggccgaa gcggtcggcg aggtagccga agacgtggtg gttggtcacc 80820
agggcacgcc ggtcctcggg gatggcggcg aaggacttct ccatccatcc cgtgaggtcg 80880
gcgagttgtc cgtcgtaccg ctcggcgttc tcccggacct tcttctcgtc gacgccctcc 80940
acatgctcgg cgacctggtc ggcgatcagg ccggcggcct cgcgtacgcg gtcggggtcg 81000
gtccagaagt gcggatcggg cttgccggcg tcctcttcgg ggccgccgtc ctgtccggca 81060
tggaaggtga gcgggtcggc cgcctcaccc gcggcgaagg cggccactcc ggactcgcgg 81120
gcagcctcca cgtgccgcaa cacgttctcc tccaggccga gcccgttgta gacgaccagg 81180
tcggcgttct ccaactcagc ggcctgcacg gccgagaggc cgaaggagtg cgggtcggcg 81240
ttgggcttca tcaggacact gacgccggcc tcgtccccga cgatctcccg ggtgatgtcg 81300
ccgaggatgt tggtggtcac cacgacccgg ggccgttcgt ctccgccgcc cgcgcagccg 81360
gtcgccgcac cggcgacgac gaagaatccg gccagggaca ccagcagggc acgcatccgt 81420
ggagcgccca cgcgcgcgct catcggcccg tctccaccat cagggacggc cgtacgtcga 81480
cgtcgaacgt acgggccacg cggagatcgt cgttgtagtc gatctcgtac acgcgcttgc 81540
cctcggggtc gttgaggtag gcccggctgc ggtccacctc gatcaccgga gccgcgccgc 81600
cgcccgcacc ggttccgggc agttccttga gcaggggatc ggtcacgccg gtctccttgc 81660
cggtgggtat gtcgtagccg tgcagggccc cgtcggtctc caggacgacc agcggcgagc 81720
cttcgccggc cgtgttggcg gcgaccacgg ggccggtctt cacccgggtc caggcgccct 81780
cgccggcatc caggacccag acagcgtcct tgccggcggg tgccgtgagg gtgctgctgc 81840
ccgggcggtg ccggaactcc acggcccgct cggtcttcgg cacgtcctcg ccgtacggaa 81900
tcttctccgc cgtgaaggcg ccgtcgtcct catggacgag cagagcgccg tcggcgcagc 81960
cgaggacaac cccgcgccgg gtgaccgcgt cgccctgagg ctcctcgcac tccgcctccg 82020
gagcggcgac gcgcttgccg gaacggtcca gcacggcgac cttcgccgga gccccgctct 82080
cagcggtgag tgtcaccagg tgttccgcgt acggcacgac ggcgccggcg aacgtgccgg 82140
gcagcgggga cggcgtgccc agggcgcctt tctccaggtc cgccctgcga tacacgctcg 82200
ccttcccgtc cgccgacgag gccacggtca cgcccgcgtc gccgcggatg ctcgtaccgg 82260
tgccgcccgg gagttcgccg acctccttga tcttcgcgcg gtaatagtgg acgtggtcgc 82320
cgtggtccac catccacgca ccgctgtcga gcacccgtat gccgtccggg ccgtggaagt 82380
agccgaagcg gccgtccgtg gtgagtgcgg tggcaccggg gctgcgggcg atgtcgtaca 82440
ccttgccggt gatcaggtcc agcacgcggg tctcaccgct cccggggtcg ccgagcagaa 82500
gtctggactg ctgctcggcc gcctcggtgg cgccttcgac atagccgtgc ggagtggggg 82560
acgaagaggc gggcccggaa ccggacttgg cctcgtcccc gcccccgcat ccggcggtca 82620
ggaggatcac ggccgcaagc gccggagcca gcgatgtgac gttttttcgg acgagcaagg 82680
agatcgcctc aggtttctct cggtcaggac gtggaggagg tcgcggccgg ggcgaccgcc 82740
ggttcggcaa gaccggcccg gcgcgcacgg cggcggtgcc gaagtccgga tgccaggtgg 82800
gacaggaaga agagagcgac ggcgagggcc gagacggtcg ctccggccgc ggtgcgcaga 82860
tgccaggaca ggagcaggcc gccgaaggtg gcggcggcgc cgaggagcgc cgcgaggacc 82920
atgaccgcct ggacgcttcg cgcccagggc atggccgccg cgggcggggc gatgagcaga 82980
ccgaggacga gcagcgtgcc cacgatgtgg aaggaggcca cgatggccag cgccagcagg 83040
ccgagcagca cggcatgggc gagccgggga cgcagcccga gtgtccgggc cttgcgctcg 83100
tcgaacgcga gggccaggaa agcccggtag ccgagcaccg agacggccag cgccagcagc 83160
agggctactc caagaagcag cagatcgctc ccccgcacgg cgaggacgtc tccgaacagg 83220
aagccggtga ggtccaccgc gaaggactgc gaccgcgaca cgatgatgac gccgagcgac 83280
agcatgccca cgaacagcag gccgatgccg gtgtcctggg acagtcgcgg agtccgcccg 83340
agggccgtga cgcccgccgc catcacggcc gcgctcacca ccgcccccac cagcaggttg 83400
cctcccagca gggaggcgac cgcgacgccg ggcagcagcc cgtgcgacat cgcgtcaccg 83460
aggaaggcca tcccgcgcag caccacccac gttcccgcga gggcgcatat cgccgacacc 83520
aggatcccgg cccatagggc cctctgcaca aaggccacct cgaaaggggc cgtcaaccac 83580
tccatacgag gaccatataa tgacaaccat gttcaataac aaccctccgt tcccgccggc 83640
caccgaactg cggaatgaac gcgtccggtt ccagcggctg agcgccggct accccggccg 83700
ccccgtgctc caccaactca gcgccgcaat accgccgttg gccatgaccg ccctggtcgg 83760
acccaacgga agcggcaagt cgacgctgct gggcgtcctg gccggagtga tcaccgccac 83820
atccggacaa ctccggtacg cagagggctc cccgccggct ttcgtcccgc agcgcggtgc 83880
cgtcggcgac accctccccc tcacggcgcg gcagacggtg gagatgggcc gctggggaca 83940
gcggggcctg tggcggcggc tgacccgcac ggaccggacc gcggtcgact ccgccatgga 84000
gaggctgggg gtcgccgacc tcggcgcccg ccagctcggc gagctgtcgg gcggccagcg 84060
tcagcgcgtg ctgatcgcgc agggcctcgc ccaacagtcc gacctgcttc tcctggacga 84120
gccgaccacc ggactcgacc ccgaggcgcg ggagcggatc acggccctgc tgacggacct 84180
ggtcgccgac ggcaccaccg tcgtccaggc cacccacgac ctggacgccg cccgctcggc 84240
cgacgcctgc ctgctcctgg ccgacggacg actgatcggg cagggatcac cggaggaagt 84300
actcaccccg gaggcactgg cccggatctg gcagccggcg tgaagcacga agcgctcgaa 84360
gagcctgcgc aatgatcgag gcggggtgag cggatcgccg gtcgccccct ttaccgaccg 84420
gggaatctcc cccggtccgt cccgcgcgga aacagcaccg ccgcagacgc ctgcccctgt 84480
gtcacggcga gtgaggcgaa ccaacagcgc ctcagcgggc gtcgctgctg cgcaccgtga 84540
ggatacgtac cgcgcgatct cccgtgtgag cctcgcggcc gtgccagcag cggtagttgt 84600
ccaggacgag gatttcgccc ttgtcaatta cctaccggcc tccaccatac ctacgatatt 84660
cgcgcccact ctcccattag tccgcacagg tggatgcgat gcgattgccc gctaagatat 84720
tctaacgcgt aacgcagttg agtatgcttg gcgtaatcat ggtcatagct gtttcctgtg 84780
tgaaattgtt atccgctcac aattccacac aacatacgag ccggaagcat aaagtgtaaa 84840
gcctggggtg cctaatgagt gagctaactc acattaattg cgttgcgctc actgcccgct 84900
ttccagtcgg gaaacctgtc gtgccagctg cattaatgaa tcggccaacg cgaacccctt 84960
gcggccgccc gggccgtcga ccaattctca tgtttgacag cttatcatcg aatttctgcc 85020
attcatccgc ttattatcac ttattcaggc gtagcaacca ggcgtttaag ggcaccaata 85080
actgccttaa aaaaattacg ccccgccctg ccactcatcg cagtactgtt gtaattcatt 85140
aagcattctg ccgacatgga agccatcaca aacggcatga tgaacctgaa tcgccagcgg 85200
catcagcacc ttgtcgcctt gcgtataata tttgcccatg gtgaaaacgg gggcgaagaa 85260
gttgtccata ttggccacgt ttaaatcaaa actggtgaaa ctcacccagg gattggctga 85320
gacgaaaaac atattctcaa taaacccttt agggaaatag gccaggtttt caccgtaaca 85380
cgccacatct tgcgaatata tgtgtagaaa ctgccggaaa tcgtcgtggt attcactcca 85440
gagcgatgaa aacgtttcag tttgctcatg gaaaacggtg taacaagggt gaacactatc 85500
ccatatcacc agctcaccgt ctttcattgc catacgaaat tccggatgag cattcatcag 85560
gcgggcaaga atgtgaataa aggccggata aaacttgtgc ttatttttct ttacggtctt 85620
taaaaaggcc gtaatatcca gctgaacggt ctggttatag gtacattgag caactgactg 85680
aaatgcctca aaatgttctt tacgatgcca ttgggatata tcaacggtgg tatatccagt 85740
gatttttttc tccattttag cttccttagc tcctgaaaat ctcgataact caaaaaatac 85800
gcccggtagt gatcttattt cattatggtg aaagttggaa cctcttacgt gccgatcaac 85860
gtctcatttt cgccaaaagt tggcccaggg cttcccggta tcaacaggga caccaggatt 85920
tatttattct gcgaagtgat cttccgtcac aggtatttat tcgcgataag ctcatggagc 85980
ggcgtaaccg tcgcacagga aggacagaga aagcgcggat ctgggaagtg acggacagaa 86040
cggtcaggac ctggattggg gaggcggttg ccgccgctgc tgctgacggt gtgacgttct 86100
ctgttccggt cacaccacat acgttccgcc attcctatgc gatgcacatg ctgtatgccg 86160
gtataccgct gaaagttctg caaagcctga tgggacataa gtccatcagt tcaacggaag 86220
tctacacgaa ggtttttgcg ctggatgtgg ctgcccggca ccgggtgcag tttgcgatgc 86280
cggagtctga tgcggttgcg atgctgaaac aattatcctg agaataaatg ccttggcctt 86340
tatatggaaa tgtggaactg agtggatatg ctgtttttgt ctgttaaaca gagaagctgg 86400
ctgttatcca ctgagaagcg aacgaaacag tcgggaaaat ctcccattat cgtagagatc 86460
cgcattatta atctcaggag cctgtgtagc gtttatagga agtagtgttc tgtcatgatg 86520
cctgcaagcg gtaacgaaaa cgatttgaat atgccttcag gaacaataga aatcttcgtg 86580
cggtgttacg ttgaagtgga gcggattatg tcagcaatgg acagaacaac ctaatgaaca 86640
cagaaccatg atgtggtctg tccttttaca gccagtagtg ctcgccgcag tcgagcgaca 86700
gggcgaagcc ctcgagctgg ttgccctcgc cgctgggctg gcggccgtct atggccctgc 86760
aaacgcgcca gaaacgccgt cgaagccgtg tgcgagacac cgcggccggc cgccggcgtt 86820
gtggatacct cgcggaaaac ttggccctca ctgacagatg aggggcggac gttgacactt 86880
gaggggccga ctcacccggc gcggcgttga cagatgaggg gcaggctcga tttcggccgg 86940
cgacgtggag ctggccagcc tcgcaaatcg gcgaaaacgc ctgattttac gcgagtttcc 87000
cacagatgat gtggacaagc ctggggataa gtgccctgcg gtattgacac ttgaggggcg 87060
cgactactga cagatgaggg gcgcgatcct tgacacttga ggggcagagt gctgacagat 87120
gaggggcgca cctattgaca tttgaggggc tgtccacagg cagaaaatcc agcatttgca 87180
agggtttccg cccgtttttc ggccaccgct aacctgtctt ttaacctgct tttaaaccaa 87240
tatttataaa ccttgttttt aaccagggct gcgccctgtg cgcgtgaccg cgcacgccga 87300
aggggggtgc ccccccttct cgaaccctcc cggtcgagtg agcgaggaag caccagggaa 87360
cagcacttat atattctgct tacacacgat gcctgaaaaa acttcccttg gggttatcca 87420
cttatccacg gggatatttt tataattatt ttttttatag tttttagatc ttctttttta 87480
gagcgccttg taggccttta tccatgctgg ttctagagaa ggtgttgtga caaattgccc 87540
tttcagtgtg acaaatcacc ctcaaatgac agtcctgtct gtgacaaatt gcccttaacc 87600
ctgtgacaaa ttgccctcag aagaagctgt tttttcacaa agttatccct gcttattgac 87660
tcttttttat ttagtgtgac aatctaaaaa cttgtcacac ttcacatgga tctgtcatgg 87720
cggaaacagc ggttatcaat cacaagaaac gtaaaaatag cccgcgaatc gtccagtcaa 87780
acgacctcac tgaggcggca tatagtctct cccgggatca aaaacgtatg ctgtatctgt 87840
tcgttgacca gatcagaaaa tctgatggca ccctacagga acatgacggt atctgcgaga 87900
tccatgttgc taaatatgct gaaatattcg gattgacctc tgcggaagcc agtaaggata 87960
tacggcaggc attgaagagt ttcgcgggga aggaagtggt tttttatcgc cctgaagagg 88020
atgccggcga tgaaaaaggc tatgaatctt ttccttggtt tatcaaacgt gcgcacagtc 88080
catccagagg gctttacagt gtacatatca acccatatct cattcccttc tttatcgggt 88140
tacagaaccg gtttacgcag tttcggctta gtgaaacaaa agaaatcacc aatccgtatg 88200
ccatgcgttt atacgaatcc ctgtgtcagt atcgtaagcc ggatggctca ggcatcgtct 88260
ctctgaaaat cgactggatc atagagcgtt accagctgcc tcaaagttac cagcgtatgc 88320
ctgacttccg ccgccgcttc ctgcaggtct gtgttaatga gatcaacagc agaactccaa 88380
tgcgcctctc atacattgag aaaaagaaag gccgccagac gactcatatc gtattttcct 88440
tccgcgatat cacttccatg acgacaggat agtctgaggg ttatctgtca cagatttgag 88500
ggtggttcgt cacatttgtt ctgacctact gagggtaatt tgtcacagtt ttgctgtttc 88560
cttcagcctg catggatttt ctcatacttt ttgaactgta atttttaagg aagccaaatt 88620
tgagggcagt ttgtcacagt tgatttcctt ctctttccct tcgtcatgtg acctgatatc 88680
gggggttagt tcgtcatcat tgatgagggt tgattatcac agtttattac tctgaattgg 88740
ctatccgcgt gtgtacctct acctggagtt tttcccacgg tggatatttc ttcttgcgct 88800
gagcgtaaga gctatctgac agaacagttc ttctttgctt cctcgccagt tcgctcgcta 88860
tgctcggtta cacggctgcg gcgagcgcta gtgataataa gtgactgagg tatgtgctct 88920
tcttatctcc ttttgtagtg ttgctcttat tttaaacaac tttgcggttt tttgatgact 88980
ttgcgatttt gttgttgctt tgcagtaaat tgcaagattt aataaaaaaa cgcaaagcaa 89040
tgattaaagg atgttcagaa tgaaactcat ggaaacactt aaccagtgca taaacgctgg 89100
tcatgaaatg acgaaggcta tcgccattgc acagtttaat gatgacagcc cggaagcgag 89160
gaaaataacc cggcgctgga gaataggtga agcagcggat ttagttgggg tttcttctca 89220
ggctatcaga gatgccgaga aagcagggcg actaccgcac ccggatatgg aaattcgagg 89280
acgggttgag caacgtgttg gttatacaat tgaacaaatt aatcatatgc gtgatgtgtt 89340
tggtacgcga ttgcgacgtg ctgaagacgt atttccaccg gtgatcgggg ttgctgccca 89400
taaaggtggc gtttacaaaa cctcagtttc tgttcatctt gctcaggatc tggctctgaa 89460
ggggctacgt gttttgctcg tggaaggtaa cgacccccag ggaacagcct caatgtatca 89520
cggatgggta ccagatcttc atattcatgc agaagacact ctcctgcctt tctatcttgg 89580
ggaaaaggac gatgtcactt atgcaataaa gcccacttgc tggccggggc ttgacattat 89640
tccttcctgt ctggctctgc accgtattga aactgagtta atgggcaaat ttgatgaagg 89700
taaactgccc accgatccac acctgatgct ccgactggcc attgaaactg ttgctcatga 89760
ctatgatgtc atagttattg acagcgcgcc taacctgggt atcggcacga ttaatgtcgt 89820
atgtgctgct gatgtgctga ttgttcccac gcctgctgag ttgtttgact acacctccgc 89880
actgcagttt ttcgatatgc ttcgtgatct gctcaagaac gttgatctta aagggttcga 89940
gcctgatgta cgtattttgc ttaccaaata cagcaatagc aatggctctc agtccccgtg 90000
gatggaggag caaattcggg atgcctgggg aagcatggtt ctaaaaaatg ttgtacgtga 90060
aacggatgaa gttggtaaag gtcagatccg gatgagaact gtttttgaac aggccattga 90120
tcaacgctct tcaactggtg cctggagaaa tgctctttct atttgggaac ctgtctgcaa 90180
tgaaattttc gatcgtctga ttaaaccacg ctgggagatt agataatgaa gcgtgcgcct 90240
gttattccaa aacatacgct caatactcaa ccggttgaag atacttcgtt atcgacacca 90300
gctgccccga tggtggattc gttaattgcg cgcgtaggag taatggctcg cggtaatgcc 90360
attactttgc ctgtatgtgg tcgggatgtg aagtttactc ttgaagtgct ccggggtgat 90420
agtgttgaga agacctctcg ggtatggtca ggtaatgaac gtgaccagga gctgcttact 90480
gaggacgcac tggatgatct catcccttct tttctactga ctggtcaaca gacaccggcg 90540
ttcggtcgaa gagtatctgg tgtcatagaa attgccgatg ggagtcgccg tcgtaaagct 90600
gctgcactta ccgaaagtga ttatcgtgtt ctggttggcg agctggatga tgagcagatg 90660
gctgcattat ccagattggg taacgattat cgcccaacaa gtgcttatga acgtggtcag 90720
cgttatgcaa gccgattgca gaatgaattt gctggaaata tttctgcgct ggctgatgcg 90780
gaaaatattt cacgtaagat tattacccgc tgtatcaaca ccgccaaatt gcctaaatca 90840
gttgttgctc ttttttctca ccccggtgaa ctatctgccc ggtcaggtga tgcacttcaa 90900
aaagccttta cagataaaga ggaattactt aagcagcagg catctaacct tcatgagcag 90960
aaaaaagctg gggtgatatt tgaagctgaa gaagttatca ctcttttaac ttctgtgctt 91020
aaaacgtcat ctgcatcaag aactagttta agctcacgac atcagtttgc tcctggagcg 91080
acagtattgt ataagggcga taaaatggtg cttaacctgg acaggtctcg tgttccaact 91140
gagtgtatag agaaaattga ggccattctt aaggaacttg aaaagccagc accctgatgc 91200
gacctcgttt tagtctacgt ttatctgtct ttacttaatg tcctttgtta caggccagaa 91260
agcataactg gcctgaatat tctctctggg cccactgttc cacttgtatc gtcggtctga 91320
taatcagact gggaccacgg tcccactcgt atcgtcggtc tgattattag tctgggacca 91380
cggtcccact cgtatcgtcg gtctgattat tagtctggga ccacggtccc actcgtatcg 91440
tcggtct 91447
```

1. A synthetic promoter comprising a nucleotide sequence selected from the group consisting of SEQ ID NO: 1 to SEQ ID NO: 25.

2. The synthetic promoter of claim 1, wherein the synthetic promoter is operably linked to a daptomycin biosynthetic gene cluster.

3. A gene construct for producing daptomycin, in which the synthetic promoter according to the claim 1 and a daptomycin biosynthetic gene cluster are operably linked.

4. The gene construct of claim 3,
wherein the daptomycin biosynthetic gene cluster comprises biosynthetic genes of dptA, dptBC, dptD, dptE, dptF, dptG, dptH, dptI, and dptJ; transporter protein genes of dptN, dptM, and dptP; and transcriptional regulatory factor genes of dptR1 and dptR2.

5. The gene construct of claim 3, wherein the daptomycin biosynthetic gene cluster comprises a nucleotide sequence of SEQ ID NO: 40.

6. The gene construct of claim 3, wherein the synthetic promoter replaces one or more promoters selected from the group consisting of a first promoter located at nucleotide sequence positions 75,318 to 75,707th of the daptomycin biosynthetic gene cluster; a second promoter located at nucleotide sequence positions 73,171 to 73,185th; a third promoter located at nucleotide sequence positions 26,456 to 26,528th; a fourth promoter located at nucleotide sequence positions 25,292 to 25,374th; and a fifth promoter and a sixth promoter located at nucleotide sequence positions 77,531 to 78,288th.

7. The gene construct of claim 6, wherein the synthetic promoter replacing the first promoter of the daptomycin biosynthetic gene cluster is a synthetic promoter comprising any one nucleotide sequence selected from the group consisting of B25 (SEQ ID NO: 2), A48 (SEQ ID NO: 3), A01 (SEQ ID NO: 4), A46 (SEQ ID NO: 5), A15 (SEQ ID NO: 8), A09 (SEQ ID NO: 9), B40 (SEQ ID NO: 11), A49 (SEQ ID NO: 12), B31 (SEQ ID NO: 13), B28 (SEQ ID NO: 14), A47 (SEQ ID NO: 15), A20 (SEQ ID NO: 16), B52 (SEQ ID NO: 19), R06 (SEQ ID NO: 18), A32 (SEQ ID NO: 20), R05 (SEQ ID NO: 22), A07 (SEQ ID NO: 23), A29 (SEQ ID NO: 24), and B45 (SEQ ID NO: 25).

8. The gene construct of claim 6, wherein the synthetic promoter replacing the second promoter of the daptomycin biosynthetic gene cluster is a synthetic promoter comprising any one nucleotide sequence selected from the group consisting of A12 (SEQ ID NO: 1), B25 (SEQ ID NO: 2), A48 (SEQ ID NO: 3), A01 (SEQ ID NO: 4), A46 (SEQ ID NO: 5), A31 (SEQ ID NO: 6), A26 (SEQ ID NO: 7), A15 (SEQ ID NO: 8), A10 (SEQ ID NO: 10), A49 (SEQ ID NO: 12), B31 (SEQ ID NO: 13), B28 (SEQ ID NO: 14), A47 (SEQ ID NO: 15), B24 (SEQ ID NO: 17), R06 (SEQ ID NO: 18), B52 (SEQ ID NO: 19), B42 (SEQ ID NO: 21), R05 (SEQ ID NO: 22), A07 (SEQ ID NO: 23), and B45 (SEQ ID NO: 25).

9. The gene construct of claim 6, wherein the synthetic promoter replacing the third promoter of the daptomycin biosynthetic gene cluster is a synthetic promoter comprising any one nucleotide sequence selected from the group consisting of A12 (SEQ ID NO: 1), B25 (SEQ ID NO: 2), A48 (SEQ ID NO: 3), A01 (SEQ ID NO: 4), A46 (SEQ ID NO: 5), A31 (SEQ ID NO: 6), A26 (SEQ ID NO: 7), A15 (SEQ ID NO: 8), A09 (SEQ ID NO: 9), A10 (SEQ ID NO: 10), B40 (SEQ ID NO: 11), A49 (SEQ ID NO: 12), B31 (SEQ ID NO: 13), B28 (SEQ ID NO: 14), A47 (SEQ ID NO: 15), A20 (SEQ ID NO: 16), R06 (SEQ ID NO: 18), B52 (SEQ ID NO: 19), A32 (SEQ ID NO: 20), B42 (SEQ ID NO: 21), R05 (SEQ ID NO: 22), A07 (SEQ ID NO: 23), A29 (SEQ ID NO: 24), and B45 (SEQ ID NO: 25).

10. The gene construct of claim 6, wherein the synthetic promoter replacing the fourth promoter of the daptomycin biosynthetic gene cluster is a synthetic promoter comprising any one nucleotide sequence selected from the group consisting of A12 (SEQ ID NO: 1), A48 (SEQ ID NO: 3), A46 (SEQ ID NO: 5), A31 (SEQ ID NO: 6), A26 (SEQ ID NO: 7), A15 (SEQ ID NO: 8), A09 (SEQ ID NO: 9), A10 (SEQ ID NO: 10), B40 (SEQ ID NO: 11), A49 (SEQ ID NO: 12), B31 (SEQ ID NO: 13), B28 (SEQ ID NO: 14), A47 (SEQ ID NO: 15), A20 (SEQ ID NO: 16), R06 (SEQ ID NO: 18), B52 (SEQ ID NO: 19), A32 (SEQ ID NO: 20), R05 (SEQ ID NO: 22), A07 (SEQ ID NO: 23), A29 (SEQ ID NO: 24), and B45 (SEQ ID NO: 25).

11. The gene construct of claim 6, wherein the synthetic promoter replacing the fifth promoter and the sixth promoter of the daptomycin biosynthetic gene cluster may be a synthetic promoter comprising any one nucleotide sequence selected from the group consisting of A12 (SEQ ID NO: 1), B25 (SEQ ID NO: 2), A01 (SEQ ID NO: 4), A31 (SEQ ID NO: 6), A26 (SEQ ID NO: 7), A15 (SEQ ID NO: 8), A09 (SEQ ID NO: 9), A10 (SEQ ID NO: 10), B40 (SEQ ID NO: 11), A49 (SEQ ID NO: 12), A47 (SEQ ID NO: 15), A20 (SEQ ID NO: 16), B24 (SEQ ID NO: 17), R06 (SEQ ID NO: 18), A32 (SEQ ID NO: 20), B42 (SEQ ID NO: 21), R05 (SEQ ID NO: 22), and B45 (SEQ ID NO: 25).

12. A recombinant expression vector for producing daptomycin comprising the gene construct of claim 3.

13. The recombinant expression vector of claim 12, wherein the recombinant expression vector is selected from the group consisting of a plasmid vector, a cosmid vector, a bacterial artificial chromosome (BAC) vector, and a yeast artificial chromosome (YAC) vector.

14. A transformant for producing daptomycin, transformed with the recombinant expression vector of claim 12.

15. The transformant of claim 14, wherein the transformant is a strain of the genus Streptomyces.

16. The transformant of claim 15, wherein the strain of the genus Streptomyces is selected from the group consisting of Streptomyces lividans, Streptomyces coelicolor, Streptomyces albidoflavus, and Streptomyces roseosporus.

17. The transformant of claim 14, wherein the transformant is additionally transformed with a recombinant expression vector containing genes encoding fatty acid ligase (dptE) and acyl carrier protein (dptF).

18. A method for producing daptomycin, comprising culturing the transformant for producing daptomycin of claim 14.

19. The method of claim 18, wherein the method is a method of culturing a transformant in a culture medium containing decanoic acid.


SEQUENCE LISTING (40 sequences)

SEQ ID NO: 1 - DNA, 269 bp, synthetic construct
  misc_feature 1..269: note = A12 promoter
  source 1..269: mol_type = other DNA
atcgcttgcaggcgaggtccagcgacgagcgtcgcacgctgccacagcaggacaggtcgcaggtgagcgtgcactgacggcacctcgtcgggactgctgagcacggtcacggcagccgagtggagcacgcgtgagggagcgacgcaggtgacggtctcagcgccatcctgtgcgagtgaccagcacactacggagcggacgagtacggcttgtcagtactgaccctctagtgctacggtcaataaggcgaaacactcaggaggaagact

SEQ ID NO: 2 - DNA, 269 bp, synthetic construct
  misc_feature 1..269: note = B25 promoter
  source 1..269: mol_type = other DNA
cgctatgccacgcaggttagcagcctggcacgtgagctgccgatgagtctaggcagcagaagagccggataggcacgagctcagtgacgaggccggtgaccgtcacggacggtactagccgaggagggctgagcgagggtgcgtgagaccgcgagcggacggtccacgagtgcggacgcagtgcagctgcgtggagcgacccccaatgcttgacaagagcgttcaaacgcatctagcattcgctacaaagccccgccaggaggtacagc

SEQ ID NO: 3 - DNA, 269 bp, synthetic construct
  misc_feature 1..269: note = A48 promoter
  source 1..269: mol_type = other DNA
tcagcaaggctcacctacggccagctcgacacctggctcacccagacctgccaacgcctccacgcctgctacctgtccccatccgacgaggccacctcgccaccgccacggtcccacctactcatcaccgtctccgtcactcagggactgaagcacctcacgcgcactcaccgcaccttgcaccacaggcacgcagtcttcgagaccccttgacgccccactcgccttacacgtacactctaaagcatcccgtatcgaggaggtcaagc

SEQ ID NO: 4 - DNA, 269 bp, synthetic construct
  misc_feature 1..269: note = A01 promoter
  source 1..269: mol_type = other DNA
cgctatgccacgcaggttcgacgtgagcgtcacagcggtcgcggtggatggtgaggcggatactgaagggtggtgcgcggtccatggtggatggcagggcgaacggacacgcaacggaggcagggtggtagtgcggagaggctccaggtgatggtccgtgtgtagagggccagaggaggtcggcggtagtgtcccgtaggtaacttcatttggcgtgcatgaactcaacgtgctatccttcctaaaaaggccccgccaggaggtacagc

SEQ ID NO: 5 - DNA, 269 bp, synthetic construct
  misc_feature 1..269: note = A46 promoter
  source 1..269: mol_type = other DNA
gtaggtgagccttgctgacggtcgaagaggcgtgcgggtcgtacgcaagtcggacacggacgtagtcgcgaggtgagtggtgatgcaagtggtaccagccgacagagtcgtccgcagtagcagacggctgtggatcgatagtgcgggtagagcgtgtggagagcgtgacccgtggatcatggcatgcgtcgaggtaccgtcacattgggttgccagcccgttgctggacgtactacggttgaagcctccccggaatcaggaggacttcc

SEQ ID NO: 6 - DNA, 268 bp, synthetic construct
  misc_feature 1..268: note = A31 promoter
  source 1..268: mol_type = other DNA
taccacgcgtacagctacgacggctcggccaacgtccgccgtctcctccgtcacgtcgctgctcacgcggtactggctcagctgcgccagctggactcggctcagtcggtcccacccttcgtgcatcagggtccagcgaccggtgacgctgcacgccgtggctgctagtcggccgactccccgttgccacctggaccgacgtactgagcttgcctgaagaacccacatccagctaaagtaagccaacctgacctttgaggagggctca

SEQ ID NO: 7 - DNA, 268 bp, synthetic construct
  misc_feature 1..268: note = A26 promoter
  source 1..268: mol_type = other DNA
taccacgcgtacagctacatgcggaggaacagagtggccagccgttgccacgacgtccggacggcacgacacccccgcaggtacgatcagtggcacgcgaggcctgcgacagcgccaagaccggatacgacaggaaccgtggcaacagtccgtagacaccgcagccagcgcagatcgcgggaggagatccagatcgacgccctgatgacttgacgctcgagaaggttcacgattagcatgccggttacggacctttgaggagggctca

SEQ ID NO: 8 - DNA, 269 bp, synthetic construct
  misc_feature 1..269: note = A15 promoter
  source 1..269: mol_type = other DNA
acctcgcctgcaagcgatccccatatctcccgctggtcacgtctgccgactagacggtggcgcacaccctggttgctcaagcgtctgcggcccatcctctcaaggtgtgctccggactgtgccaggtctggcgccttccactccgcgccgtgataccgttgggtagtggtcgctctgcgtactggcctcgtcctacagccgcgaagtacttgacacatacctgctaagccttctaatctgcttgcgtactctaccacaggaggtaacta

SEQ ID NO: 9 - DNA, 269 bp, synthetic construct
  misc_feature 1..269: note = A09 promoter
  source 1..269: mol_type = other DNA
actggtgaatgatgggcactgtcgcacgagagcgtcagacggacctagagcctagaccctcgctcacctcgcacaatcagacggctctgggacgctggctgccacgagcgagacgtcacgtctggagcagcactggcgcagcctaccgccacgcatccatcccgatccgacgagcgagcgggtgaccgctctggctcagacgcagccgcttgaccacatggtcccgggcagtatacggtggctgcgaccccaaccaaaggaggcagtgt

SEQ ID NO: 10 - DNA, 269 bp, synthetic construct
  misc_feature 1..269: note = A10 promoter
  source 1..269: mol_type = other DNA
atcgcttgcaggcgaggtgcgatccactcagagctgctgacagtccaccgtcgtctgaccgttcgcgaccactccggactggccagcgtgtccgaggcctcagtcgctgtcgtgagataggcttgcagggacgccactagcgacggtcgtggacaggtgacacgcgcatcaaggacggcgatgtctccgtagctagcggagaacacctcttgactcgcctacccgccgctcgctaaatttgctgtagccaaacactcaggaggaagact

SEQ ID NO: 11 - DNA, 269 bp, synthetic construct
  misc_feature 1..269: note = B40 promoter
  source 1..269: mol_type = other DNA
acctcgcctgcaagcgatagctcacacggccatccgcatccacacaacggcgctgaccacgctctggcacgacgcctacgacggtccagttggcagcagcgactgcgacctcccagaccccgcttgcgaggctcacacccagaccgcagtcacgacttgcgaccgcagcctgcggacctaggacctgactgcacgctcgtcaccaccatttgtccacccttctccttagctggtaccatccaggttcgctctaccacaggaggtaacta

SEQ ID NO: 12 - DNA, 269 bp, synthetic construct
  misc_feature 1..269: note = A49 promoter
  source 1..269: mol_type = other DNA
tgcccatcattcaccagtaaccgagacggcatgtgggaccctggccgagaggagccaccgtagggagaagtaaggcggagcgagcagggatggcacggagaggaggtgagcggtgcgagcagtcgtacggacacggaggacacgcggtggtgacgccgggcatcgccgcaccgaaggagccgtgacaggctctcggcactatagtccccttgacgagcccgagagagtgcagctaaggtcccacgttcttacgaacgaggaggccccat

SEQ ID NO: 13 - DNA, 269 bp, synthetic construct
  misc_feature 1..269: note = B31 promoter
  source 1..269: mol_type = other DNA
tcagcaaggctcacctactagacgcgtgcggcggccttgccgtggtggtacgtcggagtgcgtgagggtgtggtttgcgatagggcgaactggcgacagtgctcggcgacggctgggatgttgggttgtggttcagggctgccagtgcgttggtcctacgggcttaggtggttggcgcgtgtgactggacgactcggacattaaccacattgacagcctgatgccgcggcgattacgatcgctaatcacccgtatcgaggaggtcaagc

SEQ ID NO: 14 - DNA, 269 bp, synthetic construct
  misc_feature 1..269: note = B28 promoter
  source 1..269: mol_type = other DNA
gtaggtgagccttgctgatgacccaccagccacacctcgcctacgcccgtgccacaccgacgcgctcaacacccaccagtcatcacaacgccacgccgtacaccacagccaccacttcacgagccgagtgcacggcacacacgtcgccacgaacgcacgacaacgaccacgtccacacgcaccgacggcagtgccgagacactgatgcattgacaagaatcacggaaacaccttacactcgatgagtggccggaatcaggaggacttcc

SEQ ID NO: 15 - DNA, 269 bp, synthetic construct
  misc_feature 1..269: note = A47 promoter
  source 1..269: mol_type = other DNA
actggtgaatgatgggcagtccgtcctgctagcgagccgtctcgtccccaccgcacgtgaggcacacctacctcctccgtacgccgaactctccgctcgcgcgagtctgctacgtcggctagtcgctggctcatcgcagctgtatcttgcttcgttcgtacctcggacgcctgtcgctggcgctgacctcggcactgctgcgaatgcatttgacttcgatctagggaaccaggtacgatagaaaataatccaaccaaaggaggcagtgt

SEQ ID NO: 16 - DNA, 269 bp, synthetic construct
  misc_feature 1..269: note = A20 promoter
  source 1..269: mol_type = other DNA
acctcgcctgcaagcgatggagctgggtgcgacgggacattgcatgccggtacgctggatcgctgggatcgcgacgcagtgctgccgtggagcggtgccgtgcagtggtcgcacctctccagcgtcgcgagcagagcgtgccgcagccttgcccagtactctacgcgcccactgggctgtcgccacctgacggcacttgacgggatgtgttgcctccgactgttcacctcacctattatgcctccacactctaccacaggaggtaacta

SEQ ID NO: 17 - DNA, 269 bp, synthetic construct
  misc_feature 1..269: note = B24 promoter
  source 1..269: mol_type = other DNA
atcgcttgcaggcgaggtctggtcgggttcgtgtcgcggagagccgtggtggtaagggtcggggtgccggctgtctggtgtgggggtggcgcgttcgcaccgtagtcgttgcgaccgtcgaagcgtgcctgtgcctccctgttctcgtgcacagcccggtgtcggggtcgcagtcggactcggtccgagctccatccacaagcgaatacttgccgccccttccgtctggatgttaagttaatctcagacaaacactcaggaggaagact

SEQ ID NO: 18 - DNA, 200 bp, synthetic construct
  misc_feature 1..200: note = R06 promoter
  source 1..200: mol_type = other DNA
cgacgcttgtcgctaccagaatcattcccgaacggtgctgccataaggctccgcagagcagctaagcgataccacaccaacgagtgtaacgtgccgaatcgttgtcgacatcaccgacacctgcttgactccgtccttggttggccccgtgcaaagcgtcatgctagactacatgactggcctatcataggaggcagttt

SEQ ID NO: 19 - DNA, 269 bp, synthetic construct
  misc_feature 1..269: note = B52 promoter
  source 1..269: mol_type = other DNA
tgcccatcattcaccagtggaccacagcggcatcggacagcgtgcaaccctcgcaaccaggtcccccgacgacacctacgtgcagccacaacacagccacagccgccagcacggtgaccccacctctgccgacccacccagatccgagcttggacacccactgcgagccacctgaccgacaacggtcgccaacgctacagacttcctgtttgacaaggccaatgcagccacactacccttaacgcgacttacgaacgaggaggccccat

SEQ ID NO: 20 - DNA, 269 bp, synthetic construct
  misc_feature 1..269: note = A32 promoter
  source 1..269: mol_type = other DNA
gtagctgtacgcgtggtaccaggaggcgctgcacgcaagagccgacctcggacggacccactcagagcatcgaggttcgtaggaggcaaggcggtcgagacacgacagaccgctagaggcagcagggtggccaacaggccgcatagcagcgtccacagggaacacggttggaccagcgcaggcactccaacgacacgcggtcccacgccttggcccgagggccgaccgggtgctattttcctcagaacaagaatacgaggaggccccat

SEQ ID NO: 21 - DNA, 269 bp, synthetic construct
  misc_feature 1..269: note = B42 promoter
  source 1..269: mol_type = other DNA
cgctatgccacgcaggttgtcaggcacgtaggtgccagaccgatcgcccaacccgtcacactccactcgactctactatgactcatccctacactcgctacctagctcacgaggcctagcgctcaagtggctagcacctgcgccactcggctctcacgtgcaagcccgtgcaccttcacgtgcgactcacgcagtggtcaccccaggacttgtccggagcggccgaagggtactatgatagaggccagtgccccgccaggaggtacagc

SEQ ID NO: 22 - DNA, 200 bp, synthetic construct
  misc_feature 1..200: note = R05 promoter
  source 1..200: mol_type = other DNA
ttcactcgcgtccacagactggagcgtgcaccgactcgcacagcgtcggtacggtgcgtgtggaacgcattagtgaactggtccgacagactctcggtcacacgactgatcagcaggtaccgtcttgcaacacagtacccttgacgcctcacttgtaccagccctaacatgcctcctccgttggcttaaggaggccccat

SEQ ID NO: 23 - DNA, 269 bp, synthetic construct
  misc_feature 1..269: note = A07 promoter
  source 1..269: mol_type = other DNA
gtaggtgagccttgctgagcgagtgccatcggacggactagccagaggtagagcgacgattgcgaccgtggtcgatgagctggttcggagccgacgagccgtcgcgtgactacgctgacggcgtgaccacgagcgagcgtgatggcgactgtgcaccgcagagtgccgcaccgggctacatacgacgacacgcgtctggagaatctaagttgacgggacggtacggtgtgcggtaacgtctccccggtgccggaatcaggaggacttcc

SEQ ID NO: 24 - DNA, 269 bp, synthetic construct
  misc_feature 1..269: note = A29 promoter
  source 1..269: mol_type = other DNA
tcagcaaggctcacctacctcctgcctacgcacggccaccttcgagcgccttgcactacgccactgcgcatccactatccctgcctgcacggacactctgactggcacggtcgagctctggtcgcacatcccgctctcgtcctccacgcccccttcgagatcctagccctgcacctgtaccgcgcagctccgtcgttgcaccctaacacttgacgcaacgccttccgtccgcataatctgttcggctgtccgtatcgaggaggtcaagc

SEQ ID NO: 25 - DNA, 269 bp, synthetic construct
  misc_feature 1..269: note = B45 promoter
  source 1..269: mol_type = other DNA
actggtgaatgatgggcactggtccggatcggtgcaggcggtgcactcgtggagtgggtgggggtgacggctgactggtctgcgggtggtgagctcgccacgtagtcggtgtcgagtctgcccagacgctgtgcctcactgatcccgtgcgacgcacggtgtcgcggtcggacgctccggacgtagctccgtctcgttcaaacagcccattgacacggttcactgcgcgcttttatgatgtacagccctccaaccaaaggaggcagtgt

SEQ ID NO: 26 - protein, 371 aa, synthetic construct
  REGION 1..371: note = dptR2_regulatory gene 2
MALASERQEFILATVRERGTVRLADLVERLGVTAVTVRRDVTILADRGLVSRVHGGVTLPYRGPTADERATRGVFTHGRLPDQAFVGMVVPTVEYYWPAVIQGAQSAVAAAGGRLVLRASAYDAAEDRRQVTGLLDRGVRTLLVAPTNTGEAGQDLLRWLGSLNVPVILVERLPPPALPTLPLDAVTTAHSLGAGLAVRHLVTLGHHRIAFVTARFSPTTKALREGWQETTASLGLPSGDGLAHDVPSYGSPGWADAYDAALRSCREAGATALFVHSDGEAVGLVERAHEHGLAVPEDLAVITYDDEVAAAADPPLTAVRPQKHRLGVLAAEMALARAVDPVERPVHRVELWPNLIIRASCGGTAPVSAGR

SEQ ID NO: 27 - protein, 341 aa, synthetic construct
  REGION 1..341: note = dptR1_transcriptional regulator 1
MTIPPVGRTGWDTQWQALGLGADELRVYEALLDAPVPASRTALAQHLGLTVRRVTNALDHLAERRFTHPARGAGLPVAMAPATALRNLIHLHQAELLHRSAELEELSGSVDRIAAQLLSSVHTPRATGIETVRGGAAIAARVASLLVSASEEIALLDRPPYASSEPDGMPVPLDMAEPVRRGVRVRVVVDREGLSFHGRARGLGDLAVQGVQIRVGTDLPTKLITVDRRVTLLPPTDAADPTASALVVSDALLSNALVPLFESVWERALPIGSVTHDQITDGDRELLTMLASGLKDEAMARRLDIHVHTVRRRITRLMQVLNAETRFQAGVQAALRGWLTG

SEQ ID NO: 28 - protein, 245 aa, synthetic construct
  REGION 1..245: note = dptJ_Tryptophan 1,3-dioxygenase
MTAQDTRTTGSDGGGRGATYHESPTYGELLRLEDLLNVAHLRDAAAPVLFLATHQSAEIWFGIVLRHLEEIRAALTDDDPDTALHLLPRLPEIFELLVRHFDMLATLSTEEFGKIRAGLGTASGFQSAQYREIEFLCGLRDHRHISTPGFTETERRRLRERARQPSVAEAYDAFRTRCANGKDAERIGEALLRFDERVTVWRARHAALAERFLGPLEGTAGTAGADYLWRVTRHRLFPPEAWGAG

SEQ ID NO: 29 - protein, 328 aa, synthetic construct
  REGION 1..328: note = dptI_Glutamine 3-methyl transferase
MTVHDYHVTVKEQHPALFELLDPARLVAVTDEPWVTEGNEFDDDHTGRGVSYRCAQQHGEARRTGIETILGMFAGPGGLRDMGRVLDVLGGEGLLSRVWRQLAGAGDGDSVPLVTGDLSGHMVAAALRSGLPAVRQPADRMLQRDHCLDGVLFAYGTHHVDRSVRPRMLTEASRVLAPGGRVVLHDFAEGSPEERWFREVVHPRSLAGHAYDHFTAHEMTGYLADAGFTDITVGPVYDPMTLTGETEESALARLVSYMTSMYGILPDGDRSNERTEAALRDIFRFSAGDLPEDVPRDEAVLELTVRPHGNAFRAELPRIALVAHGRKP

SEQ ID NO: 30 - protein, 271 aa, synthetic construct
  REGION 1..271: note = dptH_alpha/beta Hydrolase
MRATSRMIQVNGARIACSDSGCGDPVLMIAGTGSTGRVWDAYQVPDLHAAGFRTITFTNRGVPPSDECERGFTLADLAADTAALIEQVAGGPCRVVGTSLGAQVAQEVALARPDLVTQAVFMATRGRTDAMRAAATRAAAALYDSGVELPPAYAAAVRALQNLSPHTLRDRHQVEDWLPLFEYAERDGPGVRAQLELGLLPDRLADYRDITVPCLVIAFEDDVVTPPYLGREVADAIPGARFETVPRCGHYGYLEDASAVNKILRDFFRTS

SEQ ID NO: 31 - protein, 75 aa, synthetic construct
  REGION 1..75: note = dptG_mbtH family protein
MANPFENNDGSYLVLVNDEGQYSLWPAFADVPAGWTVTFGESSRQECLDHINENWTDMRPKSLIRQMENDRTTAA

SEQ ID NO: 32 - protein, 2379 aa, synthetic construct
  REGION 1..2379: note = dptD_daptomycin non-ribosomal peptide synthetase D
MTQRAMEDILPLTPLQEGLLFHSVYDEQSVDVYTVQVVVDLEGPVDPEALRAAAAALLRRHANLRAAFRYERLQRPVQIIPREVAVPWEHTDVAKLEGAEQKAEIERLLHDQRWRRFDLTAPPLLRFLLVRTGHDRHRFALTFHHILMDGWSMPVLLRELITLYRTGDETALPWVRPYRDYLAWISRRDRDEAGRAWSKALAGVDEATLVAPGADRAAEPPLWTESRLEPDLAATLAARAREFGVTLNTLVQAAWALVLGRLTGRDDVVFGVTVSGRPPELAGVEDMVGLFINTVPLRAELLPHESLRDFTVRLQREQIQLLDHQYERLAVIQRLAGRTELFDTVMVFENYPVAAASSAGADGPAAEPRVADVHVRDAMHYPLGLLVLPGPPLRLRFGHRPSALPAERVTTIRDSLVRALELMADQPDLAVGRADILGEEEKQHLLTGLNDTHRDVPPLTVPGMIEAQAARTPGRPAVHARDGELSYAELNARANRLARHLAAAGVGPEQYVTLLLPLSARMVVAALAVMKTGAAYVPVDPEYPADRIAYMLGDIGPALVLTDSRSAAAMPAGPARVLTLDDDALDTGVRALPEHDLGTDGIAPLPDQPAYVIYTSGSTGRPKGVVILHRSVTGYLLRTIEEYPEAAGKAFVHSPVSFDLTVGALYAPLVSGGCLRLGSFTDDKILDLGEDSPTFMKATPSHLAVLDSLPDEISPTGAITLGGEQLLSETLDPWRARHPGVTVFNVYGPTETTINCAEHRIAPGTTLPPGPVPIGRPLWNTRLYVLDGGLRVVPTGVAGELYVAGAGLARGYLGRPGLTAERFVACPFGAPGERMYRTGDLVRWRTDGTLEFVGRVDDQVKVRGFRIELGEVEATVAATPGVARAIVAVREDRPGDQRLVAYVTPADVDPTGGLPSAVTAHAAARLPAYMVPSAVVVLHEVPLTPNGKINRAALPAPEAVSGAGFRAPGTAREEVLCGLFAEVLGLERVGTADDFFELGGHSLLATRLVSRVRSVLGVELGVRALFDAPTPGRLDRLLGERSGAPVRAPLTARERTGRDPLSYAQQRLWFLHELEGHGATYNIPLALRLTGPLDVTALEAALTDVVARHESLRTLIARDGTGTAWQHILPTGDPRARITLEAVPLHRDELAGRLAEAARHPFDLTAEIPVRATVFRTERDDHTLLVVTHHIASDRWSREPFLRDLSAAYAARRAHSAPELPPLSVQYADYAAWQRDVLGTEDDGTSEMAGQLAHWRGRLAGLPQGLDLPTDRPRRPDVGRRGGRCRLEIPAALHRDIVTLARVTSTTVFMVVQAALAGLLSRLGAGTDIPIGTPIAGRTDEATEHLIGFFVNTLVLRTDVSGDPTFAELLARVRATDLDAYAHQDVPFERLVEVLNPERSLLRHPLFQILLAFQNTEDRSISDRPGTLLPDLQVTEQPLDAGTAKFDLAFAFTERPPEKGEPSGITGIVEYHADLYDEGTVRQIADCFVQFLDAAVHAPGTRVDAVGLLPEHTLHKLLTRSRGTVTGLPPATLPELFEARVAAHPGHIAVEVAGRRPATTTYDALNRRANRLARLLTDRGVRPEQRVAIALPRSADLVTAWLGILKAGAVCVPVDPAYPDDRIAHMAADAAPALLIASAATRDRMLPTGIPVLDLDDPAVTAALAAAPDGNPRGTGLLPAHPAYVIYTSGSTGTPKGVVVTHEGIPALAATQQEALRAGPGDRVLQLVSTSFDASVWDLCSALLSGATLVLAPDADLFGDELAAALTAHRITHVTLPPAALAAVPAGAAPPRLTVTVTGDVCGPQLVDRWAGGERRILNGYGPTEVTVGATYAVCERTGDGAPVPIGAPWPDQRVYVLDHRLRPVPAGCVGEIYVAGAGLARGYLGRPGQTAERFVADPFGAPGERMYRTGDLARRRSDGHLLFEGRADTQVKIRGFRVELAEIEAALASHPGVEDAVVTVYDDGLGDQRLVAYVTGGPGTPSAAALRAHLASRLPRHMVPGDVLTLDALPLTANGKVDRTALPGPGTQTAAPGRAPQSPQERVLCALFADVLGRETVGVDEGFFDLGGHSLLATRLAARVRAALGVEISVRTLFEAPTPALLASACTADAAAYDPFETVLPLRRTGSRPPLFCVHAGMGLSWAYAGLLSHLDADVPVYGLQARRLTAPGGLPGSVEEMAEDYAGEIRRLCPDGPYRLLGWSFGGTVAHAVATRLQQQGHTVELLAVLDAYPVTGARPDAEVDEQRIVADYLAQLGSPVAPERLEGDAWLPEFLEFVRRTDGPARDFDAGRILAMKDVFLNNARLTRRFTPGVFTGDMVFFASARPGSEQAAERVGLWHPHVTGDLDLHLIDCAHEEMTDPAALTRIGPVLAARLGAGT

SEQ ID NO: 33 - protein, 7338 aa, synthetic construct
  REGION 1..7338: note = dptBC_daptomycin non-ribosomal peptide synthetase BC
MNRRSKVVEEILPVSALQEGLLFHSSFAAADGVDVYAGQLAFDLVGAVDTGRLRAAVESLVARHGVLRSSYRQARSGEWVAVVARRVATPWRAVDARDGATDAAAVAREERWRPFDLGRAPLARFVLVRTDDDRFRFVITYHHVILDGWSLPVLLRELLALYGSGADPSVLPPVRPYGDFLRWAAARDDAAAETAWRDALTGLDEPSLVAPGASPDGVVPASVHAELDKAGTENLAAWARHRGITQATAVRAAWALVLGQHTGRDDVVFGVTVSGRPAELAGAEHMVGLFINTVPLRTVLDPADTLGTFAARLQAEQTTLLEHQHVRLSDIQRWAGHKELFDTIVVFENYPIGHSGPGSIRTDDFTVTATEGSDATHYPLTLTAVPGETLRLKLDHRPDLVDTTTATALLRRVTRVLETATDDTGHTLARLDLLDDDERHRLLRGWNDTTREQPPTYYHQEFEEQARRRPHDTALVFTSTSWTYEELNDRANRLARLLVAAGAGSDDFVALAFPRSAESVVAILAVLKAGAAYLPLDMDQPAERLTGILADAHPTVVLTTTTATPLPHPGRTLVLDSPTTARALAAAPAHNLTDADRRTPLNARNAAYIIHTSGSTGRPKGVVIEHRSLANLFHDHRRALIEPHAAGGSRLKAGLTASLSFDTSWEGLICLAAGHELHLIDDDTRRDAERVAELIDRQRIDVIDVTPSFAQQLVETGILDEGRHHPAAFMLGGEGVDAKLWTRLSDVPGVTSYNYYGPTEFTVDALACTVGIAPRPVIGHPLDNTAAYILDGFLRPVPEGVAGELYLAGTQLARGYAGRPGLTAERFVACPFGAPGERMYRTGDLVRRSPGGVVEYLGRVDDQIKLRGFRIEPAEIELALAGHPAVAQNVVLLHRSATGEARLVAYVVPGTPVDPRELTGHLAARLPAYMVPSAFVLLDTLPLTPNGKLDRGALPEPAFGTAPRPERPRTPVEEILCGLYADVLGLPSFGADDDFFDAGGHSLLASKLVSRIRTNLKTELNVRALFEHRTVSSLATALHRAAQAGPALTAGPRPARIPLSYAQRRLWFLNRLDRDSAAYNMPVALRLRGPLDSTAMCAALTDVAERHEALRTVFEEDRDGAHQIVLPATGLGPLLTVTGADGTTLRALITEFVRRPFDLAAEIPFRAALFRVGDEEHVLVVVLHHIAGDGWSMGPLARDVAEAYRARAAGRAPDWEPLPVQYADYALWQREVLGAEDDETGELSAQLAHWRTRLAGAPAELTLPTDRPRPAVASTAGDRVEFTVPAGLHQALADLARAHGATVFMVVQAALAVLLSRLGAGDDIPIGTPVAGRTDEATEELIGFFVNTLVLRTDVSGDPTFAELLARVRATDLDAYAHQDVPFERLVEVLNPERSLARHPLFQVMLTFNVPDMDGVGSALGNLGELEVSGEAIRTDQTKVDLAFTCTEMYAADGAASGMRGVLEYRLDVFGAVQARETTERLVRVLEGVVSGGGGVSVSGVDVLGVGERERLLGWGVGGPVPVVPGGGLVGLFEERVRADADAVAVRGAGVVWSYGELNARVNVVARWLVGRGVGAECGVGVVMGRGVDVVVMLLAVAKAGGFYVPVDPEWPVERVGWVLADAGVGLVVVGEGLSHVVGDFPGGEVFEFSRVVRESCLVELVAADGVEVRNVTDGERASRLLPGHPLYVVYTSGSTGRPKGVVVTHASVGGYLARGRDVYAGAVGGVGFVHSSLAFDLTVTVLFTPLVSGGCVVLGELDESAQGVGASFVKVTPSHLGLLGELEGVVAGNGMLLVGGEALSGGALREWRERNPGVVVVNAYGPTELTVNCAEFLIAPGEEVPDGPVPIGRPFAGQRMFVLDAALRVVPVGVVGELYVAGVGLARGYLGRAGLTAERFVACPFGAPGERMYRTGDLVRWRVDGALEFVGRADDQVKVRGFRVELGEVEGAVAAHPDVVRAVVVVREDRPGDHRLVAYVTGVDTGGLSSAVMRAVAERLPAYMVPSAVVVLDEIPLTPNGKVDRAALPVPGVEAGAGYRAPVSPREEVLCGLFAEVLGLERVGVDDDFFGLGGHSLLATRLISRVRAVLGVEAGVRALFEAPTVSRLERLLRERSALGVRVPLVARERTGREPLSFAQQRLWFLEELEGPGAAYNIPMALRLAGVLDVEALHQALIDVIARHESLRTLIAQDAGTAWQHILPVDDPRTRPGLPLVDIGADALQERLDEAAGRPFDLAADLPVRATVFRLTDNDHILLVVAHHVAFDAMSRVPFIRNVKRAFEARTNGAAPDWRPLPVQYADYAAWQRDVLGTEDDESSELSAQLAYWRTQLASLPAELALPTDRARPAVASYEGGKVEFTVPAGVYDGLVALARAEGVTVFMVVQAALAALLSRLGAGDDIPIGTPIAGRTDQATEDLIGFFVNTLVLRTDVSGDPTFAELLARVRATDLDAYAHQDIPFERLVEAVNPERSLARHPLFQVMLTFDNTIDREVTEGFAGLGVEGLPLGAGAVKFDLLFGLSEVGGELRGAVEYRCDLFDHPTVAQLAERLVRVLERVASDASVRTGELPVVGEAERARVLTEWNDTGVPGVPETFLELFEAQVAARGDAPAVVYEGEVLSYRELDARANRLAGLLVGRGAGPEHFVGVALPRGLDLIVALLAVLKSGAAYVPLDPEYPAERLVHMVTDAAPVVVVTSTDVRTLRTVPRVELDDEATRATLVAAPATGPDVKMSASHPAYVIYTSGSTGRPKGVVISHGSLANFLAWAREDLGAERLRHVVLSTSLSFDVSVVELFAPLSCGGTVEIVRNLLALVDRPGRWSASLVSGVPSAFAQLLEAGLDRADVGMIALAGEALSARDVRRVRAVLPGARVANFYGPTEATVYATAWYGDTPMDAAAPMGRPLRNTCVYVLDDGLRVVPVGVVGELYVAGVGLARGYLGRVGLTAERFVACPFGARGERMYRTGDLVRWRVDGTLEFVGRADDQVKVRGFRVELGEVEGAVAAHPDVVRAVVVVREDRPGDHRLVAYVTGVDTGGLSSAVMRAVAERLPAYMVPSAVVVLDEIPLTPNGKVDRAGLPVPVVSVAGFCAPSSPREEVLCGLFAEVLGVERVGVDDGFFDLGGDSILSIQLVARARRAGLELSVRDVFEGRTVRALAAVVRGSDAGAVGVVGGAEIVLPGVGEVERWPVVEWLAERGGGSLGGVVRGFNQSVVLAVPAGLVWEELRVLLGAVRDRHEAWRLRVLDSGALCVDGVVPDDGSWIVRCDLSGMGVDGQVDAVRAAAVEARAWLDPSVGRVVRAVWLERGGDRSGVLVLVAHHLVVDGVSWRVVLGDLAEGWAQVRSGGRVELGVVGTSLRGWAAALAEQGRRGERAGEVELWSRMVRGADVLVGSRAVDGAVDVFGGVVSVDSRASVSVSRALLTEVPSVLGVGVQEVLLAAFGLAVARWRGRGGPVVVDVEGHGRNEDAVRGADLSRTVGWFTSVYPVRVPVESASWDEVRAGGPVVGRVVREVKETLRSLPDQGLGYGILRYLDPEHGPALARHATPQFGFNYLGRFTTGTDDTGDEGMTDWVPVSGPFAVGAGQDPELPVAHAVEFNAITLDTPEGPRLGVTWSWPTTLLPESRIRELARYWDEALEGLVEHARHPEAGGLTPSDVTLVEVNQVELDRLQAGVAGGAEEILPVSALQEGLLFHSALASGGVDVYVGQLVFDLVGPVDVDRLRAAVEGLVARHGVLRSGYRQLRSGEWVAVVARQVDLPWQSIDVRDGGIDGLVEEERWRRFDMGRGPLARFVLIRTHDDRFRFVITYHHVVLDGWSVPVLLRELLALYGSSGDVSVLPGVRSYGDFLRWVAARDAAAAEGAWRRALTGLEEPSLVAPGVSRDGVVPAAFHGAVDGDLSQKIVAWARGRGVTVASVVQAAWALVLGRLMGRDDVVFGVTVSGRPAEVVGVEDMVGLFVNTIPLRARLDPAESLGGFVERLQREQTELLEHQHVRLAEVQRWAGHKELFDVGMVFDNYPVSSESPEAEFQISRTGGYNGTHYALNLVASMHGLELELEIGYRPDVFDAGRVREVWGWLVRVLEGVVSGGGGVSVSGVDVLGVGERERLLGWGVGGPVPVVPGGGLVGLFEERVRADADAVAVRGAGVVWSYGELNARVNVVARWLVGRGVGAECGVGVVMGRGVDVVVMLLAVAKAGGFYVPVDPEWPVERVGWVLADAGVGLVVVGEGLSHVVGDFPGGEVFEFSRVVRESCLVELVAADGVEVRNVTDGERASRLLPGHPLYVVYTSGSTGRPKGVVVTHASVGGYLARGRDVYAGAVGGVGFVHSSLAFDLTVTVLFTPLVSGGCVVLGELDESAQGVGASFVKVTPSHLGLLGELEGVVAGNGMLLVGGEALSGGALREWRERNPGVVVVNAYGPTELTVNCAEFLIAPGEEVPDGPVPIGRPFAGQRMFVLDAALRVVPVGVVGELYVAGVGLARGYLGRVGLTAERFVACPFGVPGERMYRTGDLVRWRVDGALEFVGRADDQVKVRGFRVELGEVEGAVAAHPDVVRAVVVVREDRPGDHRLVAYVTAGGVGGDGLRSAISGLVAERLPAYMVPSAVVVLDEIPLTPNGKVDRAALPVPEVEAGTGYRAPVSPREEVLCGLFAEVLGVERVGVDDDFFELGGHSLLATRLISRVRAVLGVEAGVRALFEAPTVSRLERLLRERSGLGVRVPLVARERTGREPLSFAQQRLWFLEELEGPGAAYNIPMALRLAGVLDVEALHQALIDVIARHESLRTLIAQDAGTAWQHILPVDDPRTRPGLPLVDIGADALQERLDEAAGRPFDLAADLPVRATVFRLTDNDHILLLVLHHIAGDGWSMGPLARDLSTAYSARAAGAASAWRPLSVQYADYAAWQRDVLGTEDDESSELSAQLAYWRTQLASLPAELALPTDRARPAVATYRGGRIEFTIPADVHRSLADLARAEGVTVFMVVQAALAALLSRLGAGDDIPIGTPIAGRTDQATEDLIGFFVNTLVLRTDVSGDPTFAELLARVRATDLDAYAHQDIPFERLVEAVNPERSLARHPLFQVMLAFNNAETSTPLPMAEGLAASRQDIEPGVAKFDLALYCNESRGETGDHQGIRSVFEYRRDLWDEDTVRQLADRFLHVLAAFAAAPEQRASSVDVLRAGERDQLLHEWNDTAAALPPALLPQLFEEQVRRTPHDVALVSGNIRLTYAELDARANRLAHLLLARGAAPETFVAVALPRTEELLVALLAVQKTGAGHLPLDPGFPAERLSYMLDDARPAVVLTTEDISARIPGGSHVVLDSEQVTGELHDHPATSPAGRGNPAGPAYVIYTSGSTGQPKGVVVPSAALVNFLADMVPRLGLRGGDRLLSVTTVGFDIAALELFVPLLSGATVVLADGETVRDPALARQTCEDHGVTMVQATPSWWHGMLADAGDSLRGVHAVVGGEALSPGLRDALTRGARSVTNMYGPTETTIWSTSAGQAAGDSAPPSIGTPILNTRVYVLDAALCVVPPGVAGELYIAGDGLARGYLGRAGLTAERFVACPFGAPGERMYRTGDLVRWRVDGALEFVGRADDQVKVRGFRVELGEVEGAVAAHPDVVRAVVVVREDRPGDHRLVAYVTGVDTGGLSSAVMRAVAERLPAYMVPSAVVVLDEIPLTPNGKVDRAALPVPGVEAGAGYRAPVSPREEVLCGLFAEVLGVERVGVDDDFFGLGGHSLLATRLISRVRAVLGVEAGVRALFEAPTVSRLERLLRERSGLGVRVPLVARERTGREPLSFAQQRLWFLEELEGPGAAYNIPMALRLAGVLDVEALHQALIDVIARHESLRTLIARDSDGTARQQVLPVGDPAARPALPVVQTDADTLVAKLNEAVGRPFDLTAEMPLRATVFRVADEDHALLLVFHHIAGDGWSTGLLARDLSTAYAARLEGRDPQLPPLPVQYADYAAWQRDVLGTEDDESSELSAQLAYWRTQLADLPAELALPADRVRPARASYEGGRVGFTVPAGVLRDLTRLARVEGVTVFMVVQAALAALLSRLGAGDDIPIGTPIAGRTDQATEDLIGFFVNTLVLRTDVSGDPTFAELLARVRATDLDAYAHQDIPFERLVEAVNPERSLARHPLFQVMLAFDNTADGGPVEDFPGLSAAGLPLGAGAAKFDLLFGLSEVGGELRGAVEYRCDLFDHPTAARIAERLVRVLERVAADASVRLGELPVVSDAERACVLTEWNDTAVPGVTGTLSALFEARAAARGDAPAVVYEGEELSYRELNTRANRLAHVLAEHGAGPERFVGVALPRSPDLVVALLAVVKSGAAYVPLDPEYPADRLAYMAGDAAPVAVLTRGDVELPGSVPRIGLDDTEIRATLATAPGTNPGTPVTEAHPAYMIYTSGSTGRPKGVVVSHGAIVNRLAWMQAEYRLDATDRVLQKTPAGFDVSVWEFFWPLLEGAVLVFARPGGHRDAAYLAGLIERERITTAHFVPSMLRVFLEEPGAALCTGLRRVICSGEALGTDLAVDFRAKLPVPLHNLYGPTEAAVDVTHHAYEPATGTATVPIGRPIWNIRTYVLDAALRPVPPGVPGELYLAGAGLARGYHGRPALTAERFVACPFGVPGERMYRTGDLVRWRVDGTLEFVGRADDQVKVRGFRVELGEVEGAVAAHPDVVRAVVVVREDRPGDHRLVAYVTVGGVGGDGLRSAISGLVAERLPAYMVPSAVVVLDEIPLTPNGKVDRAGLPVPVVSVAGFCAPSSPREEVLCGLFAEVLGVERVGVDDGFFDLGGDSILSIQLVARARRAGLELSVRDVFEGRTVRALAAVVRGSDAGAVGVVGGAEIVLPGVGEVERWPVVEWLAERGGGSLGGVVRGFNQSVVLAVPAGLVWEELRVLLGAVRDRHEAWRLRVLDSGALCVDGVVPDDGSWIVRCDLSGMGVDGQVDAVRAAAVEARAWLDPSVGRVVRAVWLERGGDRSGVLVLVAHHLVVDGVSWRVVLGDLAEGWAQVRSGGRVELGVVGTSLRGWAAALAEQGRRGERAGEVELWSRMVRGADVLVGSRAVDGAVDVFGGVVSVDSRASVSVSRALLTEVPSVLGVGVQEVLLAAFGLAVARWRGRGGPVVVDVEGHGRNEDAVRGADLSRTVGWFTSVYPVRVPVESASWDEVRAGGPVVGRVVREVKETLRSLPDQGLGYGILRYLDPEHGPALARHATPQFGFNYLGRFTTGTDETTTADALDRAPAWSLLARSAAGQDPELPVAHAVEFNAITLDTPEGPRLGVTWSWPTTLLPESRIRELARYWDEALEGLVEHARHPEAGGLTPSDVGLAELSFAEIELLEDDWRTQG

SEQ ID NO: 34 - protein, 5830 aa, synthetic construct
  REGION 1..5830: note = dptA_daptomycin non-ribosomal peptide synthetase A
MDMQSQRLGVTAAQQSVWLAGQLADDHRLYHCAAYLSLTGSIDPRTLGTAVRRTLDETEALRTRFVPQDGELLQILEPGAGQLLLEADFSGDPDPERAAHDWMHAALAAPVRLDRAGTATHALLTLGPSRHLLYFGYHHIALDGYGALLHLRRLAHVYTALSNGDDPGPCPFGPLAGVLTEEAAYRDSDNHRRDGEFWTRSLAGADEAPGLSEREAGALAVPLRRTVELSGERTEKLAASAAATGARWSSLLVAATAAFVRRHAAADDTVIGLPVTARLTGPALRTPCMLANDVPLRLDARLDAPFAALLADTTRAVGTLARHQRFRGEELHRNLGGVGRTAGLARVTVNVLAYVDNIRFGDCRAVVHELSSGPVRDFHINSYGTPGTPDGVQLVFSGNPALYTATDLADHQERFLRFLDAVTADPDLPTGRHRLLSPGTRARLLDDSRGTERPVPRATLPELFAEQARRTPDAPAVQHDGTVLTYRDLHRSVERAAGRLAGLGLRTEDVVALALPKSAESVAILLGIQRAGAAYVPLDPTHPAERLARVLDDTRPRYLVTTGHIDGLSHPTPQLAAADLLREGGPEPAPGRPAPGNAAYIIQTSGSTGRPKGVVVTHEGLATLAADQIRRYRTGPDARVLQFISPGFDVFVSELSMTLLSGGCLVIPPDGLTGRHLADFLAAEAVTTTSLTPGALATMPATDLPHLRTLIVGGEVCPPEIFDQWGRGRDIVNAYGPTETTVEATAWHRDGATHGPVPLGRPTLNRRGYVLDPALEPVPDGTTGELYLAGEGLARGYVAAPGPTAERFVADPFGPPGSRMYRTGDLVRRRSGGMLEFVGRADGQVKLRGFRIELGEVQAALTALPGVRQAGVLIREDRPGDPRLVGYIVPAPGAEPDAGELRAALARTLPPHMVPWALVPLPALPLTSNGKLDRAALPVPAARAGGSGQRPVTPQEKTLCALFADVLGVTEVATDDVFFELGGHSLNGTRLLARIRTEFGTDLTLRDLFAFPTVAGLLPLLDDNGRQHTTPPLPPRPERLPLSHAQQRLWFLDQVEGPSPAYNIPTAVRLEGPLDIPALAVALQDVTNRHEPLRTLLAEDSEGPHQVILPPEAARPELTHSTVAPGDLAAALAEAARRPFDLAGEIPLKAHLFGCGPDDHTLLLLVHHTAGDGASVEVLVRDLAHAYGARRAGDAPHFEPLPLQYADHTLRRRHLLDDPSDSTQLDHWRDALAGLPEQLELPTDHTRPAVPTRRGEAIAFTVPEHTHHTLRAMAQAHGVTVFMVMQAALAALLSRHGAGHDIPLGTPVAGRSDDGTEDLVGFFVNTLVLRNDVSGDPTFAELVSRVRAANLDAYAYQDVPFERLVDVLKPERSLSWHPLFQIMIAYNGPATNDTADGSRFAGLTSRVHAVHTGMSKFDLSFFLTEHADGLGIDGALEFSTDLFTRITAERLVQRYLTVLEQAAGAPDRPISSYELLGDDERALLAQWNDTAHPTPPGTVLDLLESRAARTPDRPAVVENDHVLTYADLHTRANRLARHLITAHGVGPERLVAVALPRSAELLVALLAVLKTGAAYVPLDLTHPAERTAVVLDDCRPAVILTDAGAARELPRRDIPQLRLDEPEVHAAIAEQPGGPVTDRDRTCVTPVSGEHVAYVIYTSGSTGRPKGVAVEHRSLADFVRYSVTAYPGAFDVTLLHSPVTFDLTVTSLFPPLVVGGAIHVADLTEACPPSLAAAGGPTFVKATPSHLPLLTHEATWAASAKVLLVGGEQLLGRELDKWRAGSPEAVVFNDYGPTEATVNCVDFRIDPGQPIGAGPVAIGRPLRNTRVFVLDGGLRAVPVGVVGELHVAGEGLARGYLGQPGLTAERFVACPFGDAGERMYRTGDLVRWRADGMLEFVGRVDDQVKVRGFRIELGEVEAAVAACPGVDRSVVVVREDRPGDRRLVAYVTAAGDEAEGLAPLIVETAAGRLPGYMVPSAVVVLDEIPLTPNGKVDRAALPAPRVAPAAEFRVTGSPREEALCALFAEVLGVERVGVDDGFFDLGGDSILSIQLVARARRAGLEVSVRDVFEHRTVRALAGVVRESGGVAAAVVDSGVGAVERWPVVEWLAERGGGGLGGAVRAFNQSVVVATPAGITWDELRTVLDAVRERHDAWRLRVVDSGDGAWSLRVDAPAPGGEPDWITRHGMASADLEEQVNAVRAAAVEARSRLDPLTGRMVRAVWLDRGPDRRGVLVLVAHHLVVDGVSWRIVLGDLGEAWTQARAGGHVRLDTVGTSLRGWAAALAEQGRHGARATEANLWAQMVHGSDPLVGPRAVDPSVDVFGVVESVGSRASVGVSRALLTEVPSVLGVGVQEVLLAAFGLAVTRWRGRGGSVVVDVEGHGRNEDAVPGADLSRTVGWFTSIYPVRLPLEPAAWDEIRAGGPAVGRTVREIKECLRTLPDQGLGYGILRYLDPENGPALAQHPTPHFGFNYLGRVSVSADAASLDEGDAHADGLGGLVGGRAAADSDEEQWADWVPVSGPFAVGAGQDPVLPVAHAVEFNAITLDTPDGPRLSVTWSWPTTLLSESRIRELARFWDEALEGLVAHARRPDAGGLTPSDLPLVALDHAELEALQADVTGGVHDILPVSPLQEGLLFHSSFAADGVDVYVGQLTFDLTGPVDADHLHAVVESLVTRHDVLRTGYRQAQSGEWIAVVARQVHTPWQYIHTLDTDADTLTNDERWRPFDMTQGPLARFTLARINDTHFRFIVTYHHVILDGWSVAVLIRELFTTYRDTALGRRPEVPYSPPRRDFMAWLAERDQTAAGQAWRSALAGLAEPTVLALGTEGSGVIPEVLEEEISEELTSELVAWARGRGVTVASVVQAAWALVLGRLVGRDDVVFGLTVSGRPAEVAGVEDMVGLFVNTIPLRARMDPAESLGAFVERLQREQTELLEHQHVRLAEVQRWAGHKELFDVGMVFENYPMDSLLQDSLFHGSGLQIDGIQGADATHFALNLAVVPLPAMRFRLGYRPDVFDAGRVRELWGWIVRALECVVCERDVPVSGVDVLGAGERETLLGWGAGAEPGVRALPGAGAGAGAGLVGLFEERVRTDPDAVAVRGAGVEWSYAELNARANAVARWLIGRGVGPERGVGVVMDRGPDVVAMLLAVAKSGGFYLPVDPQWPTERIDWVLADAGIDLAVVGENLAAAVEAVRDCEVVDYAQIARETRLNEQAATDAGDVTDGERVSALLSGHPLYVIYTSGSTGLPKGVVVTHASVGAYLRRGRNAYRGAADGLGHVHSSLAFDLTVTVLFTPLVSGGCVTLGDLDDTANGLGATFLKATPSHLPLLGQLDRVLAPDATLLLGGEALTAGALHHWRTHHPHTTVINAYGPTELTVNCAEYRIPPGHCLPDGPVPIGRPFTGHHLFVLDPALRLTPPDTIGELYVAGDGLARGYLGRPDLTAERFVACPFRSPGERMYRTGDLARWRSDGTLEFIGRADDQVKIRGFRIELGEVEAAVAAHPHVARAIAVVREDRPGDQRLVAYVTGSDPSGLSSAVTDTVAGRLPAYMVPSAVVVLDQIPLTPNGKVDRAALPAPGTASGTTSRAPGTAREEILCTLFADVLGLDQVGVDEDFFDLGGHSLLATRLTSRIRSALGIDLGVRALFKAPTVGRLDQLLQQQTTSLRAPLVARERTGCEPLSFAQQRLWFLHQLEGPNAAYNIPMALRLTGRLDLTALEAALTDVIARHESLRTVIAQDDSGGVWQNILPTDDTRTHLTLDTMPVDAHTLQNRVDEAARHPFDLTTEIPLRATVFRVTDDEHVLLLVLHHIAGDGWSMAPLAHDLSAAYTVRLEHHAPQLPALAVQYADYAAWQRDVLGTENNTSSQLSTQLDYWYSKLEGLPAELTLPTSRVRPAVASHACDRVEFTVPHDVHQGLTALARTQGATVFMVVQAALAALLSRLGAGTDIPIGTPIAGRTDQAMENLIGLFVNTLVLRTDVSGDPTFAELLARVRTTALDAYAHQDIPFERLVEAINPERSLTRHPLFQVMLAFNNTDRRSALDALDAMPGLHARPADVLAVTSPYDLAFSFVETPGSTEMPGILDYATDLFDRSTAEAMTERLVRLLAEIARRPELSVGDIGILSADEVKALSPEAPPAAEELHTSTLPELFEEQVAARGHAVAVVCEGEELSYKELNARANRLARVLMERGAGPERFVGVALPRGLDLIVALLAVTKTGAAYVPLDPEYPTDRLAYMVTDANPTAVVTSTDVHIPLIAPRIELDDEAIRTELAAAPDTAPCVGSGPAHPAYVIYTSGSTGRPKGVVISHANVVRLFTACSDSFDFGPDHVWTLFHSYAFDFSVWEIWGALLHGGRLVVVPFEVTRSPAEFLALLAEQQVTLLSQTPSAFHQLTEAARQEPARCAGLALRHVVFGGEALDPSRLRDWFDLPLGSRPTLVNMYGITETTVHVTVLPLEDRATSLSGSPIGRPLADLQVYVLDERLRPVPPGTVGEMYVAGAGLARGYLGRPALTAERFVADPNSRSGGRLYRTGDLAKVRPDGGLEYVGRGDRQVKIRGFRIELGEIEAALVTHAGVVQAVVLVRDEQTDDQRLVAHVVPALPHRAPTLAELHEHLAATLPAYMVPSAYRTLDELPLTANGKLDRAALAGQWQGGTRTRRLPRTPQEEILCELFADVLRLPAAGADDDFFALGGHSLLATRLLSAVRGTLGVELGIRDLFAAPTPAGLATVLAASGTALPPVTRIDRRPERLPLSFAQRRLWFLSKLEGPSATYNIPVAVRLTGALDVPALRAALGDVTARHESLRTVFPDDGGEPRQLVLPHAEPPFLTHEVTVGEVAEQAASATGYAFDITSDTPLRATLLRVSPEEHVLVVVIHHIAGDGWSMGPLVRDLVTAYRARTRGDAPEYTPLPVQYADYALWQHAVAGDEDAPDGRTARRLGYWREMLAGLPEEHTLPADRPRPVRSSHRGGRVRFELPAGVHRSLLAVARDRRATLFMVVQAALAGLLSRLGAGDDIPIGTPVAGRGDEALDDVVGFFVNTLVLRTNLAGDPSFADLVDRVRTADLDAFAHQDVPFERLVEALAPRRSLARHPLFQIWYTLTNADQDITGQALNALPGLTGDEYPLGASAAKFDLSFTFTEHRTPDGDAAGLSVLLDYSSDLYDHGTAAALGHRLTGFFAALAADPTAPLGTVPLLTDDERDRILGDWGSGTHTPLPPRSVAEQIVRRAALDPDAVAVITAEEELSYRELERLSGETARLLADRGIGRESLVAVALPRTAGLVTTLLGVLRTGAAYLPLDTGYPAERLAHVLSDARPDLVLTHAGLAGRLPAGLAPTVLVDEPQPPAAAAPAVPTSPSGDHLAYVIHTSGSTGRPKGVAIAESSLRAFLADAVRRHDLTPHDRLLAVTTVGFDIAGLELFAPLLAGAAIVLADEDAVRDPASITSLCARHHVTVVQATPSWWRAMLDGAPADAAARLEHVRILVGGEPLPADLARVLTATGAAVTNVYGPTEATIWATAAPLTAGDDRTPGIGTPLDNWRVHILDAALGPVPPGVPGEIHIAGSGLARGYLRRPDLTAERFVANPFAPGERMYRTGDLGRFRPDGTLEHLGRVDDQVKVRGFRIELGDVEAALARHPDVGRAAAAVRPDHRGQGRLVAYVVPRPGTRGPDAGELRETVRELLPDYMVPSAQVTLTTLPHTPNGKLDRAALPAPVFGTPAGRAPATREEKILAGLFADILGLPDVGADSGFFDLGGDSVLSIQLVSRARREGLHITVRDVFEHGTVGALAAAALPAPADDADDTVPGTDVLPSISDDEFEEFELELGLEGEEEQW

SEQ ID NO: 35 - protein, 89 aa, synthetic construct
  REGION 1..89: note = dptF_Acyl carrier protein
MNPPEAVSTPSEVTAWITGQIAEFVNETPDRIAGDAPLTDHGLDSVSGVALCAQVEDRYGIEVDPELLWSVPTLNEFVQALMPQLADRT

SEQ ID NO: 36 - protein, 597 aa, synthetic construct
  REGION 1..597: note = dptE_Fatty acid ligase
MSESRCAGQGLVGALRTWARTRARETAVVLVRDTGTTDDTASVDYGQLDEWARSIAVTLRQQLAPGGRALLLLPSGPEFTAAYLGCLYAGLAAVPAPLPGGRHFERRRVAAIAADSGAGVVLTVAGETASVHDWLTETTAPATRVVAVDDRAALGDPAQWDDPGVAPDDVALIQYTSGSTGNPKGVVVTHANLLANARNLAEACELTAATPMGGWLPMYHDMGLLGTLTPALYLGTTCVLMSSTAFIKRPHLWLRTIDRFGLVWSSAPDFAYDMCLKRVTDEQIAGLDLSRWRWAGNGAEPIRAATVRAFGERFARYGLRPEALTAGYGLAEATLFVSRSQGLHTARVATAALERHEFRLAVPGEAAREIVSCGPVGHFRARIVEPGGHRVLPPGQVGELVLQGAAVCAGYWQAKEETEQTFGLTLDGEDGHWLRTGDLAALHEGNLHITGRCKEALVIRGRNLYPQDIEHELRLQHPELESVGAAFTVPAAPGTPGLMVVHEVRTPVPADDHPALVSALRGTINREFGLDAQGIALVSRGTVLRTTSGKVRRGAMRDLCLRGELNIVHADKGWHAIAGTAGEDIAPTDHAPHPHPA

SEQ ID NO: 37 - protein, 289 aa, synthetic construct
  REGION 1..289: note = dptN_ABC-type transporter gene
MLTTRRTGPGTSPVADGPGWRGGGAGIGTQFRVLTGRQFRIIYGDRRIALFSLLQPIIMLMLFSQVLGRMANPEIFPPGVRYLDYLVPALLLTTGIGSAQGGGLGLVRDMESGMMVRLRVMPVRLPLVLVARSLADLARVALQLVALLACAMGPLGYRPAGGVSGIVGATLLALLVAWSLIWVFLALAAWLRSIEVLSSIGFLVTFPLMFASSAFVPLDILPGWLRVIATVNPLTYAVEASRDLALDHSALGAALAAVGTSLALLAVTGLLAVRGLRRPPGAGGPHRTP

SEQ ID NO: 38 - protein, 319 aa, synthetic construct
  REGION 1..319: note = dptM_ABC-type transporter gene
MGGSAIRTRQLTKHFGAVQALVGVDLEVPAGSVLGLLGHNGAGKTTLIQILSTVLPPSGGSAEVAGFDIVRDARRVRACIGVTGQFAALDEHLSGLANLVLISRLLGARPREARRRAAELVEQFGLTEAADRPMRTYSGGMRRRIDLAASLVARPSVLFLDEPTTGLDPVSRTALWETVEGLVAEGTTVLLTTQYLDEADRLADRIAVLSSGHVVTVGTAAELKAAGTRSVRLTFGSAADLESAEGALRLEGLGLTTDPVSRTVSLPLAATAELAGIFRILGAAGVELAELALKEPTLDDVYLSLAESWETTSGGTVRC

SEQ ID NO: 39 - protein, 206 aa, synthetic construct
  REGION 1..206: note = dptP_Transporter gene
MSWMNDVLTAVSDMNPVTRFALASVFAFAESGLGAGMAVPGEVAVLALSAGTEGTRPLLALFLVVTLSSSAGDHIGYFLGIRYGQRMRETRLVRRIGQHHWDRAQELCHRYGARAVFLTRLLPVVRTLTPATAGVGSVRYLRFLPASLAGAAMWSALYVSAGTLVSTSLREAESVLSTILWALLGVAAAFTLAIVWWRRRHRRRSS

SEQ ID NO: 40 - DNA, 91447 bp, synthetic construct
  misc_feature 1..91447: note = BAC-dpt*
  source 1..91447: mol_type = other DNA
gtctgataatcagactgggaccacggtcccactcgtatcgtcggtctgattattagtctgggaccacggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaatcgccctatagtgagtcgtattacaattcacactagtggtaccaacccaggcaggtagcacagtgcgcgcccactgccttacggatagcacgacgcatcttcgccgcagcgttgttcccacgggcgatagcagcacgccgcttcgcatggctcttcacactgcgctgtgcctgataggtggcgtggtgctgagcacagcgcccaccatgagtagcccagtccctacagtcgagacaccgggtacgcatagggcaccccccccccccttccggggacacccccctatccgtcgacacccccctacctactcacgtgagtaggctgggcgcggcagcgctagaagcgaccgcccgtcatgaagtgggcagtgagccaacccatgaaggccagtagcgcaaagcgtcgagcacgcacccaacccgaaggcttgcctgtactgccgctttgcgtggcgaaccatgaccagacgtgttcgctcagcgtgtcgcccggcttcttattgaagagcgctcgaccctcaatgacgccgaacgcggcaagccatgcgagccaagcgattgtgtatccgctcattgagccccccgacggattcgaaccgccgaccccctgattacgaaacaggcgctctaaccgaactgagctagggaggcaacgccggtcgctcaaccctgggagagatcaggggagtgaccggcctgagtgggtagcgctcgacgccgttccttagcggtgcttgatgcgtggcgcgtaaacccctctacctatatgaaggttgtcggcaacctgattcggggtcacagcgaagggcgcccgggtgacgctgtgatgaagtggcgtgaagtctggattacccaatgagaagtcattaggaatgtgtgtttcgagtcattccgtcacagcatcacttcccccaggtcaacggctcgccccgctgtgctgtggtgacggagtcactgttacttaggctaactaactaaaccgagacaagagaaatgggccgaccgggggaccggccgacccttcccgctgccccaggaagcccgctgtgcgcccgtctcagcgcctaacaggcttcccgggtgtctcgctacgccgctacgtcttccgtgccgtcctgggcgtcgtcttcgtcgtcgtcggtcggcggcttcgcccacgtgatcgaagcgcgcttctcgatgggcgttccctgccccctgcccgtagtcgacttcgtgacaacgatcttgtctacgaagagcccgacgaacacgcgcttgtcgtctactgacgcgcgcccccaccacgacttagggccggtcgggtcagcgtcggcgtcttcggggaaccattggtcaaggggaagcttcggggcttcggcggcttcaagttcggcaagccgctcttccgccccttgctgccggagcgtcagcgctgcctgttgcttccggaagtgcttcctgccaacgggtccgtcgtacgcgcctgccgcgcggtcttcgtacagctcttcaagggcgttcagggcgtcggcgcgctccgcaacaaggttcgcccgttcgccgctcttctcaggcgcctcagtgagcttgccgaagcgtcgggcggcttcccacagaagcgccaacgtctcttcgtcgccttcggcgtgcctgatcttgttgaagatgcgttccgcaacgaacttgtcgagtgccgccatgctgacgttgcacgtgccttcgtgctgcccaggtgcggacgggtcgaccaccttccggcgacggcagcggtaagagtccttgatcgattcttccccgcgcttcgaagtcatgacggcgccacactcgcagtacagcttgtccatggcggacagaatggcttgcccccgggaaagccccttgccgcgccccctgccgtccaaccacgcctgaagctcataccactcagcgggctcgatgatcggtccgcaatcaagctcgaccggccggagcgtgatcgggtcgcgctgaatgcggtaaccctcaatcttcgtggtcggcgtgccgtccggcttcttcttgtagatcacctcagcggcgaagcccgcaatacgcgggtcccgaaggattcgcataacggttgccgggtcccaggcgcttgaagcggtcttcttcccaatcgtctcgccccgggtcggcacggcgtcagcgtccatgcgcttacaaagccccgtgatgctgcccgggtgaatggcggcttgactgcccggcttgaagggaaggtgtttgtgcgtcttgatctcacgccaccaccaccggattacgtcgggctcgaactcgaagggtccggtaaggggagtggtcgagtgcgcaagcttgttgatgacgacattgaccattcggccgttgcgcgtgatctccttcgtctccgaaacaagctcgaagccgtaaggcgccttcccgccgacgtacccgcccaattcgcgctgaaggttcttcgtgtcgagaatcttcgccgacttcagcgaagattctttgtgcgacgcgtcgagccgcataatcaggtgaatcaggtccatgacgtttccctgccggaagacgccttcctgagtggaaacaatcgtcacgcccagggcgagcaattccgagacaatcggaatcgcgtccatgaccttcaggcgcgagaagcgcgacacgtcatagacaatgatcatgttgagccgcccggcgcggcattcgttcaggatgcgttcgaactccgggcgctccgccgtcccgaacgccgacgtgcccggcgcttcgctgaaatgcccgacgaacctgaaccggcccccgtcgcgctcgacttcgcgctgaaggtcggccgccttgtcttcgttggcgctacgctgtgtcgctgggcttgctgcgctcgaattctcgcgctcgcgcgactgacggtcgtaagcacccgcgtacgtgtccaccccggtcacaaccccttgtgtcatgtcggcgaccctacgcccccaactgagagaactcaaaggttaccccagttggggcactactcccgaaaaccgcttctgacctgggaaaacgtgaagccccggggcatccgctgagggttgccgccggggcttcggtgtgtccgtcagtacgggccatagaggggcgtcgtgtaacccacccagggcgattgctccggcacggggaagccgcgccacgccttcgggacgtcagcggctcgccccgtgcgctcccacatctgccacgtctgaaggcagtgggcgagccagtcatacgccgccgaccgcgaacgccattcgagcgtcaccggctcgccgtcgtcgtcctggggcagcgcgcagtaatcgcccatgacttcatcgaagatcacgaacggcggattcttgcgcacgacccaggttccgtttttccggcgctgtagcgcgcgttccatgaccctgtatctcttcaccggccgacccgctcacgcgcgacgttgtacgggacggcgcccccgccgacaaggggcacttcgccccacgtccggtagcaccgcattagccacgcctgggcgccatgctgggtgacccattcgagcggcatgagggttgaggggtcgccgtcgtcgggcagcgcgcaatagcccttcatggctccgtcgaagatcacccaggggcgcacgacgggcgggttgccgtcttcggttcgctgtagttcctgccgccactcaggcggacgctgtgtgactcgataccgacgcatagggaacacgctaggggacttccctcagttgggggagagcgtgacgaaaattccaccttcgcgggcttcccaggtgcgcccccgctcgacctcaaaccggccatcggtcacaagcttcatgcgcaacgggccgaccacgttgaagagaatgtgcgcgagcgtcgtcgtgtcttcgtcggcttcggcttcctcgcgcgcccaggtgtcggcgggtgagtggcccgtgtagcacgtcggcgagtagggcacgcccagggcttccgctagttccttgtcgcacacaacgcccgggtcggcggcagtctccggggtgtcgccccaggcttcttcatacgggtactcaggcgagccgccttcagcgatcaagggcgtctcagcgtcggcttcgtggttgtgccccgggaagggcagcatgacgtctggaatcgatgataagctgtcaaacatgagaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgcccccggacgagcgtctgctccgccattcgccgtccgccgtgccaatcggatcagccgtccaaatgcgggattttcgttagtcggaggccaaacggcattgagcgtcagcatatcatcagcgagctgaagaaagacaatccccgatccgctccacgtgttgccccagcaatcagcgcgaccttgcccctccaacgtcatctcgttctccgctcatgagctcagccaatcgactggcgagcggcatcgcattcttcgcatcccgcctctggcggatgcaggaagatcaacggatctcggcccagttgacccagggctgtcgccacaatgtcgcgggagcggatcaaccgagcaaaggcatgaccgactggaccttccttctgaaggctcttctccttgagccacctgtccgccaaggcaaagcgctcacagcagtggtcattctcgagataatcgacgcgtaccaacttgccatcctgaagaatggtgcagtgtctcggcaccccatagggaacctttgccatcaactcggcaagatgcagcgtcgtgttggcatcgtgtcccacgccgaggagaagtacctgcccatcgagttcatggacacgggcgaccgggcttgcaggcgagtgaggtggcaggggcaatggatcagagatgatctgctctgcctgtggccccgctgccgcaaaggcaaatggatgggcgctgcgctttacatttggcaggcgccagaatgtgtcagagacaactccaaggtccggtgtaacgggcgacgtggcaggatcgaacggctcgtcgtccagacctgaccacgagggcatgacgagcgtccctcccggacccagcgcagcacgcagggcctcgatcagtccaagtggcccatcttcgaggggccggacgctacggaaggagctgtggaccagcagcacaccgccgggggtaaccccaaggttgagaagctgaccgatgagctcggcttttcgccattcgtattgcacgacattgcactccaccgctgatgacatcagtcgatcatagcacgatcaacggcactgttgcaaatagtcggtggtgataaacttatcatccccttttgctgatggagctgcacatgaacccattcaaaggccggcattttcagcgtgacatcattctgtgggccgtacgctggtactgcaaatacggcatcagttaccgtgagctgcaggtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatccccggggacctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccacacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattccatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttattttttttctcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggaattcccgttttaagagcttggtgagcgctaggagtcactgccaggtatcctgtgcggtatttcacaccgcatataattccacttgcaattacataaaaaattccggcggtttttcgcgtgtgactcaatgtcgaaatacctgcctaatgaacatgaacatcgcccaaatgtatttgaagacccgctgggagaagttcaagatatataagtaacaagcagccaatagtataaaaaaaaatctgagtttattacctttcctggaatttcagtgaaaaactgctaattatagagagatatcacagagttactcactaatgactaacgaaaaggtctggatagagaagttggataatccaactctttcagtgttaccacatgactttttacgcccacaacaagaaccttatacgaaacaagctacatattcgttacagctacctcagctcgatgtgcctcatgatagtttttctaacaaatacgctgtcgctttgagtgtatgggctgcattgatatatagagtaaccggtgacgatgatattgttctttatattgcgaataacaaaatcttaagattcaatattcaaccaacgtggtcatttaatgagctgtattctacaattaacaatgagttgaacaagctcaattctattgaggccaatttttcctttgacgagctagctgaaaaaattcaaagttgccaagatctggaaaggacccctcagttgttccgtttggcctttttggaaaaccaagatttcaaattagacgagttcaagcatcatttagtggactttgctttgaatttggataccagtaataatgcgcatgttttgaacttaatttataacagcttactgtattcgaatgaaagagtaaccattgttgcggaccaatttactcaatatttgactgctgcgctaagcgatccatccaattgcataactaaaatctctctgatcaccgcatcatccaaggatagtttacctgatccaactaagaacttgggctggtgcgatttcgtggggtgtattcacgacattttccaggacaatgctgaagccttcccagagagaacctgtgttgtggagactccaacactaaattccgacaagtcccgttctttcacttatcgcgacatcaaccgcacttctaacatagttgcccattatttgattaaaacaggtatcaaaagaggtgatgtagtgatgatctattcttctaggggtgtggatttgatggtatgtgtgatgggtgtcttgaaagccggcgcaaccttttcagttatcgaccctgcatatcccccagccagacaaaccatttacttaggtgttgctaaaccacgtgggttgattgttattagagctgctggacaattggatcaactagtagaagattacatcaatgatgaattggagattgtttcaagaatcaattccatcgctattcaagaaaatggtaccattgaaggtggcaaattggacaatggcgaggatgttttggctccatatgatcactacaaagacaccagaacaggtgttgtagttggaccagattccaacccaaccctatctttcacatctggttccgaaggtattcctaagggtgttcttggtagacatttttccttggcttattatttcaattggatgtccaaaaggttcaacttaacagaaaatgataaattcacaatgctgagcggtattgcacatgatccaattcaaagagatatgtttacaccattatttttaggtgcccaattgtatgtccctactcaagatgatattggtacaccgggccgtttagcggaatggatgagtaagtatggttgcacagttacccatttaacacctgccatgggtcaattacttactgcccaagctactacaccattccctaagttacatcatgcgttctttgtgggtgacattttaacaaaacgtgattgtctgaggttacaaaccttggcagaaaattgccgtattgttaatatgtacggtaccactgaaacacagcgtgcagtttcttatttcgaagttaaatcaaaaaatgacgatccaaactttttgaaaaaattgaaagatgtcatgcctgctggtaaaggtatgttgaacgttcagctactagttgttaacaggaacgatcgtactcaaatatgtggtattggcgaaataggtgagatttatgttcgtgcaggtggtttggccgaaggttatagaggattaccagaattgaataaagaaaaatttgtgaacaactggtttgttgaaaaagatcactggaattatttggataaggataatggtgaaccttggagacaattctggttaggtccaagagatagattgtacagaacgggtgatttaggtcgttatctaccaaacggtgactgtgaatgttgcggtagggctgatgatcaagttaaaattcgtgggttcagaatcgaattaggagaaatagatacgcacatttcccaacatccattggtaagagaaaacattactttagttcgcaaaaatgccgacaatgagccaacattgatcacatttatggtcccaagatttgacaagccagatgacttgtctaagttccaaagtgatgttccaaaggaggttgaaactgaccctatagttaagggcttaatcggttaccatcttttatccaaggacatcaggactttcttaaagaaaagattggctagctatgctatgccttccttgattgtggttatggataaactaccattgaatccaaatggtaaagttgataagcctaaacttcaattcccaactcccaagcaattaaatttggtagctgaaaatacagtttctgaaactgacgactctcagtttaccaatgttgagcgcgaggttagagacttatggttaagtatattacctaccaagccagcatctgtatcaccagatgattcgtttttcgatttaggtggtcattctatcttggctaccaaaatgatttttaccttaaagaaaaagctgcaagttgatttaccattgggcacaattttcaagtatccaacgataaaggcctttgccgcggaaattgacagaattaaatcatcgggtggatcatctcaaggtgaggtcgtcgaaaatgtcactgcaaattatgcggaagacgccaagaaattggttgagacgctaccaagttcgtacccctctcgagaatattttgttgaacctaatagtgccgaaggaaaaacaacaattaatgtgtttgttaccggtgtcacaggatttctgggctcctacatccttgcagatttgttaggacgttctccaaagaactacagtttcaaagtgtttgcccacgtcagggccaaggatgaagaagctgcatttgcaagattacaaaaggcaggtatcacctatggtacttggaacgaaaaatttgcctcaaatattaaagttgtattaggcgatttatctaaaagccaatttggtctttcagatgagaagtggatggatttggcaaacacagttgatataattatccataatggtgcgttagttcactgggtttatccatatgccaaattgagggatccaaatgttatttcaactatcaatgttatgagcttagccgccgtcggcaagccaaagttctttgactttgtttcctccacttctactcttgacactgaatactactttaatttgtcagataaacttgttagcgaagggaagccaggcattttagaatcagacgatttaatgaactctgcaagcgggctcactggtggatatggtcagtccaaatgggctgctgagtacatcattagacgtgcaggtgaaaggggcctacgtgggtgtattgtcagaccaggttacgtaacaggtgcctctgccaatggttcttcaaacacagatgatttcttattgagatttttgaaaggttcagtccaattaggtaagattccagatatcgaaaattccgtgaatatggttccagtagatcatgttgctcgtgttgttgttgctacgtctttgaatcctcccaaagaaaatgaattggccgttgctcaagtaacgggtcacccaagaatattattcaaagactacttgtatactttacacgattatggttacgatgtcgaaatcgaaagctattctaaatggaagaaatcattggaggcgtctgttattgacaggaatgaagaaaatgcgttgtatcctttgctacacatggtcttagacaacttacctgaaagtaccaaagctccggaactagacgataggaacgccgtggcatctttaaagaaagacaccgcatggacaggtgttgattggtctaatggaataggtgttactccagaagaggttggtatatatattgcatttttaaacaaggttggatttttacctccaccaactcataatgacaaacttccactgccaagtatagaactaactcaagcgcaaataagtctagttgcttcaggtgctggtgctcgtggaagctccgcagcagcttaaggttgagcattacgtatgatatgtccatgtacaataattaaatatgaattaggagaaagacttagcttcttttcgggtgatgtcacttaaaaactccgagaataatatataataagagaataaaatattagttattgaataagaactgtaaatcagctggcgttagtctgctaatggcagcttcatcttggtttattgtagcatgaatcatatttgcctttttttcctgtaattcaatgattcttgcttctatactatcctcaatgcaaaaccttgtgatcttcacaggtcgatactgaccaattctatgaactctatcaccactttgccattcaacactagggttccaccatgggtctaaaatgaatacttgcgaagctatatggtgcactctcagtacaatctgctctgatgccgcatagttaagccagccccgacacccgccaacacccgctgacgcgatgcgtaaggagaaaatatgggagggtaatcagccgtgtccaccaacacaacgctgtcgggcggttctataggattccgcattgcgcctacctacagggtgcctcaacggtatccgcgacttgcgatgtgcctgctatgaacatgacggggatgtggatcgcggcgtagcgggcgacgttggccacgctgtcgcgggcggccgggtcggcgaggacgtcggtgtagttgtcgaagccggtgaggacctggcggtacccgtcgaagccgagcccggagaggcggtcggcgaagaggctgaggtagaaggcgtacccgatcgggatcagcacggtacagagcagcagcagtgcgaacggtgccgccagtgcggccccggtgaagcggcgccggcgggctgcccggcgtgaccgcgcggaggccgggccagggggcgtcgtcgccggtgtggggacggctgtcatgtccggctcccttcggtgacggccaggccgcgggcccgcatcgcggcgacggtcgaggtgtgaacccggtgcagcgcccgggggatggtggtggctccggtggagaccgggccgagcgcgtcggcgatcgtggcgaaggtgctcagcgaggtcgggccccacgtccacccggggacccgggtcgcggactcgtccagcacgtcgaagacggggtcgccggtgaagtacccgtcctcgtaggcggtacgggccaccttccggttggcgaggaaagcgggcgaggggaaggtgaacgccgagccgatccggggaaccgcggggtcggtgctgagccagcgcaggaacgtcagggcggcctcgggcacctcgctctcggcggagaccccgaacgccgaccctccccagaccccggtggacgggtcgtcgccccaggtgggcaggggtgcgaccgcccagcggccctggtgggcgggcagggacttcttcagcatgcccacgccccaggtggccccgagcatcccccagagccggccgtggtgcatcgcggagatccagctctgggtgccggtggcgtcgcgcgcggcaagaccggaggacaccatgtcctgccagtactcggccacgcgcatcgtgcccgcgccgtcgatcccgacccgccacgcgttcccggcgatgtcccaccaggggtcgccggcctggcaggccatcccggcgaagaaggcgccgtcgttgagcgggaaggtggtgatccgcgcatcccggtcggccgtgcggacggccgaggcggcccggcggaactcctcccaggtacggggcactccgatgccggcccggtcgaacaggtccttgcggtagaagaacgccatcgggcagaagtccatgggcaccgcccaggtcttcccgccggggcggacgctctgccacgcggcgggcaggaagccgtcggcgagatcgtccacgtccgcgctcagatcgcgcagcccgccggtggtcgcgatctgcggcagggcctggtactcgacgtgcaggatgtccggggcgttgtgggcgcggatcgcgttggtctgctgggcgtagcccccggacgtaccggcggcgatcacgctcaggtcgacccggacgtcgcgctgggaggagttgaacgcggcgacgtaccggtccatgccggtcagccacgaccacatacggacccggacggttccggaggtccggcccgaacagccggtgagtgtgccgccggcgagaagcgctgcccccaggcccgcggaggtccgcagcagggacctccgcgatcggtgcgtccgaggggcgtgtgcgctgtcaccggcgtcggaggagaagccgagcaccaggggtttcctttcgtgcaggaaggtgttcgcacacttcagcccctggccgattttcggtcaacggtagcgaaatgaatcgatcaggatcgataggaaagcaaagctcaacgagctccggagaggccccgctcccgatccgagagccctcccgctagctcgcattcccggcaaagcgggtagcgccttcctggatcgggggcgcctgcgatagaacagacgacatccacgagaagtcgaagagaagtcgaacgacacagttcgatcagaaggaaaaggagaggccgggatggcactcgcatcggaacggcaggagttcatcctcgccaccgtgcgggaacgcggcaccgtgcgcctggccgatctggtcgagcggctgggcgtcaccgccgtgacggtccgccgggacgtcacgatcctggccgaccgggggctggtgtcgcgcgtccacggaggcgtcaccctcccctatcgcgggccgacggccgacgagcgggccacccggggcgtgttcacccacggccgcctgcccgaccaggcgttcgtcggcatggtcgtccccaccgtcgagtactactggccggcggtcatccagggcgcccagtcggcggtggccgccgccgggggacggctggtcctgcgcgcttccgcctacgacgcggcggaggaccgccgccaggtcaccggcctcctcgaccgcggtgtgcgcacgctgctcgtcgcccccaccaacaccggcgaggccggccaggacctgctgcgctggctgggctcgctgaacgtccccgtcatcctcgtcgaacggctgccgccgcccgctctgcccaccctgccgctcgacgccgtcaccaccgcccacagcctcggcgcgggcctggccgtacggcatctggtcaccctgggccatcaccgtatcgccttcgtcaccgcgcgcttcagccccaccaccaaggccctgcgcgaggggtggcaggagaccaccgcctctctcggcctgccctccggcgacggcctcgcacacgacgtaccgtcgtacggctcccccggctgggcggacgcctacgacgcggccctccgcagctgccgcgaggccggcgccaccgccctgttcgtccactccgacggtgaggccgtcggcctggtggagcgcgcccacgaacacggactcgcggtccccgaggacctcgccgtcatcacctacgacgacgaggtggcagccgccgccgaccctcccctgaccgccgtgcgcccgcagaagcaccggctcggcgtcctggccgccgaaatggccctggcacgcgccgtcgatcccgtcgaacgcccggtgcaccgagtcgagttgtggcccaacctgatcatccgtgcgtcctgcggcggcaccgcgcccgtctcggcgggacggtgaaggcgggctgagggggccggggctggctcacccggtgagccagccccggagcgcggcctgtaccccggcctggaaacgggtctccgcgttgaggacctgcatcagacgggtgatgcggcgccggaccgtgtgcacatggatgtcgaggcgccgggccatcgcctcgtccttcaggccggaggcgagcatcgtcagcaactcccggtcgccgtccgtgatctgatcatgcgtcacggagccgatggggagcgcccgttcccagaccgactcgaacagcggcaccagcgcgttgctcagcagcgcgtcgctcaccaccagtgcggaggccgtcgggtccgccgcgtccgtcggcggcaggagggtcacccgccggtcgaccgtgatcagcttcgtcggcagatccgtgcccacccggatctgcaccccctgcacggccagatcgcccaggccgcgggcgcgcccgtggaagctcagcccctcgcggtccaccacgacccggacgcgtaccccccgacgtaccggttcggccatgtcgagggggacgggcatcccgtccggctcgctcgacgcgtacggcggccggtccaggagggcgatctcctcgctcgccgacaccagcagggaggccacccgggcggcgatcgccgctccgccgcggaccgtctcgatgccggtggcgcggggggtgtgcacggagctcagcagttgggccgcgatccggtcgacggatccgctgagctcctccaactccgccgatcggtgcaggagttccgcctggtgcagatggatcagattgcgcagggcggtggcgggggccatggccacgggcagcccggcgccccgggccgggtgggtgaagcggcgctcggccaggtgatccagggcgttcgtgacccggcgcacggtcagccccaggtgctgggcgagggcggtacgggaggcgggtacgggtgcgtcgaggagcgcttcgtacacccggagttcgtccgctccgagcccgagcgcctgccattgcgtgtcccacccagtccgccccaccggcggaattgtcacctgtcaatgcaggccacacaagtgtgtacgtccacaagtgcacgcgcggaacgcattgttcccacccccgccccggtacttccatgtcggcatccgagatccgttcatgcagcaatcgcattcgggaggggcaactgtgcggagttcatcgcgcagcagatcggccggaccaggtcaggggtccggcaccacgttcgtcggccagggcgtggtggcggtactggccgccgcgggcctgttgatgacggccacgcccgccgctcacgcggacaccgccaccacgtccgccgcgtccgaggtgacgccgggtgccgggaccgggaccccctcgctcgtcacgggactcaacgaggaggccgacgccaccggcaccgcggcggacgccgcccgtggccacctcaaggcgaagaagagccgctaccgcatcgcgcacacctcggcgaaggacctggccgcggccgggaccacgaccgggaggggcggcagggagacggtccgtctccagcagaagtaccgcggcgtcacggtcctcggcggccagtacctggtccggatgacgaagaaggacggcaagcgcaccgtcaccggcacgtccggcagctacttcaccgagctgaagctcgacacggtgacgccggaggtctccgagaagaccgcgatcgagcgcgcggtcggcgccgtctccgcgcagctgggcgggggcctgctcagggccccggccaagggcgagaagcccggcggggttcagctgtcgggtacggccggtgacgtgaccgtcctgccccagggatccggtgtgctgacccggcacatcaccgtcaccggcgtcagcccggccgacggcacgccggtgaagcaggaggtgtacatcgacgcccactccggcttccccgtgatgcagtacagcggcatcaagtcgttcggcgcaccgggttcgggcgccggtacgagcagcggcgccaccggcgacgcgcccacggccgacgccaccgcggaggccacgcagcagctggtcgtcaagggctcgggcacccgctacaacggcgaggaggccgagatcaacctgtaccagggcgccgacggcgcctaccagatgtacgactacagcaagcggggcgccgacagcccgtacgacggtccgctgctgtcgacctgggacgcgcgtggccgggaggccgccacggcgtccggcacgtggcccagcggcatccagacgttccgctcggacacccccgacctgggcaaggagttcaccgactccggggcggtggacgcccactgggcggccgggaaggtctacgactactacaagaaccacttcggccgtaagagcctggacggcaaggacgggttcatgtactcgctcgtgggtgtggtgaacggcgggaagccgttcaacaacgccttctgggacggcaccaagatggtgtacggccagggcggcggcgactaccgcaccttctcggcggacaccgacgtggtcggccacgagatgacgcacggtgtcatcgagcacaccgcgaacctggtgtacgcgggccagtccggcgccgtgaacgaggcgatagccgactacttcggcaacgccatcgacctggaggccgacggccggtcgatggacgacccggacgccggtctgctcggcgaggacctgtgcaccacgctcggcccgcgcgactgcgcgctgcgggacctcaacgacggtgccaccacgtcgaaggacttcatcggcgtcagccacaacggcgacaacggtggcgtacacctcaactccacgatcttctcgggcgccctgtgggacatgcgcgaggacctcggcgccgagccggccgaccagatcgtctaccgcgcgctgtccgcgtacatgacgccgctcgacggcttcaccgagggccgcgcggcggtcatcgccgcagcgcaggagctgggcgccaccaaggcgcagacgaagatcgtgaagcgttcgttcgacgcgcacggcatcgtccccggctgggagaaggcgctcggtgtcgacacggacaccctcgtgtccaaggtcaacgtggcgggcaccggcgtcggcgccggcggtggcaagtacgccgtctcccgctccgacgaggaaggcagcgaaccgtactcggtgtggctcggcaacaccgacggcaagggcaagccgcagctggtgagcggcaacaacggcaactacaacgtctacgcgacgacggacggcaagaacgtcgcctgggccgagttctccccggggtcggtcctcttccatgtccgtccggtgggcggcggcccggccagggtcgtcggcggcgtcggagccaatgtctcgcagctcgtcctcgaaggcgacttcctcgcctacacggtgacgaacccgcagaccggccggcagcagatcggctacatcaacatcaggacccgggaggcgggcatgatcctcgccaagggcatcgaggtcacggccatgccctcgatccgggacgggaagatcgcctacgccacgatgtggatcgacggcagcgacgtccaactgggcatcggggtcctggacaccgccaccggcaccacccggatgatgccggccggagccgagggcgcgcagggcatcggccagacggccttcaccgacgacggtgtcttctggatcgaggacggcgccggcaccgaccccgccaaggccgccgtcgtgcgcgccgacgccgacggcacgaaccgcaccacggtcgtccccgaaaccggccaaggctccctgcacgcctaccagctgacggcctccgacgacgcggtcaacgtcacctcgctgccgccggccacctcatgggccaacgagaccctgccgaagctgttccaggtggcacccgacggcaagggcggcgcccagcgcgtgtcctgcaaccgcggcgaccaggtctacgcctcggccgacgagggcaagcgcgtcgtctggctggacggcacgaccggttacacgaacgtcgtgaagcgggaccgcccggcgggcgcgtgctgacctgacctgagggaaacgggagaggcggtcgaccccgatgggccgaccgcctctcctgcgtggctctacggcgacagtggccgatggagtggagtgatccaggcggcggtcagtgatccgcgaggaggccgggcagtctgcgcatgtcgtcgaagacgacggtgccggggccttcgagacggtccgcgggagtcagtcccccggcgtaggcgaaggctcgcatgcccgcggcccgggctgcctgaagaccgtactgactgtcctcgaccacggcgcacgcctcgggcacgacccccatcttccgcgcggcgtgtaggaacagatccggggccggcttgccgtgctcgacctcggtggcactgaaaatgcggccttcgaagcgcgggcggagaccggtcatccccagcgtgaaacgcatcttgtcgtggctcccgctggatgccacacaggtggggagatgcgtgagcgcgtcgagggcgtcgacgatgccctcgaccggcgtgagttcggcggcgagcgcctcgtcgtagaggggcttgaactcttcctcccagtcggccggcagacggcgcccgaggtggtcctcgatctgccgcgtcatcgaccggctcgacaggcccatgaaccggtcgacgatctcggcttcggtgagattccaccccagctcggccaggacgagtgcgtccacgcgcaccgcgatgcgttcgctgtcgacgagtacgccgtcgcagtcgaatatgacgagttcaatgggttgggccatgcccgcaggataggcgggccgtccccgtcacacgaggacggcgttcggccacgcccggcagagagtcggacggtcagctcccgtccacgtagaacccgaaaccgggttgctgcgggatcagccaggcttccgcggccggggcgagatcgacgatcagacgctccccgacgccggaggccgtggtggcgtccggagtgaggtagccgaggccgcgcagctcacggaccgccgtgcggaaggcctcgcggccctctgcagccagacgctcgtcgcggttgagcgcgaccagctcgctgatcgtggggtcctgcccggggccgtagccgacggagagttcggccaggatcccgcgggcacgccacgtcaggcgcatgtcccgcatcgcccgcgcgaagaggacggggtcatgggcgggcggggcgggaggtaaaggccaccgcgggggacgagtgtcgggggtgctcatgtgcaggtggggtccttcggtgaggctcatggtccgaacgggactgagcggcgggacggtgtgtggatcaactgtcccggaaaactcaaccacatctggcggtccgggttgttccggttcgtgtccaccaaactctgtcagggcaaccctgaggatggctggccgcatcgatccagtcggcacttcgtacggcttcaactcggcggagcaagtcgcccgccaacgtgacgctcgtgatcgcgcaaggcttctgaccaaggtcagcgcgccccctctcaggcgcgggaaccgcgcgcagtcccagcagtggtcggcggcctcggcgagccggtcctgagcgtcccaggtgaagtgccagaccgctgtgcggccacggagcgttgagctccgacggatcaccggccgcccttgcgcgtcgtgcgcggcccggtgtcgggttccggctctccataggcacaggacgaggagaggatgccgtcggggccttcggtccgcaccacccggccggcctggtcgtagacgtacggaaggtggcgtcgttgcagtcggtccaggaggtgacgcgtccatcggcgtcgcaggtgaaccgcactggcgaacctgatgagttcacggcggcgtcgaggttgccgacggtcggcagccgagcacggatgagtccgccccaggaccagctgcaggacgccggggagccccaggacggtctgcgggagcagcatctcaccggtggcgacgtccacggggtcgttcttgcacgtcttggcgcccgatggtcgccaaagcccttggtcgccttgaggtcgcccatcagccggcccgtcgatcccggcggcggaccgccgtccaccgaggcgagtcgcagtccttccttcgcacccgcgactccgtcctggaacccctccttgccggcgtggacggcctgaccggtgtcgatcccgttctgcagcccgaccgcgttggctgcggcctgaaccgccaggtcggcggcgacgttctcgatcgcctgcactgcgggctcggtcagcgtggacatgatgcaaccggccgcttcctcgcccgcctccttcaccagcttcctgaccagggtgccggtggccgtgacggtgcccgccggtatgagcgtcgacagaccgaacgtcagagtggcgacgcttgctcgacgaagggctcggaccctcgtgtgggaccggttgacggccttctgacggcacggcaaggccgtttcccgcccccggggccggggtgcgggaaccggcctgtcccgtccccggggccggggcggtgccgtcagccggcgccccacgcctccggggggaagagcctgtgccgggtgacccgccacaagtagtcggctccggcggtgccggccgtcccttcaagggggcccaggaagcgttccgccagggccgcgtggcgggcgcgccagacggtgacccgttcgtcgaacctcaggagcgcttccccgatccgttccgcgtccttcccgttggcgcatcgggtccggaaggcgtcgtaggcctccgccacggagggctggcgggcccgttcccgcagtcgccgacgttcggtttccgtgaagcccggtgtggagatgtggcggtggtcgcgcagaccgcacaggaactcgatctcccggtactgcgccgactggaagccgctcgccgtgcccagccccgcgcggatcttgccgaattcctccgtactcagcgtggccagcatgtcgaagtggcggacgagcagttcgaagatctccggcagtcgcggcagcagatgcagtgccgtgtccgggtcgtcgtccgtgagggccgcgcggatttcctccaggtggcgcagcacgatgccgaaccagatctccgccgactggtgcgtggcaaggaagaggaccggggcggccgcgtcgcgcaggtgcgcgacgttcagcaggtcctccaggcgcagcagctccccgtaggtcgggctctcgtggtacgtggcgccccggccgccaccgtcactcccggtcgtccgggtgtcctgcgctgtcatggtttgcgtccgtgggcgacgagggctatccgggggagctcggcccggaaggcattgccgtgcggacggacggtaagttccaggaccgcctcgtcgcgggggacgtcctcggggaggtcgccggccgagaaacggaagatgtcgcggagggcggcttccgtccgctcgttgctccggtcgccgtcgggcaggatgccgtacatcgaggtcatgtaggagacgagccgagccagtgcgctctcctcggtctccccggtcagggtcatcgggtcgtacacggggccgacggtgatgtcggtgaagcccgcgtcggcgaggtagccggtcatctcgtgggcggtgaagtggtcgtacgcgtggcccgcgagggagcgggggtggacgacttcgcggaaccagcgttcttcgggggatccctccgcgaagtcgtggaggacgacgcggcctccaggggccaggacccgggaggcctctgtcagcatgcgggggcgtacagagcggtcgacgtggtgagtgccgtacgcgaagagcacgccgtccaggcagtggtctcgctgcagcatgcggtcggccggctggcgtacggcgggcaggccggaccggagggctgcggccaccatgtggccgctgaggtctccggtgaccagtggcacggagtccccgtcgccggcgccggccagttgccgccacacgcggctgagcaggccttcgcctccgagtacatcgaggacacggcccatgtcgcgcagcccgccggggccggcgaacatgccgagaatcgtctcaatgccggtgcggcgggcttcgccgtgctgctgggcacagcggtaggagacgccgcggccggtgtggtcgtcgtcgaactcgtttccctccgtgacccaaggctcgtccgtgacggcgacgaggcgtgcggggtccaggagctcgaagagcgccgggtgctgttccttgacggtcacgtggtagtcgtgcacggtcatgccggggaggtaggtggtggtgcgggtttcgccggtcacgacgggcccctctctgccggtactggacaaggtcgtcgtgcctttcagctcgttcggaagaaatcgcgaagaatcttgttgaccgcgctcgcatcctcgaggtagccgtagtggccgcagcggggaacggtctcgaagcgggcgccggggatcgcgtcggccacttcgcggcccaggtacggcggggtgacgacgtcgtcctcgaacgcgatgaccaggcaggggacggtgatgtcccggtagtccgcgaggcggtcgggcagcaggccgagttccaactgcgcacggacccccggcccgtcccgttcggcgtactcgaagagtgggagccagtcctcgacctgatggcggtcccggagggtgtggggggagaggttctgcagcgcgcggacagccgccgcgtaggcggggggcagttcgacgccgctgtcgtacagggcggcggccgccctggtggcggcggcccgcatcgcgtcggtgcgaccccgggtggccatgaacaccgcctgggtcaccaggtccgggcgggccagggcgacttcctgggccacctgggcgcccagggacgtgcccacgacgcggcagggtccgcccgccacctgttcgatcagcgcggcggtgtcggcggcgaggtcggcgagggtgaagccccgctcgcactcgtcggacggcggtacgccgcgattggtgaacgtgatggtgcggaatccggccgcgtgcaggtcaggcacctggtaggcgtcccacacccggccggtactgccggtgccggcgatcatcagcaccgggtcaccgcagccgctgtcggagcaggcgatccgggcgccgttgacctggatcatcctggatgtcgccctcatgtcggccctccttccgtctccgctgtccggctgcgggtcaggccgcggtcgtccggtcgttctccatctgccggatgaggctcttggggcgcatatcggtccagttctcgttgatgtggtcgaggcattcctgccgactgctctcgccgaaggtgacggtccagcccgccgggacatcggcgaacgcgggccaaagggagtactggccctcgtcgttgaccagtacgaggtagctgccgtcgttgttctcgaaggggttggccatggcggtatgtttcctttcgtgtacggacaggggggcgcccccgtgtccggtgtcccgtgtggggtcctgggggtcaggtgccggcgcccagccgtgcggcgagcacggggccgatccgggtgagtgcggctggatcggtcatctcctcgtgtgcgcagtcgatcaggtgcaggtcgaggtcgccggtgacgtgggggtgccacaggccgacgcgttcggcggcctgctcggaaccgggccgtgcggaggcgaagaacaccatgtcgccggtgaacacgccgggtgtgaaacggcgggtgagccgggcgttgttgaggaagacgtccttcatcgcgaggatccgcccggcgtcgaagtccctcgcgggcccgtcggtgcgccgtacgaactcgaggaactccgggagccacgcgtcgccctcgaggcgctcgggggcgacgggggaaccgagctgggcgaggtagtcggcgacgatgcgctgttcgtccacctcggcgtcgggccgggccccggtgacggggtaggcgtcgaggacggcgaggagttcgacggtgtggccctgctgttgcaggcgggtcgcgacggcgtgggcgaccgtgccgccgaaggaccagccgagcagccggtacggcccatccgggcacaggcgccggatctcaccggcgtagtcctcagccatctcctcgacgctcccgggcagcccgccgggcgcggtgagcctccgggcctgcagtccgtaaaccggcacgtccgcgtccagatggctgagcaggccggcgtacgcccagctcaggcccattccggcgtggacgcagaacagcggtggccggctgcccgtgcgccgcagcggcagcaccgtctcgaacgggtcgtacgccgcggcgtccgccgtgcacgccgacgcgagcagggcaggggtcggcgcctcgaacagggtgcgcacggagatctccacgcccagcgccgcgcggacccgggccgcgaggcgagtggcgagcagcgagtgaccgcccaggtcgaagaacccctcgtccacgccgacggtctcccggccgagcacgtcggcgaacaaggcgcacagcacccgttcctgcggcgactggggtgcgcggccgggggcggcggtctgggtgccggggccgggcagcgccgtgcggtccaccttgccgttggcggtgagcggcagggcgtccagggtgaggacgtcaccgggcaccatgtgccggggcagccgggacgccaggtgggcgcgcagcgcggcggccgacggtgtgccggggccgccggtgacgtacgcgacgagccgctggtcgccgagcccgtcgtcgtacacggtgaccaccgcgtcctcgacgccggggtgcgatgcgagggccgcctcgatctcggcgagttcgacgcggaagccgcggattttgacctgcgtgtcggcgcgtccctcgaacagcaggtggccgtcgctgcggcggcgggccaggtcaccggtgcggtacatgcgctcgccgggggcgccgaaggggtcggcgacgaagcgttcggcggtctgtccggggcggcccagatagccgcgggccagtccggccccggcgacgtagatctcgccgacgcagccggcgggtacgggccggagccggtggtcgaggacgtacacacgctggtcgggccagggtgcgccgatcggcacgggcgcgccgtcaccggtccgttcgcacacggcgtaggtggcgccgacggtgacctcggtggggccgtagccgttgaggatccgccgttcgccaccggcccagcggtcgacgagttggggtccgcacacgtcgccggtgacggtgaccgtcagccgggggggtgccgcgcctgccgggacagcggccagcgcggccgggggcagggtgacgtgcgtgatgcggtgtgcggtgagcgcggcggcgagttcgtcaccgaagaggtccgcgtccggggcgaggacgagggtcgcgcccgacagcagcgcggagcacaggtcccagacggaggcgtcgaagctggtcgacaccagttgcaggacccggtctccggggcccgcgcgcagtgcctcctgctgggtggcggccagcgccgggatgccttcgtgggtgacgacgacgcccttgggtgtgccggtggagccggaggtgtagatgacgtaggcgggatgggcgggcagcagtcccgtgccgcgcggattgccgtcgggcgcggcggcgagtgcggcggtgacggccgggtcgtcgaggtccagtacggggatgccggtggggagcatgcggtcgcgggtggctgcggaggcgatgaggagcgccggggccgcgtcggcggccatgtgggcgatgcggtcgtcggggtaggcggggtcgacgggcacgcacacggcgccggccttgaggatcccgagccaggccgtcaccaggtccgcggagcggggcagggcgatcgccacgcgctgttcgggccgtacgccccggtcggtgagcagccgggcgagccggttggcccgccggttcagtgcgtcgtacgtcgtagtggcgggccggcggccggcgacctcgaccgcgatgtgaccggggtgcgccgccacccgggcctcgaacagctcgggcagggtggcgggcggcaggccggtgacagtgccgcggctgcgggtcagcagtttgtggagggtgtgttccgggagcagcccgaccgcgtcgacgcgggtgcccggggcgtggacggccgcgtcgaggaactgcacgaagcagtccgcgatctgccggacggtgccctcgtcgtacaggtcggcgtggtattcgacgattccggtgatgccggagggttcgcccttctccgggggccgctcggtgaacgcgaacgcgaggtcgaacttggccgtcccggcgtcgaggggctgttcggtgacctgcaggtcgggcagcagggtcccggggcggtcggagatgctgcggtcctcggtgttctggaaggcgagcagtatctggaagagggggtggcgcagcagtgagcgttccgggttgaggacctccaccaggcgttcgaagggcacgtcctggtgtgcgtacgcgtcgaggtcggtggcccgcacgcgcgcgaggagttcggcgaacgtcggatcgccggagacgtcggtgcgcaggacgagggtgttcacgaagaacccgatgaggtgctcggtggcctcgtcggtgcggcccgcgatcggcgtgccgatggggatgtcggtgcccgcgcccagccgcgacagcagaccggcgagggccgcctggaccaccatgaacacggtggtactggtgacgcgggcgagggtgacgatgtcgcggtgcagcgcggcggggatctccagccggcaccggccgccgcggcggccgacgtcggggcgtcgggggcggtcggtgggcaggtccaggccctgcgggaggccggcgagtctgccccgccagtgggcgagctggccggccatctcgctcgtcccgtcgtcctcggtgccgagtacgtcgcgctgccaggcggcgtagtcagcgtactgcacggacagcgggggcagttccggcgcggagtgtgcgcgccgggctgcgtaggcggcggacaggtcacggaggaacggctcgcgggaccaacggtcgcttgcgatgtggtgggtgacgacgagcagggtgtggtcgtcgcgctcggtgcggaagacggtggcgcggacggggatctcggcggtgaggtcgaaggggtggcgggccgcttcggcgaggcgcccggccagttcgtccctgtgcaggggtacggcctcaagggtgattcgggcgcgagggtcgccggtgggcaggatgtgctgccacgcggtgccggtgccgtcccgggcgatgagtgtgcgcaggctctcgtggcgggcgacgacatccgtcagggcggcttcgagggcggtcacgtcgagaggaccggtgaggcgcagcgcgagagggatgttgtatgtggcgccgtggccctcgagttcgtggaggaaccacaggcgctgctgggcgtacgacagggggtcccgcccggtgcgttcccgcgcggtcaggggggcgcggacgggggcgccggagcgttcccccaggagccggtcgaggcggccgggggtaggggcgtcgaagagggcgcggacgccgagttcgacgccgaggaccgaacggacgcgggacaccaggcgggtggcgagcagcgagtggccgccgagttcgaagaagtcgtcggccgtgccgacccgttcgaggccgaggacttcggcgaacaggccgcacagaacttcctcacgggccgtgccgggggcacggaagccggcgccggagacggcctcgggcgcgggcagggccgccctgttgatcttgccgttgggggtgaggggtacctcgtgcagtaccacgacggcggacggcaccatgtacgcgggcaggcgggcggcggcatgggcggtcaccgccgacggcaggccgccggtggggtcgacgtcggcaggtgtcacgtacgccacgagccgctggtcgccggggcggtcctcgcggacagcgacgatcgcgcgcgccacaccgggggtggcggcgacggtggcctcgacctcaccgagctcgatgcggaagccgcgtaccttgacctggtcgtcgacgcggccgacgaactccagcgtgccgtcggttctccaccgcaccaggtcaccggtgcggtacatgcgttcgcccggtgcgccgaaggggcaggccacgaagcgttcggccgtcaggccggggcgtccgagatagccgcgggccaggcccgcgccggccacgtacagctcgccggcgacgcccgtgggcacgacgcgcaggccgccgtcgaggacgtacaggcgggtgttccacaggggccggccgatggggacggggccgggaggcagggtggtgccgggggcgatgcggtgttcggcgcagttgatcgtggtctcggtggggccgtacacgttgaagacggtcacgccggggtggcgggcgcgccacgggtcgagggtctcgctcaggagttgctcaccgccgagggtgatggccccggtgggggagatctcgtcggggagggagtcgaggacggcgagatggctgggggtggccttcatgaaggtggggctgtcctcgcccaggtcgaggatcttgtcgtcggtgaacgatccgaggcgcaggcagccaccgctcaccaggggtgcgtacagcgctccgacggtgaggtcgaaggacacgggcgagtgcacgaatgccttgccggcggcttcggggtattcctcgatcgtgcgcaggaggtagccggtgacggaacggtgcaggatcacgacgcccttggggcggccggtggagcccgaggtgtagatgacgtacgcgggctggtcgggaagcggcgcgataccgtcggtgccgaggtcgtgttcgggcagggcgcgaacgcccgtgtcgagggcgtcgtcgtcgagggtgaggacgcgggccgggccggcgggcatggccgcggccgagcgggagtcggtgaggacgagcgcggggccgatgtcgccaagcatgtacgcgatgcggtcggccggatactccgggtccacgggaacgtacgcggcgccggtcttcatcacggcgagagcggccacgaccatgcgggcggagagcgggagcagcagggtgacgtactgctcggggcccacgccggccgcggcgaggtggcgtgcgagccggttggcgcgcgcgttgagttcggcgtaggagagttcgccgtcgcgggcatggaccgccggcctgccgggggtgcgggccgcctgggcctcgatcattccgggcacggtgagcgggggcacgtcgcggtgggtgtcgttgaggccggtgaggagatgctgtttctcctcctcgccgaggatgtcggccctgccgacggcgaggtccggctggtcggccatgagctccagggctcgcacgaggctgtcgcggatcgtcgtgacgcgttcggcgggcagggcgctcggccggtggccaaagcgcaggcgcagcggcgggccgggcaggaccagcagaccgagggggtagtgcatggcgtcgcgtacgtggacgtcggcgacccggggttcggccgcggggccgtcggcgccggcggaggatgcggcggcgacggggtagttctcgaagaccatcaccgtgtcgaagagttctgtccggccggcgagccgctggatgaccgccagtcgttcgtactggtggtcgaggagctgtatctgttcgcgctggaggcggacggtgaagtcccggaggctctcgtgcggcagcagctcggcacgcagcggcacggtgttgatgaagaggcccaccatgtcctcgacacctgcgagctccggcggccggccggacacggtcacgccgaacacgacgtcgtcgcggccggtgaggcggccgaggacgagcgcccaggcggcctggacgagggtgttgagggtgacgccgaactcgcgggcgcgggcggcgagcgtcgccgccaggtccggttcgagccgggactcggtccacagcggcggctcggcggcccggtcggcacccggggcgacgagggtggcctcgtcaaccccggccagtgccttggaccaggcccgcccggcctcgtcccggtcgcggcgggagatccaggccaggtagtcccggtacggccggacccagggcagggcggtctcgtcgccggtgcggtagagggtgatgagttcccgcagcaggacgggcatcgaccagccgtccatgaggatgtgatggaaagtcagcgcgaaacggtgccggtcgtggcctgtgcgcacgagcaggaaccgcagcaggggcggagccgtcagatcgaagcggcgccaccgctggtcgtgcagcaggcgttcgatctcggccttctgctcggcgccctcgagcttcgcgacgtcggtgtgctcccacggcaccgcaacctcgcgcgggatgatctgcacggggcgctgcagccgctcgtaccggaaggccgcccgcaggttggcgtgccgacgcagcagggcggccgcggcggcgcgcagtgcttcggggtcgacgggcccctcgaggtcgacgaccacctgcacggtgtacacgtcgacggactgctcgtcgtaaacactgtggaacagcagtccctcctgcagcggagtgagaggaagtatgtcctccatcgcgcgctgcgtcatccctgtgtcctccagtcgtcttcgagcagttcgatctcagcaaaggagagttccgcgaggccgacgtccgacggggtcagcccacctgcctccgggtggcgagcgtgctccacgaggccctcgagcgcctcatcccagtaccgtgcgagctcgcggatgcgcgactccgggaggagggtggtgggccaggaccacgtgaccccgagacggggaccttcgggcgtgtcgagggtgatggcgttgaactccacggcgtgggcgaccgggagctccgggtcctgacccgcagcgctccgtgccagcaggctccaggcaggcgcacggtccagggcgtccgcggtcgtcgtctcgtccgtgcccgtcgtgaaccgacccaggtagttgaacccgaactggggcgtcgcgtggcgagcgagggcaggtccgtgttccgggtccaggtaccggaggatcccgtagccgagtccctggtccgggaggctgcggagcgtctccttgacctcccggacgacccgaccgacgacgggaccacctgcccggacctcatcccaggaagccgactcgaccgggacacgcacggggtagacgctcgtgaaccagcccaccgtccgcgagaggtcggcaccgcggacggcgtcctcgttccgaccgtggccctccacgtcgacgaccacgggaccgccacgaccccgccagcgtgccacggcgaggccgaacgctgcgagcaggacctcctggacacccacaccgagcacgctggggacctcggtcaggagggcacgggagacggagacggaggcacggctgtcgacggagacgacgccaccgaagacgtcgacggcaccgtcgacggcacgggagccgacgagcacgtccgcaccgcggaccatgcgggaccagagctcgacctcgcctgcacgctccccacggcgaccctgctcggcgagagcggcagcccatccgcggagggaggtgccgacgacgccgagctcgacacgcccaccggagcggacctgggcccagccctccgccaggtcgccgaggacgacgcgccaggagacgccgtcgacgacgaggtggtgggcgacgaggacgaggacaccggaacggtcaccaccgcgctcgagccagacggcacggacgacgcgtccgacggacgggtcgagccacgcacgggcctcgacggctgcggcacggacggcgtcgacctggccgtcgacacccatgcccgagaggtcgcaccgcacgatccaggagccgtcgtcgggcacgacaccgtccacgcagagggctcccgagtcgaggacccggagccgccaggcctcgtgccggtcccggacggcacccaggaggacgcggagctcctcccagaccaggcctgccgggacagccaggaccaccgactggttgaagccacggacgacgccaccgagggatccaccgccacgctcggcgagccactcgacgaccggccagcgctcgacctcgccgacacccgggaggacgatctcagcgccaccgacgacacccaccgccccagcgtcggaaccacggacgacggcagcgagagcacgcacggtacgcccttcgaacacgtcccgcacggacagctcgagacctgcacggcgagcgcgagcgacgagctggatggacaggatcgagtcgccaccgaggtcgaagaagccgtcgtcgacgccgacgcgttccacgccgaggacttcggcgaagagtccgcagaggacttcctcacgcgggctggagggtgcgcagaaaccggcgacggagacgaccgggaccgggaggcctgcgcgatcgaccttgccgttgggggtgagcgggatctcgtcgagcacgacgaccgcggaggggaccatgtaggcagggagacgctcggcgacgagacccgagatggccgaacgcagcccgtcgccaccgacgccaccgaccgtgacgtaggcgacgaggcggtggtcaccgggacggtcctcccgcacgaccaccacggcacggacgacgtccgggtgggcagccacggcaccctcgacctcgccgagctcgacccggaaaccccggaccttgacctgatcgtctgcccgacccacgaattccagcgtcccgtcgacgcgccagcggacgaggtcaccggtccggtacatgcgctcaccggggacaccgaaggggcaggcgacgaagcgctcggcggtcagtgccgggcggccgtggtagccgcgggccaggccggcgccggccagatacagctcgccgggcacgcccggtggcacaggacgcagggcggcgtcgaggacgtaggtgcggatgttccagatggggcggccaatggggaccgtggccgtgccggtggcgggctcatacgcgtggtgggtgacatcgacagccgcttcggtcgggccgtacagattgtgcagggggacgggcagtttcgcgcggaagtccacggccaggtccgtgccgagggcctcgccgctgcatatcacccgcctcagtccggtgcagagtgccgcgccgggctcttcgaggaagacgcgcagcatggagggcacgaaatgtgccgtggtgatgcgctcgcgctcgatgagtccggccagatacgccgcgtcccggtggccgccgggccgggcgaacacgaggaccgcgccctcgagcagcggccagaagaactcccagaccgacacgtcgaaaccggccggagtcttctgcaagacacgatcggtcgcgtcgagacggtactccgcctgcatccaggcgagccggttgacgatggcgccgtgggagacgacgacgcccttggggcggccggtggatccggaggtgtagatcatgtacgcggggtgggcctcggtcaccggcgtgccggggttcgtgccgggggcggtggcgagtgtcgcgcggatctctgtgtcgtccagcccgatccgcgggacggacccggggagttcgacgtccccgcgggtcaggaccgccacgggggcagcgtcgccggccatgtacgcgagccggtcggccgggtactcggggtcgagcggtacgtaggccgcgcccgatttcacgaccgccagcagtgccactacgaggtccggactgcggggcagggccacaccgacgaaccgctcggggcctgcgccgtgctcggccaggacatgggcgaggcggttggcgcgtgtgttcagttcacggtacgacagttcttcaccctcgtacacgaccgccggcgcgtcgccccgggctgcggcccgtgcctcgaacagcgccgacagcgttcccgtcacgccggggacggcggtgtcgttccactccgtcaggacgcaggcccgctcggcgtcgctcaccacgggcagctcgcccaggcgtaccgacgcgtcggcggcgacccgctccagcacccgcaccaggcgctccgcgatccgtgcggcggtggggtgatcgaacaggtcgcaccggtactcgaccgctccccggagctcaccgccgacctccgacaggccgaacaggaggtcgaacttggcggctccggcaccgagcgggagacccgctgccgagagacccgggaagtcctccaccggtccgccatccgcggtgttgtcgaacgccagcatcacctggaacagcgggtgccgtgccagcgaccgctccgggttcacggcctcgacgagccgttcgaacgggatatcctggtgcgcgtatgcgtccaggtcggtcgcacgcacacgggcgagcagctcggcgaaggtcggatcgcccgacacgtccgtgcgcaggaccagggtgttgacgaagaatccgatcaggtcctccgtcgcctgatcggtccggcctgcgatgggcgtcccgatcgggatgtcatcgcccgcacccaggcggctcagcagcgctgcgagggcagcctggaccaccatgaacacggtcacgccctccacgcgagccagccgggtcaggtcgcgcagcacgcctgccggcaccgtgaagcccacgcgaccgccctcgtaggaggcgcgtgcggggcgcacgcggtcggcaggcagcgcgagctccgcaggcaggtccgccagctgcgtccgccagtacgccagctgcgcggagagctcggagctctcatcgtcttcggtgcccagcacatcccgctgccacgctgcgtagtcagcgtactgcaccggcaggggcgggagctgcgggtcgcgaccctccaggcgtgcggcgtaggccgtcgagaggtcccgagccaggaggccggtgctccacccgtcccctgcgatgtggtggaagacgagcaggagggcgtgatcctcgtccgcgacccggaacaccgtcgcacggagcggcatctccgcggtcaggtcgaacggccgacccaccgcctcgttgagcttggccaccagcgtgtccgcgtccgtctgcaccaccggcagcgcagggcgagctgccgggtcacccaccgggaggacctgctggcgagcggtcccgtcgctgtcccgagcgatgagcgtccgcagcgactcgtgccgtgcgatcacgtcgatcagggcctggtggagggcctccacgtcgagcacgccagcgagccggagcgccatggggatgttgtaggccgcaccgggtccctccagctcctccaggaaccagaggcgctgctgcgcgaaggagagcggctcgcgaccggtgcgctcgcgtgcgacgagcgggacacggacaccgagcccgctgcgctcgcggaggaggcgctcgaggcgcgacacggtcggggcctcgaacagcgcacggacaccggcctccacgccgaggacggcacgcacacgggagatgagccgcgtcgcgagcagcgagtggccaccgaggccgaagaagtcgtcgtcgacaccgacgcgctcgacgccgaggacctcggcgaagaggccacagaggacctcctcgcgagggctcacgggagcccggtagccggcaccagcctcgacacccgggaccggcagagcggcacggtcgaccttgccgttcggggtgagcgggatctcgtcgagcacgaccaccgcgctcgggaccatgtacgccgggagacgttcggcgaccgcacgcatgacggcggacgagaggccaccggtgtccacacccgtgacgtaggccacgaggcggtggtcacccgggcggtcctcgcggacgaccaccaccgcacggacgacgtcagggtgggcagccacagcgccctcgacctcgcccagctcgacgcggaacccgcgcaccttgacctggtcgtcggcacgacccacgaactccagggcaccgtcgacgcgccaccgcacgaggtcgccggtccggtacatgcgctcgccaggggcaccgaacgggcacgcgacgaagcgctcggcggtgagaccggcacgacccaggtacccacgggcgaggccgtcgcccgcgatgtacagctcgcctgcgacgcccggtggcacgacacacaaagcagcgtcgagcacatacacgcgagtgttgaggatgggtgtgccgatcgaagggggagcgctgtccccggcggcctgcccggcgctggtggaccagatggtcgtctccgtcgggccgtacatgttcgtgacggaccgcgcgcctcgtgtcagcgcgtcgcgcaacccggggctcagggcctcaccgcccacgacggcgtgcacgccgcgcaggctgtcgcccgcgtcggcgagcatgccgtgccaccagctcggtgtcgcctggaccatggtgacgccgtggtcctcgcacgtctggcgggccagcgccgggtcgcggaccgtctccccgtccgcgaggacgacggtggcgccgctcagtagcgggacgaagagctcgagggccgcgatgtcgaagcccacggtggtcacggacagcaggcggtcgccaccgcggagcccgagcctgggcaccatgtcggccaggaagttcaccagggcggccgacggtacgacgacgcccttgggctggccggtggatccggaggtgtagatcacgtacgccgggccggcggggttgccccggccggcgggggacgtggccgggtggtcgtggagctcgccggtcacctgctcggagtcgagtaccacatggcttccgccgggtatgcgggcgctgatgtcctccgtggtgaggaccaccgcagggcgggcgtcatccagcatgtagctgagccgctcggccgggaagccgggatccagcggcagatgtccggcacctgttttctgtacggccagcagggccaccaggagctcttcggtccggggcagggccaccgcgacgaacgtctcgggggccgcgccccgggcgagcagcaagtgggccaggcggttcgcgcgcgcgtccagctccgcgtacgtgagccggatgttccccgagacgagagcgacatcgtgcggggtgcgccgcacctgctcctcgaacagctggggcagcagtgccggggggagagcggcagccgtgtcgttccactcgtgcagcagttggtcgcgctcgcccgcccggagcacgtcgacgctgctcgcacgttgctccggggctgccgcaaaagcagcgagaacatgcaggaaccggtcggcgagctgccgcacggtgtcctcgtcccacaggtcgcggcggtactcgaagacacttctgatgccctggtggtcgcccgtctcaccgcgggattcgttgcaatacagggccagatcgaatttcgccacgcccggttcgatgtcctgccgggaggcagccaggccttcggccatgggcagcggggtgctcgtctcggcgttgttgaacgcgagcatcacctggaagagcgggtgacgggccaggctccgctccgggttcaccgcctccaccagccgctcgaacgggatatcctggtgcgcgtacgcgtccaggtccgtggcgcgcacacgcgccagcagctccgcgaaggtggggtccccgctcacgtccgtccgcaggacgagcgtgttgacgaagaacccgatcaggtcctccgtcgcctggtccgtccgacccgcgatgggcgtcccgatcgggatatcgtccccggcacccagccgcgacagcagcgcggccagcgcggcctggaccaccatgaacaccgtcaccccttccgcacgcgccaggtccgcgagcgaccggtgcacgtcagcggggatcgtgaactcgatccggccaccccggtacgtcgccacggcagggcgagcccggtcggtgggcagcgcgagctccgccgggagcgacgccagctgcgtccgccagtacgcgagctgcgcggagagctccgaggactcgtcatcctccgtccccagcacgtcccgctgccacgcggcgtagtcggcgtactggaccgacagcggacgccacgcggaagccgctccggctgcgcgagccgagtaggccgtcgacaggtcgcgagccaggggacccatgctccagccgtcacccgcgatgtggtggagcacgaggagcaggatgtgatcgttgtccgtgagccggaacaccgtggcacgcaccggcaggtccgccgccaggtcgaacggacgaccagccgcctcgtccaggcgctcctgcagggcatccgcaccgatgtcgaccagaggcagacccggacgcgtccgaggatcgtcgaccgggaggatgtgctgccacgccgtaccggcgtcctgggcgatcagcgtccggagcgactcgtggcgcgcgatcacgtcgatcagggcctggtggagcgcctccacgtcgagcacacccgcgagccgcagggccatggggatgttgtaggccgcacccggaccctccagctcctccaggaaccagagccgctgctgcgcgaaggacagcggctcacgaccggtgcgctcgcgtgcgacgagcggcacgcggaccccgagaccggagcgctcacgcagcaggcgctccaggcgcgacacggtcggggcctcgaagagggcacggacaccggcctcgacgccgagcacggcacgcacacgggagatgagacgggtcgcgagcagggagtgaccgccgagctcgaagaagtcgtcatccacaccgacccgctccacaccgaggacctcggcgaagagaccgcagaggacctcctcgcgagggctcaccggagcgcggtaccccgtcccggcctcgacctcggggaccggcagcgcagcacggtcgaccttaccgttcggggtgagggggatctcgtccaggaccacgacggcggacgggaccatgtaggcagggaggcgctcggccacgagaccggagatggccgaccgcaggccgtcaccacccacgccaccagcggtgacgtaggcgaccaagcggtggtcaccgggacgatcctcccggacgaccaccacggcacggaccacgtccgggtgcgcagcgaccgcaccctcgacctcgccgagctcgacgcggaaaccgcgcaccttgacctggtcgtcggcgcgaccgacgaactccagcgcaccgtcgacgcgccaacggaccaggtcacccgtccggtacatgcgttcgccgggcacgccgaacgggcaggcgacgaagcgctcggcggtgagaccgacgcgacccaggtaaccccgagcgaggccgacaccggcgacgtagagctcgcccacgacgcccaccgggacgacgcgcagggccgcgtcgagcacgaacatccgctggccagcgaacgggcgaccgatcgggaccgggccgtcgggcacctcctcgccaggggcgatcaggaactccgcgcagttgacggtgagctcggtgggaccgtaggcgttgacgaccacgacaccggggttgcgctcgcgccactcgcggagagccccaccggagagggcctcgccaccgacgaggagcatcccgttaccggcgacgacgccctcgagctcgccgaggagaccgaggtgggacggggtgaccttgacgaaggaggccccgacaccctgggcggactcgtcgagctcgcccagcaccacgcagccacccgagaccaacggggtgaagagcacggtgacggtcaggtcgaaggcgagggacgagtggacgaacccgacaccgcccacggcgcctgcgtagacgtcccggccacgggccaggtaaccgccgaccgaggcgtgggtgacgacgacgcccttgggacgacccgtggacccggaggtgtacacgacgtagagggggtgaccggggaggagacgggaggcgcgctcgccgtcggtgacgttgcgcacctcgacaccgtcagccgcgacgagctcgaccaggcaggactcgcgcaccacgcgggagaattcgaacacctcgccaccggggaagtcacccacgacgtgggagaggccctcaccgacgaccacgaggccgacaccggcgtcggcgaggacccagccgacgcgctcgacgggccactcggggtcgacgggcacgtagaagccaccggccttggcgacggcgagcagcatgacgaccacgtcgacaccgcgacccatgacgacgccgacaccgcactcggcaccgacgccgcgtccgacgagccagcgggcgaccacgttgacccgggcgttgagctcgccgtaggaccagacgacgcctgcaccacggacggcgacggcatcggcgtccgcacggacgcgctcttcgaacaggccgacgagaccgcctccgggcacgaccgggaccggtccgccgacgccccaaccgagcaaccgttcgcgctcgccgacaccgaggacatccacaccgctcacggagacaccgccaccgccgctgacgacgccctcgaggacccggaccagccatccccacacctcacgcacccgacccgcatcaaacacatccggccgataaccgatttccagttccagctccaggccgtgcatggaagcaacgaggttcagtgcgtagtgggttccgttgtatccgcctgttcgtgagatctggaattccgcttccggggattcagaagaaaccgggtagttgtcgaagaccattccgacatcgaagagttccttgtgcccggcccaccgctggacttccgccagccggacatgctgatgctccagcagctccgtctgctcccgctgcagccgctccacgaaaccacccagcgactccgccggatccagccgcgcccgcaacggaatggtgttcacgaacagaccgaccatgtcctccacacccaccacctcggcaggccgacccgacaccgtcaccccgaacaccacatcgtcccgacccatcaaccgccccagcaccaaggcccacgccgcctgtaccaccgacgcaaccgtcacaccacgcccgcgcgcccacgccacgatcttctgcgagaggtcgccgtcgaccgcaccgtggaacgccgccgggacgaccccgtctcgggaaacgcctggcgcgacgagcgacggctcctccaggcccgtcagcgcccgccgccatgcgccttcggcggctgcggcgtctcgcgcggcgacccatcgcaggaaatcgccgtacgagcggacccccggcagaaccgatacgtcccccgagctgccgtacagggccagcagctcacgcagcagcaccgggacggaccagccgtcgaggacgacgtggtggtacgtgatgacgaaccggaaacgatcgtcgtgcgtccggatgagcacgaagcgcgccagtggaccccggcccatgtcgaaccggcgccagcgctcctcttccaccaacccgtcgataccgccgtcgcgcacgtcgatggactgccacggcagatccacctgtcgtgcgacgaccgcaacccattcgcccgaccgcagttggcggtatcccgaccgcagcaccccgtgccgcgccaccagaccttcgacagccgcgcgcagccggtcgacgtccaccggaccgaccagatcgaacaccagctgccccacatacacgtccaccccaccagaggccaacgcgctgtggaacagcagcccctcttgcagggccgacaccggcagaatctcctccgcaccaccggcgacccccgcctgcagacggtcgagctccacctggttcacttccaccagcgtcacgtcggacggcgtgaggccgccggcttcggggtgccgggcgtgttcgaccagcccttccagggcctcgtcccagtagcgggccagctcccgtatccgggactccggcagcagcgtcgtcggccacgaccatgtcacgcccaggcgcgggccctccggggtgtccagcgtgatcgcgttgaactcgaccgcgtgcgccacgggcagttcggggtcctggccggctcccaccgcgaacggccctgacacggggacccagtccgtcatcccctcgtcaccggtgtcgtcggttccggtggtgaagcggccgaggtagttgaaaccgaactgcggggtggcatgccgggccagagcaggaccgtgctcgggatcgagatagcgcaggatgccataacccagaccctggtcaggcagcgaacgcagagtctccttcacctcacgcaccacacggcccaccaccggaccacccgcacgcacctcgtcccacgaagcggactccaccggcacacggaccggatacacactggtgaaccaaccgacagtacgagacagatcagcaccccgcacagcgtcctcattacgcccgtgcccctcaacatccaccacaaccggcccaccccggccgcgccaccgcgcgaccgccagcccgaatgccgccagcaacacctcctgcacaccaacacccagaaccgacggcacctccgtcagcaacgcacgcgacaccgacaccgacgcccgcgaatcaaccgacaccaccccgccgaaaacatccaccgcaccatccacagcacgcgaccccaccagaacatccgcaccccgaaccatccgcgaccacaactccacctcccccgcacgctcgccccgccggccctgctccgccaacgccgccgcccaaccccgcaacgacgtccccaccacacccaactccacacggccacccgaacgcacctgcgcccacccctccgccagatcccccagcaccacccgccacgacacaccgtccaccaccaggtgatgcgccaccagcaccaacacccccgaacgatcaccaccacgctccagccacaccgcccgcaccacccggcccaccgacggatccagccacgcacgcgcctccacagccgcagcccgcacagcatccacctgaccatccacacccataccgctcaggtcacaccggacaatccacgacccgtcatccggaacaacaccatcaacacacaacgccccggaatccagcacccgcaaccgccacgcctcatgccgatcccgcaccgcacccaacaacacccgcaactcctcccacaccaacccagcaggcacagcaagcacaacagactgattgaaaccccgaaccacaccacccagcgaccccccaccacgctccgccagccactccaccaccggccaccgctccacctcacccacacccggcagcacaatctcagcaccccccaccacaccaaccgccccagcgtccgaaccacgcaccacagccgccagagcacgtaccgtacggccctcgaaaacatcccgaaccgacaactccagacccgcccgacgagcccgcgccaccaactgaatcgacagaatgctgtccccgcccagatcgaagaacccatcgtccacccccacccgctcaacacccagcacctccgcgaacagaccacacaacacctcctcccgcggcgacgacggcgcacagaaccccgccaccgacaccaccggcaccggaagacccgcccggtccaccttcccgttcggcgtcaacgggatctcatccagaaccaccaccgccgacggcaccatgtacgcaggcagacgctcagcaacggcacgcatcaccgcagaggacagtccacccgtgtcaacaccggtgacatacgcaaccaaccggtgatcacccggccggtcctcacgcaccacaacaaccgcacgcaccacatcaggatgcgccgcaacagcaccctccacctcacccaactccacacggaaaccacggaccttcacctgatcatccgcacgaccaacaaactcaagcgtgccgtccacccgccaccgcaccaaatcccccgtgcgatacatacgctcaccccgcgcaccgaacggacacgccacaaaccgctccgccgtcagacccacacgcccgagatagccccgcgccagacccacacccgccacatacagctcacccaccacaccgaccggcaccacgcgcagcccgtcgtccagcacatacacacacgtgttgcgcaggggccggcccatgggggccgcggcgtccatgggggtgtcgccgtaccaggccgtggcgtagacggtggcttcggtcgggccgtagaagttggccacgcgggccccgggcagcacagcgcggacgcggcgcacgtcgcgagcggacagcgcctcgccggccagggcgatcatgcccacgtcggcccggtcgaggccggcttccagcagctgcgcgaaggccgacggcacgccgctgaccaggctcgcggaccatcggccggggcggtcgacgagggccagcagattccggacgatctcgacggtgccgccgcaggacagcggggcgaagagttcgaccacggagacgtcgaagctgagggacgtggacaacacgacgtgccggagccgctcggcacccaggtcttcccgcgcccaggcgaggaagttggccaggctgccgtggctgatgacgacgcccttggggcggcccgtggacccggaggtgtagatcacgtacgcggggtgggaggcggacatcttcacgtcgggccctgtggcgggggctgcgaccagggtggcgcgggtcgcctcgtcgtccagctcgacccggggaacggtccgcagagtacgtacgtcggtggaggtcacgaccacgacgggggcggcgtcggtgaccatgtggaccagccgctcggccgggtactccgggtccaggggaacgtacgcggcaccggacttgagcacggccagcagggccacgatcagatccagcccacgcggcagcgccacccccacgaaatgctccgggcccgcaccgcgccccaccagcagcccggccaggcggttcgcccgcgcgtcgagttcccggtacgacagaacctcaccctcgtacacgaccgccggcgcgtcaccccgggccgcgacctgcgcctcgaacaactccaggaatgtttccggcacaccggggacgcccgtgtcattccactccgtcaggacacgggcgcgctccgcctcgccgacgaccggcagttcacccgtgcgtaccgaagcgtcggaagccacgcgctccagtacccgcaccaggcgctccgcgagctgcgccaccgtcgggtggtcgaagagatcgcagcggtactccacggctccgcgcagctcgccgcccacctcggagagaccgaagagcagatcgaatttgaccgctcccgcacccagcggcaggccttccaccccgaggcccgcgaagccctccgtgacctcacggtcaatcgtgttgtcgaaggtcagcatgacctggaagagggggtggcgggcgagggagcgctcggggttgaccgcttcgaccagtcgttcgaaggggatgtcctggtgggcgtaggcgtcgaggtcggtggcccggacgcgggccagcagctccgcgaaggtcgggtcaccgctgacgtccgtgcggaggacgagggtgttcacgaagaagccgatgagatcttcggtggcctggtcggtgcggccggcgatcggcgtcccgatcgggatgtcgtcgccggcacccagccgggagaggagcgcggccagcgccgcctgcacgaccatgaagaccgtgacaccctcggcacgggcgagagccaccaggccgtcgtacacaccagcggggacggtgaactcgaccttgccgccttcgtacgaggcgacggcgggacgggcacggtccgtcgggagcgccagctcggcaggcagcgaggctagctgggtgcgccagtaggcgagctgggccgacagctcgctcgactcgtcgtcctccgtgccgagcacgtcgcgctgccaggccgcgtagtccgcgtactgcacgggcagcggacgccagtcgggggctgcgccgttcgtacgggcctcgaaggcgcgcttgacgttccggatgaacggcacacgggacatcgcgtcgaaggccacgtggtgggccacgaccaggaggatgtggtcgttgtcggtgaggcggaagaccgtggcacggaccgggaggtcggcagcgaggtcgaagggccgaccagcggcctcgtcgagccgctcctgcagggcgtcggcaccgatgtccaccagagggagaccgggacgggtgcgagggtcgtccacgggcaggatgtgctgccaggccgtacccgcatcctgcgcgatgagggtgcggaggctctcgtggcgagcgatgacgtcgatgagcgcctggtgcagcgcctcgacgtccagcacaccggccagacgcagcgccatcgggatgttgtacgcagcaccgggaccctccagctcctccaggaaccacagacgctgctgagcgaagctcagcggctcccgacccgtccgctcccgtgccaccagcggcacccgcaccccgagagcgctccgctcccgcagcagccgctccagacggctcaccgtcggcgcctcgaagagcgcacgcacacccgcctccacaccgagcaccgcacggacacgcgagatcagacgcgtcgccagcagcgagtgaccacccaacccgaagaaatcatcgtccacccccacccgctccagcccgagcacttcggcgaagagaccgcacaggacttcctcacgcgggctaacaggcgcccggtagcccgcgcccgcctccacccccggcaccggaagcgccgcccggtccaccttcccgttcggggtcagcgggatctcgtccagcacgaccacggcgctcgggaccatgtaggcagggagacgctccgcgaccgcacgcatgaccgcgctgctgaggccaccggtgtcgacacccgtcacgtacgccaccaggcggtggtcaccgggacggtcttcccggacgaccacgaccgcacgcaccacgtccgggtgggctgccacggcaccctcgacctcacccaactcgacccggaagccacgcaccttgacctggtcgtccgcacgacccacgaactccagggcgccgtccacccgccaccgcaccagatcccccgtacggtacatacgctcacccggcgcaccgaacgggcacgccacgaaccgctcggccgtgagaccggcacgaccgaggtacccccgtgcgagaccgacaccggccacgtacagctcaccgacgacacccaccgggacgacccgcagcgccgcatccagaacaaacatccgctgacccgcgaaaggacgcccgatcggcacaggcccatccggaacctcctcaccaggcgcgataaggaactcggcacagttcaccgtcagctccgtcggaccataagcattcaccaccaccacacccggattacgctcacgccactcacgcagcgcaccacccgacaacgcctcaccccccaccagcagcatgccgttccccgccaccacaccctccagctcacccagcaaacccagatgcgacggagtcaccttcacgaacgaggcacccaccccctgcgccgactcgtccaactcacccaacacaacacaaccgccagacaccaaaggcgtgaacagaaccgtcaccgtcagatcgaacgcaagcgacgaatgcacaaaccccacaccaccaacggcacccgcatacacatcccgaccacgcgccaaatacccacccaccgaagcatgcgtcaccacaacacccttcggccgccccgtcgaacccgacgtataaaccacatacaacggatgccccggcaacagacgcgacgcccgctcaccatccgtcacattccgaacctcaaccccatccgcagccaccaactccacaagacacgactcacgaacaacccgcgaaaactcgaaaacctcacccccaggaaaatcccccaccacatgcgacaacccctcccccaccacaaccagccccaccccggcatccgccagcacccaccccacccgctccaccggccactccggatccaccggcacataaaacccacccgccttcgccaccgccagcaacatcaccaccacatccaccccgcggcccatcaccacacccacaccacactccgcccccacaccccgacccaccaaccaccgcgccaccacattcacccgcgcattcaactccccataactccacaccacccccgcgccacgcacggccaccgcgtccgcgtcggcccgcacccgctcctcgaacaaccccaccaacccaccacccggcaccacaggcaccggcccacccacaccccaccccaacaacctctcccgctcacccacacccaacacatcaacccccgacacagacaccccacccccaccagaaaccacaccctccaacacccgcaccaaccgctccgtcgtttcccgggcctgtaccgcaccgaacacatcaagccggtattccagcaccccgcgcattcccgaggccgcaccgtccgcggcgtacatctccgtgcacgtgaaagcgagatccaccttggtctgatccgtccggatggcctcaccggagacctccagttcccccagattccccagcgcgcttccgaccccgtccatgtccgggacgttgaacgtcagcatgacctggaacagtggatgccgtgccagtgaccgctccgggttcaacacctcgaccagacgttcgaatggcacgtcctggtgtgcgtacgcgtcgaggtcggtggcccgcacccgcgcgaggagttcggcgaacgtcgggtcgccggacacgtcggtgcgcagcaccagcgtgttgacgaagaacccgatcagttcctccgtggcctcgtcggtgcggccggcgaccggggtgccgatggggatgtcgtcgccggcgccgagacgtgacagcaggacggcgagggcggcctggacgaccatgaagaccgtcgcgccgtgggcccgtgccaggtcggcgagggcctggtggagtccggcgggcacggtgaattcgacgcggtctccggcggtggaggcgacagcggggcgtgggcggtcggtgggcagcgtgagttctgcgggggcccctgcgaggcgggtgcgccagtgggcgagttgggcggagagttcgccggtctcgtcgtcctccgcgcccagcacctcccgctgccagagcgcgtagtcggcgtactgcacgggcagcggttcccagtcgggtgccctcccggccgcccgcgcccggtaggcctcggccacgtcgcgtgcgagcggtcccatggaccagccgtccccggcgatgtggtgcaggacgacgaccagtacatgttcctcgtcgccgacgcggaacagtgcggcgcggaaggggatctccgccgccaggtcgaagggcctgcgtacgaactcggtgatgagggcacgcagggtcgtcccgtcggccccggtcacggtgagcagagggccgaggccggtcgcgggcagcacgatctggtgggcaccgtcccggtcctcctcgaacacggtgcgcagcgcctcgtggcgttcggcgacgtcggtgagtgcggcgcacatggcggtgctgtccagggggccacgcaggcggagtgcgacgggcatgttgtacgcggcgctgtcgcggtcgagccggttgaggaaccacaggcggcgctgggcgtacgacagcgggatccgtgcggggcgcggtccggcggtgagcgcggggccggcctgcgcggcccggtggagggcggtggccagggaggagaccgtgcggtgctcgaagagggcgcggacgttgagttcggttttcaggttcgtacggatacggctgacgagtttgctggccagcagcgagtgcccgccggcgtcgaagaagtcgtcgtcggcgccgaacgagggaagcccgagcacgtcggcgtacaggccgcagaggatctcctcgacgggtgtgcgggggcgctcggggcggggcgcggtaccgaaggcgggctccggcagggcgccgcggtccagtttgccgttgggggtgagcgggagggtgtcgaggagaacgaaagccgagggcaccatgtacgcgggcagccgggcggcgaggtgcccggtgagttcccgcgggtcgacgggtgtgccggggacgacgtacgccacgaggcgagcctctccggtggcggagcggtgcaggaggacgacgttctgggcgacggcggggtggccggccagggcgagctcgatctcggcgggttcgatgcggaagccgcggagtttgatctgatcgtccacgcgtccgaggtattcgaccacgccgccgggactgcgccggacgaggtcgccggtgcggtacatgcgttcgcccggcgcgccgaaggggcaggccacgaagcgttcggccgtcaggccgggccggccggcgtagccgcgggcgagctgggtgccggcgaggtagagctcgccggcgacgccttcgggtacgggacgcaggaagccgtcgaggatgtaggcggccgtgttgtcgagggggtggccgatgacggggcggggtgcgatgccgaccgtgcaggcgagggcgtcgacggtgaattcggtggggccgtagtagttgtacgaggtgacgccggggacgtcggagagcctggtccagagtttcgcgtcgacgccttcaccgccgagcatgaaggcggcggggtggtggcggccctcgtcgaggattccggtctctacgagttgctgggcgaaggagggggtgacgtcgatgacgtcgatgcgctgccggtcgatgagttcggcgacgcgttcggcgtctcggcgggtgtcgtcgtcaataaggtgcagttcgtggccggcggccaggcagatcagaccttcccaggaggtgtcgaaggagagggaggcggtgaggccggccttgagccgtgatcctccggcggcatggggttctatgagggcgcgccgatggtcgtggaagaggttggcgagactgcggtgttcgatgacgacgcccttggggcgtccggtggagccggaggtgtggatgatgtaggcggcgttgcgggcgttgagcggggtacggcggtcggcgtcggtgaggttgtgtgcgggtgccgcagcgagggcgcgggcggtggtggggctgtcgaggacgagggtgcggccggggtgcggcagcggggtggcggtggtggtcgtgaggacgacggtcgggtgtgcgtcggcgaggatgccggtgagccgttcggcgggctggtccatgtcgagcggcaggtaggcggcgcccgctttgagtacggcgaggatggcgacgacggattccgcggaacgggggaaggcgagcgcgacgaagtcgtcggagccggcgccggcggcgacgagcaggcgggcgagccggttggcgcggtcgttgagttcttcgtacgtccaggaggtgctggtgaagacaagggccgtgtcgtggggccgcctccgcgcctgttcctcgaattcctggtggtagtaggtgggcggctgctcgcgcgtggtgtcgttccagccgcgcagcaggcggtggcgttcgtcgtcgtcgaggaggtcgaggcgggcgagggtgtgcccggtgtcgtcggtggcggtttccaggacgcgggtcacgcggcgcagcagggcggtggcggtggtggtgtcgacgaggtcggggcggtggtcgagcttgaggcgcagggtttcgccgggtacggcggtgagggtgagggggtagtgggtggcgtcggagccttcggtggcggtgacggtgaagtcgtcggtgcggatggagccggggccgctgtggccgatggggtagttctcgaagacgacaatggtgtcgaagagttctttgtgtccggcccagcgctggatgtcggagagccgcacgtgctggtgttcgaggagggtggtctgttcggcctggaggcgagcggcgaacgtgccgagggtgtcggcggggtcgaggaccgtgcgcagggggacggtgttgatgaagagtccgaccatgtgctcggcgccggcgagttcggcgggccgtccggagacggtgacgccgaacacgacgtcgtcgcggccggtgtgctggccgagaacgagggcccacgcggcgcggacggcggtggcctgggtgatgccgcggtgcctggcccaggcggcgaggttctcggtgccggccttgtcgagttcggcgtggacggaggccggcacgacgccgtcgggggaagcgccgggtgcgaccagggagggctcgtccaggccggtgagcgcgtcgcgccaggcggtttcggcggcggcgtcgtcgcgcgcggcggcccaccggagaaagtcgccgtaggggcggacgggcggcagcaccgacgggtcggcgccgcttccgtacagggcaaggagttcgcgcagcagtaccggcagcgaccagccgtcgaggatgacgtggtggtacgtgatcacgaaccggaaacggtcgtcgtcggtccgtacgagcacgaaccgagccagcggggcccggcccaggtcgaacgggcgccagcgttcctcccgggccacggcggcagcgtccgtggcaccgtcgcgggcgtcgacggcgcgccatggcgtcgcgacgcgccgcgccacgaccgcgacccactccccggagcgcgcctgacggtagcttgagcgcaggacgccgtgccgcgccacgaggctttcgacggcggcccgcagccgaccggtgtccaccgcgccgaccaggtcgaacgcgagctgtcccgcgtacacgtcgactccgtcggcggcggcgaaggagctgtggaacagcagtccttcctggagcgccgagacaggcaggatctcctcgactaccttcgaccggcggttcaccactgctcttcctccccctcgagtccgagctccagctcgaactcctcgaattcgtcgtcgctgatcgaaggcagtacgtccgtgccggggacggtgtcgtccgcgtcgtcggccggtgccggaagggccgcggcggcgagtgcgccgaccgtcccgtgctcgaacacgtctcgtacggtgatgtgcagtccttccctccgggcgcggctcacgagctggatggacagcacgctgtcgccgccgaggtcgaagaagccgctgtcggctcccacgtcgggcaggcccaggatgtccgcgaagagcccggcgaggatcttttcctcgcgggtggcgggggcgcgtccggcaggggtgccgaacacgggggcgggcagcgcggcgcggtcgagtttgccgttcggggtgtgaggcagggtggtgagagtcacctgggcggaggggaccatgtagtcaggcagaagttcgcgtaccgtctcgcgcagttcgccggcgtccggtccccgggtgccgggacgggggacgacgtacgcgacaaggcggccctggccgcggtggtcggggcggacggcggcggcggcgcgccccacgtcgggatgccgggcgagggcggcctcgacgtcgccgagttcgatgcggaagccccgtaccttgacctggtcgtccacgcggccgaggtgttcgagcgtgccgtccgggcggaaccggccgaggtcgccggtgcggtacatccgctcgccgggggcgaacgggttggcgacgaagcgttcggcggtgaggtccgggcggcgcagatagccgcgggcgagcccggacccggcgatgtggatctcgcccggaacacccgggggaacgggtccgagggccgcgtcgagtatgtggacgcgccagttgtccaggggggtgccgatgccgggtgtgcggtcgtcgccggcggtgagtggggcggcggtggcccagatggtggcttcggtgggtccgtacacgttggtgacggcggcgccggttgcggtcaggacacgggccaggtcggcgggcagcggttcgccgccgaccaggatccgtacgtgctcgagccgggcggcggcgtcggccggtgctccgtcgagcatggcccgccaccaactgggcgtggcctggacgacggtgacgtggtggcgtgcgcacagggaggtgatcgaggcggggtcgcgtacggcgtcctcgtcggccagcacgatcgcggcaccggcgaggagcggggcgaacagttcgaggccggcgatgtcgaagccgacggtggtcaccgcgagcaaccggtcgtgcggggtcaggtcgtggcgccggaccgcgtccgcgaggaaggcgcgcagggaggactcggcgatcgcgacgcccttgggcctgccggtggagccggaggtgtggatgacgtacgcgaggtggtcgcccgacggggacgtgggaaccgcgggggcggctgcggcgggcggctgcggctcgtcgacgaggacggtcggcgcgaggccggccggcagccgtccggcgaggccggcgtgggtgaggacgaggtcgggacgggcgtcggagagcacgtgcgcgagtcgctcggcggggtacccggtgtcgagcgggaggtaggcggcgccggtgcgcaggacgccgagcagggtggtgaccaggccggccgtgcggggcagggcgacggcgacgaggctctcgcggccgatcccccggtcggccagcagccgcgccgtctcaccgctgagcctttccagttcccggtacgagagttcctcttccgcggtgatgacggcgacggcgtccgggtccagcgcggcccggcggacgatctgctcggccacgctgcgcgggggcagcggggtgtgcgtaccgctgccccagtcaccgaggatgcggtcccgctcgtcgtcggtgaggagcgggacggtgcccaggggggcggtggggtcggcggccagtgctgcgaagaatccggtcagccggtggcccagtgcggcggcagtgccgtggtcgtacaggtcgctgctgtagtcgagcagaacggacaggccggcggcgtctccgtcgggggtgcggtgttcagtgaaggtgaacgacaggtcgaacttggcggcactggcccccagcgggtactcgtccccggtcaggcccgggagggcgttgagtgcctggccggtgatgtcctggtcggcgttggtgagggtgtaccagatctggaacagcgggtggcgggcgagggaacgccgtggcgcaagcgcctccacgagccgttcgaagggcacgtcctggtgcgcgaacgcgtcgaggtcggcggtcctgacccggtccaccaggtcggcgaaggacggatcccccgcgagattcgtccgaaggaccagggtattgacgaaaaacccgacgacgtcgtccagcgcttcatcgccccgcccggcgaccggggtgccgatggggatgtcgtcgcccgcgccgagccgggacaacagaccggcgagcgcagcctgcaccaccatgaacagcgtggcccgacggtcacgcgccacggccagcagactccggtgcacgccggcgggcagttcgaaccgtacccggccgccccggtgcgaggaccgaacgggccggggccggtcggcgggcagcgtgtgctcctcgggcagcccggccagcatctcgcgccagtacccgagccgacgcgccgtccggccgtccggggcgtcctcgtcgcccgcaacagcgtgttgccacagggcgtagtcggcgtactgcacgggaagcggggtgtactccggggcgtcgccccgcgttcgggcccggtaggcggtgaccaggtcacgcaccaacggccccatggaccagccgtcgccggcgatgtggtggatgaccaccacgaggacgtgttcctccggtgagacgcgcaacagggtggcccgcagcggcgtatcgctggtgatgtcgaaggcgtacccggtggcggacgccgcctgttccgccacctctccgacggtcacctcgtgcgtgaggaaggggggttcggcgtgtgggagcaccagctggcggggttcgcccccgtcgtcggggaagaccgtacgcagtgattcgtgccgtgcggtgacgtcccccagggcggcccggagagccgggacgtcgagggcgccggtgagccggacggcgaccgggatgttgtaggtggcgctgggcccttccagcttgctcaggaaccacagtcgccgctgtgcgaaggacagcgggagccgttcagggcgccggtcgatcctggtcacaggtggcagggcggtgccggaggccgccagtacggtcgcgagcccggcaggcgtgggcgcggcgaagaggtcgcggatgccgagttccacacccagggtgcccctgacagccgacaggaggcgcgtcgccagcagggaatggcctcccagggcgaagaagtcgtcgtcggccccggcggcgggcaaccggaggacgtcggcgaacaactcgcacaggatctcttcctgcggcgtccgaggcagtctccgggtgcgggttccgccctgccactgcccggccagcgccgcgcggtcgagctttccgttggccgtcagcggcagctcgtccagggtccggtacgcggacggcaccatgtacgccggcagggtcgccgcgaggtgctcgtggagttcggccagggtcggcgcccggtgcggcagcgcgggcaccacgtgcgcgacaagccgttggtcgtcggtctgctcgtcccgcaccaggaccaccgcctggacgacacccgcgtgtgtgaccagcgcggcctcgatctcgccgagttcgatccggaagccgcggatcttcacctgccggtccccgcggcccacatactccagtcccccgtcgggccgcaccttggccaggtcgcctgtgcggtacagacggccgccggaacgggaattcgggtcggccacgaaccgctcggcggtcagagcggggcgtcccagatagccgcgggccagaccggcgcctgccacgtacatctcgccgacggtgcctgggggcaccgggcggagccgttcgtcgaggacgtacacctgcagatcggccaagggccgaccgatcgggctgccggaaagactcgtcgcgcgatcctccagcgggagcaccgtgacgtggacggtggtctcggtgatgccgtacatgttcacgagcgtcggccgtgagccgagcggcaggtcgaaccagtcgcgcagtcgcgacgggtcgagcgcctcgccgccgaagaccacatgtcgcagggccagcccggcgcagcgcgccggctcctggcgggcggcctccgtcagctgatggaacgcggacggtgtctggctcagcagcgtgacctgctgctcggcgagcagcgcgaggaattcggcgggagaacgagtcacctcgaacggcacgacgacgagccgcccgccgtgaagcagcgcgccccagatctcccagaccgagaagtcgaaggcgtacgagtggaagagcgtccagacgtggtccggtccgaagtcgaaactgtcggagcatgcggtgaacaggcgtacgacattggcgtggctgatgacgacgcccttggggcgaccggtggagccggaggtgtagatgacgtaggcggggtgggcggggccgctcccgacacagggggctgtgtcgggagcggcggcgagttcggtgcggattgcctcgtcgtcgagctcgatgcggggggcgatcagggggatgtgtacgtccgttgaggtcacgaccgcggtggggttggcgtcggtgaccatgtacgcgaggcggtcggtggggtattcggggtcgagcggaacgtatgcggcgccggttttggtcacggccaggagtgccacgatgaggtccaggccacgcggtagtgccacgcccacgaaccgttcggggcctgcgccgcgctccatcagcaccctggccaggcgattcgcccgcgcgttcaactccttgtacgacagctcctctccttcgcacaccaccgcgaccgcatggccccgagccgccacctgctcctcgaacagctcaggcagtgtggaggtgtgaagttcctcggctgccgggggagcctcggggctgagggccttcacctcgtcggccgacaggatgccgatgtcgcccacggacagctcgggccggcgggcgatctccgcgaggaggcgcaccagacgttcggtcatggcctcggccgtggagcggtcgaacaggtcggttgcgtagtccaggatgccgggcatctccgtgctgccgggtgtctccacgaacgagaacgcgagatcgtaggggctggtcacagccaggacgtcggccggtcgtgcgtgaaggccgggcatggcgtcgagcgcgtcgagcgcggatcggcggtccgtgttgttgaaggcgagcatgacctggaagagggggtgccgggtgagggatcgttcggggttgatggcttctaccaggcgttcgaaggggatgtcctggtgtgcgtatgcgtcgagagcagtggtgcgcacacgggccaggagctcggcgaaggtcgggtccccggagacgtcggtgcgcagtacgagggtgttgacgaagagtccgatcaggttctccatcgcctggtcggtgcggccggcgatgggggtgccgatggggatgtcggtgccggcgccgagtcgggacagcagggccgccagggccgcctgcaccaccatgaagacggtggcgccctgggtgcgggcgagtgcggtcaggccttggtgcacgtcgtggggcacggtgaactcgacgcggtcgcatgcgtgggaggccacggcgggccggacgcgactggtggggagggtcagttcggcggggaggccttcgagtttgctgtaccagtagtcgagttgggtggagagttggctcgatgtgttgttctcggtgcccaggacgtcgcgttgccaggcggcgtagtcggcgtattggacggccagagcgggcagttgcggtgcgtggtgctcgagtctgacggtgtaggcggcggacaggtcgtgggccaggggggccatggaccagccgtcgccggcgatgtggtggagcacgagcaggaggacgtgctcgtcgtcggtgacgcggaagacggtggcgcggagggggatctcggtggtgaggtcgaacggatggcgggcggcctcgtccacccgattctgcagggtgtgcgcgtcgaccggcatggtgtcgagggtgaggtgggtgcgggtgtcgtcggtgggcaggatgttctgccacacgccgcccgaatcgtcctgggcgatgaccgttcgcaggctttcgtggcgggcgatcacatccgtcagggccgcttccagcgcggtcaggtccaggcggccggtgagtcgcagagccatggggatgttgtacgcggcgttggggccttcgagctggtggaggaaccacaggcgctgctgcgcgaacgacagcggctcacaaccggtgcgctcccgcgcgaccaggggtgcccggaggctggtggtctgttgctggagcagctggtccaggcggccgacggtcggggctttgaagagggctcggacaccgaggtcgatgccgagggccgaccggatccgtgaggtgaggcgggtggcgagcagggaatggccgccgaggtcgaagaagtcctcgtccacgccgacctgatccagaccgagtacgtcggcgaacagggtgcacaggatctcttcacgggctgtgccgggtgctcgggaggtggttccggaggcggtcccgggcgcggggagggcggcgcggtcgaccttgccgttgggggtgagggggatctggtccagtacgacgacggccgacggcaccatgtacgcgggcaggcggccggcgacggtgtccgtcaccgccgaggacaggccgctcgggtcgctgcctgtcacgtacgcgaccaggcgctggtcgccgggccggtcctcgcgtacgacggcgatggcccgcgccacgtgcggatgcgccgcgacagccgcctcgacttcgccgagttcgatgcggaagccgcggatcttgacctggtcgtcggcacggccgatgaattcgagtgttccgtcgctgcgccagcgtgcgaggtcgccggtgcggtacatgcgttcgccggggctgcggaaggggcaggccacgaagcgttcggcggtcaggtccgggcgcccgagatagccccgcgccaggccgtcaccggccacatacagttcgccgatggtgtcggggggtgtgaggcggagggcggggtcgaggacgaagaggtggtggccggtgaaggggcgtccgatggggacggggccgtcggggaggcagtggccgggggggatgcggtattcggcgcagttgacggtgagttcggtcgggccgtaggcgttgatgaccgtggtgtgggggtggtgggtgcgccagtggtgcagggcgccggcggtgagggcttcgccgccgaggaggagggtggcgtcgggggcgagtacccggtcgagttggccgagcaggggcaggtgggaaggagtggccttgaggaaggtggcgcccaggccgttggcggtgtcgtcgagatcgccgagggtgacgcagccgccggagaccaggggggtgaacagaacggtcacggtcaggtcgaacgcgagtgaggagtgcacgtggcccaggccgtcggcggcgccgcggtaggcgttgcggccgcgccgcagataggcgccgaccgaggcgtgggtgaccaccacgcccttgggcaggcccgtcgagccggaggtgtagatgacatacagcggatgcccggacagcagagccgacacgcgctccccgtccgtcacatcaccggcgtcggtggccgcctgctcgttcagccgtgtttcgcgggcgatctgcgcgtagtcgaccacctcgcagtcgcggacggcctcgaccgcagcggccaggttctcgcccacgacggccaggtcgatgccggcgtcggcgagtacccagtcgatgcgttcggtgggccattgcgggtcgacgggcaggtagaagccgccgcttttggcgaccgcgaggagcatggccaccacgtccgggccgcggtccatcaccaccccgacaccgcgctcgggtcccacgccccggccgatcagccaccgggccaccgcattcgcccgcgcgttcagctccgcgtaactccattccactcccgcgccgcgcacggccaccgcgtccgggtcggtccgcacccgctcctcgaacaacccgaccagccccgcacccgcacccgcacccgcacccggcagcgcacgcacgccgggttccgcgcccgcaccccagcccagcagcgtctcccgctcaccggcacccagcacatcgacaccggacaccggcacatcacgctcgcagaccacgcactccaaggcccggacgatccaaccccacagctcacgcacccgacccgcgtcaaacacgtccggccgatagccgagccggaatcgcatggcgggaaggggaaccactgccaggttcaaagcgaaatgcgtcgcatcggcaccctgtattccgtcgatctgcaggccactgccgtggaacagtgaatcctgcagcagggaatccatcgggtagttctcgaagaccattccgacgtcgaagagttccttgtgtccggcccagcgctggacctcggccagccggacgtgctggtgctcgagcagttccgtctgttcccgctgcagccgctccacgaaggcgcccagtgactccgccgggtccatccgggcccgcaacggaatggtgttcacgaacagaccgaccatgtcctccacacccgccacttcggcgggccggcccgacacggtcaggccgaacaccacgtcgtcccggcccaccagccgccccagcaccaacgcccaggccgcctgcaccaccgacgcgaccgtcacaccacgcccacgcgcccacgccaccagttccgaggtcagttcctcgctgatctcttcctcaaggacttcgggaatcaccccactgccctccgttccgagggcgagcactgtgggctccgcgagtccggccagcgcggaacgccatgcctgtcccgcagcggtctggtcgcgttcggcgagccacgccatgaagtcacggcgcggtggggagtacggaacctccggccggcggccgagggcggtgtcgcgataggtggtgaagagttcgcgtatgagaaccgccacggaccagccgtcgaggatgacgtggtggtacgtgacgatgaagcggaagtgggtgtcgttgatgcgtgcgagggtgaatcgtgcgagtgggccctgcgtcatgtcgaacggccgccagcgctcgtcgtttgtgagggtgtcggcgtccgtgtcgagtgtgtggatgtactgccagggggtgtggacttgtcgtgccacgacggcgatccattcgccggactgtgcctggcggtagccggtgcgcaggacgtcgtggcgtgtcaccaggctttcgaccacggcgtgcaggtggtcggcgtcgactggtccggtcaggtcgaacgtgagttgtcccacgtagacgtcgaccccgtcggcggcgaaggagctgtggaagagcagtccttcctgaagcggtgatacgggcaggatgtcgtgcacgccaccggtgacgtcggcctgcagggcctccagttccgcgtggtcgagggcgaccagcggcaggtccgagggggtcagtccgcccgcgtcgggacggcgggcgtgtgcgaccagcccttcgagggcttcgtcccagaagcgggcgagttctcgtatccgggattcggacagcagtgtcgtcggccacgaccatgtcacgctgaggcgggggccgtcgggtgtgtccagggtgatcgcgttgaactccaccgcgtgggccaccggcagaacggggtcctgacccgcgcccaccgcgaacggacccgacaccggaacccagtcggcccactgttcctcgtcggagtccgccgctgccctgccgccgacgaggccgccgagcccgtcggcatgggcgtcgccttcgtccagtgaggcagcgtccgccgagaccgagacccgtccgaggtagttgaagccgaagtgcggggtggggtgctgggcgagggcgggtccgttttcggggtcgaggtagcgcaggatgccgtagcccaggccctggtcgggcagggtgcggaggcattccttgatctcgcggacggtgcgtccgacggcgggaccgccggcgcgtatctcgtcccaggccgccggctcgagggggaggcggacggggtagatgctggtgaaccaccccacggtgcgggagaggtccgcgccgggtacggcgtcttcgttgcggccgtgaccctcgacgtccacgacgacgcttccgccgcggccgcgccagcgcgtcactgccaggccgaatgccgccagcagcacttcctgcacgcccacacccaggaccgacgggacctccgtcagcagggcacgcgacacccccaccgacgcccgtgaacccaccgactccaccacgccgaagacgtccaccgaagggtccaccgcgcgtgggccgaccagagggtccgagccgtggaccatctgtgcccacaggtttgcttcggtggcgcgggcgccgtggcggccctgttccgccagcgccgccgcccagccgcgcagcgatgtgccgaccgtgtccaaccgcacatgcccgccagcgcgtgcctgtgtccaggcttcgccgaggtcgccgaggacgatgcgccaggagacgccgtcgacgaccaggtggtgcgccaccaggaccaggactccccggcggtcgggtccacggtccagccataccgcgcggaccatccgtccggtcagtggatcgagccggctccgggcctccacggcggcggcccgcacggcgttcacctgctcctccaggtcggcgctggccatgccgtgccgggtgatccagtcgggctcaccgccgggggcgggcgcgtcgacgcgcagggaccaggcgccgtcaccggaatccactacccgtagccgccaggcgtcgtggcgttcgcgtaccgcgtccaggaccgtccgcagttcgtcccaggtgataccggccggtgtggcgaccacgacggactggttgaaggccctgaccgcaccgccgagcccaccgccaccacgctccgccagccactccaccaccggccaccgctccaccgcacccacaccggagtccacgacggcggcagcgacgcctccggactcccgcaccacaccggccagcgcccgtacggtgcggtgctcgaaaacgtcccgcaccgacacctccagacccgcccggcgcgcccgcgccaccagttgaatggacagaatgctgtctccgccgaggtcgaagaacccgtcgtccacgccgacccgttccacgcccagcacttccgcgaacagggcgcacagagcctcttcacggggtgatccggtgacgcggaactccgcggccggggcgacgcgcggcgcgggcagcgcggcacggtccaccttgccgttcggcgtcaggggaatctcgtccagtacgaccacggccgacggcaccatgtacccgggcagacggcccgcggccgtctccacgatcagcggtgccagcccctccgcctcgtcaccggcggccgtcacatacgccaccagccggcggtctcccggtcggtcctcccgtaccaccaccacggagcggtccacacccgggcaggccgcgacagcggcctccacctcgcccagctcgatccggaaaccccgcaccttgacctgatcgtcgacccggccgacgaactccagcatcccatccgcacgccaccgcaccaggtcacccgtgcggtacatccgctccccggcatcaccgaacggacacgccacgaaccgctccgccgtcagacccggctgcccgagataaccccgcgccagcccctcgcccgccacatggagctcaccgaccacaccgaccggcaccgcccgcaacccaccgtcgagcacgaacacccgcgtgttccgcaacgggcggccgatcgccaccggccccgcaccgatcggttgtcccggatcgatacggaagtccacgcagttgaccgtggcctcggtggggccgtagtcgttgaagacgacggcctccggcgacccggcccgccacttgtccagctcccttcccagcaactgctcgcccccgacgagcagcaccttcgcggacgcggcccatgtcgcctcgtgcgtgagcagtggcagatggctcggtgtggccttgacgaacgtcggcccgcccgccgcggccaggctcggtgggcacgcctcggtcaggtccgcgacatggatggcgccaccgacgaccagtggcgggaacagcgaggtcacggtgaggtcgaaggtcacggggctgtgcagcagggtgacgtcgaaggctccggggtacgcggtcacggagtaccgcacgaagtcggccagtgaacggtgttccaccgccacacccttgggccggcccgtggagccggatgtgtagatcacgtatgccacgtgctcgccgctgaccggagtgacgcacgtgcggtcccggtcggtgaccggaccccccggttgttccgcgatcgccgcgtggacctcgggttcgtcgaggcggagctgtgggatgtcgcgccgcggcagttcacgggccgcaccggcgtcggtgaggatcacggccggccggcagtcgtcgaggacgacggcggtgcgctcggcggggtgggtgaggtcgagagggacgtaggcggctccggtcttgaggaccgcgagaagtgccaccagcagctcggcggaccggggcagggcgacggcgacgagacgttcgggaccgacgccgtgggcggtgatcaggtggcgggcgagccggttggcccgggtgtgcaggtcggcgtaggtgaggacgtggtcgttctcgacgacggccggccggtcgggggtccgcgccgcacggctttcgaggagatcgagcaccgtgcctgggggggtggggtgggcggtgtcgttccattgggcgaggagtgcgcgttcgtcgtcgccgaggagttcgtacgaactgatggggcggtccggtgctccggcggcttgctccaggacggtgaggtagcgctggaccaggcgctccgcggtgatccgcgtgaagagatcggtgctgaactcgagagcgccgtcgatgccgaggccgtccgcgtgctcggtgaggaagaacgacaggtcgaacttggacatgccggtgtggacggcatggacgcggctggtgaggcccgcgaagcgggacccgtcggcggtgtcgttcgtcgccgggccgttgtacgcgatcatgatctggaagagcgggtgccaggacagggaccgctccggtttgagtacgtcgacgagacgctcgaagggaacgtcctggtaggcgtacgcgtccaggttggcggcccgcacccgcgacacgagttccgcgaacgtcgggtccccggagacgtcgttgcgcagtacgagcgtgttgacgaagaacccgacgaggtcttccgtgccgtcgtcggagcggcccgcgacgggtgttccgagggggatgtcgtggcccgcgccgtgccgcgacagcagggcggcgagcgcggcctgcatgaccatgaacacggtgacgccgtgggcctgggccatggcccgcagcgtgtggtgcgtgtgctcgggcacggtgaaggcgatcgcctcgccccggcgggtgggaacggcgggccgggtgtggtcggtgggcagttcgagctgctcgggcaggccggccagggcgtcgcgccagtggtcgagctgtgtgctgtccgacggatcgtccagcaggtgccgtcggcgcagggtgtggtcggcgtactgcaggggcagcggctcgaagtgcggggcgtcgccggcgcggcgggcgccgtaggcgtgggcgagatcgcgtacgaggacctcgacggaggctccgtcgccggcggtgtggtggacgaggagcagcagggtgtggtcgtccgggccgcagccgaacaggtgggctttgagtgggatctcaccggcgaggtcgaaggggcggcgtgcggcttcggcgagggctgcggcgagatcgccgggcgcgacggtgctgtgggtcagttcggggcgggcggcctcggggggcaggatgacctggtgggggccttcggagtcctcggcgaggagagtacgcaagggctcgtggcggttggtgacgtcctgcagggcgacagcgagggccgggatgtcgagcgggccttcgagccggacggcggtggggatgttgtacgcggggctggggccttcgacctggtcgaggaaccacagtcgctgctgcgcgtgcgacagggggaggcgctccgggcgcggaggcagcggcggggtggtgtgctgccgtccgttgtcgtccaggagcgggagaaggccggcgacggtggggaaggcgaacaggtcgcggagggtgaggtcggtgccgaactcggtcctgatccgggcgagcagccgggtgccgttgagggagtggccgccgagctcgaagaacacgtcgtccgtggcgacctccgttacgccgaggacgtcggcgaacagggcgcagagtgtcttctcctgtggggtgaccgggcgttgcccggatccgccggcgcgggcggcggggacgggaagggccgccctgtccagtttgccgttggacgtcagcggcagtgcggggagggggacgagcgcccagggcaccatgtggggcgggagggtacgggccagggccgcacggagctcaccggcgtccggttcggcgccgggcgcgggcacgatgtacccgacgagccgggggtccccggggcggtcctcgcggatcaggacgccggcctgacgtaccccggggagagcggtgagcgcggcctggacctcgccgagttcgatgcggaagccgcggagtttgacctgtccgtcggctcgtccgacgaattccagcatgccgccggagcgccgccgcaccaggtcaccggtgcggtacatgcggctgccgggcgggccgaacgggtcggcgacgaaacgctcggcggtgggcccgggagcagcgacgtagccccgggcgaggccctcgccggccaggtacagttcgccggtcgtcccgtcggggacgggttcgagcgccgggtcgaggacgtagccgcgccggttgagggtggggcggccgagcgggacggggccgtgggtggcaccgtcacggtgccaggcggtcgcctcgacggttgtctcggtgggcccgtacgcgttgacgatgtcccggccccggccccactggtcgaagatctccggcgggcagacctctccgccgacgatcagagtccgcaggtgcgggagatctgtggcgggcatggtggcgagtgcgccgggggtgagggatgtggtggtgacggcctccgcggcaaggaagtcggcgaggtgacggccggtcaggccgtccggcggtatcaccaggcagccgccggacaggagggtcatgctcagttcggagacgaagacgtcgaaccccggggagatgaactgcagtacgcgggcgtccggtcccgtgcggtagcgccggatctggtcggcggcgagggtggccagcccttcgtgagtgacgacgacacccttcggccgtccggtggagccggaggtctggatgatgtacgccgcgttgccgggtgccgggcggcccggggcgggctctgggccgccctcacggaggaggtcggcggcggccaactgcggcgtggggtgggacaggccgtcgatgtgtccggtggtgacgaggtaccggggtcgggtgtcgtcgagtacacgggccagccgctcggccggatgggtggggtccagcggcacgtaggcggcgccggcccgctggatgccgagcaggatcgcgacgctctcggcggacttggggagggcgagggcgacgacgtcctcggtacgcaggccgaggccggccagccgtccggccgcccgttcgacactccggtgcaggtcgcggtaggtgaggacggtgccgtcgtgctggacggcgggcgcgtcgggggtgcgccgggcctgttcggcgaagagttccggcaaggtggcacgcggtacggggcgttccgtgccgcgggagtcgtcgagcagccgggcgcgggtgcccggcgacaggaggcggtgtcttccggtcggcaggtccgggtcggcggtcacagcgtcgaggaagcgcaggaaccgctcctggtggtcggccagatcggtggccgtgtacagggcggggttaccgctgaagaccagctggacgccgtcgggggtgccgggggtgccgtaggagttgatgtggaagtcgcggaccggtcccgaggacaactcgtggaccacggcccggcagtcgccgaaccggatgttgtcgacatacgccaggacgttgacggtgacccgcgccaggcccgcggtgcggccgacgccccccaggttccggtggagttcttccccgcggaaccgctggtggcgcgccagcgtgccgacggcgcgggtggtgtcggcaaggagcgcggcgaacggggcatcgagccgggcgtcgaggcgcagcggcacgtcgttggcgagcatgcacggggtacgcagcgccggcccggtgagccgggcggtgacgggcaggccgatgacggtgtcgtcggcggcagcgtggcggcgtacgaacgcggcggtggcggcgacgagcagtgacgaccagcgagctccagtggccgcggccgaggcggccagcttctccgtccgttcgccggacagctccacggtgcggcgcagcgggacggcgagagcgccggcctcccgctcgctcagcccgggggcctcgtccgcaccggcgagggaccgggtccagaattccccgtcgcgccgatggttgtcggagtcacggtaggccgcctcctccgtgaggacaccggccagggggccgaacgggcaggggccggggtcgtccccgttgctgagggcggtgtagacgtgggcgaggcggcgcaggtggagcagggcaccgtagccgtcgagcgcgatgtggtggtagccgaagtacagcaggtggcgggacgggccgagggtgagcagggcgtgggtggcggtcccggcgcggtcgaggcggaccggtgcggcgagcgccgcgtgcatccagtcgtgtgccgcccgctcggggtccgggtcgccggagaagtcggcttccagcaggagctgtccggcaccgggttcgaggatctgcagcagttccccgtcctgcggtacgaaccgggtacgcagcgcctcggtctcgtcgagggtccgccggaccgccgtgccgagtgtccgcgggtcgatggacccggtgagtgacaggtacgccgcacagtggtacaggcggtggtcgtccgccagctggccggcgagccagacgctctgttgggcggcggtgacgccgaggcgctgcgactgcatgtccatctctcgcggatcccctcaggtgcggtcggccaactggggcatcagtgcctggacgaactcgttgagtgtggggacgctccacagcagctccgggtcgacctcgatcccgtagcggtcctcgacctgcgcgcagagggcaactccggagacggagtcgaggccatggtcggtcaggggtgcgtcaccggcgatccggtcgggtgtctcgttcacgaactcggcgatctgtccggtgatccacgcggtgacctcgctgggcgtgctgaccgcttcgggcgggttcatgtggagctactcctcgctgtcggcgggcaccgcggtgcccgttccagggcagggccgccctccggcgattacgcggggtgcggatgtggagcgtggtcagtgggggcgatgtcctctccggccgtgccggcgatggcgtgccagcccttgtccgcgtggacgatgttcagctccccgcggaggcagaggtcacgcatggcgccccggcggaccttgccgctggtggtacgcagtacggtgccgcggctcaccagggcgatgccctgggcgtcgagtccgaattcgcggttgatcgtcccccgcagggcgctgaccagggccgggtggtcgtcggcggggaccggggtgcggacttcgtggaccaccatcaagcccggcgtgccaggtgccgccgggacggtgaacgcggcgccgacgctctcaagttccgggtgttgcaggcggagttcgtgctcgatgtcctgcgggtacagattgcgtcctcgtatcaccagggcctctttgcagcggccggtgatgtggagattcccttcgtgcagggcggcgagatcgccggtgcgcagccagtgaccgtcctcgccgtcgagggtgaggccgaaggtctgctcggtctcctccttggcctgccagtagccggcgcagacggcggctccctggaggaccagctcgccgacctggccgggcggcagaacacggtgcccgccgggttcgacgatgcgggcgcggaagtggccgacgggaccgcagctgacgatctcccgggctgcctcgccgggtacggcgaggcggaattcgtggcgttcgagggcggcggtggcgactcgtgccgtgtgcagcccctgcgacctcgacacgaacagggtggcctcggccagcccgtagccggcggtgagcgcctcggggcgcaggccgtaccgggcgaaccgttcgccgaaggcccgtacggtggctgcccggatgggctccgcgccgttgccggcccaccgccagcgggacaggtccagcccggcgatctgctcgtcggtgacgcgcttcagacacatgtcgtacgcgaagtcgggagccgacgaccagaccaggccgaaccggtcgatggtccgtagccacaggtgcggccgtttgatgaatgccgtggagctcatcagcacgcacgtggtgccgaggtacagggccggtgtcagcgtgcccaggagccccatgtcgtggtacatgggcagccagccgcccatgggagtggcggcggtcagctcgcaggcctcggcgagattccgcgcgttcgccagcaggttggcgtgggtcacgaccacgcccttggggttgccggtcgagcccgaggtgtactggatgagagccacgtcgtcgggcgcgacgcccgggtcgtcccactgcgccgggtcgccgagcgccgcccggtcgtccacggccacgacgcgagtagccggggccgtggtctcggtcagccagtcgtggacggaggcggtctcacccgcgacggtcagcaccacgccggctccgctgtcggcggcgatggccgcgacacggcggcgttcgaagtggcgccccccgggcagcggcgccggtacggcggccagacccgcgtacaggcagccgaggtacgcggccgtgaactccgggccggacggcagcagcagaagtgcccgtccccccggcgcgagttgctgtcggagggtcaccgcgatgcttctggcccactcgtccagctgtccgtagtccaccgacgccgtgtcgtcggtggttccggtgtcccgtacgagaaccacggcagtctcccgggcacgtgtccgtgcccaggtccgcagtgcccccaccaggccctgcccggcacagcggctctcactcacggcaggcactccccctcctgactccggtcgaacgacgcatccgcgctcgcatacctgtaaggcagggagtcattccgcgtccggcttgcgaccggtcgccgctgcgtgaggtgtgccgtcccgtcatcaggcatgtgcgaatgccctgttgccgtcatgtgaggtccaaccgcttcggcggaccaaaccacttgatatggacgaaacaccaaccccccctgtaacgcctgtggttggtgtgcctgttggttcctgtgcgacttgatcgccagtatgttccacgcccctgcgaacgtcaatgggtcccggcagggacgatctttgcgtatgcggcgtttacgcacgttctcatctgcgcaaacgtcacactgggcaggtggtggggaggggtcagggcgtccggtgcgggccgcccgcacccggcgggcgccgcagcccgcgtaccgccagcagaccggtcaccgccaagagcgcaagactggtgccgacggccgcgagcgccgcgcccagcgcgctgtggtccagcgccagatcgcgggacgcctccaccgcgtacgtgaggggattgaccgtcgcgatgaccctgagccatcccggcagaatgtcgagcgggacgaaggcactcgacgcgaacatcagggggaaggtgacgaggaacccgatgctggacagcacctcgatgctccgcagccacgcggcgagggccaggaacacccagatcagcgaccacgcgacgagcaacgcgagcagcgtcgcgccgacgatccccgacacgcccccggccggccggtagcccagcggccccatcgcacaggcgagcaacgccacgagctgcagggcgacccgcgccagatcggccagcgaccgggccaccaggaccagcggcagccgtaccggcatcacccgcagccggaccatcatcccggactccatgtccctgacgagacccagcccgccgccctgcgcggaaccgatcccggtcgtcagcagcagagccggcaccaggtagtcgaggtagcgcacacccggcgggaagatctccggattggccatgcggcccagcacctgactgaacagcatgagcatgatgatcggctggagcaggctgaacagcgcgatccgccggtccccgtagatgatccggaactgccggccggtcagcacccggaactgggtgccgatccccgcacccccgccgcgccacccgggcccgtccgccaccggcgaggtccccggacccgtacgtcgtgtggtcagcaccggaccgttcccccgctcgtggtctcccagctctccgccaggctcagatacacgtcgtccagcgtgggctccttgagcgccagttcggcgagctccacgcccgccgcgccgagaatccggaagatcccggccagctcggccgttgccgccagcggcagtgacaccgtccgggacaccggatccgttgtgaggccgaggccctccaggcgcagcgctccttccgcgctctccagatccgccgcggacccgaaggtcaggcggacggaccgggtgcccgccgccttgagctccgccgccgtgccgaccgtcaccacgtggccggacgacagcaccgctatccggtccgccagccggtcggcctcgtcgaggtactgggtggtcagcagaaccgtcgtgccctcggcgaccagcccttccaccgtctcccagagtgcggtgcggctcaccgggtccagcccggtggtgggctcgtcgaggaacagcaccgagggcctggccaccagactcgccgccaggtcgatgcgccgccgcattccgccggagtaggtccgcatcggtctgtccgctgcctcggtgagaccgaattgttcgaccagttcggccgcccggcgtctggcctccctcggccgggcacccagcagccgggagatcagcaccagattggcgagcccggacagatgctcgtccagggcagcgaactgccccgtcaccccgatacaggcgcgtacccgtcgggcatcgcgcacgatgtcgaagccggcgacctcggcggacccaccggacggggggagcaccgtcgagaggatctggatcagcgtggtcttcccggcaccgttgtgtcccaggagccccagcacgctccccgcgggcacctccagatccacgccgaccagcgcctgcaccgcaccgaagtgcttggtcagctgccgggtcctgatcgcgctcccgcccatgcttctccgcgtcgtcggtgccggtgggcgtgtgcactgcccgcagtcaacgcactgtagcccacggggtagttgaccgcgatcgcctgagccgacctcggacgcccgtacacggtcaggagcgggtcccaggaacaaggccctccaccccggactcggtcacccccgcaccgtgtgcccgcgggaggacatgatgtgcctgcgggaggacatgatctgcccggcactcgaccagtggatcgcccgtgccttcgcgccggaacgtctgaccgccaccctcaccgccctgacgcacgcggctgccgcatccgatggccgcgccccggaggtcgagcaggcccggcgggcggtgagaaagggcgagaagcgtctgtctcactaccgggccgccctcgcggccggggccggccccgccgttctcacccagtggatcaacgaggcccggatctgtacggcgagcccccgtgctccgtcggcggacggggtgggtgaaggggaatggttgccgggggcggtctgcggggtccgtcgcggggtggtcgtggccgggagggaggagtcgtccagcggttccctggtaggttggtgtcccttcgttctttttcaggcgctcccaccggccgcccgggtatgcgtccggtgacgccgggaagtcgttccgctgttctcttcggtgtgttctcggaagaagggcgcgggaatccccgtggcgccgtcgggagggctccacgaacgaggagagacagagcggggctgccgtgtcgtggatgaacgatgtactgacagcggtgtcggacatgaacccggtcaccagattcgcgctggcctccgtattcgcgttcgcggaatccggccttggggccggcatggccgtccccggcgaggtcgcggtactggccctgagcgccggaacggaggggacacgcccgctgttggccctcttcctcgtcgtcacactcagcagctcggccggcgaccacatcggctacttcctcggcatccgctacgggcagcggatgcgggagacgcgcctggtacggcggattgggcagcaccactgggaccgcgcgcaagagctctgccaccggtacggggcgcgcgccgtcttcctgacccgccttttgcccgtggtacgcaccctcacccccgcgaccgccggcgtcggctccgtgcgctacctccgcttcctgcccgcctccctcgccggagccgcgatgtggtccgcgctctacgtgtccgcgggaacactggtctcgacctccctgcgcgaggccgagagcgtgctctccaccatcctgtgggccctgctgggcgttgcggccgccttcacgctcgcgatcgtgtggtggcggcgcaggcaccgccgccgcagctcgtgaccgcggcgagctcctcggcggctgtgggacggcccaggaagccgtacgacggcttgcgtttcgggcggccggcggcacaggtcttgacggtggcgtcacggtccctggagtggagcgcgcacagttccatctgcgcaccccaggcctccgtggtgaaacggttctcctcggcagggtcgtcaaggttgcaggctccgacgacagcaacgcctgccggaacacgaatccggcaggcgttgcgctcctcggggcccgaggaaaagagggaacgggacaggtcagtgaccggcgaccgtgccggacagttcgttggtgcccttcggcagggtgacggatgccagcttcttccccgattccaggtcgatggagtggagttggcgcttgcccggttcggagacgtacgccgtgtggtcccggacgaacagggtgggcctgggctgctgccagtccaggggctcggtccagtcgccgaccgcgtcgatcttcttctcgacctttccggtctccgggtcgatgacgtggagggtgccgttggtgccgagcacgagggcttcgccgtgcgggccgcgggcgagggagcggaaggagtagctggtgccgaggtcgaccagcttcatcttcgccgtacgggtgtcgatcagggatatgcgggtggggcgttccagttcggcgtcggggtcggtcttgtagtcgccgaggaggatcggggaggcgtcgctgccggcctggttgccggtgcgggcgtagtcgccgggggcgtcgaccttggtgaacttgccgtccttgtagagcaggacgccgtcctcgcagccgaagccggccacctcgccctgggcggcggcctcgccgtgcactccggggcagttctcggcgcgtgcgatctccttgttgtccttgtccaggacgagggctccggtgcgcttctcctcggtgccgagggtggtgacgagttctccgccggccagttcgatggcgacaccgtggtggggcttcgcggacgtgtaggtgcggccgtccggcttcttcccgtcggacaggtcggcggggtcgaagacgttcacctctcccgtgccgtcggtgaacaggaccgtcttgccgccgtgccggacgacgtgccccggcttggaacccttgaactcggcgtcggtgaactcctgtcgggtggcgtcgaacacgcggaagccggagtccgtggagacgacgacgtgctcgttgtcgcccgccgggttgacccggttgaagccgggcagtgcgatcgtcttcgcgagcttcagggtctcgccgtcgaggacgtacagtcccccgtcgtacgaggcgacgatcgggttcccgaccgccgcggcctcgctgctcttctgctccttgggctctttgccggaaggggcgtcgtcgttgccgttgccgccgcaggcggtcagcgccatcgacgccgccacgaccagcgccgtgcccgtgaagactctggcgcgggtgggcttgttcatgatcggtgtttcctctccggccctcaggcgcgggtttgtggggtggagcggccggcgctcgaagtgccgtgccgcggggaagctcgttcagtcgccggtgaggccctcggccatggcggaggtgttggcgcgcatcatctccaggtaggttccggcgcccttgcccttctcggtcagcgactcggagtagagcgagacgacgtccacgtcgccgcccatctcctggcgcaggacctcggcgagccgggtgggctgggaggagtcggcgaagacggtgcgcaccttggccttctccatggcctgggtgagagagcgcaggtcggaggagctgggcgaggcgagcgtggttccgctggggatgaccgcgccgatgacgcggaggccgaagcggtcggcgaggtagccgaagacgtggtggttggtcaccagggcacgccggtcctcggggatggcggcgaaggacttctccatccatcccgtgaggtcggcgagttgtccgtcgtaccgctcggcgttctcccggaccttcttctcgtcgacgccctccacatgctcggcgacctggtcggcgatcaggccggcggcctcgcgtacgcggtcggggtcggtccagaagtgcggatcgggcttgccggcgtcctcttcggggccgccgtcctgtccggcatggaaggtgagcgggtcggccgcctcacccgcggcgaaggcggccactccggactcgcgggcagcctccacgtgccgcaacacgttctcctccaggccgagcccgttgtagacgaccaggtcggcgttctccaactcagcggcctgcacggccgagaggccgaaggagtgcgggtcggcgttgggcttcatcaggacactgacgccggcctcgtccccgacgatctcccgggtgatgtcgccgaggatgttggtggtcaccacgacccggggccgttcgtctccgccgcccgcgcagccggtcgccgcaccggcgacgacgaagaatccggccagggacaccagcagggcacgcatccgtggagcgcccacgcgcgcgctcatcggcccgtctccaccatcagggacggccgtacgtcgacgtcgaacgtacgggccacgcggagatcgtcgttgtagtcgatctcgtacacgcgcttgccctcggggtcgttgaggtaggcccggctgcggtccacctcgatcaccggagccgcgccgccgcccgcaccggttccgggcagttccttgagcaggggatcggtcacgccggtctccttgccggtgggtatgtcgtagccgtgcagggccccgtcggtctccaggacgaccagcggcgagccttcgccggccgtgttggcggcgaccacggggccggtcttcacccgggtccaggcgccctcgccggcatccaggacccagacagcgtccttgccggcgggtgccgtgagggtgctgctgcccgggcggtgccggaactccacggcccgctcggtcttcggcacgtcctcgccgtacggaatcttctccgccgtgaaggcgccgtcgtcctcatggacgagcagagcgccgtcggcgcagccgaggacaaccccgcgccgggtgaccgcgtcgccctgaggctcctcgcactccgcctccggagcggcgacgcgcttgccggaacggtccagcacggcgaccttcgccggagccccgctctcagcggtgagtgtcaccaggtgttccgcgtacggcacgacggcgccggcgaacgtgccgggcagcggggacggcgtgcccagggcgcctttctccaggtccgccctgcgatacacgctcgccttcccgtccgccgacgaggccacggtcacgcccgcgtcgccgcggatgctcgtaccggtgccgcccgggagttcgccgacctccttgatcttcgcgcggtaatagtggacgtggtcgccgtggtccaccatccacgcaccgctgtcgagcacccgtatgccgtccgggccgtggaagtagccgaagcggccgtccgtggtgagtgcggtggcaccggggctgcgggcgatgtcgtacaccttgccggtgatcaggtccagcacgcgggtctcaccgctcccggggtcgccgagcagaagtctggactgctgctcggccgcctcggtggcgccttcgacatagccgtgcggagtgggggacgaagaggcgggcccggaaccggacttggcctcgtccccgcccccgcatccggcggtcaggaggatcacggccgcaagcgccggagccagcgatgtgacgttttttcggacgagcaaggagatcgcctcaggtttctctcggtcaggacgtggaggaggtcgcggccggggcgaccgccggttcggcaagaccggcccggcgcgcacggcggcggtgccgaagtccggatgccaggtgggacaggaagaagagagcgacggcgagggccgagacggtcgctccggccgcggtgcgcagatgccaggacaggagcaggccgccgaaggtggcggcggcgccgaggagcgccgcgaggaccatgaccgcctggacgcttcgcgcccagggcatggccgccgcgggcggggcgatgagcagaccgaggacgagcagcgtgcccacgatgtggaaggaggccacgatggccagcgccagcaggccgagcagcacggcatgggcgagccggggacgcagcccgagtgtccgggccttgcgctcgtcgaacgcgagggccaggaaagcccggtagccgagcaccgagacggccagcgccagcagcagggctactccaagaagcagcagatcgctcccccgcacggcgaggacgtctccgaacaggaagccggtgaggtccaccgcgaaggactgcgaccgcgacacgatgatgacgccgagcgacagcatgcccacgaacagcaggccgatgccggtgtcctgggacagtcgcggagtccgcccgagggccgtgacgcccgccgccatcacggccgcgctcaccaccgcccccaccagcaggttgcctcccagcagggaggcgaccgcgacgccgggcagcagcccgtgcgacatcgcgtcaccgaggaaggccatcccgcgcagcaccacccacgttcccgcgagggcgcatatcgccgacaccaggatcccggcccatagggccctctgcacaaaggccacctcgaaaggggccgtcaaccactccatacgaggaccatataatgacaaccatgttcaataacaaccctccgttcccgccggccaccgaactgcggaatgaacgcgtccggttccagcggctgagcgccggctaccccggccgccccgtgctccaccaactcagcgccgcaataccgccgttggccatgaccgccctggtcggacccaacggaagcggcaagtcgacgctgctgggcgtcctggccggagtgatcaccgccacatccggacaactccggtacgcagagggctccccgccggctttcgtcccgcagcgcggtgccgtcggcgacaccctccccctcacggcgcggcagacggtggagatgggccgctggggacagcggggcctgtggcggcggctgacccgcacggaccggaccgcggtcgactccgccatggagaggctgggggtcgccgacctcggcgcccgccagctcggcgagctgtcgggcggccagcgtcagcgcgtgctgatcgcgcagggcctcgcccaacagtccgacctgcttctcctggacgagccgaccaccggactcgaccccgaggcgcgggagcggatcacggccctgctgacggacctggtcgccgacggcaccaccgtcgtccaggccacccacgacctggacgccgcccgctcggccgacgcctgcctgctcctggccgacggacgactgatcgggcagggatcaccggaggaagtactcaccccggaggcactggcccggatctggcagccggcgtgaagcacgaagcgctcgaagagcctgcgcaatgatcgaggcggggtgagcggatcgccggtcgccccctttaccgaccggggaatctcccccggtccgtcccgcgcggaaacagcaccgccgcagacgcctgcccctgtgtcacggcgagtgaggcgaaccaacagcgcctcagcgggcgtcgctgctgcgcaccgtgaggatacgtaccgcgcgatctcccgtgtgagcctcgcggccgtgccagcagcggtagttgtccaggacgaggatttcgcccttgtcaattacctaccggcctccaccatacctacgatattcgcgcccactctcccattagtccgcacaggtggatgcgatgcgattgcccgctaagatattctaacgcgtaacgcagttgagtatgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgacctcgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtct